SEQUENCE LISTING (1032 sequences)

SEQ ID NO: 1 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 2 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 3 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 4 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 5 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 6 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 7 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 8 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 9 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 10 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 11 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 12 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 13 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 14 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 15 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 16 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 17 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 18 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 19 - protein, 726 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSSGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSS

SEQ ID NO: 20 - protein, 726 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSSGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSS

SEQ ID NO: 21 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSS

SEQ ID NO: 22 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYYMYWVRQAPGKGLEWMGGINPSQGGTNFNEKFKNRFTISRDNSKNTLYLQMNSLRAEDTAVYYCARRDYRFDLGFDYWGQGTLVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 23 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYYMYWVRQAPGKCLEWMGGINPSQGGTNFNEKFKNRFTISRDNSKNTLYLQMNSLRAEDTAVYYCARRDYRFDLGFDYWGQGTLVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 24 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTGVRVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 25 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTGVRVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 26 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 27 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 28 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 29 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 30 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 31 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 32 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 33 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 34 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 35 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 36 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 37 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 38 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 39 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 40 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 41 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 42 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 43 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 44 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 45 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 46 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 47 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 48 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 49 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 50 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 51 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 52 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 53 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 54 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 55 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 56 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 57 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 58 - protein, 725 aa, synthetic construct; identical to SEQ ID NO: 5

SEQ ID NO: 59 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 60 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 61 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 62 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 63 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 64 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 65 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 66 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 67 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 68 - protein, 725 aa, synthetic construct; identical to SEQ ID NO: 14

SEQ ID NO: 69 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 70 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 71 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 72 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 73 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 74 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 75 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 76 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 77 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 78 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 79 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 80 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSS

SEQ ID NO: 81 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSS

SEQ ID NO: 82 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSS

SEQ ID NO: 83 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSS

SEQ ID NO: 84 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 85 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 86 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 87 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 88 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 89 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 90 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 91 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 92 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 93 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 94 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 95 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 96 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 97 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 98 - protein, 721 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 99 - protein, 721 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 100 - protein, 721 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 101 - protein, 721 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 102 - protein, 721 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 103 - protein, 721 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 104 - protein, 721 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 105 - protein, 721 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 106 - protein, 721 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 107 - protein, 721 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 108 - protein, 721 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 109 - protein, 721 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 110 - protein, 721 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 111 - protein, 721 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 112 - protein, 721 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 113 - protein, 721 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 114 - protein, 721 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 115 - protein, 721 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 116 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREWVSVITWSGGITYYADSVKGRFTISRDNSKNTLYLQMNSLRAEDTAVYYCAGDKHQSSWYDYWGQGTLVTVSS

SEQ ID NO: 117 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREWVSVITWSGGITYYADSVKGRFTISRDNSKNTLYLQMNSLRAEDTAVYYCAGDKHQSSWYDYWGQGTLVTVSS

SEQ ID NO: 118 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREWVSVITWSGGITYYADSVKGRFTISRDNSKNTVYLQMNSLRAEDTAVYYCAGDKHQSSWYDYWGQGTLVTVSS

SEQ ID NO: 119 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREWVSVITWSGGITYYADSVKGRFTISRDNSKNTVYLQMNSLRAEDTAVYYCAGDKHQSSWYDYWGQGTLVTVSS

SEQ ID NO: 120 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREFVSVITWSGGITYYADSVKGRFTISRDNSKNTLYLQMNSLRAEDTAVYYCAGDKHQSSWYDYWGQGTLVTVSS

SEQ ID NO: 121 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREFVSVITWSGGITYYADSVKGRFTISRDNSKNTLYLQMNSLRAEDTAVYYCAGDKHQSSWYDYWGQGTLVTVSS

SEQ ID NO: 122 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREFVSVITWSGGITYYADSVKGRFTISRDNSKNTVYLQMNSLRAEDTAVYYCAGDKHQSSWYDYWGQGTLVTVSS

SEQ ID NO: 123 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREFVSVITWSGGITYYADSVKGRFTISRDNSKNTVYLQMNSLRAEDTAVYYCAGDKHQSSWYDYWGQGTLVTVSS

SEQ ID NO: 124 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREFVAVITWSGGITYYADSVKGRFTISRDNSKNTVYLQMNSLRAEDTAVYYCAGDKHQSSWYDYWGQGTLVTVSS

SEQ ID NO: 125 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREFVAVITWSGGITYYADSVKGRFTISRDNSKNTVYLQMNSLRAEDTAVYYCAGDKHQSSWYDYWGQGTLVTVSS

SEQ ID NO: 126 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREWVAVITWSGGITYYADSVKGRFTISRDNSKNTVYLQMNSLRAEDTAVYYCAGDKHQSSWYDYWGQGTLVTVSS

SEQ ID NO: 127 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGSIASIHAMGWVRQAPGKEREWVAVITWSGGITYYADSVKGRFTISRDNSKNTVYLQMNSLRAEDTAVYYCAGDKHQSSWYDYWGQGTLVTVSS

SEQ ID NO: 128 - protein, 719 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 129 - protein, 719 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 130 - protein, 725 aa, synthetic construct; identical to SEQ ID NO: 1

SEQ ID NO: 131 - protein, 725 aa, synthetic construct; identical to SEQ ID NO: 2

SEQ ID NO: 132 - protein, 725 aa, synthetic construct; identical to SEQ ID NO: 4

SEQ ID NO: 133 - protein, 725 aa, synthetic construct; identical to SEQ ID NO: 5

SEQ ID NO: 134 - protein, 725 aa, synthetic construct; identical to SEQ ID NO: 7

SEQ ID NO: 135 - protein, 725 aa, synthetic construct; identical to SEQ ID NO: 8

SEQ ID NO: 136 - protein, 725 aa, synthetic construct; identical to SEQ ID NO: 10

SEQ ID NO: 137 - protein, 725 aa, synthetic construct; identical to SEQ ID NO: 11

SEQ ID NO: 138 - protein, 725 aa, synthetic construct; identical to SEQ ID NO: 13

SEQ ID NO: 139 - protein, 725 aa, synthetic construct; identical to SEQ ID NO: 14

SEQ ID NO: 140 - protein, 725 aa, synthetic construct; identical to SEQ ID NO: 16

SEQ ID NO: 141 - protein, 725 aa, synthetic construct; identical to SEQ ID NO: 17

SEQ ID NO: 142 - protein, 725 aa, synthetic construct; identical to SEQ ID NO: 22

SEQ ID NO: 143 - protein, 725 aa, synthetic construct; identical to SEQ ID NO: 23

SEQ ID NO: 144 - protein, 721 aa, synthetic construct; identical to SEQ ID NO: 102

SEQ ID NO: 145 - protein, 721 aa, synthetic construct; identical to SEQ ID NO: 105

SEQ ID NO: 146 - protein, 721 aa, synthetic construct; identical to SEQ ID NO: 111

SEQ ID NO: 147 - protein, 721 aa, synthetic construct; identical to SEQ ID NO: 114

SEQ ID NO: 148 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 214 aa, synthetic construct; identical to SEQ ID NO: 148

SEQ ID NO: 150 - protein, 214 aa, synthetic construct; identical to SEQ ID NO: 148

SEQ ID NO: 151 - protein, 214 aa, synthetic construct; identical to SEQ ID NO: 148

SEQ ID NO: 152 - protein, 214 aa, synthetic construct; identical to SEQ ID NO: 148

SEQ ID NO: 153 - protein, 214 aa, synthetic construct; identical to SEQ ID NO: 148

SEQ ID NO: 154 - protein, 214 aa, synthetic construct; identical to SEQ ID NO: 148

SEQ ID NO: 155 - protein, 214 aa, synthetic construct; identical to SEQ ID NO: 148

SEQ ID NO: 156 - protein, 214 aa, synthetic construct; identical to SEQ ID NO: 148

SEQ ID NO: 157 - protein, 214 aa, synthetic construct; identical to SEQ ID NO: 148

SEQ ID NO: 158 - protein, 214 aa, synthetic construct; identical to SEQ ID NO: 148

SEQ ID NO: 159 - protein, 214 aa, synthetic construct; identical to SEQ ID NO: 148

SEQ ID NO: 160 - protein, 214 aa, synthetic construct; identical to SEQ ID NO: 148

SEQ ID NO: 161 - protein, 214 aa, synthetic construct; identical to SEQ ID NO: 148

SEQ ID NO: 162 - protein, 214 aa, synthetic construct; identical to SEQ ID NO: 148

SEQ ID NO: 163 - protein, 214 aa, synthetic construct; identical to SEQ ID NO: 148

SEQ ID NO: 164 - protein, 214 aa, synthetic construct; identical to SEQ ID NO: 148

SEQ ID NO: 165 - protein, 214 aa, synthetic construct; identical to SEQ ID NO: 148

SEQ ID NO: 166 - protein, 214 aa, synthetic construct; identical to SEQ ID NO: 148

SEQ ID NO: 167 - protein, 214 aa, synthetic construct; identical to SEQ ID NO: 148

SEQ ID NO: 168 - protein, 214 aa, synthetic construct; identical to SEQ ID NO: 148

SEQ ID NO: 169 - protein, 214 aa, synthetic construct; identical to SEQ ID NO: 148

SEQ ID NO: 170 - protein, 214 aa, synthetic construct; identical to SEQ ID NO: 148

SEQ ID NO: 171 - protein, 214 aa, synthetic construct; identical to SEQ ID NO: 148

SEQ ID NO: 172 - protein, 214 aa, synthetic construct; identical to SEQ ID NO: 148

SEQ ID NO: 173 - protein, 214 aa, synthetic construct; identical to SEQ ID NO: 148

SEQ ID NO: 174 - protein, 214 aa, synthetic construct; identical to SEQ ID NO: 148

SEQ ID NO: 175 - protein, 214 aa, synthetic construct; identical to SEQ ID NO: 148

SEQ ID NO: 176 - protein, 214 aa, synthetic construct; identical to SEQ ID NO: 148

SEQ ID NO: 177 - protein, 214 aa, synthetic construct; identical to SEQ ID NO: 148

SEQ ID NO: 178 - protein, 214 aa, synthetic construct; identical to SEQ ID NO: 148

SEQ ID NO: 179 - protein, 214 aa, synthetic construct; identical to SEQ ID NO: 148

SEQ ID NO: 180 - protein, 214 aa, synthetic construct; identical to SEQ ID NO: 148

SEQ ID NO: 181 - protein, 214 aa, synthetic construct; identical to SEQ ID NO: 148

SEQ ID NO: 182 - protein, 214 aa, synthetic construct; identical to SEQ ID NO: 148

SEQ ID NO: 183 - protein, 214 aa, synthetic construct; identical to SEQ ID NO: 148

SEQ ID NO: 184 - protein, 214 aa, synthetic construct; identical to SEQ ID NO: 148

SEQ ID NO: 185 - protein, 214 aa, synthetic construct; identical to SEQ ID NO: 148

SEQ ID NO: 186 - protein, 214 aa, synthetic construct; identical to SEQ ID NO: 148

SEQ ID NO: 187 - protein, 214 aa, synthetic construct; identical to SEQ ID NO: 148

SEQ ID NO: 188 - protein, 214 aa, synthetic construct; identical to SEQ ID NO: 148

SEQ ID NO: 189 - protein, 214 aa, synthetic construct; identical to SEQ ID NO: 148

SEQ ID NO: 190 - protein, 214 aa, synthetic construct; identical to SEQ ID NO: 148

SEQ ID NO: 191 - protein, 214 aa, synthetic construct; identical to SEQ ID NO: 148

SEQ ID NO: 192 - protein, 214 aa, synthetic construct; identical to SEQ ID NO: 148

SEQ ID NO: 193 - protein, 214 aa, synthetic construct; identical to SEQ ID NO: 148

SEQ ID NO: 194 - protein, 214 aa, synthetic construct; identical to SEQ ID NO: 148

SEQ ID NO: 195 - protein, 214 aa, synthetic construct; identical to SEQ ID NO: 148

SEQ ID NO: 196 - protein, 214 aa, synthetic construct; identical to SEQ ID NO: 148

SEQ ID NO: 197 - protein, 214 aa, synthetic construct; identical to SEQ ID NO: 148

SEQ ID NO: 198 - protein, 214 aa, synthetic construct; identical to SEQ ID NO: 148

SEQ ID NO: 199 - protein, 214 aa, synthetic construct; identical to SEQ ID NO: 148

SEQ ID NO: 200 - protein, 214 aa, synthetic construct; identical to SEQ ID NO: 148

SEQ ID NO: 201 - protein, 214 aa, synthetic construct; identical to SEQ ID NO: 148

SEQ ID NO: 202 - protein, 214 aa, synthetic construct; identical to SEQ ID NO: 148

SEQ ID NO: 203 - protein, 214 aa, synthetic construct; identical to SEQ ID NO: 148

SEQ ID NO: 204 - protein, 214 aa, synthetic construct; identical to SEQ ID NO: 148

SEQ ID NO: 205 - protein, 214 aa, synthetic construct; identical to SEQ ID NO: 148

SEQ ID NO: 206 - protein, 214 aa, synthetic construct; identical to SEQ ID NO: 148

SEQ ID NO: 207 - protein, 214 aa, synthetic construct; identical to SEQ ID NO: 148

SEQ ID NO: 208 - protein, 214 aa, synthetic construct; identical to SEQ ID NO: 148

SEQ ID NO: 209 - protein, 214 aa, synthetic construct; identical to SEQ ID NO: 148

SEQ ID NO: 210 - protein, 214 aa, synthetic construct; identical to SEQ ID NO: 148

SEQ ID NO: 211 - protein, 214 aa, synthetic construct; identical to SEQ ID NO: 148

SEQ ID NO: 212 - protein, 214 aa, synthetic construct; identical to SEQ ID NO: 148

SEQ ID NO: 213 - protein, 214 aa, synthetic construct; identical to SEQ ID NO: 148

SEQ ID NO: 214 - protein, 214 aa, synthetic construct; identical to SEQ ID NO: 148

SEQ ID NO: 215 - protein, 214 aa, synthetic construct; identical to SEQ ID NO: 148

SEQ ID NO: 216 - protein, 214 aa, synthetic construct; identical to SEQ ID NO: 148

SEQ ID NO: 217 - protein, 214 aa, synthetic construct; identical to SEQ ID NO: 148

SEQ ID NO: 218 - protein, 214 aa, synthetic construct; identical to SEQ ID NO: 148

SEQ ID NO: 219 - protein, 214 aa, synthetic construct; identical to SEQ ID NO: 148

SEQ ID NO: 220 - protein, 214 aa, synthetic construct; identical to SEQ ID NO: 148

SEQ ID NO: 221 - protein, 214 aa, synthetic construct; identical to SEQ ID NO: 148

SEQ ID NO: 222 - protein, 214 aa, synthetic construct; identical to SEQ ID NO: 148

SEQ ID NO: 223 - protein, 214 aa, synthetic construct; identical to SEQ ID NO: 148

SEQ ID NO: 224 - protein, 214 aa, synthetic construct; identical to SEQ ID NO: 148

SEQ ID NO: 225 - protein, 214 aa, synthetic construct; identical to SEQ ID NO: 148

SEQ ID NO: 226 - protein, 214 aa, synthetic construct; identical to SEQ ID NO: 148

SEQ ID NO: 227 - protein, 214 aa, synthetic construct; identical to SEQ ID NO: 148

SEQ ID NO: 228 - protein, 214 aa, synthetic construct; identical to SEQ ID NO: 148

SEQ ID NO: 229 - protein, 214 aa, synthetic construct; identical to SEQ ID NO: 148

SEQ ID NO: 230 - protein, 214 aa, synthetic construct; identical to SEQ ID NO: 148

SEQ ID NO: 231 - protein, 214 aa, synthetic construct; identical to SEQ ID NO: 148

SEQ ID NO: 232 - protein, 214 aa, synthetic construct; identical to SEQ ID NO: 148

SEQ ID NO: 233 - protein, 214 aa, synthetic construct; identical to SEQ ID NO: 148

SEQ ID NO: 234 - protein, 214 aa, synthetic construct; identical to SEQ ID NO: 148

SEQ ID NO: 235 - protein, 214 aa, synthetic construct; identical to SEQ ID NO: 148

SEQ ID NO: 236 - protein, 214 aa, synthetic construct; identical to SEQ ID NO: 148

SEQ ID NO: 237 - protein, 214 aa, synthetic construct; identical to SEQ ID NO: 148

SEQ ID NO: 238 - protein, 214 aa, synthetic construct; identical to SEQ ID NO: 148

SEQ ID NO: 239 - protein, 214 aa, synthetic construct; identical to SEQ ID NO: 148

SEQ ID NO: 240 - protein, 214 aa, synthetic construct; identical to SEQ ID NO: 148

SEQ ID NO: 241 - protein, 214 aa, synthetic construct; identical to SEQ ID NO: 148

SEQ ID NO: 242 - protein, 214 aa, synthetic construct; identical to SEQ ID NO: 148

SEQ ID NO: 243 - protein, 214 aa, synthetic construct; identical to SEQ ID NO: 148

SEQ ID NO: 244 - protein, 214 aa, synthetic construct; identical to SEQ ID NO: 148

SEQ ID NO: 245 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 218 aa, synthetic construct; identical to SEQ ID NO: 245

SEQ ID NO: 247 - protein, 218 aa, synthetic construct; identical to SEQ ID NO: 245

SEQ ID NO: 248 - protein, 218 aa, synthetic construct; identical to SEQ ID NO: 245

SEQ ID NO: 249 - protein, 218 aa, synthetic construct; identical to SEQ ID NO: 245

SEQ ID NO: 250 - protein, 218 aa, synthetic construct; identical to SEQ ID NO: 245

SEQ ID NO: 251 - protein, 218 aa, synthetic construct; identical to SEQ ID NO: 245

SEQ ID NO: 252 - protein, 218 aa, synthetic construct; identical to SEQ ID NO: 245

SEQ ID NO: 253 - protein, 218 aa, synthetic construct; identical to SEQ ID NO: 245

SEQ ID NO: 254 - protein, 218 aa, synthetic construct; identical to SEQ ID NO: 245

SEQ ID NO: 255 - protein, 218 aa, synthetic construct; identical to SEQ ID NO: 245

SEQ ID NO: 256 - protein, 218 aa, synthetic construct; identical to SEQ ID NO: 245

SEQ ID NO: 257 - protein, 218 aa, synthetic construct; identical to SEQ ID NO: 245

SEQ ID NO: 258 - protein, 218 aa, synthetic construct; identical to SEQ ID NO: 245

SEQ ID NO: 259 - protein, 218 aa, synthetic construct; identical to SEQ ID NO: 245

SEQ ID NO: 260 - protein, 218 aa, synthetic construct; identical to SEQ ID NO: 245

SEQ ID NO: 261 - protein, 218 aa, synthetic construct; identical to SEQ ID NO: 245

SEQ ID NO: 262 - protein, 218 aa, synthetic construct; identical to SEQ ID NO: 245

SEQ ID NO: 263 - protein, 214 aa, synthetic construct; identical to SEQ ID NO: 148

SEQ ID NO: 264 - protein, 214 aa, synthetic construct; identical to SEQ ID NO: 148

SEQ ID NO: 265 - protein, 214 aa, synthetic construct; identical to SEQ ID NO: 148

SEQ ID NO: 266 - protein, 214 aa, synthetic construct; identical to SEQ ID NO: 148

SEQ ID NO: 267 - protein, 214 aa, synthetic construct; identical to SEQ ID NO: 148

SEQ ID NO: 268 - protein, 214 aa, synthetic construct; identical to SEQ ID NO: 148

SEQ ID NO: 269 - protein, 214 aa, synthetic construct; identical to SEQ ID NO: 148

SEQ ID NO: 270 - protein, 214 aa, synthetic construct; identical to SEQ ID NO: 148

SEQ ID NO: 271 - protein, 214 aa, synthetic construct; identical to SEQ ID NO: 148

SEQ ID NO: 272 - protein, 214 aa, synthetic construct; identical to SEQ ID NO: 148

SEQ ID NO: 273 - protein, 214 aa, synthetic construct; identical to SEQ ID NO: 148

SEQ ID NO: 274 - protein, 214 aa, synthetic construct; identical to SEQ ID NO: 148

SEQ ID NO: 275 - protein, 214 aa, synthetic construct; identical to SEQ ID NO: 148

SEQ ID NO: 276 - protein, 214 aa, synthetic construct; identical to SEQ ID NO: 148

SEQ ID NO: 277 - protein, 214 aa, synthetic construct; identical to SEQ ID NO: 148

SEQ ID NO: 278 - protein, 214 aa, synthetic construct; identical to SEQ ID NO: 148

SEQ ID NO: 279 - protein, 214 aa, synthetic construct; identical to SEQ ID NO: 148

SEQ ID NO: 280 - protein, 214 aa, synthetic construct; identical to SEQ ID NO: 148

SEQ ID NO: 281 - protein, 214 aa, synthetic construct; identical to SEQ ID NO: 148

SEQ ID NO: 282 - protein, 214 aa, synthetic construct; identical to SEQ ID NO: 148

SEQ ID NO: 283 - protein, 214 aa, synthetic construct; identical to SEQ ID NO: 148

SEQ ID NO: 284 - protein, 214 aa, synthetic construct; identical to SEQ ID NO: 148

SEQ ID NO: 285 - protein, 214 aa, synthetic construct; identical to SEQ ID NO: 148

SEQ ID NO: 286 - protein, 214 aa, synthetic construct; identical to SEQ ID NO: 148

SEQ ID NO: 287 - protein, 214 aa, synthetic construct; identical to SEQ ID NO: 148

SEQ ID NO: 288 - protein, 214 aa, synthetic construct; identical to SEQ ID NO: 148

SEQ ID NO: 289 - protein, 214 aa, synthetic construct; identical to SEQ ID NO: 148

SEQ ID NO: 290 - protein, 214 aa, synthetic construct; identical to SEQ ID NO: 148

SEQ ID NO: 291 - protein, 218 aa, synthetic construct; identical to SEQ ID NO: 245

SEQ ID NO: 292 - protein, 218 aa, synthetic construct; identical to SEQ ID NO: 245

SEQ ID NO: 293 - protein, 218 aa, synthetic construct; identical to SEQ ID NO: 245

SEQ ID NO: 294 - protein, 218 aa, synthetic construct; identical to SEQ ID NO: 245

SEQ ID NO: 295 - protein, 123 aa, synthetic construct [sequence omitted]

SEQ ID NO: 296 - protein, 123 aa, synthetic construct
EVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSS

SEQ ID NO: 297 - skipped

SEQ ID NO: 298 - protein, 107 aa, synthetic construct [sequence omitted]

SEQ ID NO: 299 - protein, 108 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 300 - protein, 108 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 301 - protein, 120 aa, synthetic construct [sequence omitted]

SEQ ID NO: 302 - protein, 120 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSS

SEQ ID NO: 303 - protein, 120 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSS

SEQ ID NO: 304 - protein, 120 aa, synthetic construct [sequence omitted]

SEQ ID NO: 305 - protein, 120 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSS

SEQ ID NO: 306 - protein, 120 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSS

SEQ ID NO: 307 - protein, 120 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSS

SEQ ID NO: 308 - protein, 120 aa, synthetic construct
EVQLVESGGGLVQPGGSLRLSCAASGYTFTNYYMYWVRQAPGKGLEWMGGINPSQGGTNFNEKFKNRFTISRDNSKNTLYLQMNSLRAEDTAVYYCARRDYRFDLGFDYWGQGTLVTVSS

SEQ ID NO: 309 - protein, 120 aa, synthetic construct
EVQLVESGGGLVQPGGSLRLSCAASGYTFTNYYMYWVRQAPGKCLEWMGGINPSQGGTNFNEKFKNRFTISRDNSKNTLYLQMNSLRAEDTAVYYCARRDYRFDLGFDYWGQGTLVTVSS

SEQ ID NO: 310 - protein, 120 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTGVRVSS

SEQ ID NO: 311 - protein, 120 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTGVRVSS

SEQ ID NO: 312 - protein, 120 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSS

SEQ ID NO: 313 - protein, 120 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSS

SEQ ID NO: 314 - protein, 120 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSS

SEQ ID NO: 315 - protein, 120 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSS

SEQ ID NO: 316 - protein, 120 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSS

SEQ ID NO: 317 - protein, 120 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSS

SEQ ID NO: 318 - protein, 120 aa, synthetic construct
EVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSS

SEQ ID NO: 319 - protein, 120 aa, synthetic construct
EVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSS

SEQ ID NO: 320 - protein, 120 aa, synthetic construct
EVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSS

SEQ ID NO: 321 - protein, 120 aa, synthetic construct
EVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSS

SEQ ID NO: 322 - protein, 120 aa, synthetic construct
EVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSS

SEQ ID NO: 323 - protein, 120 aa, synthetic construct
EVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDMGFDYWGQGTTVTVSS

SEQ ID NO: 324 - protein, 120 aa, synthetic construct
EVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDLGFDYWGQGTTVTVSS

SEQ ID NO: 325 - protein, 120 aa, synthetic construct
EVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDMGFDYWGQGTTVTVSS

SEQ ID NO: 326 - protein, 120 aa, synthetic construct
EVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDMGFDYWGQGTTVTVSS

SEQ ID NO: 327 - protein, 120 aa, synthetic construct
EVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDLGFDYWGQGTTVTVSS

SEQ ID NO: 328 - protein, 119 aa, synthetic construct
EVQLVESGGGLVQPGGSLRLSCAASGSIASIHAMGWVRQAPGKEREWVSVITWSGGITYYADSVKGRFTISRDNSKNTLYLQMNSLRAEDTAVYYCAGDKHQSSWYDYWGQGTLVTVSS

SEQ ID NO: 329 - protein, 119 aa, synthetic construct
EVQLVESGGGLVQPGGSLRLSCAASGSIASIHAMGWVRQAPGKEREWVSVITWSGGITYYADSVKGRFTISRDNSKNTVYLQMNSLRAEDTAVYYCAGDKHQSSWYDYWGQGTLVTVSS

SEQ ID NO: 330 - protein, 119 aa, synthetic construct
EVQLVESGGGLVQPGGSLRLSCAASGSIASIHAMGWVRQAPGKEREFVSVITWSGGITYYADSVKGRFTISRDNSKNTLYLQMNSLRAEDTAVYYCAGDKHQSSWYDYWGQGTLVTVSS

SEQ ID NO: 331 - protein, 119 aa, synthetic construct
EVQLVESGGGLVQPGGSLRLSCAASGSIASIHAMGWVRQAPGKEREFVSVITWSGGITYYADSVKGRFTISRDNSKNTVYLQMNSLRAEDTAVYYCAGDKHQSSWYDYWGQGTLVTVSS

SEQ ID NO: 332 - protein, 119 aa, synthetic construct
EVQLVESGGGLVQPGGSLRLSCAASGSIASIHAMGWVRQAPGKEREFVAVITWSGGITYYADSVKGRFTISRDNSKNTVYLQMNSLRAEDTAVYYCAGDKHQSSWYDYWGQGTLVTVSS

SEQ ID NO: 333 - protein, 119 aa, synthetic construct
EVQLVESGGGLVQPGGSLRLSCAASGSIASIHAMGWVRQAPGKEREWVAVITWSGGITYYADSVKGRFTISRDNSKNTVYLQMNSLRAEDTAVYYCAGDKHQSSWYDYWGQGTLVTVSS

SEQ ID NO: 334 - protein, 118 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 335 - protein, 111 aa, synthetic construct [sequence omitted]

SEQ ID NO: 336 - protein, 112 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 337 - protein, 112 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 338 - protein, 113 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSS

SEQ ID NO: 339 - protein, 111 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIK

SEQ ID NO: 340 - protein, 112 aa, synthetic construct
EIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 341 - protein, 112 aa, synthetic construct
EIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 342 - protein, 112 aa, synthetic construct
EIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 343 - protein, 112 aa, synthetic construct
EIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 344 - protein, 112 aa, synthetic construct
EIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 345 - protein, 112 aa, synthetic construct
EIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 346 - protein, 112 aa, synthetic construct
DIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 347 - protein, 112 aa, synthetic construct
DIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 348 - protein, 111 aa, synthetic construct
EIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 349 - protein, 108 aa, synthetic construct
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 350 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 353 - protein, 107 aa, synthetic construct [sequence omitted]

SEQ ID NO: 354 - protein, 252 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 355 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 356 - protein, 252 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 357 - protein, 252 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 358 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 359 - protein, 252 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 360 - protein, 253 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSSGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSS

SEQ ID NO: 361 - protein, 251 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSS

SEQ ID NO: 362 - protein, 252 aa, synthetic construct
EVQLVESGGGLVQPGGSLRLSCAASGYTFTNYYMYWVRQAPGKGLEWMGGINPSQGGTNFNEKFKNRFTISRDNSKNTLYLQMNSLRAEDTAVYYCARRDYRFDLGFDYWGQGTLVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 363 - protein, 252 aa, synthetic construct
EVQLVESGGGLVQPGGSLRLSCAASGYTFTNYYMYWVRQAPGKCLEWMGGINPSQGGTNFNEKFKNRFTISRDNSKNTLYLQMNSLRAEDTAVYYCARRDYRFDLGFDYWGQGTLVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 364 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTGVRVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 365 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTGVRVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 366 - protein, 252 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 367 - protein, 252 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 368 - protein, 252 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 369 - protein, 252 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 370 - protein, 252 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 371 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 372 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 373 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 374 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 375 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 376 - protein, 252 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 377 - protein, 252 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 378 - protein, 252 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 379 - protein, 252 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 380 - protein, 252 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGGGTKVEIKR

SEQ ID NO: 381 - protein, 252 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 382 - protein, 252 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 383 - protein, 252 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLRFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 384 - protein, 252 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 385 - protein, 252 aa, synthetic construct
QVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 386 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 387 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 388 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 389 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 390 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 391 - protein, 252 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 392 - protein, 252 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 393 - protein, 252 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLRFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 394 - protein, 252 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 395 - protein, 252 aa, synthetic construct
QVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPSTLSLSPGERATLSCRASKGVSTSGYSYLHWYQQKPGQAPRLLIYLASYLESGVPARFSGSGSGTDFTLTISGLEPEDFAVYYCQHSRDLPLTFGCGTKVEIKR

SEQ ID NO: 396 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 397 - protein, 252 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 398 - protein, 252 aa, synthetic construct
EVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 399 - protein, 252 aa, synthetic construct
EVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 400 - protein, 252 aa, synthetic construct
EVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 401 - protein, 252 aa, synthetic construct
EVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 402 - protein, 252 aa, synthetic construct
EVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGGGTKVEIKR

SEQ ID NO: 403 - protein, 252 aa, synthetic construct
EVQLLESGGGLVQPGGSLRLSCKASGYTFTNYYMYWVRQAPGKGLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 404 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 405 - protein, 252 aa, synthetic construct
QVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 406 - protein, 252 aa, synthetic construct
EVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 407 - protein, 252 aa, synthetic construct
EVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 408 - protein, 252 aa, synthetic construct
EVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 409 - protein, 252 aa, synthetic construct
EVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 410 - protein, 252 aa, synthetic construct
EVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSEIVLTQSPATLSVSPGERATLSCRASKGVSTSGYSYLHWYQQKPGQAPRLLIYLASYLESGIPARFSGSGSGTEFTLTISSLQSEDFAVYYCQHSRDLPLTFGCGTKVEIKR

SEQ ID NO: 411 - protein, 252 aa, synthetic construct
EVQLLESGGGLVQPGGSLRLSCKASGYTFTNYYMYWVRQAPGKCLEWMGGINPSQGGTNFNEKFKNRVTLSTDSSKNTAYLQMNSLRAEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 412 - protein, 251 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 413 - protein, 251 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSS

SEQ ID NO: 414 - protein, 251 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSS

SEQ ID NO: 415 - protein, 251 aa, synthetic construct
EIVLTQSPSTLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 416 - protein, 252 aa, synthetic construct
EVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKR

SEQ ID NO: 417 - protein, 252 aa, synthetic construct
EVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 418 - protein, 252 aa, synthetic construct
EVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 419 - protein, 252 aa, synthetic construct
EVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 420 - protein, 252 aa, synthetic construct
EVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 421 - protein, 252 aa, synthetic construct
EVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIKR

SEQ ID NO: 422 - protein, 251 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 423 - protein, 251 aa, synthetic construct
EVQLVESGGGLVQPGGSLRLSCAASGYTFTNYGMNWVRQAPGKCLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGCGTKVEIKR

SEQ ID NO: 424 - protein, 119 aa, synthetic construct; identical to SEQ ID NO: 328

SEQ ID NO: 425 - protein, 119 aa, synthetic construct; identical to SEQ ID NO: 329

SEQ ID NO: 426 - protein, 119 aa, synthetic construct; identical to SEQ ID NO: 330

SEQ ID NO: 427 - protein, 119 aa, synthetic construct; identical to SEQ ID NO: 331

SEQ ID NO: 428 - protein, 119 aa, synthetic construct; identical to SEQ ID NO: 332

SEQ ID NO: 429 - protein, 119 aa, synthetic construct; identical to SEQ ID NO: 333

SEQ ID NO: 430 - protein, 246 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 431 - protein, 123 aa, synthetic construct [sequence omitted]

SEQ ID NO: 432 - protein, 107 aa, synthetic construct [sequence omitted]

SEQ ID NO: 433 - protein, 5 aa, synthetic construct
NYGMN

SEQ ID NO: 434 - protein, 17 aa, synthetic construct
WINTYTGEPTYAADFKR

SEQ ID NO: 435 - protein, 14 aa, synthetic construct
YPHYYGSSHWYFDV

SEQ ID NO: 436 - protein, 11 aa, synthetic construct
SASQDISNYLN

SEQ ID NO: 437 - protein, 7 aa, synthetic construct
FTSSLHS

SEQ ID NO: 438 - protein, 9 aa, synthetic construct
QQYSTVPWT

SEQ ID NO: 439 - protein, 8 aa, synthetic construct
GYTFTNYG

SEQ ID NO: 440 - protein, 8 aa, synthetic construct
INTYTGEP

SEQ ID NO: 441 - protein, 16 aa, synthetic construct
AKYPHYYGSSHWYFDV

SEQ ID NO: 442 - protein, 6 aa, synthetic construct
QDISNY

SEQ ID NO: 443 - protein, 7 aa, synthetic construct
GYTFTNY

SEQ ID NO: 444 - protein, 6 aa, synthetic construct
NTYTGE

SEQ ID NO: 445 - protein, 5 aa, synthetic construct
NYYMY

SEQ ID NO: 446 - protein, 17 aa, synthetic construct
GINPSNGGTNFNEKFKN

SEQ ID NO: 447 - protein, 11 aa, synthetic construct
RDYRFDMGFDY

SEQ ID NO: 448 - protein, 15 aa, synthetic construct
RASKGVSTSGYSYLH

SEQ ID NO: 449 - protein, 7 aa, synthetic construct
LASYLES

SEQ ID NO: 450 - protein, 9 aa, synthetic construct
QHSRDLPLT

SEQ ID NO: 451 - protein, 8 aa, synthetic construct
GYTFTNYY

SEQ ID NO: 452 - protein, 8 aa, synthetic construct
INPSNGGT

SEQ ID NO: 453 - protein, 13 aa, synthetic construct
ARRDYRFDMGFDY

SEQ ID NO: 454 - protein, 10 aa, synthetic construct
KGVSTSGYSY

SEQ ID NO: 455 - protein, 6 aa, synthetic construct
NPSNGG

SEQ ID NO: 456 - protein, 17 aa, synthetic construct
GINPSQGGTNFNEKFKN

SEQ ID NO: 457 - protein, 11 aa, synthetic construct
RDYRFDLGFDY

SEQ ID NO: 458 - protein, 8 aa, synthetic construct
INPSQGGT

SEQ ID NO: 459 - protein, 13 aa, synthetic construct
ARRDYRFDLGFDY

SEQ ID NO: 460 - protein, 6 aa, synthetic construct
NPSQGG

SEQ ID NO: 461 - protein, 17 aa, synthetic construct
GINPSRGGTNFNEKFKN

SEQ ID NO: 462 - protein, 8 aa, synthetic construct
INPSRGGT

SEQ ID NO: 463 - protein, 6 aa, synthetic construct
NPSRGG

SEQ ID NO: 464 - protein, 120 aa, synthetic construct [sequence omitted]

SEQ ID NO: 465 - protein, 120 aa, synthetic construct; identical to SEQ ID NO: 312

SEQ ID NO: 466 - protein, 120 aa, synthetic construct; identical to SEQ ID NO: 310

SEQ ID NO: 467 - protein, 120 aa, synthetic construct; identical to SEQ ID NO: 318

SEQ ID NO: 468 - protein, 120 aa, synthetic construct; identical to SEQ ID NO: 319

SEQ ID NO: 469 - protein, 120 aa, synthetic construct; identical to SEQ ID NO: 306

SEQ ID NO: 470 - protein, 120 aa, synthetic construct; identical to SEQ ID NO: 308

SEQ ID NO: 471 - protein, 112 aa, synthetic construct; identical to SEQ ID NO: 336

SEQ ID NO: 472 - protein, 112 aa, synthetic construct; identical to SEQ ID NO: 342

SEQ ID NO: 473 - protein, 112 aa, synthetic construct; identical to SEQ ID NO: 343

SEQ ID NO: 474 - protein, 112 aa, synthetic construct; identical to SEQ ID NO: 340

SEQ ID NO: 475 - protein, 112 aa, synthetic construct; identical to SEQ ID NO: 346

SEQ ID NO: 476 - protein, 112 aa, synthetic construct; identical to SEQ ID NO: 337

SEQ ID NO: 477-655 - skipped (179 entries)

SEQ ID NO: 656 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 657 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 658 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 659 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 660 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSQGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 661 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSRGGTNFNEKFKNRVTLTTDSSTSTAYMELSSLRSEDTAVYYCARRDYRFDMGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 662 - protein, 723 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTNYYMYWVRQAPGQCLEWMGGINPSNGGTNFNEKFKNRVTLTTDSSTSTAYMELSSLRSEDTAVYYCARRDYRFDLGFDYWGQGTTVTVSSGGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 663 - protein, 591 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGSIASIHAMGWVRQAPGKEREFVAVITWSGGITYYADSVKGRFTISRDNSKNTVYLQMNSLRPEDTAVYYCAGDKHQSSWYDYWGQGTLVTVSS

SEQ ID NO: 664 - protein, 591 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGSIASIHAMGWVRQAPGKEREFVAVITWSGGITYYADSVKGRFTISRDNSKNTVYLQMNSLRPEDTALYYCAGDKHQSSWYDYWGQGTLVTVSS

SEQ ID NO: 665 - protein, 591 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGSIASIHAMGWFRQAPGKEREFVAVITWSGGITYYADSVKGRFTISRDNSKNTVYLQMNSLRPEDTAVYYCAGDKHQSSWYDYWGQGTLVTVSS

SEQ ID NO: 666 - protein, 591 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 667 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 668 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 669 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 670 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 671 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 672 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 673 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 674 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 675 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 676 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 677 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVQSGAEVKKPGASVKVSCKASGYTFTNYYMYWVRQAPGQGLEWMGGINPSRGGTNFNEKFKNRVTLTTDSSTSTAYMELSSLRSEDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 678 - protein, 976 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSEVQLVQSGAEVKKPGASVKVSCKASGYTFTNYYMYWVRQAPGQGLEWMGGINPSRGGTNFNEKFKNRVTLTTDSSTSTAYMELSSLRSEDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 679 - protein, 214 aa, synthetic construct; identical to SEQ ID NO: 148

SEQ ID NO: 680 - protein, 214 aa, synthetic construct; identical to SEQ ID NO: 148

SEQ ID NO: 681 - protein, 214 aa, synthetic construct; identical to SEQ ID NO: 148

SEQ ID NO: 682 - protein, 214 aa, synthetic construct; identical to SEQ ID NO: 148

SEQ ID NO: 683 - protein, 214 aa, synthetic construct; identical to SEQ ID NO: 148

SEQ ID NO: 684 - protein, 214 aa, synthetic construct; identical to SEQ ID NO: 148

SEQ ID NO: 685 - protein, 214 aa, synthetic construct; identical to SEQ ID NO: 148

SEQ ID NO: 686 - protein, 214 aa, synthetic construct; identical to SEQ ID NO: 148

SEQ ID NO: 687 - protein, 214 aa, synthetic construct; identical to SEQ ID NO: 148

SEQ ID NO: 688 - protein, 214 aa, synthetic construct; identical to SEQ ID NO: 148

SEQ ID NO: 689 - protein, 214 aa, synthetic construct; identical to SEQ ID NO: 148

SEQ ID NO: 690 - protein, 214 aa, synthetic construct; identical to SEQ ID NO: 148

SEQ ID NO: 691 - protein, 214 aa, synthetic construct; identical to SEQ ID NO: 148

SEQ ID NO: 692 - protein, 214 aa, synthetic construct; identical to SEQ ID NO: 148

SEQ ID NO: 693 - protein, 214 aa, synthetic construct; identical to SEQ ID NO: 148

SEQ ID NO: 694 - protein, 214 aa, synthetic construct; identical to SEQ ID NO: 148

SEQ ID NO: 695 - protein, 214 aa, synthetic construct; identical to SEQ ID NO: 148

SEQ ID NO: 696 - protein, 214 aa, synthetic construct; identical to SEQ ID NO: 148

SEQ ID NO: 697 - protein, 214 aa, synthetic construct; identical to SEQ ID NO: 148

SEQ ID NO: 698 - protein, 214 aa, synthetic construct; identical to SEQ ID NO: 148

SEQ ID NO: 699 - protein, 214 aa, synthetic construct; identical to SEQ ID NO: 148

SEQ ID NO: 700 - protein, 214 aa, synthetic construct; identical to SEQ ID NO: 148

SEQ ID NO: 701 - protein, 214 aa, synthetic construct; identical to SEQ ID NO: 148

SEQ ID NO: 702 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 703 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 704 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 705 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 706 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 707 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 708 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 709 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEVQLVQSGAEVKKPGASVKVSCKASGYTFTNYYMYWVRQAPGQGLEWMGGINPSNGGTNFNEKFKNRVTLTTDSSTSTAYMELSSLRSEDTAVYYCARRDYRFDMGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 710 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 711 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEVQLVQSGAEVKKPGASVKVSCKASGYTFTNYYMYWVRQAPGQGLEWMGGINPSQGGTNFNEKFKNRVTLTTDSSTSTAYMELSSLRSEDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 712 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEVQLVQSGAEVKKPGASVKVSCKASGYTFTNYYMYWVRQAPGQGLEWMGGINPSRGGTNFNEKFKNRVTLTTDSSTSTAYMELSSLRSEDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 713 - protein, 697 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSEVQLVQSGAEVKKPGASVKVSCKASGYTFTNYYMYWVRQAPGQGLEWMGGINPSRGGTNFNEKFKNRVTLTTDSSTSTAYMELSSLRSEDTAVYYCARRDYRFDLGFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 714 - protein, 214 aa, synthetic construct; identical to SEQ ID NO: 148

SEQ ID NO: 715 - protein, 214 aa, synthetic construct; identical to SEQ ID NO: 148

SEQ ID NO: 716 - protein, 214 aa, synthetic construct; identical to SEQ ID NO: 148

SEQ ID NO: 717 - protein, 214 aa, synthetic construct; identical to SEQ ID NO: 148

SEQ ID NO: 718 - protein, 214 aa, synthetic construct; identical to SEQ ID NO: 148

SEQ ID NO: 719 - protein, 214 aa, synthetic construct; identical to SEQ ID NO: 148

SEQ ID NO: 720 - protein, 214 aa, synthetic construct; identical to SEQ ID NO: 148

SEQ ID NO: 721 - protein, 214 aa, synthetic construct; identical to SEQ ID NO: 148

SEQ ID NO: 722 - protein, 214 aa, synthetic construct; identical to SEQ ID NO: 148

SEQ ID NO: 723 - protein, 214 aa, synthetic construct; identical to SEQ ID NO: 148

SEQ ID NO: 724 - protein, 214 aa, synthetic construct; identical to SEQ ID NO: 148

SEQ ID NO: 725 - protein, 214 aa, synthetic construct; identical to SEQ ID NO: 148

SEQ ID NO: 726 - protein, 216 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 727 - protein, 216 aa, synthetic construct; identical to SEQ ID NO: 726

SEQ ID NO: 728 - protein, 216 aa, synthetic construct; identical to SEQ ID NO: 726

SEQ ID NO: 729 - protein, 216 aa, synthetic construct; identical to SEQ ID NO: 726

SEQ ID NO: 730 - protein, 216 aa, synthetic construct; identical to SEQ ID NO: 726

SEQ ID NO: 731 - protein, 216 aa, synthetic construct; identical to SEQ ID NO: 726

SEQ ID NO: 732 - protein, 216 aa, synthetic construct
DIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 733 - protein, 216 aa, synthetic construct; identical to SEQ ID NO: 732

SEQ ID NO: 734 - protein, 216 aa, synthetic construct; identical to SEQ ID NO: 732

SEQ ID NO: 735 - protein, 216 aa, synthetic construct; identical to SEQ ID NO: 732

SEQ ID NO: 736 - protein, 216 aa, synthetic construct; identical to SEQ ID NO: 732

SEQ ID NO: 737 - protein, 216 aa, synthetic construct; identical to SEQ ID NO: 732

SEQ ID NO: 738 - protein, 119 aa, synthetic construct
EVQLVESGGGLVQPGGSLRLSCAASGSIASIHAMGWVRQAPGKEREFVAVITWSGGITYYADSVKGRFTISRDNSKNTVYLQMNSLRPEDTAVYYCAGDKHQSSWYDYWGQGTLVTVSS

SEQ ID NO: 739 - protein, 119 aa, synthetic construct
EVQLVESGGGLVQPGGSLRLSCAASGSIASIHAMGWVRQAPGKEREFVAVITWSGGITYYADSVKGRFTISRDNSKNTVYLQMNSLRPEDTALYYCAGDKHQSSWYDYWGQGTLVTVSS

SEQ ID NO: 740 - protein, 119 aa, synthetic construct
EVQLVESGGGLVQPGGSLRLSCAASGSIASIHAMGWFRQAPGKEREFVAVITWSGGITYYADSVKGRFTISRDNSKNTVYLQMNSLRPEDTAVYYCAGDKHQSSWYDYWGQGTLVTVSS

SEQ ID NO: 741 - protein, 119 aa, synthetic construct
EVQLVESGGGLVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 742 - protein, 120 aa, synthetic construct
EVQLVQSGAEVKKPGASVKVSCKASGYTFTNYYMYWVRQAPGQGLEWMGGINPSRGGTNFNEKFKNRVTLTTDSSTSTAYMELSSLRSEDTAVYYCARRDYRFDLGFDYWGQGTTVTVSS

SEQ ID NO: 743 - protein, 111 aa, synthetic construct
DIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGCGTKVEIK

SEQ ID NO: 744 - protein, 111 aa, synthetic construct
DIQLTQSPSSLSASVGDRVTITCRASKGVSTSGYSYLHWYQQKPGKAPKLLIYLASYLESGVPSRFSGSGSGTDFTLTISSLQPEDFATYYCQHSRDLPLTFGGGTKVEIK

SEQ ID NO: 745 - protein, 448 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 746 - protein, 214 aa, synthetic construct
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 747 - protein, 498 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 748 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 749 - protein, 218 aa, synthetic construct; identical to SEQ ID NO: 245

SEQ ID NO: 750 - protein, 504 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 751 - protein, 504 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 752 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 753 - protein, 214 aa, synthetic construct; identical to SEQ ID NO: 148

SEQ ID NO: 754 - protein, 714 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTGVRVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 755 - protein, 214 aa, synthetic construct; identical to SEQ ID NO: 148

SEQ ID NO: 756 - protein, 714 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTGVRVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 757 - protein, 214 aa, synthetic construct; identical to SEQ ID NO: 148

SEQ ID NO: 758 - protein, 717 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSGGGGSGGGGSGGGGSGGGGSDIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFR

SEQ ID NO: 759 - protein, 214 aa, synthetic construct; identical to SEQ ID NO: 148

SEQ ID NO: 760 - protein, 717 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSGGGGSGGGGSGGGGSGGGGSDIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFR

SEQ ID NO: 761 - protein, 214 aa, synthetic construct; identical to SEQ ID NO: 148

SEQ ID NO: 762 - protein, 717 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLLESGGVLVQPGGSLRLSCAASGFTFSNFGMTWVRQAPGKCLEWVSGISGGGRDTYFADSVKGRFTISRDNSKNTLYLQMNSLKGEDTAVYYCVKWGNIYFDYWGQGTLVTVSSGGGGSGGGGSGGGGSGGGGSDIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGCGTVVDFR

SEQ ID NO: 763 - protein, 214 aa, synthetic construct; identical to SEQ ID NO: 148

SEQ ID NO: 764 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 765 - protein, 214 aa, synthetic construct; identical to SEQ ID NO: 148

SEQ ID NO: 766 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 767 - protein, 214 aa, synthetic construct; identical to SEQ ID NO: 148

SEQ ID NO: 768 - protein, 578 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGEVQLQESGGGLVQPGGSLRLSCAASGFTFSSYWMYWLRQAPGKGLEWVSSINSDSSSTYYRDSVKGRFTISRDNAKNTLYLQMNSLKSEDTAVYYCAKDPGGYAKGQGTQVTVSS

SEQ ID NO: 769 - protein, 214 aa, synthetic construct; identical to SEQ ID NO: 148

SEQ ID NO: 770 - protein, 562 aa, synthetic construct
SDTGRPFVEMYSEIPEIIHMTEGRELVIPCRVTSPNITVTLKKFPLDTLIPDGKRIIWDSRKGFIISAATYKEIGLLTCEATVNGHLYKTNYLTHRQTNTGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 771 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 772 - protein, 595 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAVSGNIYNRNFMGWFRQAPGKGREGVSAIYTGTSRTYYADSVKGRFTISRDNAKNTVYLQMNSLRPEDTAVYYCAADLRDGFWDTGVWNTWGQGTLVTVSS

SEQ ID NO: 773 - protein, 214 aa, synthetic construct; identical to SEQ ID NO: 148

SEQ ID NO: 774 - protein, 450 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLRSDGSFFLYSKLTVDKSRWQQGNVFSCSVMHEALHNHYTQKSLSLSPGK

SEQ ID NO: 775 - protein, 218 aa, synthetic construct; identical to SEQ ID NO: 245

SEQ ID NO: 776 - protein, 450 aa, synthetic construct
EVQLVESGGGLVQPGGSLRLSCAASGFTISDYWIHWVRQAPGKGLEWVAGITPAGGYTYYADSVKGRFTISADTSKNTAYLQMNSLRAEDTAVYYCARFVFFLPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTEPPSRDELTKNQVSLTCLVKGFYPSDIAVEWESNGQPENNYKTTPPVLDSDGSFFLLSVLTVDKSRWQQGNVFSCSVMHEALHNHYTQKSLSLSPGK

SEQ ID NO: 777 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQGYGNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 778 - protein, 713 aa, synthetic construct
QVQLVESGGGLVQPGGSLRLSCAASGFAFSSYDMSWVRQAPGKCLEWVATISGGGRYTYYPDSVKGRFTISRDNSKNNLYLQMNSLRAEDTAVYYCAVRYGETWFAYWGQGTLVTVSSGGGGSGGGGSGGGGSGGGGSDIQMTQSPSSLSASVGDRVTITCRASQDINTYLAWFQQKPGKAPKSLIYRANRLVSGVPSRFSGSGSGTDFTLTISSLQPEDMATYYCLQYDEFPLTFGCGTKLELKGGGASGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 779 - protein, 214 aa, synthetic construct; identical to SEQ ID NO: 148

SEQ ID NO: 780 - protein, 681 aa, synthetic construct
EVQLVESGGGLVQPGGSLRLSCAASGFVFSNYDMSWVRQAPGKRLEWVATISGGGGYTYYSDSVKGRFTISRDNAKNSLYLQMNSLRAEDTAVYYCASPYGHYGFEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 781 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 782 - protein, 586 aa, synthetic construct
QVQLVESGGGLVQPGGSLRLSCATSGSRRSIYAMGWFRQAPGKGLEFVAGIGWAYATTYYADSVKGRFTISRDNTKNTLYLQMNSLRAEDTAVYYCAADLDHSGFDYWGQGTLVTVSSGGGGSGGGGSGGGGSEVQLVQSGGGVVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 783 - protein, 214 aa, synthetic construct; identical to SEQ ID NO: 148

SEQ ID NO: 784 - protein, 450 aa, synthetic construct
QVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 785 - protein, 218 aa, synthetic construct; identical to SEQ ID NO: 245

SEQ ID NO: 786 - protein, 450 aa, synthetic construct
QVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 787 - protein, 218 aa, synthetic construct; identical to SEQ ID NO: 245

SEQ ID NO: 788 - protein, 504 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 789 - protein, 504 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 790 - protein, 504 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSQGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 791 - protein, 504 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQCLEWMGGINPSRGGTNFNEKFKNRVTLTTDSSTTTAYMELKSLQFDDTAVYYCARRDYRFDLGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGCGTKVEIKR

SEQ ID NO: 792 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 793 - protein, 325 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 794 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 795 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 796 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 797 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 798 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 799 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 800 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 801 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 802 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 803 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 804 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 805 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 806 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 807 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 808 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 809 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 810 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 811 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 812 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 813 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 814 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 815 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 816 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 817 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 818 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 819 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 820 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 821 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 822 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 823 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 824 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 826 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 827 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 828 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 829 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 830 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 831 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 832 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 833 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 834 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPG

SEQ ID NO: 835 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPG

SEQ ID NO: 836 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 837 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 838 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 839 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 840 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 841 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 842 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 843 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 844 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 845 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 846 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 847 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 848 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 849 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 850 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 851 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 852 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 853 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 854 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 855 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 856 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 857 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 858 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 859 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 860 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 861 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 862 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 863 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 864 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 865 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 866 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 867 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 868 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 869 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 870 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 871 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 872 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 873 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 874 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 875 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 876 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 877 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 878 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 879 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 880 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 881 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 882 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 883 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 884 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 885 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 886 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 887 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 888 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 889 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 890 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 891 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 892 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 893 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 894 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 895 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 896 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPG

SEQ ID NO: 897 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 898 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 899 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 900 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 901 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 902 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 903 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 904 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 905 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 906 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 907 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 908 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 909 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 910 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 911 - protein, 330 aa, Homo sapiens [sequence omitted]

SEQ ID NO: 912 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 913 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 914 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 915 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 916 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 917 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 918 - protein, 330 aa, synthetic construct; identical to SEQ ID NO: 350

SEQ ID NO: 919 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 920 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 921 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 922 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 923 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 924 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 925 - protein, 330 aa, synthetic construct; identical to SEQ ID NO: 351

SEQ ID NO: 926 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 927 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 928 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 929 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 930 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 931 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 932 - protein, 330 aa, synthetic construct; identical to SEQ ID NO: 352

SEQ ID NO: 933 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 934 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 935 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 936 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 937 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 938 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 939 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 940 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 941 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 942 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 943 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 944 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 945 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 946 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 947 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 948 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 949 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 950 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 951 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 952 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 953 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 954 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 955 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 956 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 957 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 958 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 959 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 960 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 961 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 962 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 963 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 964 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 965 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 966 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 967 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 968 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 969 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 970 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 971 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 972 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 973 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 974 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 975 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 976 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 977 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 978 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 979 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 980 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 981 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 982 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 983 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 984 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 985 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 986 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 987 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 988 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 989 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 990 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 991 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 992 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 993 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 994 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 995 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 996 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 997 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 998 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 999 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1000 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1001 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1002 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1003 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1004 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1005 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1006 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1007 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1008 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1009 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1010 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1011 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1012 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 1013 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 1014 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHRDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 1015 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHRDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 1016 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1017 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1018 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1019 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1020 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1021 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 1022 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1023 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 1024 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1025 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 1026 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 1027 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 1028 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1029 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1030 - protein, 20 aa, synthetic construct
GGGGSGGGGSGGGGSGGGGS

SEQ ID NO: 1031 - protein, 5 aa, Homo sapiens
SRDEL

SEQ ID NO: 1032 - protein, 5 aa, Homo sapiens
SREEM

(19) United States
(12) Patent Application Publication
(10) Pub. No.: US 2026/0109769 A1
(43) Pub. Date: Apr. 23, 2026

(54) PD-1 BNDING PROTEINS, VEGF AND PD-1 BISPECIFIC BINDING PROTEINS, COMPOSITIONS, AND METHODS OF USE

(71) Applicant: Paragon Therapeutics, Inc., Waltham, MA (US)

(72) Inventors: Byong Ha Kang, Waltham, MA (US); Hussam Hisham Shaheen, Waltham, MA (US); Gopalan Raghunathan, Waltham, MA (US); Ghassan Najib Fayad, Waltham, MA (US); Mohammad Murshid Alam, Waltham, MA (US)

(73) Assignee: Paragon Therapeutics, Inc., Waltham, MA (US)

(21) Appl. No.: 19/420,662

(22) Filed: Dec. 15, 2025

Related U.S. Application Data
(63) Continuation of application No. PCT/US2025/047219, filed on Sep. 19, 2025.
(60) Provisional application No. 63/697,436, filed on Sep. 20, 2024.
(60) Provisional application No. 63/719,870, filed on Nov. 13, 2024.
(60) Provisional application No. 63/788,196, filed on Apr. 14, 2025.
(60) Provisional application No. 63/831,020, filed on Jun. 26, 2025.

(51) Int. Cl. C07K 16/28; A61K 39/00; C07K 16/22

(52) U.S. Cl. CPC C07K 16/2818; C07K 16/22; A61K 2039/505; C07K 2317/31; C07K 2317/73; C07K 2317/77; C07K 2317/92; C07K 2317/94

(57) ABSTRACT

Bispecific antibodies that bind VEGF and PD-1, pharmaceutical compositions thereof, and methods of use thereof, including methods of inhibiting VEGF and PD-1 biological activities.

### RELATED APPLICATIONS

This application is a continuation of international application number PCT/US2025/047219, filed Sep. 19, 2025, which claims benefit of U.S. provisional application No. 63/697,436, filed Sep. 20, 2024, 63/719,870, filed Nov. 13, 2024, 63/788,196, filed Apr. 14, 2025, and 63/831,020, filed Jun. 26, 2025. The entire content of PCT/US2025/047219 is incorporated herein by reference.

### REFERENCE TO AN ELECTRONIC SEQUENCE LISTING

The contents of the electronic sequence listing (C181870000US00-SEQ-LJG.xml; Size: 1,070,409 bytes; and Date of Creation: Dec. 19, 2025) are herein incorporated by reference in their entirety.

### BACKGROUND

Vascular endothelial cell growth factor (VEGF) is a potent mitogen for vascular endothelial cells, has been reported as a pivotal regulator of both normal and abnormal angiogenesis. VEGF is a master regulator of angiogenesis during growth and development, as well as in disease states such as cancer, diabetes, and macular degeneration. It is also required for the cyclical blood vessel proliferation in the female reproductive tract and for bone growth and cartilage formation. Cancer is among the most common indications for current VEGF therapies, with FDA and EMA-approved therapies targeting various kinds of cancer. Some examples of current VEGF therapies to address these diseases and conditions include axitinib, bevacizumab, brolucizumab, cabozantinib, lapatinib, lenvatinib, pazopanib, ponatinib, ramucirumab, ranibizumab, regorafenib, sorafenib, sunitinib, and vandetanib.

Programmed death receptor 1 (PD-1) is an immunoinhibitory receptor primarily expressed on activated lymphocytes (e.g., peripheral CD4+ and CD8+ T cells, B cells and monocytes). Interaction with its ligands has been shown to attenuate T-cell responses both in vitro and in vivo.

The role of PD-1 in cancer is well established in the literature. The ligands for PD-1 (Programmed death-ligand 1 (PD-L1) and Programmed death-ligand 2 (PD-L2)) are constitutively expressed or can be induced in a variety of cell types, including non-hematopoietic tissues as well as various tumor types. Blockade of the interaction between PD-1 and PD-L1, for example, has been shown to enhance tumor-specific CD8+ T-cell immunity and may therefore be helpful in clearance of tumor cells by the immune system. Overall, the PD-1/PD-L1 pathway is a well-validated target for the development of antibody therapeutics for cancer treatment.

### SUMMARY

The instant disclosure relates, in part, to PD-1 binding proteins and bispecific binding proteins that bind (1) VEGF and (2) PD-1.

In one aspect, provided are programmed death receptor 1 (PD-1) binding proteins comprising an anti-PD1 immunoglobulin heavy chain variable domain (anti-PD-1 VH) comprising complementary determining regions: CDR-H1 comprising the amino acid sequence of SEQ ID NO: 445; CDR-H2 comprising the amino acid sequence of SEQ ID NO: 456, 461, or 446; and CDR-H3 comprising the amino acid sequence of SEQ ID NO: 457 or 447; CDR-H1 comprising the amino acid sequence of SEQ ID NO: 451; CDR-H2 comprising the amino acid sequence of SEQ ID NO: 458, 462, or 452; and CDR-H3 comprising the amino acid sequence of SEQ ID NO: 459 or 453; or CDR-H1 comprising the amino acid sequence of SEQ ID NO: 443; CDR-H2 comprising the amino acid sequence of SEQ ID NO: 460, 463, or 455; and CDR-H3 comprising the amino acid sequence of SEQ ID NO: 457 or 447, provided that: if CDR-H2 comprises the amino acid sequence of SEQ ID NO: 446, then CDR-H3 does not comprise the amino acid sequence of 447; if CDR-H2 comprises the amino acid sequence of SEQ ID NO: 452, then CDR-H3 does not comprise the amino acid sequence of 453; and if CDR-H2 comprises the amino acid sequence of SEQ ID NO: 455, then CDR-H3 does not comprise the amino acid sequence of 447.

In some embodiments, the CDR-H1, CDR-H2, and CDR-H3 comprise the amino acid sequences of SEQ ID NOs 445, 456, and 457, respectively; SEQ ID NOs 451, 458, and 459, respectively; or SEQ ID NOs 443, 460, and 457, respectively. In some embodiments, the PD-1 binding protein further comprises an anti-PD1 immunoglobulin light chain variable domain (anti-PD-1 VL) comprising complementarity-determining regions: CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450. In some embodiments, the anti-PD-1 VH and anti-PD-1 VL have amino acid sequences that are at least 85% identical to that of: SEQ ID NOs: 302 and 335, respectively; SEQ ID NOs: 302 and 336, respectively; SEQ ID NOs: 302 and 340, respectively; SEQ ID NOs: 302 and 342, respectively; SEQ ID NOs: 302 and 343, respectively; SEQ ID NOs: 302 and 346, respectively; SEQ ID NOs: 306 and 337, respectively; SEQ ID NOs: 306 and 339, respectively; SEQ ID NOs: 306 and 341, respectively; SEQ ID NOs: 306 and 344, respectively; SEQ ID NOs: 306 and 345, respectively; SEQ ID NOs: 306 and 347, respectively; SEQ ID NOs: 308 and 340, respectively; SEQ ID NOs: 309 and 341, respectively; SEQ ID NOs: 310 and 336, respectively; SEQ ID NOs: 311 and 337, respectively; SEQ ID NOs: 313 and 336, respectively; SEQ ID NOs: 313 and 342, respectively; SEQ ID NOs: 313 and 343, respectively; SEQ ID NOs: 316 and 337, respectively; SEQ ID NOs: 316 and 344, respectively; SEQ ID NOs: 316 and 345, respectively; SEQ ID NOs: 318 and 336, respectively; SEQ ID NOs: 318 and 340, respectively; SEQ ID NOs: 318 and 346, respectively; SEQ ID NOs: 319 and 336, respectively; SEQ ID NOs: 319 and 340, respectively; SEQ ID NOs: 319 and 346, respectively; SEQ ID NOs: 320 and 337, respectively; SEQ ID NOs: 320 and 341, respectively; SEQ ID NOs: 320 and 347, respectively; SEQ ID NOs: 320 and 743, respectively; SEQ ID NOs: 321 and 337, respectively; SEQ ID NOs: 321 and 341, respectively; or SEQ ID NOs: 321 and 347, respectively. In some embodiments, the anti-PD-1 VH and anti-PD-1 VL have amino acid sequences of: SEQ ID NOs: 302 and 335, respectively; SEQ ID NOs: 302 and 336, respectively; SEQ ID NOs: 302 and 340, respectively; SEQ ID NOs: 302 and 342, respectively; SEQ ID NOs: 302 and 343, respectively; SEQ ID NOs: 302 and 346, respectively; SEQ ID NOs: 306 and 337, respectively; SEQ ID NOs: 306 and 339, respectively; SEQ ID NOs: 306 and 341, respectively; SEQ ID NOs: 306 and 344, respectively; SEQ ID NOs: 306 and 345, respectively; SEQ ID NOs: 306 and 347, respectively; SEQ ID NOs: 308 and 340, respectively; SEQ ID NOs: 309 and 341, respectively; SEQ ID NOs: 310 and 336, respectively; SEQ ID NOs: 311 and 337, respectively; SEQ ID NOs: 313 and 336, respectively; SEQ ID NOs: 313 and 342, respectively; SEQ ID NOs: 313 and 343, respectively; SEQ ID NOs: 316 and 337, respectively; SEQ ID NOs: 316 and 344, respectively; SEQ ID NOs: 316 and 345, respectively; SEQ ID NOs: 318 and 336, respectively; SEQ ID NOs: 318 and 340, respectively; SEQ ID NOs: 318 and 346, respectively; SEQ ID NOs: 319 and 336, respectively; SEQ ID NOs: 319 and 340, respectively; SEQ ID NOs: 319 and 346, respectively; SEQ ID NOs: 320 and 337, respectively; SEQ ID NOs: 320 and 341, respectively; SEQ ID NOs: 320 and 347, respectively; SEQ ID NOs: 320 and 743, respectively; SEQ ID NOs: 321 and 337, respectively; SEQ ID NOs: 321 and 341, respectively; or SEQ ID NOs: 321 and 347, respectively.

In some embodiments, the CDR-H1, CDR-H2, and CDR-H3 comprise the amino acid sequences of SEQ ID NOs 445, 456, and 447, respectively; SEQ ID NOs 451, 458, and 453, respectively; or SEQ ID NOs 443, 460, and 447, respectively. In some embodiments, the PD-1 binding protein further comprises an anti-PD1 immunoglobulin light chain variable domain (anti-PD-1 VL) comprising complementarity-determining regions: CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450. In some embodiments, the anti-PD-1 VH and anti-PD-1 VL have amino acid sequences that are at least 85% identical to that of: SEQ ID NOs: 303 and 336, respectively; SEQ ID NOs: 325 and 347, respectively; or SEQ ID NOs: 325 and 743, respectively. In some embodiments, the anti-PD-1 VH and anti-PD-1 VL have amino acid sequences of: SEQ ID NOs: 303 and 336, respectively; SEQ ID NOs: 325 and 347, respectively; or SEQ ID NOs: 325 and 743, respectively.

In some embodiments, the CDR-H1, CDR-H2, and CDR-H3 comprise the amino acid sequences of SEQ ID NOs 445, 461, and 447, respectively; SEQ ID NOs 451, 462, and 453, respectively; or SEQ ID NOs 443, 463, and 447, respectively. In some embodiments, the PD-1 binding protein further comprises an anti-PD1 immunoglobulin light chain variable domain (anti-PD-1 VL) comprising complementarity-determining regions: CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450. In some embodiments, the anti-PD-1 VH and anti-PD-1 VL have amino acid sequences that are at least 85% identical to that of: SEQ ID NOs: 326 and 347, respectively; or SEQ ID NOs: 326 and 743, respectively. In some embodiments, the anti-PD-1 VH and anti-PD-1 VL have amino acid sequences of: SEQ ID NOs: 326 and 347, respectively; or SEQ ID NOs: 326 and 743, respectively.

In some embodiments, the CDR-H1, CDR-H2, and CDR-H3 comprise the amino acid sequences of SEQ ID NOs 445, 446, and 457, respectively; SEQ ID NOs 451, 452, and 459, respectively; or SEQ ID NOs 443, 455, and 457, respectively. In some embodiments, the PD-1 binding protein further comprises an anti-PD1 immunoglobulin light chain variable domain (anti-PD-1 VL) comprising complementarity-determining regions: CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450. In some embodiments, the anti-PD-1 VH and anti-PD-1 VL have amino acid sequences that are at least 85% identical to that of: SEQ ID NOs: 327 and 347, respectively; or SEQ ID NOs: 327 and 743, respectively. In some embodiments, the anti-PD-1 VH and anti-PD-1 VL have amino acid sequences of: SEQ ID NOs: 327 and 347, respectively; or SEQ ID NOs: 327 and 743, respectively.

In some embodiments, the CDR-H1, CDR-H2, and CDR-H3 comprise the amino acid sequences of SEQ ID NOs 445, 461, and 457, respectively; SEQ ID NOs 451, 462, and 459, respectively; or SEQ ID NOs 443, 463, and 457, respectively. In some embodiments, the PD-1 binding protein further comprises an anti-PD1 immunoglobulin light chain variable domain (anti-PD-1 VL) comprising complementarity-determining regions: CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450. In some embodiments, the anti-PD-1 VH and anti-PD-1 VL have amino acid sequences that are at least 85% identical to that of: SEQ ID NOs: 303 and 335, respectively; SEQ ID NOs: 303 and 342, respectively; SEQ ID NOs: 303 and 343, respectively; SEQ ID NOs: 307 and 337, respectively; SEQ ID NOs: 307 and 344, respectively; SEQ ID NOs: 307 and 345, respectively; SEQ ID NOs: 314 and 336, respectively; SEQ ID NOs: 314 and 342, respectively; SEQ ID NOs: 314 and 343, respectively; SEQ ID NOs: 317 and 337, respectively; SEQ ID NOs: 317 and 344, respectively; SEQ ID NOs: 317 and 345, respectively; or SEQ ID NOs: 324 and 347, respectively. In some embodiments, the anti-PD-1 VH and anti-PD-1 VL have amino acid sequences of: SEQ ID NOs: 303 and 335, respectively; SEQ ID NOs: 303 and 342, respectively; SEQ ID NOs: 303 and 343, respectively; SEQ ID NOs: 307 and 337, respectively; SEQ ID NOs: 307 and 344, respectively; SEQ ID NOs: 307 and 345, respectively; SEQ ID NOs: 314 and 336, respectively; SEQ ID NOs: 314 and 342, respectively; SEQ ID NOs: 314 and 343, respectively; SEQ ID NOs: 317 and 337, respectively; SEQ ID NOs: 317 and 344, respectively; SEQ ID NOs: 317 and 345, respectively; or SEQ ID NOs: 324 and 347, respectively.

In some embodiments, the anti-PD-1 VH has a framework having an amino acid sequence at least 85% identical to that within any one of SEQ ID NO: 464-470 and the anti-PD-1 VL has a framework having an amino acid sequence at least 85% identical to that within any one of SEQ ID NO: 471-476.

In some embodiments, the anti-PD-1 VH has a framework having an amino acid sequence of that within any one of SEQ ID NO: 464-470 and the anti-PD-1 VL has a framework having an amino acid sequence of that within any one of SEQ ID NO: 471-476.

In some embodiments, the anti-PD-1 VH has a framework having an amino acid sequence of that within SEQ ID NO: 464 and the anti-PD-1 VL has a framework having an amino acid sequence of that within SEQ ID NO: 471.

In some embodiments, the anti-PD-1 VH has a framework having an amino acid sequence of that within SEQ ID NO: 465 and the anti-PD-1 VL has a framework having an amino acid sequence of that within SEQ ID NO: 472.

In some embodiments, the anti-PD-1 VH has a framework having an amino acid sequence of that within SEQ ID NO: 465 and the anti-PD-1 VL has a framework having an amino acid sequence of that within SEQ ID NO: 471.

In some embodiments, the anti-PD-1 VH has a framework having an amino acid sequence of that within SEQ ID NO: 465 and the anti-PD-1 VL has a framework having an amino acid sequence of that within SEQ ID NO: 473.

In some embodiments, the anti-PD-1 VH has a framework having an amino acid sequence of that within SEQ ID NO: 464 and the anti-PD-1 VL has a framework having an amino acid sequence of that within SEQ ID NO: 472.

In some embodiments, the anti-PD-1 VH has a framework having an amino acid sequence of that within SEQ ID NO: 464 and the anti-PD-1 VL has a framework having an amino acid sequence of that within SEQ ID NO: 473.

In some embodiments, the anti-PD-1 VH has a framework having an amino acid sequence of that within SEQ ID NO: 464 and the anti-PD-1 VL has a framework having an amino acid sequence of that within SEQ ID NO: 474.

In some embodiments, the anti-PD-1 VH has a framework having an amino acid sequence of that within SEQ ID NO: 464 and the anti-PD-1 VL has a framework having an amino acid sequence of that within SEQ ID NO: 475.

In some embodiments, the anti-PD-1 VH has a framework having an amino acid sequence of that within SEQ ID NO: 467 and the anti-PD-1 VL has a framework having an amino acid sequence of that within SEQ ID NO: 471.

In some embodiments, the anti-PD-1 VH has a framework having an amino acid sequence of that within SEQ ID NO: 467 and the anti-PD-1 VL has a framework having an amino acid sequence of that within SEQ ID NO: 474.

In some embodiments, the anti-PD-1 VH has a framework having an amino acid sequence of that within SEQ ID NO: 467 and the anti-PD-1 VL has a framework having an amino acid sequence of that within SEQ ID NO: 475.

In some embodiments, the anti-PD-1 VH has a framework having an amino acid sequence of that within SEQ ID NO: 468 and the anti-PD-1 VL has a framework having an amino acid sequence of that within SEQ ID NO: 471.

In some embodiments, the anti-PD-1 VH has a framework having an amino acid sequence of that within SEQ ID NO: 468 and the anti-PD-1 VL has a framework having an amino acid sequence of that within SEQ ID NO: 474.

In some embodiments, the anti-PD-1 VH has a framework having an amino acid sequence of that within SEQ ID NO: 468 and the anti-PD-1 VL has a framework having an amino acid sequence of that within SEQ ID NO: 475.

In some embodiments, the anti-PD-1 VH has a framework having an amino acid sequence of that within SEQ ID NO: 466 and the anti-PD-1 VL has a framework having an amino acid sequence of that within SEQ ID NO: 471.

In some embodiments, the anti-PD-1 VH has a framework having an amino acid sequence of that within SEQ ID NO: 470 and the anti-PD-1 VL has a framework having an amino acid sequence of that within SEQ ID NO: 474.

In some embodiments, the anti-PD-1 VH has a framework having an amino acid sequence of that within SEQ ID NO: 469 and the anti-PD-1 VL has a framework having an amino acid sequence of that within SEQ ID NO: 476.

In some embodiments, the anti-PD-1 VH has a framework having an amino acid sequence of that within SEQ ID NO: 465 except for one amino acid substitution; and the anti-PD-1 VL has a framework having an amino acid sequence of that within SEQ ID NO: 472 except for one amino acid substitution; the anti-PD-1 VH has a framework having an amino acid sequence of that within SEQ ID NO: 465 except for one amino acid substitution; and the anti-PD-1 VL has a framework having an amino acid sequence of that within SEQ ID NO: 471 except for one amino acid substitution; the anti-PD-1 VH has a framework having an amino acid sequence of that within SEQ ID NO: 465 except for one amino acid substitution; and the anti-PD-1 VL has a framework having an amino acid sequence of that within SEQ ID NO: 473 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 472 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 473 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 474 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 467 except for one amino acid substitution; and the anti-PD-1 VL has a framework having an amino acid sequence of that within SEQ ID NO: 471 except for one amino acid substitution; the anti-PD-1 VH has a framework having an amino acid sequence of that within SEQ ID NO: 467 except for one amino acid substitution; and the anti-PD-1 VL has a framework having an amino acid sequence of that within SEQ ID NO: 474 except for one amino acid substitution; the anti-PD-1 VH has a framework having an amino acid sequence of that within SEQ ID NO: 467 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 471 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 474 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 466 except for one amino acid substitution; and the anti-PD-1 VL has a framework having an amino acid sequence of that within SEQ ID NO: 471 except for one amino acid substitution; or the anti-PD-1 VH has a framework having an amino acid sequence of that within SEQ ID NO: 470 except for one amino acid substitution; and the anti-PD-1 VL has a framework having an amino acid sequence of that within SEQ ID NO: 474 except for one amino acid substitution, wherein the one amino acid substitution in the VH is from a non-cysteine residue to a cysteine residue and the one amino acid substitution in the VL is from a non-cysteine residue to a cysteine residue.

In some embodiments, the one amino acid substitution in the anti-PD-1 VH is at residue H44 and the one amino acid substitution in the anti-PD-1 VL is at residue L100, wherein numbering is according to Kabat.

In one aspect provided are PD-1 binding proteins comprising (a) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 456, and 447, respectively; (2) SEQ ID NOs 451, 458, and 453, respectively; or (3) SEQ ID NOs 443, 460, and 457, respectively, and (b) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively. In some embodiments, (a) the anti-PD-1 VH has an amino acid sequence at least 85% identical to that of SEQ ID NO: 325; and (b) the anti-PD-1 VL has an amino acid sequence at least 85% identical to that of SEQ ID NO: 347. In some embodiments, (a) the anti-PD-1 VH has an amino acid sequence of SEQ ID NO: 325; and (b) the anti-PD-1 VL has an amino acid sequence of SEQ ID NO: 347.

In one aspect, provided are a PD-1 binding proteins comprising (a) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 456, and 457, respectively; (2) SEQ ID NOs 451, 458, and 459, respectively; or (3) SEQ ID NOs 443, 460, and 457, respectively, and (b) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively. In some embodiments, (a) the anti-PD-1 VH has an amino acid sequence at least 85% identical to that of SEQ ID NO: 320; and (b) the anti-PD-1 VL has an amino acid sequence at least 85% identical to that of SEQ ID NO: 347. In some embodiments, (a) the anti-PD-1 VH has an amino acid sequence of SEQ ID NO: 320; and (b) the anti-PD-1 VL has an amino acid sequence of SEQ ID NO: 347.

In one aspect, provided are PD-1 binding proteins comprising (a) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 461, and 457, respectively; (2) SEQ ID NOs 451, 462, and 459, respectively; or (3) SEQ ID NOs 443, 463, and 457, respectively, and (b) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively. In some embodiments, (a) the anti-PD-1 VH has an amino acid sequence at least 85% identical to that of SEQ ID NO: 324; and (b) the anti-PD-1 VL has an amino acid sequence at least 85% identical to that of SEQ ID NO: 347. In some embodiments, (a) the anti-PD-1 VH has an amino acid sequence of SEQ ID NO: 324; and (b) the anti-PD-1 VL has an amino acid sequence of SEQ ID NO: 347.

In one aspect, provided are PD-1 binding proteins comprising (a) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 461, and 447, respectively; (2) SEQ ID NOs 451, 462, and 453, respectively; or (3) SEQ ID NOs 443, 463, and 447, respectively, and (b) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively. In some embodiments, (a) the anti-PD-1 VH has an amino acid sequence at least 85% identical to that of SEQ ID NO: 326; and (b) the anti-PD-1 VL has an amino acid sequence at least 85% identical to that of SEQ ID NO: 347. In some embodiments, (a) the anti-PD-1 VH has an amino acid sequence of SEQ ID NO: 326; and (b) the anti-PD-1 VL has an amino acid sequence of SEQ ID NO: 347.

In one aspect, provided are PD-1 binding proteins comprising an anti-PD-1 immunoglobulin heavy chain variable domain (anti-PD-1 VH) comprising complementary determining regions CDR-H1, CDR-H2, and CDR-H3 which comprise the amino acid sequences of SEQ ID NOs 445, 446, and 457, respectively; SEQ ID NOs 451, 452, and 459, respectively; or SEQ ID NOs 443, 455, and 457, respectively, and an anti-PD1 immunoglobulin light chain variable domain (anti-PD-1 VL) comprising complementary determining regions CDR-L1, CDR-L2, and CDR-L3 which comprise the amino acid sequences of SEQ ID NOs 448, 449, and 450, respectively; SEQ ID NO: 454, LAS, and SEQ ID NO: 450, respectively; or SEQ ID NOs 448, 449, and 450 respectively.

In some embodiments, the anti-PD-1 VH has an amino acid sequence at least 85% identical to that of SEQ ID NO: 327; and the anti-PD-1 VL has an amino acid sequence at least 85% identical to that of SEQ ID NO: 347.

In some embodiments, the anti-PD-1 VH has an amino acid sequence of SEQ ID NO: 327; and the anti-PD-1 VL has an amino acid sequence of SEQ ID NO: 347.

In some embodiments, the anti-PD-1 VH has a framework having an amino acid sequence of that within SEQ ID NO: 467 except for up to one amino acid substitution; and the anti-PD-1 VL has a framework having an amino acid sequence of that within SEQ ID NO: 475 except for up to one amino acid substitution.

In some embodiments, the one amino acid substitutions in the anti-PD-1 VH and the anti-PD-1 VL are collectively H44-L100 cysteine mutations.

In one aspect, provided are programmed death receptor 1 (PD-1) binding proteins comprising an anti-PD1 immunoglobulin heavy chain variable domain (anti-PD-1 VH) and an anti-PD1 immunoglobulin light chain variable domain (anti-PD-1 VL), wherein: the anti-PD-1 VH comprises complementary determining regions CDR-H1 comprising the amino acid sequence of SEQ ID NO: 445; CDR-H2 comprising the amino acid sequence of SEQ ID NO: 446; and CDR-H3 comprising the amino acid sequence of SEQ ID NO: 447; CDR-H1 comprising the amino acid sequence of SEQ ID NO: 451; CDR-H2 comprising the amino acid sequence of SEQ ID NO: 452; and CDR-H3 comprising the amino acid sequence of SEQ ID NO: 453; or CDR-H1 comprising the amino acid sequence of SEQ ID NO: 443; CDR-H2 comprising the amino acid sequence of SEQ ID NO: 455; and CDR-H3 comprising the amino acid sequence of SEQ ID NO: 447; the anti-PD-1 VL comprises complementary determining regions CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450, and wherein the anti-PD-1 VH has a framework having an amino acid sequence of that within SEQ ID NO: 465; and the anti-PD-1 VL has a framework having an amino acid sequence of that within SEQ ID NO: 472, the anti-PD-1 VH has a framework having an amino acid sequence of that within SEQ ID NO: 465; and the anti-PD-1 VL has a framework having an amino acid sequence of that within SEQ ID NO: 471, the anti-PD-1 VH has a framework having an amino acid sequence of that within SEQ ID NO: 465; and the anti-PD-1 VL has a framework having an amino acid sequence of that within SEQ ID NO: 473, the anti-PD-1 VH has a framework having an amino acid sequence of that within SEQ ID NO: 464; and the anti-PD-1 VL has a framework having an amino acid sequence of that within SEQ ID NO: 472, the anti-PD-1 VH has a framework having an amino acid sequence of that within SEQ ID NO: 464; and the anti-PD-1 VL has a framework having an amino acid sequence of that within SEQ ID NO: 473, the anti-PD-1 VH has a framework having an amino acid sequence of that within SEQ ID NO: 464; and the anti-PD-1 VL has a framework having an amino acid sequence of that within SEQ ID NO: 474, the anti-PD-1 VH has a framework having an amino acid sequence of that within SEQ ID NO: 464; and the anti-PD-1 VL has a framework having an amino acid sequence of that within SEQ ID NO: 475, the anti-PD-1 VH has a framework having an amino acid sequence of that within SEQ ID NO: 467; and the anti-PD-1 VL has a framework having an amino acid sequence of that within SEQ ID NO: 471, the anti-PD-1 VH has a framework having an amino acid sequence of that within SEQ ID NO: 467; and the anti-PD-1 VL has a framework having an amino acid sequence of that within SEQ ID NO: 474, the anti-PD-1 VH has a framework having an amino acid sequence of that within SEQ ID NO: 467; and the anti-PD-1 VL has a framework having an amino acid sequence of that within SEQ ID NO: 475, the anti-PD-1 VH has a framework having an amino acid sequence of that within SEQ ID NO: 468; and the anti-PD-1 VL has a framework having an amino acid sequence of that within SEQ ID NO: 471, the anti-PD-1 VH has a framework having an amino acid sequence of that within SEQ ID NO: 468; and the anti-PD-1 VL has a framework having an amino acid sequence of that within SEQ ID NO: 474, the anti-PD-1 VH has a framework having an amino acid sequence of that within SEQ ID NO: 468; and the anti-PD-1 VL has a framework having an amino acid sequence of that within SEQ ID NO: 475, the anti-PD-1 VH has a framework having an amino acid sequence of that within SEQ ID NO: 468; and the anti-PD-1 VL has a framework having an amino acid sequence of that within SEQ ID NO: 475, the anti-PD-1 VH has a framework having an amino acid sequence of that within SEQ ID NO: 466; and the anti-PD-1 VL has a framework having an amino acid sequence of that within SEQ ID NO: 471, or the anti-PD-1 VH has a framework having an amino acid sequence of that within SEQ ID NO: 470; and the anti-PD-1 VL has a framework having an amino acid sequence of that within SEQ ID NO: 474.

In one aspect, provided are programmed death receptor 1 (PD-1) binding proteins comprising an anti-PD1 immunoglobulin heavy chain variable domain (anti-PD-1 VH) and an anti-PD1 immunoglobulin light chain variable domain (anti-PD-1 VL), wherein the anti-PD-1 VH comprises complementary determining regions CDR-H1 comprising the amino acid sequence of SEQ ID NO: 445; CDR-H2 comprising the amino acid sequence of SEQ ID NO: 446; and CDR-H3 comprising the amino acid sequence of SEQ ID NO: 447; CDR-H1 comprising the amino acid sequence of SEQ ID NO: 451; CDR-H2 comprising the amino acid sequence of SEQ ID NO: 452; and CDR-H3 comprising the amino acid sequence of SEQ ID NO: 453; or CDR-H1 comprising the amino acid sequence of SEQ ID NO: 443; CDR-H2 comprising the amino acid sequence of SEQ ID NO: 455; and CDR-H3 comprising the amino acid sequence of SEQ ID NO: 447, the anti-PD-1 VL comprises complementary determining regions CDR-LT comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-LT comprising the amino acid sequence of SEQ ID NO: 454; CDR-L2 comprising the amino acid sequence of LAS; and CDR-L3 comprising the amino acid sequence of SEQ ID NO: 450; or CDR-LT comprising the amino acid sequence of SEQ ID NO: 448; CDR-L2 comprising the amino acid sequence of SEQ ID NO: 449; and CDR-L3 comprising the amino acid sequence of SEQ ID NO: 450, and wherein the anti-PD-1 VH has a framework having an amino acid sequence of that within SEQ ID NO: 465 except for one amino acid substitution; and the anti-PD-1 VL has a framework having an amino acid sequence of that within SEQ ID NO: 472 except for one amino acid substitution; the anti-PD-1 VH has a framework having an amino acid sequence of that within SEQ ID NO: 465 except for one amino acid substitution; and the anti-PD-1 VL has a framework having an amino acid sequence of that within SEQ ID NO: 471 except for one amino acid substitution; the anti-PD-1 VH has a framework having an amino acid sequence of that within SEQ ID NO: 465 except for one amino acid substitution; and the anti-PD-1 VL has a framework having an amino acid sequence of that within SEQ ID NO: 473 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 472 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 473 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 474 except for one amino acid substitution; the anti-PD-1 VH has a framework having an amino acid sequence of that within SEQ ID NO: 464 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 467 except for one amino acid substitution; and the anti-PD-1 VL has a framework having an amino acid sequence of that within SEQ ID NO: 471 except for one amino acid substitution; the anti-PD-1 VH has a framework having an amino acid sequence of that within SEQ ID NO: 467 except for one amino acid substitution; and the anti-PD-1 VL has a framework having an amino acid sequence of that within SEQ ID NO: 474 except for one amino acid substitution; the anti-PD-1 VH has a framework having an amino acid sequence of that within SEQ ID NO: 467 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 471 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 474 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 468 except for one amino acid substitution; and the anti-PD-1 VL has a framework having an amino acid sequence of that within SEQ ID NO: 475 except for one amino acid substitution; the anti-PD-1 VH has a framework having an amino acid sequence of that within SEQ ID NO: 466 except for one amino acid substitution; and the anti-PD-1 VL has a framework having an amino acid sequence of that within SEQ ID NO: 471 except for one amino acid substitution; or the anti-PD-1 VH has a framework having an amino acid sequence of that within SEQ ID NO: 470 except for one amino acid substitution; and the anti-PD-1 VL has a framework having an amino acid sequence of that within SEQ ID NO: 474 except for one amino acid substitution, wherein the one amino acid substitution in the VH is from a non-cysteine residue to a cysteine residue and the one amino acid substitution in the VL is from a non-cysteine residue to a cysteine residue.

In some embodiments, the one amino acid substitution in the anti-PD-1 VH is at residue H44 and the one amino acid substitution in the anti-PD-1 VL is at residue L100, wherein numbering is according to Kabat.

In one aspect, provided are programmed death receptor 1 (PD-1) binding proteins comprising an anti-PD1 immunoglobulin heavy chain variable domain (anti-PD-1 VH) and an anti-PD1 immunoglobulin light chain variable domain (anti-PD-1 VL), wherein: the anti-PD-1 VH comprises complementary determining regions CDR-H1 comprising the amino acid sequence of SEQ ID NO: 445; CDR-H2 comprising the amino acid sequence of SEQ ID NO: 446; and CDR-H3 comprising the amino acid sequence of SEQ ID NO: 447; CDR-H1 comprising the amino acid sequence of SEQ ID NO: 451; CDR-H2 comprising the amino acid sequence of SEQ ID NO: 452; and CDR-H3 comprising the amino acid sequence of SEQ ID NO: 453; or CDR-H1 comprising the amino acid sequence of SEQ ID NO: 443; CDR-H2 comprising the amino acid sequence of SEQ ID NO: 455; and CDR-H3 comprising the amino acid sequence of SEQ ID NO: 447; the anti-PD-1 VL comprises complementary determining regions CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450; CDR-L1 comprising the amino acid sequence of SEQ ID NO: 454; CDR-L2 comprising the amino acid sequence of LAS; and CDR-L3 comprising the amino acid sequence of SEQ ID NO: 450; or CDR-L1 comprising the amino acid sequence of SEQ ID NO: 448; CDR-L2 comprising the amino acid sequence of SEQ ID NO: 449; and CDR-L3 comprising the amino acid sequence of SEQ ID NO: 450, and wherein the anti-PD-1 VH and anti-PD-1 VL have amino acid sequences that are at least 85% identical to that of: SEQ ID NOs: 301 and 335, respectively; SEQ ID NOs: 301 and 336, respectively; SEQ ID NOs: 301 and 342, respectively; SEQ ID NOs: 301 and 343, respectively; SEQ ID NOs: 301 and 348, respectively; SEQ ID NOs: 305 and 337, respectively; SEQ ID NOs: 305 and 344, respectively; SEQ ID NOs: 305 and 345, respectively; SEQ ID NOs: 312 and 335, respectively; SEQ ID NOs: 312 and 336, respectively; SEQ ID NOs: 312 and 342, respectively; SEQ ID NOs: 312 and 343, respectively; SEQ ID NOs: 315 and 337, respectively; SEQ ID NOs: 315 and 344, respectively; SEQ ID NOs: 315 and 345, respectively; SEQ ID NOs: 322 and 346, respectively; SEQ ID NOs: 323 and 347, respectively; or SEQ ID NOs: 323 and 743, respectively.

In some embodiments, the anti-PD-1 VH and anti-PD-1 VL have amino acid sequences of: SEQ ID NOs: 301 and 335, respectively; SEQ ID NOs: 301 and 336, respectively; SEQ ID NOs: 301 and 342, respectively; SEQ ID NOs: 301 and 343, respectively; SEQ ID NOs: 301 and 348, respectively; SEQ ID NOs: 305 and 337, respectively; SEQ ID NOs: 305 and 344, respectively; SEQ ID NOs: 305 and 345, respectively; SEQ ID NOs: 312 and 335, respectively; SEQ ID NOs: 312 and 336, respectively; SEQ ID NOs: 312 and 342, respectively; SEQ ID NOs: 312 and 343, respectively; SEQ ID NOs: 315 and 337, respectively; SEQ ID NOs: 315 and 344, respectively; SEQ ID NOs: 315 and 345, respectively; SEQ ID NOs: 322 and 346, respectively; SEQ ID NOs: 323 and 347, respectively; or SEQ ID NOs: 323 and 743, respectively.

In some embodiments, the PD-1 binding protein further comprises an Fc region.

In some embodiments, the Fc region comprises a means for extending the half-life of the PD-1 binding protein.

In some embodiments, the half-life extending means comprises an Fc modification.

In some embodiments, the Fc modification is selected from the group consisting of M252Y/S254T/T256E (YTE), M428L/N434S (LS), M428L/N434A (LA), H433K/N434F (KF), and L309D/Q311H/N434S (DHS).

In some embodiments, the Fc modification is M252Y/S254T/T256E (YTE) or M428L/N434S (LS).

In some embodiments, the Fc modification is M428L/N434S (LS).

In some embodiments, the Fc region is an IgG1, IgG2, or IgG4 Fc region.

In some embodiments, the PD-1 binding protein is an antibody or antigen-binding fragment thereof.

In some embodiments, the PD-1 binding protein is a human or humanized antibody or antigen-binding fragment thereof.

In some embodiments, the antigen-binding fragment is a Fab, a F(ab′)2, a Fab′, a single-chain Fv (scFv), an Fv fragment, a Fd fragment, or a diabody.

In one aspect, provided are bispecific proteins comprising a PD-1 binding protein of any one of claims 1-48, further comprising a VEGF-binding region comprising an anti-VEGF immunoglobulin heavy chain variable domain (anti-VEGF VH) comprising complementary determining regions CDR-H1 comprising the amino acid sequence of SEQ ID NO: 433; CDR-H2 comprising the amino acid sequence of SEQ ID NO: 434; and CDR-H3 comprising the amino acid sequence of SEQ ID NO: 435; CDR-H1 comprising the amino acid sequence of SEQ ID NO: 439; CDR-H2 comprising the amino acid sequence of SEQ ID NO: 440; and CDR-H3 comprising the amino acid sequence of SEQ ID NO: 441; or CDR-H1 comprising the amino acid sequence of SEQ ID NO: 443; CDR-H2 comprising the amino acid sequence of SEQ ID NO: 444; and CDR-H3 comprising the amino acid sequence of SEQ ID NO: 435.

In some embodiments, the bispecific protein further comprises an anti-VEGF immunoglobulin light chain variable domain (anti-VEGF VL) comprising complementarity-determining regions: CDR-LT comprising the amino acid sequence of SEQ ID NO: 436; CDR-L2 comprising the amino acid sequence of SEQ ID NO: 437; and CDR-L3 comprising the amino acid sequence of SEQ ID NO: 438; CDR-LT comprising the amino acid sequence of SEQ ID NO: 442; CDR-L2 comprising the amino acid sequence of FTS; and CDR-L3 comprising the amino acid sequence of SEQ ID NO: 438; or CDR-LT comprising the amino acid sequence of SEQ ID NO: 436; CDR-L2 comprising the amino acid sequence of SEQ ID NO: 437; and CDR-L3 comprising the amino acid sequence of SEQ ID NO: 438.

In some embodiments, the anti-VEGF VH has an amino acid sequence at least 85% identical to that of SEQ ID NO: 431; and the anti-VEGF VL has an amino acid sequence at least 85% identical to that of SEQ ID NO: 432.

In some embodiments, the anti-VEGF VH has an amino acid sequence of SEQ ID NO: 431; and the anti-VEGF-1 VL has an amino acid of SEQ ID NO: 432.

In one aspect, provided are bispecific proteins comprising a PD-1 binding region and a VEGF-binding region, wherein (a) the PD-1 binding region comprises (i) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 456, and 447, respectively; (2) SEQ ID NOs 451, 458, and 453, respectively; or (3) SEQ ID NOs 443, 460, and 447, respectively, and (ii) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively; and (b) the VEGF-binding region comprises (i) an anti-VEGF immunoglobulin heavy chain variable domain (anti-VEGF VH) comprising complementary determining regions CDR-H1, CDR-H2, and CDR-H3 which comprise the amino acid sequences of (1) SEQ ID NOs: 433, 434, and 435, respectively; (2) SEQ ID NOs: 439, 440, and 441, respectively; or (3) SEQ ID NOs: 443, 444, and 435, respectively, and (ii) an anti-VEGF immunoglobulin light chain variable domain (anti-VEGF VL) comprising complementary determining regions CDR-L1, CDR-L2, and CDR-L3 which comprise the amino acid sequences of (1) SEQ ID NOs: 436, 437, and 438, respectively; (2) SEQ ID NO: 442, FTS, and SEQ ID NO: 438, respectively; or (3) SEQ ID NOs: 436, 437, and 438, respectively. In some embodiments, (a) the anti-PD-1 VH has an amino acid sequence at least 85% identical to that of SEQ ID NO: 325; and the anti-PD-1 VL has an amino acid sequence at least 85% identical to that of SEQ ID NO: 347; and (b) the anti-VEGF VH has an amino acid sequence at least 85% identical to that of SEQ ID NO: 431; and the anti-VEGF-1 VL has an amino acid sequence at least 85% identical to that of SEQ ID NO: 432. In some embodiments, (a) the anti-PD-1 VH has an amino acid sequence of SEQ ID NO: 325; and the anti-PD-1 VL has an amino acid sequence of SEQ ID NO: 347; and (b) the anti-VEGF VH has an amino acid sequence of SEQ ID NO: 431; and the anti-VEGF-1 VL has an amino acid sequence of SEQ ID NO: 432.

In one aspect, provided are bispecific proteins comprising a PD-1 binding region and a VEGF-binding region, wherein (a) the PD-1 binding region comprises (i) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 456, and 457, respectively; (2) SEQ ID NOs 451, 458, and 459, respectively; or (3) SEQ ID NOs 443, 460, and 457, respectively, and (b) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively; and (b) the VEGF-binding region comprises (i) an anti-VEGF immunoglobulin heavy chain variable domain (anti-VEGF VH) comprising complementary determining regions CDR-H1, CDR-H2, and CDR-H3 which comprise the amino acid sequences of (1) SEQ ID NOs: 433, 434, and 435, respectively; (2) SEQ ID NOs: 439, 440, and 441, respectively; or (3) SEQ ID NOs: 443, 444, and 435, respectively, and (ii) an anti-VEGF immunoglobulin light chain variable domain (anti-VEGF VL) comprising complementary determining regions CDR-L1, CDR-L2, and CDR-L3 which comprise the amino acid sequences of (1) SEQ ID NOs: 436, 437, and 438, respectively; (2) SEQ ID NO: 442, FTS, and SEQ ID NO: 438, respectively; or (3) SEQ ID NOs: 436, 437, and 438, respectively. In some embodiments, (a) the anti-PD-1 VH has an amino acid sequence at least 85% identical to that of SEQ ID NO: 320; and the anti-PD-1 VL has an amino acid sequence at least 85% identical to that of SEQ ID NO: 347; and (b) the anti-VEGF VH has an amino acid sequence at least 85% identical to that of SEQ ID NO: 431; and the anti-VEGF-1 VL has an amino acid sequence at least 85% identical to that of SEQ ID NO: 432. In some embodiments, (a) the anti-PD-1 VH has an amino acid sequence of SEQ ID NO: 320; and the anti-PD-1 VL has an amino acid sequence of SEQ ID NO: 347; and (b) the anti-VEGF VH has an amino acid sequence of SEQ ID NO: 431; and the anti-VEGF-1 VL has an amino acid sequence of SEQ ID NO: 432.

In one aspect, provided are bispecific proteins comprising a PD-1 binding region and a VEGF-binding region, wherein (a) the PD-1 binding region comprises (i) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 461, and 457, respectively; (2) SEQ ID NOs 451, 462, and 459, respectively; or (3) SEQ ID NOs 443, 463, and 457, respectively, and (ii) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively; and (a) the VEGF-binding region comprises (i) an anti-VEGF immunoglobulin heavy chain variable domain (anti-VEGF VH) comprising complementary determining regions CDR-H1, CDR-H2, and CDR-H3 which comprise the amino acid sequences of (1) SEQ ID NOs: 433, 434, and 435, respectively; (2) SEQ ID NOs: 439, 440, and 441, respectively; or (3) SEQ ID NOs: 443, 444, and 435, respectively, and (ii) an anti-VEGF immunoglobulin light chain variable domain (anti-VEGF VL) comprising complementary determining regions CDR-L1, CDR-L2, and CDR-L3 which comprise the amino acid sequences of (1) SEQ ID NOs: 436, 437, and 438, respectively; (2) SEQ ID NO: 442, FTS, and SEQ ID NO: 438, respectively; or (3) SEQ ID NOs: 436, 437, and 438, respectively. In some embodiments, (a) the anti-PD-1 VH has an amino acid sequence at least 85% identical to that of SEQ ID NO: 324; and the anti-PD-1 VL has an amino acid sequence at least 85% identical to that of SEQ ID NO: 347; and (b) the anti-VEGF VH has an amino acid sequence at least 85% identical to that of SEQ ID NO: 431; and the anti-VEGF-1 VL has an amino acid sequence at least 85% identical to that of SEQ ID NO: 432. In some embodiments, (a) the anti-PD-1 VH has an amino acid sequence of SEQ ID NO: 324; and the anti-PD-1 VL has an amino acid sequence of SEQ ID NO: 347; and (b) the anti-VEGF VH has an amino acid sequence of SEQ ID NO: 431; and the anti-VEGF-1 VL has an amino acid sequence of SEQ ID NO: 432.

In one aspect, provided are bispecific proteins comprising a PD-1 binding region and a VEGF-binding region, wherein (a) the PD-1 binding region comprises (i) an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 461, and 447, respectively; (2) SEQ ID NOs 451, 462, and 453, respectively; or (3) SEQ ID NOs 443, 463, and 447, respectively, and (ii) an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively; and (a) the VEGF-binding region comprises (i) an anti-VEGF immunoglobulin heavy chain variable domain (anti-VEGF VH) comprising complementary determining regions CDR-H1, CDR-H2, and CDR-H3 which comprise the amino acid sequences of (1) SEQ ID NOs: 433, 434, and 435, respectively; (2) SEQ ID NOs: 439, 440, and 441, respectively; or (3) SEQ ID NOs: 443, 444, and 435, respectively, and (ii) an anti-VEGF immunoglobulin light chain variable domain (anti-VEGF VL) comprising complementary determining regions CDR-L1, CDR-L2, and CDR-L3 which comprise the amino acid sequences of (1) SEQ ID NOs: 436, 437, and 438, respectively; (2) SEQ ID NO: 442, FTS, and SEQ ID NO: 438, respectively; or (3) SEQ ID NOs: 436, 437, and 438, respectively. In some embodiments, (a) the anti-PD-1 VH has an amino acid sequence at least 85% identical to that of SEQ ID NO: 326; and the anti-PD-1 VL has an amino acid sequence at least 85% identical to that of SEQ ID NO: 347; and (b) the anti-VEGF VH has an amino acid sequence at least 85% identical to that of SEQ ID NO: 431; and the anti-VEGF-1 VL has an amino acid sequence at least 85% identical to that of SEQ ID NO: 432. In some embodiments, (a) the anti-PD-1 VH has an amino acid sequence of SEQ ID NO: 326; and the anti-PD-1 VL has an amino acid sequence of SEQ ID NO: 347; and (b) the anti-VEGF VH has an amino acid sequence of SEQ ID NO: 431; and the anti-VEGF-1 VL has an amino acid sequence of SEQ ID NO: 432.

In one aspect, provided are bispecific proteins comprising a PD-1 binding region and a VEGF-binding region, wherein the PD-1 binding region comprises an anti-PD-1 immunoglobulin heavy chain variable domain (anti-PD-1 VH) comprising complementary determining regions CDR-H1, CDR-H2, and CDR-H3 which comprise the amino acid sequences of (1) SEQ ID NOs 445, 446, and 457, respectively; (2) SEQ ID NOs 451, 452, and 459, respectively; or (3) SEQ ID NOs 443, 455, and 457, respectively, and (an anti-PD1 immunoglobulin light chain variable domain (anti-PD-1 VL) comprising complementary determining regions CDR-L1, CDR-L2, and CDR-L3 which comprise the amino acid sequences of (1) SEQ ID NOs 448, 449, and 450, respectively; (2) SEQ ID NO: 454, LAS, and SEQ ID NO: 450, respectively; or (3) SEQ ID NOs 448, 449, and 450 respectively; and the VEGF-binding region comprises an anti-VEGF immunoglobulin heavy chain variable domain (anti-VEGF VH) comprising complementary determining regions CDR-H1, CDR-H2, and CDR-H3 which comprise the amino acid sequences of (1) SEQ ID NOs: 433, 434, and 435, respectively; (2) SEQ ID NOs: 439, 440, and 441, respectively; or (3) SEQ ID NOs: 443, 444, and 435, respectively, and an anti-VEGF immunoglobulin light chain variable domain (anti-VEGF VL) comprising complementary determining regions CDR-L1, CDR-L2, and CDR-L3 which comprise the amino acid sequences of (1) SEQ ID NOs: 436, 437, and 438, respectively; (2) SEQ ID NO: 442, FTS, and SEQ ID NO: 438, respectively; or (3) SEQ ID NOs: 436, 437, and 438, respectively.

In some embodiments, the anti-PD-1 VH has an amino acid sequence at least 85% identical to that of SEQ ID NO: 327; and the anti-PD-1 VL has an amino acid sequence at least 85% identical to that of SEQ ID NO: 347; and the anti-VEGF VH has an amino acid sequence at least 85% identical to that of SEQ ID NO: 431; and the anti-VEGF-1 VL has an amino acid sequence at least 85% identical to that of SEQ ID NO: 432.

In some embodiments, the anti-PD-1 VH has an amino acid sequence of SEQ ID NO: 327; and the anti-PD-1 VL has an amino acid sequence of SEQ ID NO: 347; and the anti-VEGF VH has an amino acid sequence of SEQ ID NO: 431; and the anti-VEGF-1 VL has an amino acid sequence of SEQ ID NO: 432.

In some embodiments, the bispecific protein further comprises an Fc region.

In some embodiments, the Fc region comprises a means for extending the half-life of the PD-1 binding protein.

In some embodiments, the half-life extending means comprises an Fc modification.

In some embodiments, the Fc modification is selected from the group consisting of M252Y/S254T/T256E (YTE), M428L/N434S (LS), M428L/N434A (LA), H433K/N434F (KF), and L309D/Q311H/N434S (DHS).

In some embodiments, the Fc modification is M252Y/S254T/T256E (YTE) or M428L/N434S (LS).

In some embodiments, the Fc modification is M428L/N434S (LS).

In some embodiments, the Fc region is an IgG1, IgG2, or IgG4 Fc region.

In some embodiments, the bispecific protein is an antibody or antigen-binding fragment thereof.

In some embodiments, the bispecific protein is a human or humanized antibody or antigen-binding fragment thereof.

In some embodiments, the antigen-binding fragment is a Fab, a F(ab′)2, a Fab′, a single-chain Fv (scFv), an Fv fragment, a Fd fragment, or a diabody.

In some embodiments, bispecific protein comprises two PD-1 binding regions and two VEGF binding regions.

In some embodiments, the bispecific protein comprises two PD-1 binding regions and one VEGF binding region.

In some embodiments, the bispecific protein comprises one PD-1 binding region and two VEGF binding regions.

In some embodiments, each PD-1 binding region is an scFv.

In some embodiments, each VEGF binding region comprises an anti-VEGF VH on a first polypeptide chain and an anti-VEGF VL on a second polypeptide chain, an anti-VEGF VH and an anti-VEGF VL within a Fab, or an anti-VEGF VH and an anti-VEGF VL within a CrossFab.

In some embodiments, each VEGF binding region is an scFv.

In some embodiments, each PD-1 binding region comprises an anti-PD1 VH on a first polypeptide chain and an anti-PD1 VL on a second polypeptide chain, an anti-PD1 VH and an anti-PD1 VL within a Fab, or an anti-PD1 VH and an anti-PD1 VL within a CrossFab.

In some embodiments, each PD-1 binding region is a VHH.

In some embodiments, each VEGF binding region comprises an anti-VEGF VH on a first polypeptide chain and an anti-VEGF VL on a second polypeptide chain, an anti-VEGF VH and an anti-VEGF VL within a Fab, or an anti-VEGF VH and an anti-VEGF VL within a CrossFab.

In one aspect, provided are bispecific proteins comprising at least one PD-1 binding region and at least one VEGF-binding region, comprising a first polypeptide chain having the sequence of SEQ ID NO: 92 and a second polypeptide chain having the sequence of SEQ ID NO: 239.

In one aspect, provided are bispecific proteins comprising at least one PD-1 binding region and at least one VEGF-binding region, comprising a first polypeptide chain having the sequence of SEQ ID NO: 73 and a second polypeptide chain having the sequence of SEQ ID NO: 220.

In one aspect, provided are bispecific proteins comprising at least one PD-1 binding region and at least one VEGF-binding region, comprising a first polypeptide chain having the sequence of SEQ ID NO: 91 and a second polypeptide chain having the sequence of SEQ ID NO: 238.

In one aspect, provided are bispecific proteins comprising at least one PD-1 binding region and at least one VEGF-binding region, comprising a first polypeptide chain having the sequence of SEQ ID NO: 94 and a second polypeptide chain having the sequence of SEQ ID NO: 241.

In one aspect, provided are bispecific proteins comprising at least one PD-1 binding region and at least one VEGF-binding region, comprising a first polypeptide chain having the sequence of SEQ ID NO: 96 and a second polypeptide chain having the sequence of SEQ ID NO: 243.

In some embodiments, the bispecific protein forms a dimer.

In various aspects, provided are isolated nucleic acids encoding one or more chains of a PD-1 binding protein or a bispecific protein as disclosed herein, expression vectors comprising said isolated nucleic acids, and host cells comprising said isolated nucleic acids or expression vectors.

In various aspects, provided are sets of isolated nucleic acids collectively encoding a PD-1 binding protein or a bispecific protein as disclosed herein, sets of expression vectors collectively comprising said sets of isolated nucleic acids, and host cells comprising said sets of isolated nucleic acids or said sets of expression vectors.

In one aspect, provided are pharmaceutical compositions comprising a PD-1 binding protein or bispecific protein as disclosed herein and a pharmaceutically acceptable carrier.

In one aspect, provided are methods comprising a step of administering to a subject in need thereof an effective amount of a PD-1 binding protein, bispecific protein, or pharmaceutical composition as disclosed herein.

In some embodiments, the subject has or is at risk of having a disease or condition associated with aberrant PD-1 and/or VEGF expression or signaling.

In some embodiments, the disease or condition is a cancer.

In some embodiments, the cancer is a lung cancer.

In some embodiments, the lung cancer is non-small cell lung cancer.

In some embodiments, the cancer is a gastrointestinal cancer.

In some embodiments, the gastrointestinal cancer is colorectal cancer, biliary cancer, gastric cancer, or hepatocellular cancer.

In some embodiments, the cancer is a reproductive cancer.

In some embodiments, the reproductive cancer is cervical cancer, endometrial cancer, or ovarian cancer.

In some embodiments, the step of administering comprises systemic administration of the PD-1 binding protein, bispecific protein, or pharmaceutical composition.

In some embodiments, systemic administration comprises intravenous administration of the PD-1 binding protein, bispecific protein, or pharmaceutical composition.

In some embodiments, systemic administration comprises subcutaneous administration of the PD-1 binding protein, bispecific protein, or pharmaceutical composition.

### BRIEF DESCRIPTION OF THE DRAWINGS

These and other features, aspects, and advantages of the present invention will become better understood with regard to the following description, and accompanying drawing, where:

FIGS. 1A-1CC depict binding activity of the indicated antibodies to PD-1 expressing cells.

FIGS. 2A-2D depict the surface plasmon resonance (SPR) curves (FIGS. 2A-2C) and PD-1/PD-L1 blockade assay results (FIG. 2D) for the indicated antibodies.

FIG. 3A depicts binding activity of the indicated antibodies to human VEGF determined by ELISA.

FIG. 3B depicts binding activity of the indicated antibodies to human PD-1 determined by ELISA.

FIG. 3C depicts binding activity of the indicated antibodies to cynomolgus PD-1 determined by ELISA.

FIGS. 4A-4D depict the SPR curves for binding of the indicated antibodies to human VEGF.

FIGS. 5A-5D depict the SPR curves for binding of the indicated antibodies to human PD-1.

FIGS. 6A-6D depict the SPR curves for binding of the indicated antibodies to cynomolgus PD-1.

FIGS. 7A-7F depict SEC-MALS complex profiles for the indicated antibodies.

FIG. 8 depicts the heavy and light chain sequences (SEQ ID NOs: 128 and 275, respectively) and format for ivonescimab (“AK112”). The variable domains from penpulimab (“AK105”) are underlined.

FIG. 9 demonstrates that bispecific antibodies can enhance PD-1 binding through VEGF daisy chaining.

FIGS. 10A-10KK depict the PD-1 reporter assay results for the indicated antibodies.

FIGS. 11A-11V depict VEGF reporter assay results for the indicated antibodies.

FIGS. 12A-120 depict inhibition of VEGF-mediated proliferation of human umbilical vein endothelial cells (HUVECs) by the indicated antibodies.

FIGS. 13A-13S depict PD-1 internalization results for the indicated antibodies.

FIG. 14A depicts a PD-1-VEGF (scFv) bispecific reverse construct.

FIGS. 14B-14E depict results of PD-1 binding (FIG. 14B), PD-1 internalization (FIG. 14D), PD-1 reporter (FIG. 14C), and VEGF reporter (FIG. 14E) assays for the PD-1-VEGF (scFv) bispecific reverse construct.

FIGS. 15A-15R depict data from T cell activation assessed in human PBMC and hepatocellular carcinoma co-culture assays with the indicated antibodies.

FIGS. 16A-16F depict data from T cell activation assessed in human monocyte derived dendritic cell and CD4+ T cell mixed lymphocyte reaction assays with the indicated antibodies.

FIG. 17A depicts a graph of viscosity at varying protein concentrations at constant pH of 6.0.

FIG. 17B depicts a graph of percent monomer by analytical size exclusion chromatography (SEC) at varying protein concentrations.

FIGS. 17C-17D depict stability data at 40° C. (FIG. 17C) and 25° C. (FIG. 17D).

FIGS. 18A-18C depict pharmacokinetic data of the indicated antibodies.

FIG. 19 depicts anti-tumor activity of the indicated antibodies.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a,” “an,” and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

An “effective amount” or “therapeutically effective amount” as used herein refers to an amount of therapeutic compound, such as an anti-VEGF antibody, administered to an individual, either as a single dose or as part of a series of doses, which is effective to produce or contribute to a desired therapeutic effect, either alone or in combination with another therapeutic modality. Examples of a desired therapeutic effect is enhancing an immune response, slowing or delaying tumor development; stabilization of disease; amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

The terms “recipient”, “individual”, “subject”, “host”, and “patient”, are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel. As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating, slowing or preventing a symptom thereof. As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s) ±one standard deviation of that value(s).

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “k_{d}” (sec-1), as used herein, refers to the dissociation rate constant of a particular antibody-antigen interaction. This value is also referred to as the k_{off} value.

The term “kₐ” (M-1×sec-1), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “K_{A}” (M-1), as used herein, refers to the association equilibrium constant of a particular antibody-antigen interaction. K_{A}=kₐ/k_{d}.

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (VL) and one constant region (CL). The heavy chain consists of one variable region (VH) and at least three constant regions (CH1, CH2 and CH3). The variable regions determine the binding specificity of the antibody. Each variable region contains three hypervariable regions known as complementarity determining regions (CDRs) flanked by four relatively conserved regions known as framework regions (FRs). The extent of the FRs and CDRs has been defined (Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). The three CDRs, referred to as CDR1, CDR2, and CDR3, contribute to the antibody binding specificity. Naturally occurring antibodies have been used as starting material for engineered antibodies, such as chimeric antibodies and humanized antibodies. Examples of antibody-based antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

A “VEGF antibody” or “VEGF specific antibody” is an antibody, as provided herein, which specifically binds to VEGF. A “VEGF binder” or “VEGF specific binder” is a binder, as provided herein, which specifically binds to VEGF.

A “PD-1 antibody,” “PD1 antibody” or “PD1 specific antibody” is an antibody, as provided herein, which specifically binds to PD1. A “PD-1 binder,” “PD1 binder” or “PD1 specific binder” is an binder, as provided herein, which specifically binds to PD1.

The term “epitope” means a portion of an antigen that specifically binds to an antibody.

The term “hypervariable region” or “HVR,” as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “bispecific antibody” is an antibody that comprises two different antigen binding domains that each bind different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with 8-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al. (1998) FEBS Letters 414:521-526 and Muyldermans et al. (2001) Trends in Biochem. Sci. 26:230-245, each of which is incorporated by reference in its entirety. Single domain antibodies are also known as sdAbs, VHH, or nanobodies. sdAbs are fairly stable and easy to express as fusion partner with the Fc chain of an antibody (Harmsen M M, De Haard H J (2007) “Properties, production, and applications of camelid single-domain antibody fragments” Appl. Microbiol Biotechnol. 77(1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses (Altschul et al. (1990) J. Mol. Biol. 215:403-410) is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman (1981) Adv. Appl. Math. 2:482, by the homology alignment algorithm of Needleman & Wunsch (1970) J. Mol. Biol. 48:443, by the search for similarity method of Pearson & Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### PD-1 Antibodies and Bispecific Anti-VEGF/PD-1 Antibodies

### Basic Antibody Structure

The recognized immunoglobulin (antibody) genes include the kappa, lambda, alpha, gamma, delta, epsilon, and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called α, δ, ε, γ, and μ, respectively.

An exemplary immunoglobulin structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2, and CH3 domains respectively from the N- to C-terminus. The light chain comprises of the VL and CL domains from N- to C-terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or derived from an immunoglobulin based construct such as a diabody or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody, or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six hypervariable regions (HVRs); three in the VH (H1, H2, and H3), and three in the VL (L1, L2, and L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. HVRs are also referred to as CDRs, and these terms are used herein interchangeably in reference to portions of the variable region that form the antigen-binding regions. This particular region has been described by Kabat et al. (1983) U.S. Dept. of Health and Human Services, Sequences of Proteins of Immunological Interest and by Chothia et al. (1987) J Mol Biol 196:901-917, where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al. (1997) J. Mol. Biol., 273:927-948 (“Chothia” numbering scheme); MacCallum et al. (1996) J. Mol. Biol. 262:732-745 (“Contact” numbering scheme); Lefranc et al. (2003) Dev. Comp. Immunol. 27:55-77 (“IMGT” numbering scheme); and Honegge and Plückthun (2001) J. Mol. Biol. 309:657-70 (“AHo” numbering scheme); each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at bioinf.org.uk/abs/abnum/, and described in Abhinandan and Martin (2008) Immunology, 45:3832-3839, incorporated by reference in its entirety.

TABLE 1
Residues in CDRs according to Kabat and Chothia numbering schemes.
Table 1

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding and other amino acid residues, which are not directly involved in the binding. The epitope to which an antibody binds can be determined using known techniques for epitope determination such as, for example, testing for antibody binding to VEGF variants with different point-mutations or to chimeric VEGF variants.

To screen for antibodies which bind to an epitope on a target antigen bound by an antibody of interest (e.g., VEGF or PD-1), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human antibodies are antibodies which possess an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al. (1986) Nature 321:522-525; Riechmann et al. (1988) Nature 332:323-329; and Presta (1992) Curr. Op. Struct. Biol. 2:593-596, each of which is incorporated by reference in its entirety.

The two or more different epitopes may be epitopes on the same antigen (e.g., a single VEGF) or on different antigens (e.g., different VEGF molecules, or a VEGF molecule and a non-VEGF molecule). In some embodiments, a multi-specific antibody binds two different epitopes (i.e., a “bispecific antibody”). In some embodiments, a multi-specific antibody binds three different epitopes (i.e., a “trispecific antibody”).

Anti-VEGF or PD-1 antibodies can include those described herein such as the clones set forth in the drawings and/or tables. In some embodiments, the binding protein comprises an alternative scaffold. In some embodiments, the binding protein consists of an alternative scaffold. In some embodiments, the binding protein consists essentially of an alternative scaffold. In some embodiments, the binding protein comprises an antibody fragment. In some embodiments, the binding protein consists of an antibody fragment. In some embodiments, the binding protein consists essentially of an antibody fragment.

In some embodiments the bispecific antibodies are monoclonal antibodies.

In some embodiments the bispecific antibodies are polyclonal antibodies.

In some embodiments the bispecific antibodies are produced by hybridomas. In other embodiments, the bispecific antibodies are produced by recombinant cells engineered to express the desired variable and constant domains.

In some embodiments the bispecific antibodies may be single chain antibodies or other antibody derivatives retaining the antigen specificity and the lower hinge region or a variant thereof.

In some embodiments the bispecific antibodies may be polyfunctional antibodies, recombinant antibodies, human antibodies, humanized antibodies, fragments or variants thereof. In particular embodiments, the antibody fragment or a derivative thereof is selected from a Fab fragment, a Fab′₂ fragment, a CDR, and scFv.

In some embodiments, the bispecific antibodies are capable of forming an immune complex. For example, an immune complex can be a tumor cell covered by bispecific antibodies.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

### Bispecific Antibody Structure

The present application provides proteins that bind VEGF and PD-1, as well as compositions (e.g., pharmaceutical compositions thereof). Bispecific antibodies disclosed herein can be of any structure known in the art.

Disclosed bispecific binding proteins can take any format, including but not limited to those described herein.

In one exemplary bispecific format, the bispecific binding protein comprises (a) two immunoglobulin heavy chains which each comprise, from N- to C-terminus: (1) a heavy chain variable domain, (2) an Fc (hinge-CH2-CH3) domain, which immunoglobulin heavy chains are each linked at their C-termini to an scFv; and (b) two immunoglobulin light chains which each comprise, from N- to C-terminus: (1) a light chain variable domain and (2) a light chain constant domain. Linkers (e.g., amino acid linkers) may be used between any of the aforementioned domains within a given immunoglobulin chain, within an scFv, and/or between the immunoglobulin heavy chain and the scFv. In this format, a heavy chain variable domain and a light chain variable domain together form a first binding region, and the scFv comprises a second binding region. In some embodiments, the first binding region is a VEGF binding region and the second binding region is a PD-1 binding region. In some embodiments, the first binding region is a PD-1 binding region and the second binding region is a VEGF binding region. Thus, for example, in this format, the bispecific binding protein comprises two VEGF binding regions and two PD-1 binding regions per construct.

However, additional formats, such as formats comprising different numbers of each binding region, are also contemplated, e.g., two VEGF binding regions and one PD-1 binding region, one VEGF binding region and two PD-1 binding regions, or one VEGF binding region and one PD-1 binding region.

Another format is a bispecific binding protein that includes a first immunoglobulin heavy chain, a second immunoglobulin heavy chain and an immunoglobulin light chain. In this format, (1) the first immunoglobulin heavy chain includes a first Fc (hinge-CH2-CH3) domain, a first variable heavy chain domain and an optional first CH1 heavy chain domain, (2) the immunoglobulin light chain includes a variable light chain domain and a constant light chain domain; and (3) together with the first immunoglobulin heavy chain, the immunoglobulin light chain forms an antigen-binding site that binds VEGF. In this format, the second immunoglobulin heavy chain comprises a second Fc (hinge-CH2-CH3) domain, a second variable heavy chain domain and a second CH1 heavy chain domain that may pair with an immunoglobulin light chain identical to the one that pairs with the first immunoglobulin heavy chain, except that when immunoglobulin light chain is paired with the second immunoglobulin heavy chain, the resulting antigen binding site binds to PD-1.

In some embodiments, the bispecific binding protein is in the Triomab form, which is a trifunctional, bispecific binding protein that maintains an IgG-like shape. This chimera consists of two half antibodies, each with one light and one heavy chain, that originate from two parental antibodies.

In some embodiments, the bispecific binding protein is the KiH Common Light Chain (LC) form, which involves the knobs-into-holes (KIHs) technology. The KIH involves engineering C_{H}3 domains to create either a “knob” or a “hole” in each heavy chain to promote heterodimerization. The concept behind the “Knobs-into-Holes (KiH)” Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structured-guided phage library screening (Atwell et al. (1997) “Stable heterodimers from remodeling the domain interface of a homodimer using a phage display library,” J. Mol. Biol. 270(1):26-35). X-ray crystal structures of KiH Fc variants (Elliott et al. (2014) “Antiparallel conformation of knob and hole aglycosylated half-antibody homodimers is mediated by a CH2-CH3 hydrophobic interaction,” J. Mol. Biol. 426(9):1947-57; Mimoto et al. (2014) “Crystal structure of a novel asymmetrically engineered Fc variant with improved affinity for FcgammaRs,” Mol. Immunol. 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

In some embodiments, the bispecific binding protein is in the Orthogonal Fab interface (Ortho-Fab) form. In the ortho-Fab IgG approach (Lewis et al. (2014) “Generation of bispecific IgG antibodies by structure-based design of an orthogonal Fab interface,” Nat. Biotechnol. 32(2):191-8), structure-based regional design introduces complementary mutations at the LC and HC_{VH-CH1} interface in only one Fab, without any changes being made to the other Fab.

In some embodiments, the bispecific binding protein is in the 2-in-1 Ig format. In some embodiments, the bispecific binding protein is in the ES form, which is a heterodimeric construct containing two different Fabs binding to targets 1 and target 2 fused to the Fc. Heterodimerization is ensured by electrostatic steering mutations in the Fc. In some embodiments, the bispecific binding protein is in the κλ-Body form, which is a heterodimeric construct with two different Fabs fused to Fc stabilized by heterodimerization mutations: Fab1 targeting antigen 1 contains kappa LC, while second Fab targeting antigen 2 contains lambda LC.

In some embodiments, the bispecific binding protein is in Fab Arm Exchange form (antibodies that exchange Fab arms by swapping a heavy chain and attached light chain (half-molecule) with a heavy-light chain pair from another molecule, which results in bispecific antibodies). In some embodiments, the bispecific binding protein is in the SEED Body form. The strand-exchange engineered domain (SEED) platform was designed to generate asymmetric and bispecific binding protein-like molecules, a capability that expands therapeutic applications of natural antibodies. This protein engineered platform is based on exchanging structurally related sequences of immunoglobulin within the conserved CH3 domains. The SEED design allows efficient generation of AG/GA heterodimers, while disfavoring homodimerization of AG and GA SEED CH3 domains. (Muda M. et al. (2011) Protein Eng. Des. Sel. 24(5):447-54). In some embodiments, the bispecific binding protein is in the LuZ-Y form, in which a leucine zipper is used to induce heterodimerization of two different HCs. (Wranik et al. (2012) J. Biol. Chem. 287:43331-9).

In some embodiments, the bispecific binding protein is in the Cov-X-Body form. In bispecific CovX-Bodies, two different peptides are joined together using a branched azetidinone linker and fused to the scaffold binding protein under mild conditions in a site-specific manner. Whereas the pharmacophores are responsible for functional activities, the binding protein scaffold imparts long half-life and Ig-like distribution. The pharmacophores can be chemically optimized or replaced with other pharmacophores to generate optimized or unique bispecific antibodies. (Doppalapudi et al. (2010) PNAS 107(52): 22611-22616).

In some embodiments, the bispecific binding protein is in an Oasc-Fab heterodimeric form that includes Fab binding to target 1, and scFab binding to target 2 fused to Fc. Heterodimerization is ensured by mutations in the Fc.

In some embodiments, the bispecific binding protein is in a DuetMab form, which is an heterodimeric construct containing two different Fabs binding to antigens 1 and 2, and Fc stabilized by heterodimerization mutations. Fab 1 and 2 contain differential S—S bridges that ensure correct LC and HC pairing.

In some embodiments, the bispecific binding protein is in a CrossmAb form, which is an heterodimeric construct with two different Fabs binding to targets 1 and 2, fused to Fc stabilized by heterodimerization. CL and CH1 domains and VH and VL domains are switched, e.g., CH1 is fused in-line with VL, while CL is fused in-line with VH.

In some embodiments, the bispecific binding protein is in a Fit-Ig form, which is a homodimeric construct where Fab binding to antigen 2 is fused to the N terminus of HC of Fab that binds to antigen 1. The construct contains wild-type Fc.

Tables 2A-2B lists sequences of heavy chains and light chains that, in combination, can bind to VEGF and/or PD-1.

### Sequences of PD-1 Antibodies and Bispecific Anti-VEGF/PD-1 Binding Proteins

Provided herein are binding proteins that bind programmed death receptor 1 (PD-1). PD-1 binding proteins generally comprise one or more PD-1 binding regions, as further discussed herein.

Also provided herein are bispecific binding proteins that bind vascular endothelial growth factor (VEGF) and programmed death receptor 1 (PD-1). Provided bispecific binding proteins generally comprise one or more VEGF binding regions and one or more PD-1 binding regions.

### Exemplary Characteristic Sequences of VEGF Binding Regions

VEGF binding regions are capable of binding to VEGF. In some embodiments, VEGF binding regions are capable of binding to an epitope of human VEGF, such as a human VEGF-A isoform. In some embodiments, VEGF binding regions are capable of binding to all human VEGF-A isoforms. In some embodiments, VEGF binding regions antagonize VEGF, e.g., by blocking binding of VEGF to its receptor.

In some embodiments, VEGF binding regions comprise a heavy chain variable domain comprising complementarity determining regions CDR-H1, CDR-H2, and CDR-H3 with sequences as shown in Table 2. In some embodiments, VEGF binding regions further comprise a light chain variable domain comprising complementarity determining regions CDR-L1, CDR-L2, and CDR-L3 with sequences as shown in Table 2.

In some embodiments, VEGF binding regions comprise a heavy chain variable domain with a heavy chain variable domain sequence as shown in Table 2. In some embodiments, VEGF binding regions comprise a heavy chain variable domain which is a variant of the heavy chain variable sequence shown in Table 2, in that the heavy chain variable domain has (1) CDR-H1, CDR-H2, and CDR-H3 with sequences as shown in Table 2 and (2) an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the heavy chain variable domain sequence shown in Table 2.

In some embodiments, VEGF binding regions comprise a heavy chain variable domain as described herein in Table 2 (or a variant thereof, as described herein) and further comprise a light chain variable region which has (1) CDR-L1, CDR-L2, and CDR-L3 with sequences as shown in Table 2 and (2) an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the light chain variable domain sequence shown in Table 2.

TABLE 2
Characteristic sequences of exemplary VEGF binding regions

| Heavy chain | Light chain |
|---|---|
| Variable domain | Variable domain |
| EVQLVESGGGLVQPGGSLRLSCAASGYTFTNY | DIQMTQSPSSLSASVGDRVTITCSASQDISNY |
| GMNWVRQAPGKGLEWVGWINTYTGEPTYAADF | LNWYQQKPGKAPKVLIYFTSSLHSGVPSRFSG |
| KRRFTFSLDTSKSTAYLQMNSLRAEDTAVYYC | SGSGTDFTLTISSLQPEDFATYYCQQYSTVPW |
| AKYPHYYGSSHWYFDVWGQGTLVTVSS | TFGQGTKVEIK |
| (SEQ ID NO: 431) | (SEQ ID NO: 432) |
| Kabat CDRs | Kabat CDRs |
| CDR-H1: NYGMN | CDR-L1: SASQDISNYLN |
| (SEQ ID NO: 433) | (SEQ ID NO: 436) |
| CDR-H2: WINTYTGEPTYAADFKR | CDR-L2: FTSSLHS |
| (SEQ ID NO: 434) | (SEQ ID NO: 437) |
| CDR-H3: YPHYYGSSHWYFDV | CDR-L3: QQYSTVPWT |
| (SEQ ID NO: 435) | (SEQ ID NO: 438) |
| IMGT CDRs | IMGT CDRs |
| CDR-H1: GYTFTNYG | CDR-L1: QDISNY |
| (SEQ ID NO: 439) | (SEQ ID NO: 442) |
| CDR-H2: INTYTGEP | CDR-L2: FTS |
| (SEQ ID NO: 440) | CDR-L3: QQYSTVPWT |
| CDR-H3: AKYPHYYGSSHWYFDV | (SEQ ID NO: 438) |
| (SEQ ID NO: 441) | |
| Chothia CDRs | Chothia CDRs |
| CDR-H1: GYTFTNY | CDR-L1: SASQDISNYLN |
| (SEQ ID NO: 443) | (SEQ ID NO: 436) |
| CDR-H2: NTYTGE | CDR-L2: FTSSLHS |
| (SEQ ID NO: 444) | (SEQ ID NO: 437) |
| CDR-H3: YPHYYGSSHWYFDV | CDR-L3: QQYSTVPWT |
| (SEQ ID NO: 435) | (SEQ ID NO: 438) |

In some embodiments, VEGF binding regions comprise a heavy chain variable domain comprising complementarity determining regions CDR-H1, CDR-H2, and CDR-H3 with sequences within a heavy chain variable domain sequence shown in Table 5A. In some embodiments, VEGF binding regions further comprise a light chain variable domain comprising complementarity determining regions CDR-L1, CDR-L2, and CDR-L3 with sequences as shown within a light chain variable domain sequence in Table 6A.

In some embodiments, VEGF binding regions comprise a heavy chain variable domain with a heavy chain variable sequence as shown in Table 5A. In some embodiments, VEGF binding regions comprise a heavy chain variable domain which is a variant of the heavy chain variable sequence shown in Table 5A, in that the heavy chain variable domain has (1) CDR-H1, CDR-H2, and CDR-H3 with sequences within a given heavy chain variable domain sequence shown in Table 5A and (2) an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the same heavy chain variable domain sequence shown in Table 5A.

In some embodiments, VEGF binding regions comprise a heavy chain variable domain as described herein in Table 5A (or a variant thereof, as described herein) and further comprise a light chain variable region which has (1) CDR-L1, CDR-L2, and CDR-L3 within a light chain variable domain sequence as shown in Table 6A and (2) an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the same light chain variable domain sequence shown in Table 6A.

### Exemplary Characteristic Sequences of PD-1-Binding Regions

PD-1 binding regions are capable of binding to PD-1. In some embodiments, PD-1 binding regions are capable of binding to an epitope of human PD-1. In some embodiments, PD-1 binding regions antagonize PD-1, e.g., by blocking binding of PD-1 to PD-L1.

In some embodiments, PD-1 binding regions comprise a heavy chain variable domain comprising complementarity determining regions CDR-H1, CDR-H2, and CDR-H3 with sequences as shown for a given set in Table 3A. In some embodiments, PD-1 binding regions further comprise a light chain variable domain comprising complementarity determining regions CDR-L1, CDR-L2, and CDR-L3 with sequences as shown in the same set in Table 3A.

TABLE 3A
Exemplary sequences of complementarity-determining regions
within PD-1 binding regions

| | |
|---|---|
| Heavy chain | Light chain |

Set 1

| | |
|---|---|
| Kabat CDRs | Kabat CDRs |
| CDR-H1: NYYMY | CDR-L1: RASKGVSTSGYSYLH |
| (SEQ ID NO: 445) | (SEQ ID NO: 448) |
| CDR-H2: GINPSNGGTNFNEKFKN | CDR-L2: LASYLES |
| (SEQ ID NO: 446) | (SEQ ID NO: 449) |
| CDR-H3: RDYRFDMGFDY | CDR-L3: QHSRDLPLT |
| (SEQ ID NO: 447) | (SEQ ID NO: 450) |
| IMGT CDRs | IMGT CDRs |
| CDR-H1: GYTFTNYY | CDR-L1: KGVSTSGYSY |
| (SEQ ID NO: 451) | (SEQ ID NO: 454) |
| CDR-H2: INPSNGGT | CDR-L2: LAS |
| (SEQ ID NO: 452) | CDR-L3: QHSRDLPLT |
| CDR-H3: ARRDYRFDMGFDY | (SEQ ID NO: 450) |
| (SEQ ID NO: 453) | |
| Chothia CDRs | Chothia CDRs |
| CDR-H1: GYTFTNY | CDR-L1: RASKGVSTSGYSYLH |
| (SEQ ID NO: 443) | (SEQ ID NO: 448) |
| CDR-H2: NPSNGG | CDR-L2: LASYLES |
| (SEQ ID NO: 455) | (SEQ ID NO: 449) |
| CDR-H3: RDYRFDMGFDY | CDR-L3: QHSRDLPLT |
| (SEQ ID NO: 447) | (SEQ ID NO: 450) |

Set 2

| | |
|---|---|
| Kabat CDRs | Kabat CDRs |
| CDR-H1: NYYMY | CDR-L1: RASKGVSTSGYSYLH |
| (SEQ ID NO: 445) | (SEQ ID NO: 448) |
| CDR-H2: GINPSQGGTNFNEKFKN | CDR-L2: LASYLES |
| (SEQ ID NO: 456) | (SEQ ID NO: 449) |
| CDR-H3: RDYRFDLGFDY | CDR-L3: QHSRDLPLT |
| (SEQ ID NO: 457) | (SEQ ID NO: 450) |
| IMGT CDRs | IMGT CDRs |
| CDR-H1: GYTFTNYY | CDR-L1: KGVSTSGYSY |
| (SEQ ID NO: 451) | (SEQ ID NO: 454) |
| CDR-H2: INPSQGGT | CDR-L2: LAS |
| (SEQ ID NO: 458) | CDR-L3: QHSRDLPLT |
| CDR-H3: ARRDYRFDLGFDY | (SEQ ID NO: 450) |
| (SEQ ID NO: 459) | |
| Chothia CDRs | Chothia CDRs |
| CDR-H1: GYTFTNY | CDR-L1: RASKGVSTSGYSYLH |
| (SEQ ID NO: 443) | (SEQ ID NO: 448) |
| CDR-H2: NPSQGG | CDR-L2: LASYLES |
| (SEQ ID NO: 460) | (SEQ ID NO: 449) |
| CDR-H3: RDYRFDLGFDY | CDR-L3: QHSRDLPLT |
| (SEQ ID NO: 457) | (SEQ ID NO: 450) |

Set 3

| | |
|---|---|
| Kabat CDRs | Kabat CDRs |
| CDR-H1: NYYMY | CDR-L1: RASKGVSTSGYSYLH |
| (SEQ ID NO: 445) | (SEQ ID NO: 448) |
| CDR-H2: GINPSQGGTNFNEKFKN | CDR-L2: LASYLES |
| (SEQ ID NO: 456) | (SEQ ID NO: 449) |
| CDR-H3: RDYRFDMGFDY | CDR-L3: QHSRDLPLT |
| (SEQ ID NO: 447) | (SEQ ID NO: 450) |
| IMGT CDRs | IMGT CDRs |
| CDR-H1: GYTFTNYY | CDR-L1: KGVSTSGYSY |
| (SEQ ID NO: 451) | (SEQ ID NO: 454) |
| CDR-H2: INP SQGGT | CDR-L2: LAS |
| (SEQ ID NO: 458) | CDR-L3: QHSRDLPLT |
| CDR-H3: ARRDYRFDMGFDY | (SEQ ID NO: 450) |
| (SEQ ID NO: 453) | |
| Chothia CDRs | Chothia CDRs |
| CDR-H1: GYTFTNY | CDR-L1: RASKGVSTSGYSYLH |
| (SEQ ID NO: 443) | (SEQ ID NO: 448) |
| CDR-H2: NPSQGG | CDR-L2: LASYLES |
| (SEQ ID NO: 460) | (SEQ ID NO: 449) |
| CDR-H3: RDYRFDMGFDY | CDR-L3: QHSRDLPLT |
| (SEQ ID NO: 447) | (SEQ ID NO: 450) |

Set 4

| | |
|---|---|
| Kabat CDRs | Kabat CDRs |
| CDR-H1: NYYMY | CDR-L1: RASKGVSTSGYSYLH |
| (SEQ ID NO: 445) | (SEQ ID NO: 448) |
| CDR-H2: GINPSRGGTNFNEKFKN | CDR-L2: LASYLES |
| (SEQ ID NO: 461) | (SEQ ID NO: 449) |
| CDR-H3: RDYRFDMGFDY | CDR-L3: QHSRDLPLT |
| (SEQ ID NO: 447) | (SEQ ID NO: 450) |
| IMGT CDRs | IMGT CDRs |
| CDR-H1: GYTFTNYY | CDR-L1: KGVSTSGYSY |
| (SEQ ID NO: 451) | (SEQ ID NO: 454) |
| CDR-H2: INPSRGGT | CDR-L2: LAS |
| (SEQ ID NO: 462) | CDR-L3: QHSRDLPLT |
| CDR-H3: ARRDYRFDMGFDY | (SEQ ID NO: 450) |
| (SEQ ID NO: 453) | |
| Chothia CDRs | Chothia CDRs |
| CDR-H1: GYTFTNY | CDR-L1: RASKGVSTSGYSYLH |
| (SEQ ID NO: 443) | (SEQ ID NO: 448) |
| CDR-H2: NPSRGG | CDR-L2: LASYLES |
| (SEQ ID NO: 463) | (SEQ ID NO: 449) |
| CDR-H3: RDYRFDMGFDY | CDR-L3: QHSRDLPLT |
| (SEQ ID NO: 447) | (SEQ ID NO: 450) |

Set 5

| | |
|---|---|
| Kabat CDRs | Kabat CDRs |
| CDR-H1: NYYMY | CDR-L1: RASKGVSTSGYSYLH |
| (SEQ ID NO: 445) | (SEQ ID NO: 448) |
| CDR-H2: GINPSNGGTNFNEKFKN | CDR-L2: LASYLES |
| (SEQ ID NO: 446) | (SEQ ID NO: 449) |
| CDR-H3: RDYRFDLGFDY | CDR-L3: QHSRDLPLT |
| (SEQ ID NO: 457) | (SEQ ID NO: 450) |
| IMGT CDRs | IMGT CDRs |
| CDR-H1: GYTFTNYY | CDR-L1: KGVSTSGYSY |
| (SEQ ID NO: 451) | (SEQ ID NO: 454) |
| CDR-H2: INPSNGGT | CDR-L2: LAS |
| (SEQ ID NO: 452) | CDR-L3: QHSRDLPLT |
| CDR-H3: ARRDYRFDLGFDY | (SEQ ID NO: 450) |
| (SEQ ID NO: 459) | |
| Chothia CDRs | Chothia CDRs |
| CDR-H1: GYTFTNY | CDR-L1: RASKGVSTSGYSYLH |
| (SEQ ID NO: 443) | (SEQ ID NO: 448) |
| CDR-H2: NPSNGG | CDR-L2: LASYLES |
| (SEQ ID NO: 455) | (SEQ ID NO: 449) |
| CDR-H3: RDYRFDLGFDY | CDR-L3: QHSRDLPLT |
| (SEQ ID NO: 457) | (SEQ ID NO: 450) |

Set 6

| | |
|---|---|
| Kabat CDRs | Kabat CDRs |
| CDR-H1: NYYMY | CDR-L1: RASKGVSTSGYSYLH |
| (SEQ ID NO: 445) | (SEQ ID NO: 448) |
| CDR-H2: GINPSRGGTNFNEKFKN | CDR-L2: LASYLES |
| (SEQ ID NO: 461) | (SEQ ID NO: 449) |
| CDR-H3: RDYRFDLGEFDY | CDR-L3: QHSRDLPLT |
| (SEQ ID NO: 457) | (SEQ ID NO: 450) |
| IMGT CDRs | IMGT CDRs |
| CDR-H1: GYTFTNYY | CDR-L1: KGVSTSGYSY |
| (SEQ ID NO: 451) | (SEQ ID NO: 454) |
| CDR-H2: INPSRGGT | CDR-L2: LAS |
| (SEQ ID NO: 462) | CDR-L3: QHSRDLPLT |
| CDR-H3: ARRDYRFDLGFDY | (SEQ ID NO: 450) |
| (SEQ ID NO: 459) | |
| Chothia CDRs | Chothia CDRs |
| CDR-H1: GYTFTNY | CDR-L1: RASKGVSTSGYSYLH |
| (SEQ ID NO: 443) | (SEQ ID NO: 448) |
| CDR-H2: NPSRGG | CDR-L2: LASYLES |
| (SEQ ID NO: 463) | (SEQ ID NO: 449) |
| CDR-H3: RDYRFDLGFDY | CDR-L3: QHSRDLPLT |
| (SEQ ID NO: 457) | (SEQ ID NO: 450) |

In some embodiments, PD-1 binding regions comprise a heavy chain variable domain comprising complementarity determining regions CDR-H1, CDR-H2, and CDR-H3 with sequences within a heavy chain variable domain sequence shown in Table 5B. In some embodiments, PD-1 binding regions further comprise a light chain variable domain comprising complementarity determining regions CDR-L1, CDR-L2, and CDR-L3 with sequences as shown within a light chain variable domain sequence in Table 6B.

In some embodiments, PD-1 binding regions comprise a heavy chain variable domain with a heavy chain variable sequence as shown in Table 5B. In some embodiments, PD-1 binding regions comprise a heavy chain variable domain which is a variant of the heavy chain variable sequence shown in Table 5B, in that the heavy chain variable domain has (1) CDR-H1, CDR-H2, and CDR-H3 with sequences within a given heavy chain variable domain sequence shown in Table 5B and (2) an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the same heavy chain variable domain sequence shown in Table 5B.

In some embodiments, PD-1 binding regions comprise a heavy chain variable domain as described herein in Table 5B (or a variant thereof, as described herein) and further comprise a light chain variable region which has (1) CDR-L1, CDR-L2, and CDR-L3 within a light chain variable domain sequence as shown in Table 6B and (2) an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the same light chain variable domain sequence shown in Table 6B.

Tables 3B and 3C depict exemplary VH and VL sequences, respectively, of PD-1 binding regions, which illustrate representative heavy chain variable domain and light chain variable domain framework sequences within PD-1 binding regions of the present disclosure. Exemplary CDR sequences (as defined by Kabat) within these VH and VL sequences are shown within boxes. However, it is understood by those of ordinary skill in the art that the exact borders between CDRs and framework regions may vary depending on the antibody annotation system used. For example, CDRs may be defined as described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Other CDR definitions, such as according to IMGT, Chothia, Contact residues, may also be used. The exact residue numbers which encompass a particular CDR will vary depending on the sequence and size of the CDR. Those skilled in the art can routinely determine which residues comprise a particular CDR given the variable region amino acid sequence of the antibody.

In some embodiments, PD-1 binding regions comprise: (1) a set of CDRs as described herein for PD-1 binding regions, (2) a heavy chain variable domain having a sequence that has at least 75%, at least 80%, at least 85%, at least 90%, at least 92.5%, at least 95%, at least 97.5%, at least 98%, or at least 99% amino acid sequence identity with the sequence of a VH sequence shown in Table 3B, and (3) a light chain variable domain having a sequence that has at least 75%, at least 80%, at least 85%, at least 90%, at least 92.5%, at least 95%, at least 97.5%, at least 98%, or at least 99% amino acid sequence identity with the sequence of a VL sequence shown in Table 3C.

In some embodiments, PD-1 binding regions comprise a heavy chain variable domain framework sequence as shown within a VH sequence shown in Table 3B and a light chain variable domain framework sequence as shown within a VL sequence as shown in Table 3C.

In some embodiments, PD-1 binding regions comprise a heavy chain variable domain framework sequence as shown within a VH sequence shown in Table 3B and a variant of a light chain variable domain framework sequence as shown within a VL sequence as shown in Table 3C. In some embodiments, PD-1 binding regions comprise a variant of a heavy chain variable domain framework sequence as shown within a VH sequence shown in Table 3B and a light chain variable domain framework sequence as shown within a VL sequence as shown in Table 3C. In some embodiments, PD-1 binding regions comprise a variant of a heavy chain variable domain framework sequence as shown within a VH sequence shown in Table 3B and a variant of a light chain variable domain framework sequence as shown within a VL sequence as shown in Table 3C.

Examples of variants of disclosed framework sequences include, but are not limited to: (1) sequences that have at least 75%, at least 80%, at least 85%, at least 90%, at least 92.5%, at least 95%, at least 97.5%, at least 98%, or at least 99% amino acid sequence identity across framework region residues (not including CDR residues, according to any definition of CDRs used by those of skill in the art) with a disclosed framework sequence and (2) sequences that are nearly identical to a disclosed framework sequence except for a specific framework mutation or set of framework mutations (e.g., up to four, three, two, or one amino acid substitutions, deletions, or insertions within a given immunoglobulin framework region) as discussed herein. Non-limiting examples of specific framework mutations include stabilizing mutations and/or cysteine mutations (e.g., at H44 and/or L100).

Any combination of the heavy chain framework sequences and light chain framework sequences disclosed herein may be used in PD-1 binding regions of the present disclosure. Non-limiting examples of pairs of heavy chain and light chain framework sequences that can be used within PD-1 binding regions include:
(1) VH0 and VL0 framework regions (within SEQ ID NOs: 464 and 471, respectively), or variants thereof;
(2) VH1 and VL1 framework regions (within SEQ ID NOs: 465 and 472, respectively), or variants thereof;
(3) VH1 and VL0 framework regions (within SEQ ID NOs: 465 and 471, respectively), or variants thereof;
(4) VH1 and VL2 framework regions (within SEQ ID NOs: 465 and 473, respectively), or variants thereof;
(5) VH0 and VL1 framework regions (within SEQ ID NOs: 464 and 472, respectively), or variants thereof;
(6) VH0 and VL2 framework regions (within SEQ ID NOs: 464 and 473, respectively), or variants thereof;
(7) VH0 and VL3 framework regions (within SEQ ID NOs: 464 and 474, respectively), or variants thereof;
(8) VH0 and VL4 framework regions (within SEQ ID NOs: 464 and 475, respectively), or variants thereof;
(9) VH3 and VL0 framework regions (within SEQ ID NOs: 467 and 471, respectively), or variants thereof;
(10) VH3 and VL3 framework regions (within SEQ ID NOs: 467 and 474, respectively), or variants thereof;
(11) VH3 and VL4 framework regions (within SEQ ID NOs: 467 and 475, respectively), or variants thereof;
(12) VH4 and VL0 framework regions (within SEQ ID NOs: 468 and 471, respectively), or variants thereof;
(13) VH4 and VL3 framework regions (within SEQ ID NOs: 468 and 474, respectively), or variants thereof;
(14) VH4 and VL4 framework regions (within SEQ ID NOs: 468 and 475, respectively), or variants thereof;
(15) VH2 and VL0 framework regions (within SEQ ID NOs: 466 and 471, respectively), or variants thereof;
(16) VH6 and VL framework regions (within SEQ ID NOs: 470 and 474, respectively), or variants thereof; and
(17) VH5 and VL5 framework regions (within SEQ ID NOs: 469 and 476, respectively), or variants thereof;

Any of the framework sequences disclosed herein may be combined with any set of CDRs for PD-1 binding regions as described herein, or any of the CDRs within the VH and VL sequences shown with Tables 5B and 6B.

TABLE 3B
Exemplary VH sequences of PD-1 binding regions

| | SEQ | |
|---|---|---|
| | ID | |
| VH | NO | Sequence |
| VH0 | 464 | 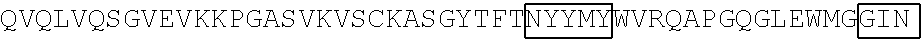 |
| | | 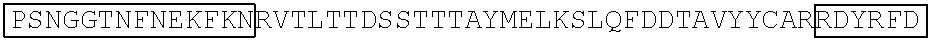 |
| | | 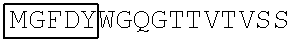 |
| VH1 | 465 | 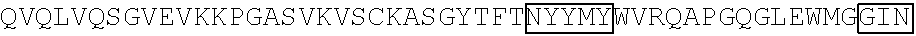 |
| | | 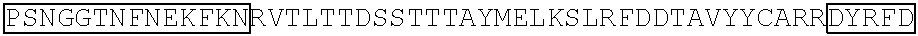 |
| | | 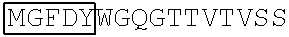 |
| VH2 | 466 | 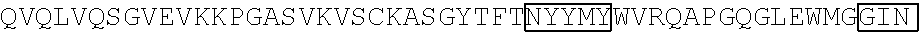 |
| | | 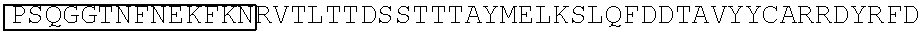 |
| | | 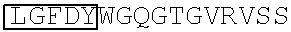 |
| VH3 | 467 | 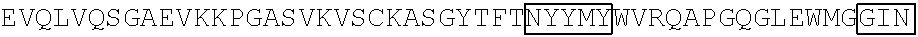 |
| | | 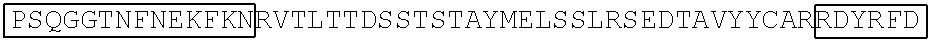 |
| | | 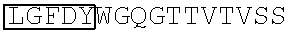 |
| VH4 | 468 | 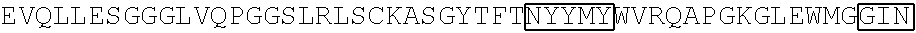 |
| | | 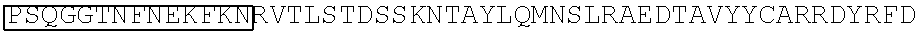 |
| | | 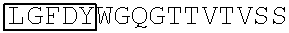 |
| VH5 | 469 | 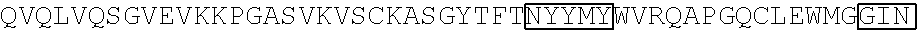 |
| | | 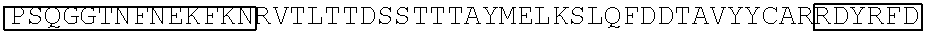 |
| | | 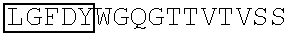 |
| VH6 | 470 | 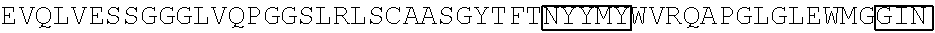 |
| | | 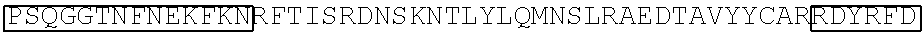 |
| | | 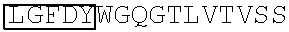 |

TABLE 3C
Exemplary VL sequences of PD-1 binding regions

| | SEQ | |
|---|---|---|
| | ID | |
| VL | NO | Sequence |
| VL0 | 471 | 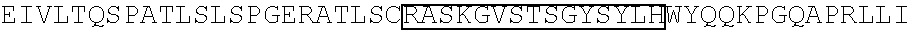 |
| | | 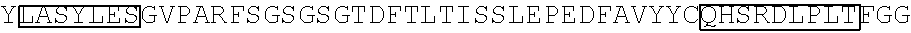 |
| | | GTKVEIKR |
| VL1 | 472 | 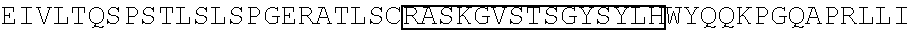 |
| | | 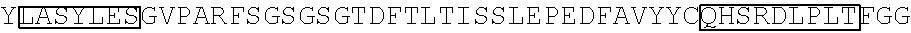 |
| | | GTKVEIKR |
| VL2 | 473 | 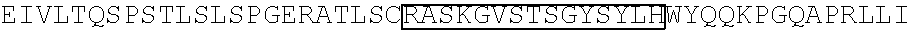 |
| | | 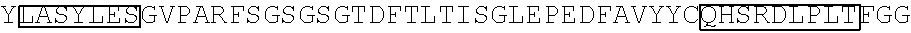 |
| | | GTKVEIKR |
| VL3 | 474 | 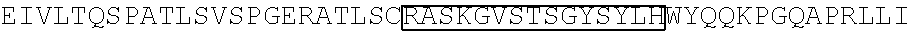 |
| | | 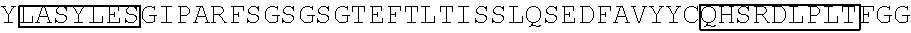 |
| | | GTKVEIKR |
| VL4 | 475 | 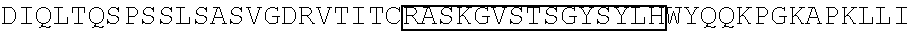 |
| | | 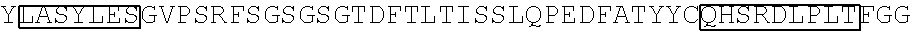 |
| | | GTKVEIKR |
| VL5 | 476 | EIVLTQSPATLSLSPGERATLSCRASKGVSTSGYSYLHWYQQKPGQAPRLLIY |
| | | 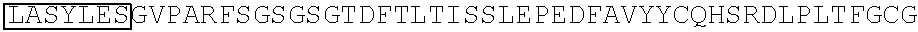 |
| | | TKVEIKR |

### Heavy Chain and Light Chain Domains

In some embodiments, the binding protein comprises a heavy chain comprising an amino acid sequence having at least 80% sequence identity with an amino acid sequence according to any one of SEQ ID NOs: 1-147, 656-678, and 702-713; and a light chain sequence comprising a sequence having at least 80% sequence identity to the amino acid sequence of any one of SEQ ID NOs: 148-194, 679-701, and 714-737. In some embodiments, the binding protein comprises a heavy chain comprising an amino acid sequence having at least 85% sequence identity with an amino acid sequence according to any one of SEQ ID NOs: 1-147, 656-678, and 702-713; and a light chain sequence comprising a sequence having at least 85% sequence identity to the amino acid sequence of any one of SEQ ID NOs: 148-194, 679-701, and 714-737. In some embodiments, the binding protein comprises a heavy chain comprising an amino acid sequence having at least 90% sequence identity with an amino acid sequence according to any one of SEQ ID NOs: 1-147, 656-678, and 702-713; and a light chain sequence comprising a sequence having at least 90% sequence identity to the amino acid sequence of any one of SEQ ID NOs: 148-194, 679-701, and 714-737. In some embodiments, the binding protein comprises a heavy chain comprising an amino acid sequence having at least 95% sequence identity with an amino acid sequence according to any one of SEQ ID NOs: 1-147, 656-678, and 702-713; and a light chain sequence comprising a sequence having at least 90% sequence identity to the amino acid sequence of any one of SEQ ID NOs: 148-194, 679-701, and 714-737. In some embodiments, the binding protein comprises a heavy chain comprising an amino acid sequence having at least 96% sequence identity with an amino acid sequence according to any one of SEQ ID NOs: 1-147, 656-678, and 702-713; and a light chain sequence comprising a sequence having at least 96% sequence identity to the amino acid sequence of any one of SEQ ID NOs: 148-194, 679-701, and 714-737. In some embodiments, the binding protein comprises a heavy chain comprising an amino acid sequence having at least 97% sequence identity with an amino acid sequence according to any one of SEQ ID NOs: 1-147, 656-678, and 702-713; and a light chain sequence comprising a sequence having at least 97% sequence identity to the amino acid sequence of any one of SEQ ID NOs: 148-194, 679-701, and 714-737. In some embodiments, the binding protein comprises a heavy chain comprising an amino acid sequence having at least 98% sequence identity with an amino acid sequence according to any one of SEQ ID NOs: 1-147, 656-678, and 702-713; and a light chain sequence comprising a sequence having at least 98% sequence identity to the amino acid sequence of any one of SEQ ID NOs: 148-194, 679-701, and 714-737. In some embodiments, the binding protein comprises a heavy chain comprising an amino acid sequence having at least 99% sequence identity with an amino acid sequence according to any one of SEQ ID NOs: 1-147, 656-678, and 702-713; and a light chain sequence comprising a sequence having at least 99% sequence identity to the amino acid sequence of any one of SEQ ID NOs: 148-194, 679-701, and 714-737. In some embodiments, the binding protein comprises a heavy chain comprising an amino acid sequence having 100% sequence identity with an amino acid sequence according to any one of SEQ ID NOs: 1-147, 656-678, and 702-713; and a light chain sequence comprising a sequence having 100% sequence identity with the amino acid sequence of any one of SEQ ID NOs: 148-194, 679-701, and 714-737.

Provided herein, in certain embodiments, are bispecific binding proteins that bind vascular endothelial growth factor (VEGF) and programmed death receptor 1 (PD-1), comprising: a) a heavy chain sequence comprising a sequence having at least 90% sequence identity to the amino acid sequence of any one of SEQ ID NOs: 22-23, 60, 62, 65-67, 70-72, 75-77, 142-143, 667-678, and 702-713; and b) a light chain sequence comprising a sequence having at least 80% sequence identity to the amino acid sequence of any one of SEQ ID NOs: 148-194, 679-701, and 714-737.

Further provided herein, in certain embodiments, are bispecific binding proteins that bind vascular endothelial growth factor (VEGF) and programmed death receptor 1 (PD-1), comprising: a) a heavy chain sequence comprising a sequence having at least 95% sequence identity to the amino acid sequence of any one of SEQ ID NOs: 1-21, 24-57, 58, 59, 61, 63, 64, 68, 69, 73, 74, 78-97, 130-141, 144-147, and 656-666; and b) a light chain sequence comprising a sequence having at least 80% sequence identity to the amino acid sequence of any one of SEQ ID NOs: 148-194, 679-701, and 714-737.

Further provided herein, in certain embodiments, are bispecific binding proteins that bind vascular endothelial growth factor (VEGF) and programmed death receptor 1 (PD-1), comprising: a) a heavy chain sequence comprising the amino acid sequence of any one of SEQ ID NOs: 98-127 and 129; and b) a light chain sequence comprising a sequence having at least 80% sequence identity to the amino acid sequence of any one of SEQ ID NOs: 148-194, 679-701, and 714-737.

TABLE 4A
Sequences of Heavy Chains and Light Chains of Multispecific Binding Proteins

| | SEQ ID | | SEQ | |
|---|---|---|---|---|
| Construct Name | NO | Heavy Chain | ID NO | Light Chain |
| PAL054-0101.1L | 1 | EVQLVESGGGLVQPGGSLRLSCAASGYTFTNY | 148 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0101.1La | 2 | EVQLVESGGGLVQPGGSLRLSCAASGYTFTNY | 149 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0101.1Lb | 3 | EVQLVESGGGLVQPGGSLRLSCAASGYTFTNY | 150 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVLHEALHSHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0102.1L | 4 | EVQLVESGGGLVQPGGSLRLSCAASGYTFTNY | 151 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0102.1La | 5 | EVQLVESGGGLVQPGGSLRLSCAASGYTFTNY | 152 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0102.1Lb | 6 | EVQLVESGGGLVQPGGSLRLSCAASGYTFTNY | 153 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVLHEALHSHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0103.1L | 7 | EVQLVESGGGLVQPGGSLRLSCAASGYTFTNY | 154 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0103.1La | 8 | EVQLVESGGGLVQPGGSLRLSCAASGYTFTNY | 155 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0103.1Lb | 9 | EVQLVESGGGLVQPGGSLRLSCAASGYTFTNY | 156 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVLHEALHSHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0104.1L | 10 | EVQLVESGGGLVQPGGSLRLSCAASGYTFTNY | 157 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0104.1La | 11 | EVQLVESGGGLVQPGGSLRLSCAASGYTFTNY | 158 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0104.1Lb | 12 | EVQLVESGGGLVQPGGSLRLSCAASGYTFTNY | 159 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVLHEALHSHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0105.1L | 13 | EVQLVESGGGLVQPGGSLRLSCAASGYTFTNY | 160 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0105.1La | 14 | EVQLVESGGGLVQPGGSLRLSCAASGYTFTNY | 161 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0105.1Lb | 15 | EVQLVESGGGLVQPGGSLRLSCAASGYTFTNY | 162 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVLHEALHSHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0106.1L | 16 | EVQLVESGGGLVQPGGSLRLSCAASGYTFTNY | 163 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0106.1La | 17 | EVQLVESGGGLVQPGGSLRLSCAASGYTFTNY | 164 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0106.1Lb | 18 | EVQLVESGGGLVQPGGSLRLSCAASGYTFTNY | 165 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVLHEALHSHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0107.1L | 19 | EVQLVESGGGLVQPGGSLRLSCAASGYTFTNY | 166 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKSSGGGGSGGGGSGGGGSGGGGSQV | | |
| | | QLVQSGVEVKKPGASVKVSCKASGYTFTNYYM | | |
| | | YWVRQAPGQGLEWMGGINPSQGGTNFNEKFKN | | |
| | | RVTLTTDSSTTTAYMELKSLQFDDTAVYYCAR | | |
| | | RDYRFDLGFDYWGQGTTVTVSS | | |
| PAL054-0107.1La | 20 | EVQLVESGGGLVQPGGSLRLSCAASGYTFTNY | 167 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKSSGGGGSGGGGSGGGGSGGGGSQV | | |
| | | QLVQSGVEVKKPGASVKVSCKASGYTFTNYYM | | |
| | | YWVRQAPGQGLEWMGGINPSQGGTNFNEKFKN | | |
| | | RVTLTTDSSTTTAYMELKSLQFDDTAVYYCAR | | |
| | | RDYRFDLGFDYWGQGTTVTVSS | | |
| PAL054-0108.1La | 21 | EVQLVESGGGLVQPGGSLRLSCAASGYTFTNY | 168 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | CGTKVEIKGGGGSGGGGSGGGGSGGGGSQVQL | | |
| | | VQSGVEVKKPGASVKVSCKASGYTFTNYYMYW | | |
| | | VRQAPGQCLEWMGGINPSQGGTNFNEKFKNRV | | |
| | | TLTTDSSTTTAYMELKSLQFDDTAVYYCARRD | | |
| | | YRFDLGFDYWGQGTTVTVSS | | |
| PAL054-0109.1La | 22 | EVQLVESGGGLVQPGGSLRLSCAASGYTFTNY | 169 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGYTFTNYYMYWVRQ | | |
| | | APGKGLEWMGGINPSQGGTNFNEKFKNRFTIS | | |
| | | RDNSKNTLYLQMNSLRAEDTAVYYCARRDYRF | | |
| | | DLGFDYWGQGTLVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGGGTKVEIKR | | |
| PAL054-0110.1La | 23 | EVQLVESGGGLVQPGGSLRLSCAASGYTFTNY | 170 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGYTFTNYYMYWVRQ | | |
| | | APGKCLEWMGGINPSQGGTNFNEKFKNRFTIS | | |
| | | RDNSKNTLYLQMNSLRAEDTAVYYCARRDYRF | | |
| | | DLGFDYWGQGTLVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGCGTKVEIKR | | |
| PAL054-0111.1La | 24 | EVQLVESGGGLVQPGGSLRLSCAASGYTFTNY | 171 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTGVRVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0112.1La | 25 | EVQLVESGGGLVQPGGSLRLSCAASGYTFTNY | 172 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTGVRVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0113.1La | 26 | EVQLVESGGGLVQPGGSLRLSCAASGYTFTNY | 173 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0114.1L | 27 | EVQLVESGGGLVQPGGSLRLSCAASGYTFTNY | 174 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0114.1La | 28 | EVQLVESGGGLVQPGGSLRLSCAASGYTFTNY | 175 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0115.1La | 29 | EVQLVESGGGLVQPGGSLRLSCAASGYTFTNY | 176 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGGGTKVEIKR | | |
| PAL054-0116.1L | 30 | EVQLVESGGGLVQPGGSLRLSCAASGYTFTNY | 177 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0116.1La | 31 | EVQLVESGGGLVQPGGSLRLSCAASGYTFTNY | 178 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0117.1La | 32 | EVQLVESGGGLVQPGGSLRLSCAASGYTFTNY | 179 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGGGTKVEIKR | | |
| PAL054-0118.1La | 33 | EVQLVESGGGLVQPGGSLRLSCAASGYTFTNY | 180 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0119.1La | 34 | EVQLVESGGGLVQPGGSLRLSCAASGYTFTNY | 181 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0120.1La | 35 | EVQLVESGGGLVQPGGSLRLSCAASGYTFTNY | 182 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGGGIKVEIKR | | |
| PAL054-0121.1La | 36 | EVQLVESGGGLVQPGGSLRLSCAASGYTFTNY | 183 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0122.1La | 37 | EVQLVESGGGLVQPGGSLRLSCAASGYTFTNY | 184 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGGGTKVEIKR | | |
| PAL054-0123.1La | 38 | EVQLVESGGGLVQPGGSLRLSCAASGYTFTNY | 185 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0124.1La | 39 | EVQLVESGGGLVQPGGSLRLSCAASGYTFTNY | 186 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0125.1La | 40 | EVQLVESGGGLVQPGGSLRLSCAASGYTFTNY | 187 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGGGTKVEIKR | | |
| PAL054-0126.1La | 41 | EVQLVESGGGLVQPGGSLRLSCAASGYTFTNY | 188 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0127.1La | 42 | EVQLVESGGGLVQPGGSLRLSCAASGYTFTNY | 189 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGGGTKVEIKR | | |
| PAL054-0128.1La | 43 | EVQLVESGGGLVQPGGSLRLSCAASGYTFTNY | 190 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0129.1La | 44 | EVQLVESGGGLVQPGGSLRLSCAASGYTFTNY | 191 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0130.1La | 45 | EVQLVESGGGLVQPGGSLRLSCAASGYTFTNY | 192 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLRFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGCGTKVEIKR | | |
| PAL054-0131.1La | 46 | EVQLVESGGGLVQPGGSLRLSCAASGYTFTNY | 193 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0132.1La | 47 | EVQLVESGGGLVQPGGSLRLSCAASGYTFTNY | 194 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGCGTKVEIKR | | |
| PAL054-0133.1La | 48 | EVQLVESGGGLVQPGGSLRLSCAASGYTFTNY | 195 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0134.1La | 49 | EVQLVESGGGLVQPGGSLRLSCAASGYTFTNY | 196 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0135.1La | 50 | EVQLVESGGGLVQPGGSLRLSCAASGYTFTNY | 197 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGCGTKVEIKR | | |
| PAL054-0136.1La | 51 | EVQLVESGGGLVQPGGSLRLSCAASGYTFTNY | 198 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0137.1La | 52 | EVQLVESGGGLVQPGGSLRLSCAASGYTFTNY | 199 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGCGTKVEIKR | | |
| PAL054-0138.1La | 53 | EVQLVESGGGLVQPGGSLRLSCAASGYTFTNY | 200 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0139.1La | 54 | EVQLVESGGGLVQPGGSLRLSCAASGYTFTNY | 201 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0140.1La | 55 | EVQLVESGGGLVQPGGSLRLSCAASGYTFTNY | 202 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLRFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGCGTKVEIKR | | |
| PAL054-0141.1La | 56 | EVQLVESGGGLVQPGGSLRLSCAASGYTFTNY | 203 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0142.1La | 57 | EVQLVESGGGLVQPGGSLRLSCAASGYTFTNY | 204 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPSTLSLSPGERATLSCRASK | | |
| | | GVSTSGYSYLHWYQQKPGQAPRLLIYLASYLE | | |
| | | SGVPARFSGSGSGTDFTLTISGLEPEDFAVYY | | |
| | | CQHSRDLPLTFGCGTKVEIKR | | |
| PAL054-0143.1La | 58 | EVQLVESGGGLVQPGGSLRLSCAASGYTFTNY | 205 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0144.1La | 59 | EVQLVESGGGLVQPGGSLRLSCAASGYTFTNY | 206 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGGGTKVEIKR | | |
| PAL054-0145.1La | 60 | EVQLVESGGGLVQPGGSLRLSCAASGYTFTNY | 207 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGGGTKVEIKR | | |
| PAL054-0146.1La | 61 | EVQLVESGGGLVQPGGSLRLSCAASGYTFTNY | 208 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQGLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0147.1La | 62 | EVQLVESGGGLVQPGGSLRLSCAASGYTFTNY | 209 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQGLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGGGIKVEIKR | | |
| PAL054-0148.1L | 63 | EVQLVESGGGLVQPGGSLRLSCAASGYTFTNY | 210 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQGLEWMGGINPSQGGTNFNEKFKNRVTLI | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGGGTKVEIKR | | |
| PAL054-0148.1La | 64 | EVQLVESGGGLVQPGGSLRLSCAASGYTFTNY | 211 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQGLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGGGTKVEIKR | | |
| PAL054-0149.1La | 65 | EVQLVESGGGLVQPGGSLRLSCAASGYTFTNY | 212 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLLES | | |
| | | GGGLVQPGGSLRLSCKASGYTFTNYYMYWVRQ | | |
| | | APGKGLEWMGGINPSQGGTNFNEKFKNRVTLS | | |
| | | TDSSKNTAYLQMNSLRAEDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0150.1La | 66 | EVQLVESGGGLVQPGGSLRLSCAASGYTFTNY | 213 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLLES | | |
| | | GGGLVQPGGSLRLSCKASGYTFTNYYMYWVRQ | | |
| | | APGKGLEWMGGINPSQGGTNFNEKFKNRVTLS | | |
| | | TDSSKNTAYLQMNSLRAEDTAVYYCARRDYRF | | |
| | | DLGFDYWGQGTTVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGGGTKVEIKR | | |
| PAL054-0151.1La | 67 | EVQLVESGGGLVQPGGSLRLSCAASGYTFTNY | 214 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLLES | | |
| | | GGGLVQPGGSLRLSCKASGYTFTNYYMYWVRQ | | |
| | | APGKGLEWMGGINPSQGGTNFNEKFKNRVILS | | |
| | | TDSSKNTAYLQMNSLRAEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGGGTKVEIKR | | |
| PAL054-0152.1La | 68 | EVQLVESGGGLVQPGGSLRLSCAASGYTFTNY | 215 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0153.1La | 69 | EVQLVESGGGLVQPGGSLRLSCAASGYTFTNY | 216 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGCGTKVEIKR | | |
| PAL054-0154.1La | 70 | EVQLVESGGGLVQPGGSLRLSCAASGYTFTNY | 217 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0155.1La | 71 | EVQLVESGGGLVQPGGSLRLSCAASGYTFTNY | 218 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0156.1La | 72 | EVQLVESGGGLVQPGGSLRLSCAASGYTFTNY | 219 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGCGTKVEIKR | | |
| PAL054-0157.1L | 73 | EVQLVESGGGLVQPGGSLRLSCAASGYTFTNY | 220 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0157.1La | 74 | EVQLVESGGGLVQPGGSLRLSCAASGYTFTNY | 221 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSQGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0158.1La | 75 | EVQLVESGGGLVQPGGSLRLSCAASGYTFTNY | 222 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLLES | | |
| | | GGGLVQPGGSLRLSCKASGYTFTNYYMYWVRQ | | |
| | | APGKCLEWMGGINPSQGGTNFNEKFKNRVTLS | | |
| | | TDSSKNTAYLQMNSLRAEDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0159.1La | 76 | EVQLVESGGGLVQPGGSLRLSCAASGYTFTNY | 223 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLLES | | |
| | | GGGLVQPGGSLRLSCKASGYTFTNYYMYWVRQ | | |
| | | APGKCLEWMGGINPSQGGTNFNEKFKNRVTLS | | |
| | | TDSSKNTAYLQMNSLRAEDTAVYYCARRDYRF | | |
| | | DLGFDYWGQGTTVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGCGTKVEIKR | | |
| PAL054-0160.1La | 77 | EVQLVESGGGLVQPGGSLRLSCAASGYTFTNY | 224 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLLES | | |
| | | GGGLVQPGGSLRLSCKASGYTFTNYYMYWVRQ | | |
| | | APGKCLEWMGGINPSQGGTNFNEKFKNRVTLS | | |
| | | TDSSKNTAYLQMNSLRAEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0161.1L | 78 | EVQLVESGGGLVQPGGSLRLSCAASGYTFTNY | 225 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSNGGTNFNEKFKNRV | | |
| | | TLTTDSSTTTAYMELKSLQFDDTAVYYCARRD | | |
| | | YRFDMGFDYWGQGTTVTVSS | | |
| PAL054-0161.1La | 79 | EVQLVESGGGLVQPGGSLRLSCAASGYTFTNY | 226 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSNGGTNFNEKFKNRV | | |
| | | TLTTDSSTTTAYMELKSLQFDDTAVYYCARRD | | |
| | | YRFDMGFDYWGQGTTVTVSS | | |
| PAL054-0162.1L | 80 | EVQLVESGGGLVQPGGSLRLSCAASGYTFTNY | 227 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSRGGTNFNEKFKNRV | | |
| | | TLTTDSSTTTAYMELKSLQFDDTAVYYCARRD | | |
| | | YRFDLGFDYWGQGTTVTVSS | | |
| PAL054-0162.1La | 81 | EVQLVESGGGLVQPGGSLRLSCAASGYTFTNY | 228 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSRGGTNFNEKFKNRV | | |
| | | TLTTDSSTTTAYMELKSLQFDDTAVYYCARRD | | |
| | | YRFDLGFDYWGQGTTVTVSS | | |
| PAL054-0163.1L | 82 | EVQLVESGGGLVQPGGSLRLSCAASGYTFTNY | 229 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSNGGTNFNEKFKNRV | | |
| | | TLTTDSSTTTAYMELKSLRFDDTAVYYCARRD | | |
| | | YRFDMGFDYWGQGTTVTVSS | | |
| PAL054-0163.1La | 83 | EVQLVESGGGLVQPGGSLRLSCAASGYTFTNY | 230 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PATLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSNGGTNFNEKFKNRV | | |
| | | TLTTDSSTTTAYMELKSLRFDDTAVYYCARRD | | |
| | | YRFDMGFDYWGQGTTVTVSS | | |
| PAL054-0164.1L | 84 | EVQLVESGGGLVQPGGSLRLSCAASGYTFTNY | 231 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | IDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PSTLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSNGGTNFNEKFKNRV | | |
| | | TLTTDSSTTTAYMELKSLQFDDTAVYYCARRD | | |
| | | YRFDMGFDYWGQGTTVTVSS | | |
| PAL054-0164.1La | 85 | EVQLVESGGGLVQPGGSLRLSCAASGYTFTNY | 232 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLILSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEIVLTQS | | |
| | | PSTLSLSPGERATLSCRASKGVSTSGYSYLHW | | |
| | | YQQKPGQAPRLLIYLASYLESGVPARFSGSGS | | |
| | | GTDFTLTISSLEPEDFAVYYCQHSRDLPLTFG | | |
| | | GGTKVEIKGGGGSGGGGSGGGGSGGGGSQVQL | | |
| | | VQSGVEVKKPGASVKVSCKASGYTFTNYYMYW | | |
| | | VRQAPGQGLEWMGGINPSNGGTNFNEKFKNRV | | |
| | | TLTTDSSTTTAYMELKSLQFDDTAVYYCARRD | | |
| | | YRFDMGFDYWGQGTTVTVSS | | |
| PAL054-0165.1L | 86 | EVQLVESGGGLVQPGGSLRLSCAASGYTFTNY | 233 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQGLEWMGGINPSNGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGGGTKVEIKR | | |
| PAL054-0165.1La | 87 | EVQLVESGGGLVQPGGSLRLSCAASGYTFTNY | 234 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQGLEWMGGINPSNGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGGGTKVEIKR | | |
| PAL054-0166.1L | 88 | EVQLVESGGGLVQPGGSLRLSCAASGYTFTNY | 235 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSNGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0166.1La | 89 | EVQLVESGGGLVQPGGSLRLSCAASGYTFTNY | 236 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSNGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0167.1L | 90 | EVQLVESGGGLVQPGGSLRLSCAASGYTFTNY | 237 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSRGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0167.1La | 91 | EVQLVESGGGLVQPGGSLRLSCAASGYTFTNY | 238 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSRGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0168.1L | 92 | EVQLVESGGGLVQPGGSLRLSCAASGYTFTNY | 239 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSQGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0168.1La | 93 | EVQLVESGGGLVQPGGSLRLSCAASGYTFTNY | 240 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSQGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0169.1L | 94 | EVQLVESGGGLVQPGGSLRLSCAASGYTFTNY | 241 | DIQMTQSPSSLSASVDGRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSRGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0169.1La | 95 | EVQLVESGGGLVQPGGSLRLSCAASGYTFTNY | 242 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSRGGTNFNEKFKNRVTLT | | |
| | | TDSSTSTAYMELSSLRSEDTAVYYCARRDYRF | | |
| | | DMGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0170.1L | 96 | EVQLVESGGGLVQPGGSLRLSCAASGYTFTNY | 243 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSNGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0170.1La | 97 | EVQLVESGGGLVQPGGSLRLSCAASGYTFTNY | 244 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVQS | | |
| | | GAEVKKPGASVKVSCKASGYTFTNYYMYWVRQ | | |
| | | APGQCLEWMGGINPSNGGTNFNEKFKNRVTLT | | |
| | | TDSSTSTAYMELSSLRSEDTAVYYCARRDYRF | | |
| | | DLGFDYWGQGTTVTVSSGGGGSGGGGSGGGGS | | |
| | | GGGGSDIQLTQSPSSLSASVGDRVTITCRASK | | |
| | | GVSTSGYSYLHWYQQKPGKAPKLLIYLASYLE | | |
| | | SGVPSRFSGSGSGTDFTLTISSLQPEDFATYY | | |
| | | CQHSRDLPLTFGCGTKVEIKR | | |
| PAL054-0201.1L | 98 | QVQLVQSGVEVKKPGASVKVSCKASGYTFTNY | 245 | EIVLTQSPATLSLSPGERATLSCRASKGVSTSGY |
| | | YMYWVRQAPGQGLEWMGGINPSNGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDMGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLILSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0201.1La | 99 | QVQLVQSGVEVKKPGASVKVSCKASGYTFTNY | 246 | EIVLTQSPATLSLSPGERATLSCRASKGVSTSGY |
| | | YMYWVRQAPGQGLEWMGGINPSNGGTNFNEKF | | SYLHWYQQKPGQAPRLLIYLASYLESGVPARFSG |
| | | KNRVTLITDSSTTTAYMELKSLQFDDTAVYYC | | SGSGTDFTLTISSLEPEDFAVYYCQHSRDLPLTF |
| | | ARRDYRFDMGEDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0201.1Lb | 100 | QVQLVQSGVEVKKPGASVKVSCKASGYTFTNY | 247 | EIVLTQSPATLSLSPGERATLSCRASKGVSTSGY |
| | | YMYWVRQAPGQGLEWMGGINPSNGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDMGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLIVD | | |
| | | KSRWQQGNVFSCSVLHEALHSHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0202.1L | 101 | QVQLVQSGVEVKKPGASVKVSCKASGYTFTNY | 248 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0202.1La | 102 | QVQLVQSGVEVKKPGASVKVSCKASGYTFTNY | 249 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0202.1Lb | 103 | QVQLVQSGVEVKKPGASVKVSCKASGYTFTNY | 250 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVLHEALHSHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0203.1L | 104 | QVQLVQSGVEVKKPGASVKVSCKASGYTFTNY | 251 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLILSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0203.1La | 105 | QVQLVQSGVEVKKPGASVKVSCKASGYTFTNY | 252 | EIVLTQSPATLSLSPGERATLSCRASKGVSTSGY |
| | | YMYWVRQAPGQGLEWMGGINPSRGGINFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0203.1Lb | 106 | QVQLVQSGVEVKKPGASVKVSCKASGYTFTNY | 253 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVLHEALHSHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0204.1L | 107 | QVQLVQSGVEVKKPGASVKVSCKASGYTFTNY | 254 | EIVLTQSPATLSLSPGERATLSCRASKGVSTSGY |
| | | YMYWVRQAPGQGLEWMGGINPSNGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDMGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0204.1La | 108 | QVQLVQSGVEVKKPGASVKVSCKASGYTFTNY | 255 | EIVLTQSPATLSLSPGERATLSCRASKGVSTSGY |
| | | YMYWVRQAPGQGLEWMGGINPSNGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDMGEDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLILSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLIVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0204.1Lb | 109 | QVQLVQSGVEVKKPGASVKVSCKASGYTFTNY | 256 | EIVLTQSPATLSLSPGERATLSCRASKGVSTSGY |
| | | YMYWVRQAPGQGLEWMGGINPSNGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDMGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVLHEALHSHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0205.1L | 110 | QVQLVQSGVEVKKPGASVKVSCKASGYTFTNY | 257 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLITDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0205.1La | 111 | QVQLVQSGVEVKKPGASVKVSCKASGYTFTNY | 258 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0205.1Lb | 112 | QVQLVQSGVEVKKPGASVKVSCKASGYTFTNY | 259 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVLHEALHSHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0206.1L | 113 | QVQLVQSGVEVKKPGASVKVSCKASGYTFTNY | 260 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0206.1La | 114 | QVQLVQSGVEVKKPGASVKVSCKASGYTFTNY | 26 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLITDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLILSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0206.1Lb | 115 | QVQLVQSGVEVKKPGASVKVSCKASGYTFTNY | 262 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLMI | | |
| | | SRTPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVLHEALHSHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0301.1L | 116 | EVQLVESGGGLVQPGGSLRLSCAASGYTFTNY | 263 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREWVSVITWSGGITYYADSVKGRFTIS | | |
| | | RDNSKNTLYLQMNSLRAEDTAVYYCAGDKHQS | | |
| | | SWYDYWGQGTLVTVSS | | |
| PAL054-0301.1La | 117 | EVQLVESGGGLVQPGGSLRLSCAASGYTFTNY | 264 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREWVSVITWSGGITYYADSVKGRFTIS | | |
| | | RDNSKNTLYLQMNSLRAEDTAVYYCAGDKHQS | | |
| | | SWYDYWGQGTLVTVSS | | |
| PAL054-0302.1L | 118 | EVQLVESGGGLVQPGGSLRLSCAASGYTFTNY | 265 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREWVSVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0302.1La | 119 | EVQLVESGGGLVQPGGSLRLSCAASGYTFTNY | 266 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREWVSVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0303.1L | 120 | EVQLVESGGGLVQPGGSLRLSCAASGYTFTNY | 267 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREFVSVITWSGGITYYADSVKGRFTIS | | |
| | | RDNSKNTLYLQMNSLRAEDTAVYYCAGDKHQS | | |
| | | SWYDYWGQGTLVTVSS | | |
| PAL054-0303.1La | 121 | EVQLVESGGGLVQPGGSLRLSCAASGYTFTNY | 268 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREFVSVITWSGGITYYADSVKGRFTIS | | |
| | | RDNSKNTLYLQMNSLRAEDTAVYYCAGDKHQS | | |
| | | SWYDYWGQGTLVTVSS | | |
| PAL054-0304.1L | 122 | EVQLVESGGGLVQPGGSLRLSCAASGYTFTNY | 269 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREFVSVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0304.1La | 123 | EVQLVESGGGLVQPGGSLRLSCAASGYTFTNY | 270 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREFVSVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0305.1L | 124 | EVQLVESGGGLVQPGGSLRLSCAASGYTFTNY | 271 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREFVAVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0305.1La | 125 | EVQLVESGGGLVQPGGSLRLSCAASGYTFTNY | 272 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREFVAVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0306.1L | 126 | EVQLVESGGGLVQPGGSLRLSCAASGYTFTNY | 273 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREWVAVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0306.1La | 127 | EVQLVESGGGLVQPGGSLRLSCAASGYTFTNY | 274 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGSIASIHAMGWVRQ | | |
| | | APGKEREWVAVITWSGGITYYADSVKGRFTIS | | |
| | | RDNSKNTVYLQMNSLRAEDTAVYYCAGDKHQS | | |
| | | SWYDYWGQGTLVTVSS | | |
| PAL054-0001.1L | 128 | EVQLVESGGGLVQPGGSLRLSCAASGYTFTNY | 275 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGFAFSSYDMSWVRQ | | |
| | | APGKGLDWVATISGGGRYTYYPDSVKGRFTIS | | |
| | | RDNSKNNLYLQMNSLRAEDTALYYCANRYGEA | | |
| | | WFAYWGQGTLVTVSSGGGGSGGGGSGGGGSGG | | |
| | | GGSDIQMTQSPSSMSASVGDRVTFTCRASQDI | | |
| | | NTYLSWFQQKPGKSPKTLIYRANRLVSGVPSR | | |
| | | FSGSGSGQDYTLTISSLQPEDMATYYCLQYDE | | |
| | | FPLTFGAGTKLELKR | | |
| PAL054-0001.1La | 129 | EVQLVESGGGLVQPGGSLRLSCAASGYTFTNY | 276 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGFAFSSYDMSWVRQ | | |
| | | APGKGLDWVATISGGGRYTYYPDSVKGRFTIS | | |
| | | RDNSKNNLYLQMNSLRAEDTALYYCANRYGEA | | |
| | | WFAYWGQGTLVTVSSGGGGSGGGGSGGGGSGG | | |
| | | GGSDIQMTQSPSSMSASVGDRVTFTCRASQDI | | |
| | | NTYLSWFQQKPGKSPKTLIYRANRLVSGVPSR | | |
| | | FSGSGSGQDYTLTISSLQPEDMATYYCLQYDE | | |
| | | FPLTFGAGTKLELKR | | |
| PAL054-0101.1L | 130 | EVQLVESGGGLVQPGGSLRLSCAASGYTFTNY | 277 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0101.1La | 131 | EVQLVESGGGLVQPGGSLRLSCAASGYTFTNY | 278 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0102.1L | 132 | EVQLVESGGGLVQPGGSLRLSCAASGYTFTNY | 279 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0102.1La | 133 | EVQLVESGGGLVQPGGSLRLSCAASGYTFTNY | 280 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0103.1L | 134 | EVQLVESGGGLVQPGGSLRLSCAASGYTFTNY | 281 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0103.1La | 135 | EVQLVESGGGLVQPGGSLRLSCAASGYTFTNY | 282 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQGLEWMGGINPSRGGINFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGGGTKVEIKR | | |
| PAL054-0104.1L | 136 | EVQLVESGGGLVQPGGSLRLSCAASGYTFTNY | 283 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0104.1La | 137 | EVQLVESGGGLVQPGGSLRLSCAASGYTFTNY | 284 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSNGGTNFNEKFKNRVTLT | | |
| | | TDSSTTTAYMELKSLQFDDTAVYYCARRDYRF | | |
| | | DMGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0105.1L | 138 | EVQLVESGGGLVQPGGSLRLSCAASGYTFTNY | 285 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0105.1La | 139 | EVQLVESGGGLVQPGGSLRLSCAASGYTFTNY | 286 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSQGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0106.1L | 140 | EVQLVESGGGLVQPGGSLRLSCAASGYTFTNY | 287 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0106.1La | 141 | EVQLVESGGGLVQPGGSLRLSCAASGYTFTNY | 288 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSQVQLVQS | | |
| | | GVEVKKPGASVKVSCKASGYTFTNYYMYWVRQ | | |
| | | APGQCLEWMGGINPSRGGTNFNEKFKNRVTLT | | |
| | | TDSSTTTAYMELKSLQFDDTAVYYCARRDYRF | | |
| | | DLGFDYWGQGTTVTVSSGGGGSGGGGSGGGGS | | |
| | | GGGGSEIVLTQSPATLSLSPGERATLSCRASK | | |
| | | GVSTSGYSYLHWYQQKPGQAPRLLIYLASYLE | | |
| | | SGVPARFSGSGSGTDFTLTISSLEPEDFAVYY | | |
| | | CQHSRDLPLTFGCGTKVEIKR | | |
| PAL054-0109.1La | 142 | EVQLVESGGGLVQPGGSLRLSCAASGYTFTNY | 289 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGYTFTNYYMYWVRQ | | |
| | | APGKGLEWMGGINPSQGGTNFNEKFKNRFTIS | | |
| | | RDNSKNTLYLQMNSLRAEDTAVYYCARRDYRF | | |
| | | DLGFDYWGQGTLVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGGGTKVEIKR | | |
| PAL054-0110.1La | 143 | EVQLVESGGGLVQPGGSLRLSCAASGYTFTNY | 290 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGKGGGGSGGGGSGGGGSGGGGSEVQLVES | | |
| | | GGGLVQPGGSLRLSCAASGYTFTNYYMYWVRQ | | |
| | | APGKCLEWMGGINPSQGGTNFNEKFKNRFTIS | | |
| | | RDNSKNTLYLQMNSLRAEDTAVYYCARRDYRF | | |
| | | DLGFDYWGQGTLVTVSSGGGGSGGGGSGGGGS | | |
| | | GGGGSEIVLTQSPATLSVSPGERATLSCRASK | | |
| | | GVSTSGYSYLHWYQQKPGQAPRLLIYLASYLE | | |
| | | SGIPARFSGSGSGTEFTLTISSLQSEDFAVYY | | |
| | | CQHSRDLPLTFGCGTKVEIKR | | |
| PAL054-0202.1La | 144 | QVQLVQSGVEVKKPGASVKVSCKASGYTFTNY | 291 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0203.1La | 145 | QVQLVQSGVEVKKPGASVKVSCKASGYTFTNY | 292 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KGLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGQGTKVEIKR | | |
| PAL054-0205.1La | 146 | QVQLVQSGVEVKKPGASVKVSCKASGYTFTNY | 293 | EIVLTQSPATLSLSPGERATLSCRASKGVSTSGY |
| | | YMYWVRQAPGQGLEWMGGINPSQGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLILSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0206.1La | 147 | QVQLVQSGVEVKKPGASVKVSCKASGYTFTNY | 294 | EIVLTQSPATLSLSPGERATLSCRASKGVSTSGY |
| | | YMYWVRQAPGQGLEWMGGINPSRGGTNFNEKF | | SYLHWYQQKPGQAPRLLIYLASYLESGVPARFSG |
| | | KNRVTLTTDSSTTTAYMELKSLQFDDTAVYYC | | SGSGTDFTLTISSLEPEDFAVYYCQHSRDLPLTF |
| | | ARRDYRFDLGFDYWGQGTTVTVSSASTKGPSV | | GGGTKVEIKRTVAAPSVFIFPPSDEQLKSGTASV |
| | | FPLAPSSKSTSGGTAALGCLVKDYFPEPVTVS | | VCLLNNFYPREAKVQWKVDNALQSGNSQESVTEQ |
| | | WNSGALTSGVHTFPAVLQSSGLYSLSSVVTVP | | DSKDSTYSLSSTLTLSKADYEKHKVYACEVTHQG |
| | | SSSLGTQTYICNVNHKPSNTKVDKKVEPKSCD | | LSSPVTKSFNRGEC |
| | | KTHTCPPCPAPEAAGGPSVFLFPPKPKDTLYI | | |
| | | TREPEVTCVVVDVSHEDPEVKFNWYVDGVEVH | | |
| | | NAKTKPREEQYNSTYRVVSVLTVLHQDWLNGK | | |
| | | EYKCKVSNKALPAPIEKTISKAKGQPREPQVY | | |
| | | TLPPSRDELTKNQVSLTCLVKGFYPSDIAVEW | | |
| | | ESNGQPENNYKTTPPVLDSDGSFFLYSKLTVD | | |
| | | KSRWQQGNVFSCSVMHEALHNHYTQKSLSLSP | | |
| | | GKGGGGSGGGGSGGGGSGGGGSEVQLVESGGG | | |
| | | LVQPGGSLRLSCAASGYTFTNYGMNWVRQAPG | | |
| | | KCLEWVGWINTYTGEPTYAADFKRRFTFSLDT | | |
| | | SKSTAYLQMNSLRAEDTAVYYCAKYPHYYGSS | | |
| | | HWYFDVWGQGTLVTVSSGGGGSGGGGSGGGGS | | |
| | | GGGGSDIQMTQSPSSLSASVGDRVTITCSASQ | | |
| | | DISNYLNWYQQKPGKAPKVLIYFTSSLHSGVP | | |
| | | SRFSGSGSGTDFTLTISSLQPEDFATYYCQQY | | |
| | | STVPWTFGCGTKVEIKR | | |
| PAL054-0171.1L | 656 | EVQLVESGGGLVQPGGSLRLSCAASGYTFTNY | 679 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSQGGTNFNEKFKNRVTLTT | | |
| | | DSSTSTAYMELSSLRSEDTAVYYCARRDYRFD | | |
| | | LGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0171.1La | 657 | EVQLVESGGGLVQPGGSLRLSCAASGYTFTNY | 680 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSQGGTNFNEKFKNRVILIT | | |
| | | DSSTSTAYMELSSLRSEDTAVYYCARRDYRFD | | |
| | | LGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0172.1L | 658 | EVQLVESGGGLVQPGGSLRLSCAASGYTFTNY | 681 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSNGGTNFNEKFKNRVTLTT | | |
| | | DSSTSTAYMELSSLRSEDTAVYYCARRDYRFD | | |
| | | MGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0172.1La | 659 | EVQLVESGGGLVQPGGSLRLSCAASGYTFTNY | 682 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSNGGTNFNEKFKNRVTLTT | | |
| | | DSSTSTAYMELSSLRSEDTAVYYCARRDYRFD | | |
| | | MGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0173.1L | 660 | EVQLVESGGGLVQPGGSLRLSCAASGYTFTNY | 683 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSQGGTNFNEKFKNRVTLTT | | |
| | | DSSTSTAYMELSSLRSEDTAVYYCARRDYRFD | | |
| | | MGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0174.1L | 661 | EVQLVESGGGLVQPGGSLRLSCAASGYTFTNY | 684 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSRGGTNFNEKFKNRVTLTT | | |
| | | DSSTSTAYMELSSLRSEDTAVYYCARRDYRFD | | |
| | | MGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0175.1L | 662 | EVQLVESGGGLVQPGGSLRLSCAASGYTFTNY | 685 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVQSG | | |
| | | AEVKKPGASVKVSCKASGYTFTNYYMYWVRQA | | |
| | | PGQCLEWMGGINPSNGGTNFNEKFKNRVTLTT | | |
| | | DSSTSTAYMELSSLRSEDTAVYYCARRDYRFD | | |
| | | LGFDYWGQGTTVTVSSGGGGSGGGGSGGGGSG | | |
| | | GGGSDIQLTQSPSSLSASVGDRVTITCRASKG | | |
| | | VSTSGYSYLHWYQQKPGKAPKLLIYLASYLES | | |
| | | GVPSRFSGSGSGTDFTLTISSLQPEDFATYYC | | |
| | | QHSRDLPLTFGCGTKVEIK | | |
| PAL054-0307.1L | 663 | EVQLVESGGGLVQPGGSLRLSCAASGYTFTNY | 686 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVESG | | |
| | | GGLVQPGGSLRLSCAASGSIASIHAMGWVRQA | | |
| | | PGKEREFVAVITWSGGITYYADSVKGRFTISR | | |
| | | DNSKNTVYLQMNSLRPEDTAVYYCAGDKHQSS | | |
| | | WYDYWGQGTLVTVSS | | |
| PAL054-0308.1L | 664 | EVQLVESGGGLVQPGGSLRLSCAASGYTFTNY | 687 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVESG | | |
| | | GGLVQPGGSLRLSCAASGSIASIHAMGWVRQA | | |
| | | PGKEREFVAVITWSGGITYYADSVKGRFTISR | | |
| | | DNSKNTVYLQMNSLRPEDTALYYCAGDKHQSS | | |
| | | WYDYWGQGTLVTVSS | | |
| PAL054-0309.1L | 665 | EVQLVESGGGLVQPGGSLRLSCAASGYTFTNY | 688 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVESG | | |
| | | GGLVQPGGSLRLSCAASGSIASIHAMGWERQA | | |
| | | PGKEREFVAVITWSGGITYYADSVKGRFTISR | | |
| | | DNSKNTVYLQMNSLRPEDTAVYYCAGDKHQSS | | |
| | | WYDYWGQGTLVTVSS | | |
| PAL054-0310.1L | 666 | EVQLVESGGGLVQPGGSLRLSCAASGYTFTNY | 689 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGGGSGGGGSGGGGSGGGGSEVQLVESG | | |
| | | GGLVQPGGSLRLSCAASGSIASIHAMGWFRQA | | |
| | | PGKEREFVAVITWSGGITYYADSVKGRFTISR | | |
| | | DNSKNTVYLQMNSLRPEDTALYYCAGDKHQSS | | |
| | | WYDYWGQGTLVTVSS | | |
| PAL054-0501.1L | 667 | EVQLVESGGGLVQPGGSLRLSCAASGYTFTNY | 690 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSEIVLTQSPAT | | |
| | | LSLSPGERATLSCRASKGVSTSGYSYLHWYQQ | | |
| | | KPGQAPRLLIYLASYLESGVPARFSGSGSGTD | | |
| | | FTLTISSLEPEDFAVYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSQVQLVQSGVEVKKPGASVKV | | |
| | | SCKASGYTFTNYYMYWVRQAPGQGLEWMGGIN | | |
| | | PSNGGTNFNEKFKNRVTLTTDSSTTTAYMELK | | |
| | | SLQFDDTAVYYCARRDYRFDMGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0501.1La | 668 | EVQLVESGGGLVQPGGSLRLSCAASGYTFTNY | 691 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSEIVLTQSPAT | | |
| | | LSLSPGERATLSCRASKGVSTSGYSYLHWYQQ | | |
| | | KPGQAPRLLIYLASYLESGVPARFSGSGSGTD | | |
| | | FTLTISSLEPEDFAVYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSQVQLVQSGVEVKKPGASVKV | | |
| | | SCKASGYTFTNYYMYWVRQAPGQGLEWMGGIN | | |
| | | PSNGGTNFNEKFKNRVTLTTDSSTTTAYMELK | | |
| | | SLQFDDTAVYYCARRDYRFDMGEDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0502.1L | 669 | EVQLVESGGGLVQPGGSLRLSCAASGYTFTNY | 692 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSEIVLTQSPAT | | |
| | | LSLSPGERATLSCRASKGVSTSGYSYLHWYQQ | | |
| | | KPGQAPRLLIYLASYLESGVPARFSGSGSGTD | | |
| | | FTLTISSLEPEDFAVYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLILSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSQVQLVQSGVEVKKPGASVKV | | |
| | | SCKASGYTFTNYYMYWVRQAPGQGLEWMGGIN | | |
| | | PSQGGTNFNEKFKNRVTLTTDSSTTTAYMELK | | |
| | | SLQFDDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0502.1La | 670 | EVQLVESGGGLVQPGGSLRLSCAASGYTFTNY | 693 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSEIVLTQSPAT | | |
| | | LSLSPGERATLSCRASKGVSTSGYSYLHWYQQ | | |
| | | KPGQAPRLLIYLASYLESGVPARFSGSGSGTD | | |
| | | FTLTISSLEPEDFAVYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSQVQLVQSGVEVKKPGASVKV | | |
| | | SCKASGYTFTNYYMYWVRQAPGQGLEWMGGIN | | |
| | | PSQGGTNFNEKFKNRVTLTTDSSTTTAYMELK | | |
| | | SLQFDDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0503.1L | 671 | EVQLVESGGGLVQPGGSLRLSCAASGYTFTNY | 694 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSEIVLTQSPAT | | |
| | | LSLSPGERATLSCRASKGVSTSGYSYLHWYQQ | | |
| | | KPGQAPRLLIYLASYLESGVPARFSGSGSGTD | | |
| | | FTLTISSLEPEDFAVYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSQVQLVQSGVEVKKPGASVKV | | |
| | | SCKASGYTFTNYYMYWVRQAPGQGLEWMGGIN | | |
| | | PSRGGTNFNEKFKNRVTLTTDSSTTTAYMELK | | |
| | | SLQFDDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0503.1La | 672 | EVQLVESGGGLVQPGGSLRLSCAASGYTFTNY | 695 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSEIVLTQSPAT | | |
| | | LSLSPGERATLSCRASKGVSTSGYSYLHWYQQ | | |
| | | KPGQAPRLLIYLASYLESGVPARFSGSGSGTD | | |
| | | FTLTISSLEPEDFAVYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSQVQLVQSGVEVKKPGASVKV | | |
| | | SCKASGYTFTNYYMYWVRQAPGQGLEWMGGIN | | |
| | | PSRGGTNFNEKFKNRVTLTTDSSTTTAYMELK | | |
| | | SLQFDDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0504.1L | 673 | EVQLVESGGGLVQPGGSLRLSCAASGYTFTNY | 696 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSDIQLTQSPSS | | |
| | | LSASVGDRVTITCRASKGVSTSGYSYLHWYQQ | | |
| | | KPGKAPKLLIYLASYLESGVPSRFSGSGSGTD | | |
| | | FTLTISSLQPEDFATYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSEVQLVQSGAEVKKPGASVKV | | |
| | | SCKASGYTFTNYYMYWVRQAPGQGLEWMGGIN | | |
| | | PSNGGTNFNEKFKNRVTLTTDSSTSTAYMELS | | |
| | | SLRSEDTAVYYCARRDYRFDMGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0504.1La | 674 | EVQLVESGGGLVQPGGSLRLSCAASGYTFTNY | 697 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSDIQLTQSPSS | | |
| | | LSASVGDRVTITCRASKGVSTSGYSYLHWYQQ | | |
| | | KPGKAPKLLIYLASYLESGVPSRFSGSGSGTD | | |
| | | FTLTISSLQPEDFATYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLILSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSEVQLVQSGAEVKKPGASVKV | | |
| | | SCKASGYTFTNYYMYWVRQAPGQGLEWMGGIN | | |
| | | PSNGGTNFNEKFKNRVTLTTDSSTSTAYMELS | | |
| | | SLRSEDTAVYYCARRDYRFDMGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0505.1L | 675 | EVQLVESGGGLVQPGGSLRLSCAASGYTFTNY | 698 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSDIQLTQSPSS | | |
| | | LSASVGDRVTITCRASKGVSTSGYSYLHWYQQ | | |
| | | KPGKAPKLLIYLASYLESGVPSRFSGSGSGTD | | |
| | | FTLTISSLQPEDFATYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSEVQLVQSGAEVKKPGASVKV | | |
| | | SCKASGYTFTNYYMYWVRQAPGQGLEWMGGIN | | |
| | | PSQGGTNFNEKFKNRVTLITDSSTSTAYMELS | | |
| | | SLRSEDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0505.1La | 676 | EVQLVESGGGLVQPGGSLRLSCAASGYTFTNY | 699 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSDIQLTQSPSS | | |
| | | LSASVGDRVTITCRASKGVSTSGYSYLHWYQQ | | |
| | | KPGKAPKLLIYLASYLESGVPSRFSGSGSGTD | | |
| | | FTLTISSLQPEDFATYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSEVQLVQSGAEVKKPGASVKV | | |
| | | SCKASGYTFTNYYMYWVRQAPGQGLEWMGGIN | | |
| | | PSQGGTNFNEKFKNRVILITDSSTSTAYMELS | | |
| | | SLRSEDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0506.1L | 677 | EVQLVESGGGLVQPGGSLRLSCAASGYTFTNY | 700 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LMISRTPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSDIQLTQSPSS | | |
| | | LSASVGDRVTITCRASKGVSTSGYSYLHWYQQ | | |
| | | KPGKAPKLLIYLASYLESGVPSRFSGSGSGTD | | |
| | | FTLTISSLQPEDFATYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSEVQLVQSGAEVKKPGASVKV | | |
| | | SCKASGYTFTNYYMYWVRQAPGQGLEWMGGIN | | |
| | | PSRGGTNFNEKFKNRVTLTTDSSTSTAYMELS | | |
| | | SLRSEDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |
| PAL054-0506.1La | 678 | EVQLVESGGGLVQPGGSLRLSCAASGYTFTNY | 701 | DIQMTQSPSSLSASVGDRVTITCSASQDISNYLN |
| | | GMNWVRQAPGKGLEWVGWINTYTGEPTYAADF | | WYQQKPGKAPKVLIYFTSSLHSGVPSRFSGSGSG |
| | | KRRFTFSLDTSKSTAYLQMNSLRAEDTAVYYC | | TDFTLTISSLQPEDFATYYCQQYSTVPWTFGQGT |
| | | AKYPHYYGSSHWYFDVWGQGTLVTVSSASTKG | | KVEIKRTVAAPSVFIFPPSDEQLKSGTASVVCLL |
| | | PSVFPLAPSSKSTSGGTAALGCLVKDYFPEPV | | NNFYPREAKVQWKVDNALQSGNSQESVTEQDSKD |
| | | TVSWNSGALTSGVHTFPAVLQSSGLYSLSSVV | | STYSLSSTLTLSKADYEKHKVYACEVTHQGLSSP |
| | | TVPSSSLGTQTYICNVNHKPSNTKVDKKVEPK | | VTKSFNRGEC |
| | | SCDKTHTCPPCPAPEAAGGPSVFLFPPKPKDT | | |
| | | LYITREPEVTCVVVDVSHEDPEVKFNWYVDGV | | |
| | | EVHNAKTKPREEQYNSTYRVVSVLTVLHQDWL | | |
| | | NGKEYKCKVSNKALPAPIEKTISKAKGQPREP | | |
| | | QVYTLPPSRDELTKNQVSLTCLVKGFYPSDIA | | |
| | | VEWESNGQPENNYKTTPPVLDSDGSFFLYSKL | | |
| | | TVDKSRWQQGNVFSCSVMHEALHNHYTQKSLS | | |
| | | LSPGGGSGGGGSGGGGSGGGGSDIQLTQSPSS | | |
| | | LSASVGDRVTITCRASKGVSTSGYSYLHWYQQ | | |
| | | KPGKAPKLLIYLASYLESGVPSRFSGSGSGTD | | |
| | | FTLTISSLQPEDFATYYCQHSRDLPLTFGGGT | | |
| | | KVEIKRTVAAPSVFIFPPSDEQLKSGTASVVC | | |
| | | LLNNFYPREAKVQWKVDNALQSGNSQESVTEQ | | |
| | | DSKDSTYSLSSTLTLSKADYEKHKVYACEVTH | | |
| | | QGLSSPVTKSFNRGECGGSSGSGSGSTGTSSS | | |
| | | GTGTSAGTTGTSASTSGSGSGGGGGSGGGGSA | | |
| | | GGTATAGASSGSEVQLVQSGAEVKKPGASVKV | | |
| | | SCKASGYTFTNYYMYWVRQAPGQGLEWMGGIN | | |
| | | PSRGGTNFNEKFKNRVTLTTDSSTSTAYMELS | | |
| | | SLRSEDTAVYYCARRDYRFDLGFDYWGQGTTV | | |
| | | TVSSASTKGPSVFPLAPSSKSTSGGTAALGCL | | |
| | | VKDYFPEPVTVSWNSGALTSGVHTFPAVLQSS | | |
| | | GLYSLSSVVTVPSSSLGTQTYICNVNHKPSNT | | |
| | | KVDKKVEPKSCDKTHT | | |

TABLE 4B
Sequences of Heavy Chains and Light Chains of Multispecific Binding Proteins

| Construct | SEQ | | SEQ | | SEQ | |
|---|---|---|---|---|---|---|
| Name | ID NO | Heavy Chain | ID NO | Light Chain 1 | ID NO | Light Chain 2 |
| PAL054- | 702 | EVQLVESGGGLVQPGGSLRLS | 714 | DIQMTQSPSSLSASVGDRVTI | 726 | EIVLTQSPATLSLSPGERATL |
| 0401.1L | | CAASGYTFTNYGMNWVRQAPG | | TCSASQDISNYLNWYQQKPGK | | SCRASKGVSTSGYSYLHWYQQ |
| | | KGLEWVGWINTYTGEPTYAAD | | APKVLIYFTSSLHSGVPSRES | | KPGQAPRLLIYLASYLESGVP |
| | | FKRRFTFSLDTSKSTAYLQMN | | GSGSGTDFTLTISSLQPEDFA | | ARFSGSGSGTDFTLTISSLEP |
| | | SLRAEDTAVYYCAKYPHYYGS | | TYYCQQYSTVPWTFGQGTKVE | | EDFAVYYCQHSRDLPLTFGGG |
| | | SHWYFDVWGQGTLVTVSSAST | | IKRTVAAPSVFIFPPSDEQLK | | TKVEIKSSASTKGPSVEPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLMISRTPEVTCVVVDVS | | | | |
| | | HEDPEVKFNWYVDGVEVHNAK | | | | |
| | | TKPREEQYNSTYRVVSVLIVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSQVQLVQSGVEVKK | | | | |
| | | PGASVKVSCKASGYTFTNYYM | | | | |
| | | YWVRQAPGQGLEWMGGINPSN | | | | |
| | | GGTNFNEKFKNRVTLTTDSST | | | | |
| | | TTAYMELKSLQFDDTAVYYCA | | | | |
| | | RRDYRFDMGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 703 | EVQLVESGGGLVQPGGSLRLS | 715 | DIQMTQSPSSLSASVGDRVTI | 727 | EIVLTQSPATLSLSPGERATL |
| 0401.1La | | CAASGYTFTNYGMNWVRQAPG | | TCSASQDISNYLNWYQQKPGK | | SCRASKGVSTSGYSYLHWYQQ |
| | | KGLEWVGWINTYTGEPTYAAD | | APKVLIYFTSSLHSGVPSRES | | KPGQAPRLLIYLASYLESGVP |
| | | FKRRFTFSLDTSKSTAYLQMN | | GSGSGTDFTLTISSLQPEDFA | | ARFSGSGSGTDFTLTISSLEP |
| | | SLRAEDTAVYYCAKYPHYYGS | | TYYCQQYSTVPWTFGQGTKVE | | EDFAVYYCQHSRDLPLTFGGG |
| | | SHWYFDVWGQGTLVTVSSAST | | IKRTVAAPSVFIFPPSDEQLK | | TKVEIKSSASTKGPSVEPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLYITREPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSQVQLVQSGVEVKK | | | | |
| | | PGASVKVSCKASGYTFTNYYM | | | | |
| | | YWVRQAPGQGLEWMGGINPSN | | | | |
| | | GGTNFNEKFKNRVTLTTDSST | | | | |
| | | TTAYMELKSLQFDDTAVYYCA | | | | |
| | | RRDYRFDMGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLILSKADYEKH | | | | |
| | | KVYACEVTHQGLSSPVTKSEN | | | | |
| | | RGEC | | | | |
| PAL054- | 704 | EVQLVESGGGLVQPGGSLRLS | 716 | DIQMTQSPSSLSASVGDRVTI | 728 | EIVLTQSPATLSLSPGERATL |
| 0402.1L | | CAASGYTFTNYGMNWVRQAPG | | TCSASQDISNYLNWYQQKPGK | | SCRASKGVSTSGYSYLHWYQQ |
| | | KGLEWVGWINTYTGEPTYAAD | | APKVLIYFTSSLHSGVPSRFS | | KPGQAPRLLIYLASYLESGVP |
| | | FKRRFTFSLDTSKSTAYLQMN | | GSGSGTDFTLTISSLQPEDFA | | ARFSGSGSGTDFTLTISSLEP |
| | | SLRAEDTAVYYCAKYPHYYGS | | TYYCQQYSTVPWTFGQGTKVE | | EDFAVYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLMISRTPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSQVQLVQSGVEVKK | | | | |
| | | PGASVKVSCKASGYTFTNYYM | | | | |
| | | YWVRQAPGQGLEWMGGINPSQ | | | | |
| | | GGTNFNEKFKNRVTLTTDSST | | | | |
| | | TTAYMELKSLQFDDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLILSKADYEKH | | | | |
| | | KVYACEVTHQGLSSPVTKSEN | | | | |
| | | RGEC | | | | |
| PAL054- | 705 | EVQLVESGGGLVQPGGSLRLS | 717 | DIQMTQSPSSLSASVGDRVTI | 729 | EIVLTQSPATLSLSPGERATL |
| 0402.1La | | CAASGYTFTNYGMNWVRQAPG | | TCSASQDISNYLNWYQQKPGK | | SCRASKGVSTSGYSYLHWYQQ |
| | | KGLEWVGWINTYTGEPTYAAD | | APKVLIYFTSSLHSGVPSRFS | | KPGQAPRLLIYLASYLESGVP |
| | | FKRRFTFSLDTSKSTAYLQMN | | GSGSGTDFTLTISSLQPEDFA | | ARFSGSGSGTDFTLTISSLEP |
| | | SLRAEDTAVYYCAKYPHYYGS | | TYYCQQYSTVPWTFGQGTKVE | | EDFAVYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLYITREPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSQVQLVQSGVEVKK | | | | |
| | | PGASVKVSCKASGYTFTNYYM | | | | |
| | | YWVRQAPGQGLEWMGGINPSQ | | | | |
| | | GGTNFNEKFKNRVTLTTDSST | | | | |
| | | TTAYMELKSLQFDDTAVYYCA | | | | |
| | | RRDYRFDLGEDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLILSKADYEKH | | | | |
| | | KVYACEVTHQGLSSPVTKSEN | | | | |
| | | RGEC | | | | |
| PAL054- | 706 | EVQLVESGGGLVQPGGSLRLS | 718 | DIQMTQSPSSLSASVGDRVTI | 730 | EIVLIQSPATLSLSPGERATL |
| 0403.1L | | CAASGYTFTNYGMNWVRQAPG | | TCSASQDISNYLNWYQQKPGK | | SCRASKGVSTSGYSYLHWYQQ |
| | | KGLEWVGWINTYTGEPTYAAD | | APKVLIYFTSSLHSGVPSRFS | | KPGQAPRLLIYLASYLESGVP |
| | | FKRRFTFSLDTSKSTAYLQMN | | GSGSGTDFTLTISSLQPEDFA | | ARFSGSGSGTDFTLTISSLEP |
| | | SLRAEDTAVYYCAKYPHYYGS | | TYYCQQYSTVPWTFGQGTKVE | | EDFAVYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLMISRTPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGE | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSQVQLVQSGVEVKK | | | | |
| | | PGASVKVSCKASGYTFTNYYM | | | | |
| | | YWVRQAPGQGLEWMGGINPSR | | | | |
| | | GGTNFNEKFKNRVTLTTDSST | | | | |
| | | TTAYMELKSLQFDDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 707 | EVQLVESGGGLVQPGGSLRLS | 719 | DIQMTQSPSSLSASVGDRVTI | 731 | EIVLTQSPATLSLSPGERATL |
| 0403.1La | | CAASGYTFTNYGMNWVRQAPG | | TCSASQDISNYLNWYQQKPGK | | SCRASKGVSTSGYSYLHWYQQ |
| | | KGLEWVGWINTYTGEPTYAAD | | APKVLIYFTSSLHSGVPSRES | | KPGQAPRLLIYLASYLESGVP |
| | | FKRRFTFSLDTSKSTAYLQMN | | GSGSGTDFTLTISSLQPEDFA | | ARFSGSGSGTDFTLTISSLEP |
| | | SLRAEDTAVYYCAKYPHYYGS | | TYYCQQYSTVPWTFGQGTKVE | | EDFAVYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLYITREPEVTCVVVDVS | | | | |
| | | HEDPEVKFNWYVDGVEVHNAK | | | | |
| | | TKPREEQYNSTYRVVSVLIVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSQVQLVQSGVEVKK | | | | |
| | | PGASVKVSCKASGYTFTNYYM | | | | |
| | | YWVRQAPGQGLEWMGGINPSR | | | | |
| | | GGTNFNEKFKNRVTLTTDSST | | | | |
| | | TTAYMELKSLQFDDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 708 | EVQLVESGGGLVQPGGSLRLS | 720 | DIQMTQSPSSLSASVGDRVTI | 732 | DIQLTQSPSSLSASVGDRVTI |
| 0404.1L | | CAASGYTFTNYGMNWVRQAPG | | TCSASQDISNYLNWYQQKPGK | | TCRASKGVSTSGYSYLHWYQQ |
| | | KGLEWVGWINTYTGEPTYAAD | | APKVLIYFTSSLHSGVPSRFS | | KPGKAPKLLIYLASYLESGVP |
| | | FKRRFTFSLDTSKSTAYLQMN | | GSGSGTDFTLTISSLQPEDFA | | SRFSGSGSGTDFTLTISSLQP |
| | | SLRAEDTAVYYCAKYPHYYGS | | TYYCQQYSTVPWTFGQGTKVE | | EDFATYYCQHSRDLPLTFGGG |
| | | SHWYFDVWGQGTLVTVSSAST | | IKRTVAAPSVEIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLILSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLMISRTPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSEVQLVQSGAEVKK | | | | |
| | | PGASVKVSCKASGYTFTNYYM | | | | |
| | | YWVRQAPGQGLEWMGGINPSN | | | | |
| | | GGTNFNEKFKNRVTLTTDSST | | | | |
| | | STAYMELSSLRSEDTAVYYCA | | | | |
| | | RRDYRFDMGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 709 | EVQLVESGGGLVQPGGSLRLS | 721 | DIQMTQSPSSLSASVGDRVTI | 733 | DIQLTQSPSSLSASVGDRVTI |
| 0404.1La | | CAASGYTFTNYGMNWVRQAPG | | TCSASQDISNYLNWYQQKPGK | | TCRASKGVSTSGYSYLHWYQQ |
| | | KGLEWVGWINTYTGEPTYAAD | | APKVLIYFTSSLHSGVPSRFS | | KPGKAPKLLIYLASYLESGVP |
| | | FKRRFTFSLDTSKSTAYLQMN | | GSGSGTDFTLTISSLQPEDFA | | SRFSGSGSGTDFTLTISSLQP |
| | | SLRAEDTAVYYCAKYPHYYGS | | TYYCQQYSTVPWTFGQGTKVE | | EDFATYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLYITREPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSEVQLVQSGAEVKK | | | | |
| | | PGASVKVSCKASGYTFTNYYM | | | | |
| | | YWVRQAPGQGLEWMGGINPSN | | | | |
| | | GGTNFNEKFKNRVTLTTDSST | | | | |
| | | STAYMELSSLRSEDTAVYYCA | | | | |
| | | RRDYRFDMGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 710 | EVQLVESGGGLVQPGGSLRLS | 722 | DIQMTQSPSSLSASVGDRVTI | 734 | DIQLTQSPSSLSASVGDRVTI |
| 0405.1L | | CAASGYTFTNYGMNWVRQAPG | | TCSASQDISNYLNWYQQKPGK | | TCRASKGVSTSGYSYLHWYQQ |
| | | KGLEWVGWINTYTGEPTYAAD | | APKVLIYFTSSLHSGVPSRFS | | KPGKAPKLLIYLASYLESGVP |
| | | FKRRFTFSLDTSKSTAYLQMN | | GSGSGTDFTLTISSLQPEDFA | | SRFSGSGSGTDFTLTISSLQP |
| | | SLRAEDTAVYYCAKYPHYYGS | | TYYCQQYSTVPWTFGQGTKVE | | EDFATYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLMISRTPEVTCVVVDVS | | | | |
| | | HEDPEVKFNWYVDGVEVHNAK | | | | |
| | | TKPREEQYNSTYRVVSVLIVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLIVDK | | | | |
| | | SRWQQGNVFSCSVMHEALHNH | | | | |
| | | YTQKSLSLSPGGGSGGGGSGG | | | | |
| | | GGSGGGGSEVQLVQSGAEVKK | | | | |
| | | PGASVKVSCKASGYTFTNYYM | | | | |
| | | YWVRQAPGQGLEWMGGINPSQ | | | | |
| | | GGTNFNEKFKNRVTLTTDSST | | | | |
| | | STAYMELSSLRSEDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 711 | EVQLVESGGGLVQPGGSLRLS | 723 | DIQMTQSPSSLSASVGDRVTI | 735 | DIQLTQSPSSLSASVGDRVTI |
| 0405.1La | | CAASGYTFTNYGMNWVRQAPG | | TCSASQDISNYLNWYQQKPGK | | TCRASKGVSTSGYSYLHWYQQ |
| | | KGLEWVGWINTYTGEPTYAAD | | APKVLIYFTSSLHSGVPSRES | | KPGKAPKLLIYLASYLESGVP |
| | | FKRRFTFSLDTSKSTAYLQMN | | GSGSGTDFTLTISSLQPEDFA | | SRFSGSGSGTDFTLTISSLQP |
| | | SLRAEDTAVYYCAKYPHYYGS | | TYYCQQYSTVPWTFGQGTKVE | | EDFATYYCQHSRDLPLTFGGG |
| | | SHWYFDVWGQGTLVTVSSAST | | IKRTVAAPSVFIFPPSDEQLK | | TKVEIKSSASTKGPSVFPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLILSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLYITREPEVTCVVVDVS | | | | |
| | | HEDPEVKENWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLIVDK | | | | |
| | | SRWQQGNVFSCSVMHEALHNH | | | | |
| | | YTQKSLSLSPGGGSGGGGSGG | | | | |
| | | GGSGGGGSEVQLVQSGAEVKK | | | | |
| | | PGASVKVSCKASGYTFTNYYM | | | | |
| | | YWVRQAPGQGLEWMGGINPSQ | | | | |
| | | GGTNFNEKFKNRVTLTTDSST | | | | |
| | | STAYMELSSLRSEDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 712 | EVQLVESGGGLVQPGGSLRLS | 724 | DIQMTQSPSSLSASVGDRVTI | 736 | DIQLTQSPSSLSASVGDRVTI |
| 0406.1L | | CAASGYTFTNYGMNWVRQAPG | | TCSASQDISNYLNWYQQKPGK | | TCRASKGVSTSGYSYLHWYQQ |
| | | KGLEWVGWINTYTGEPTYAAD | | APKVLIYFTSSLHSGVPSRES | | KPGKAPKLLIYLASYLESGVP |
| | | FKRRFTFSLDTSKSTAYLQMN | | GSGSGTDFTLTISSLQPEDFA | | SRFSGSGSGTDFTLTISSLQP |
| | | SLRAEDTAVYYCAKYPHYYGS | | TYYCQQYSTVPWTFGQGTKVE | | EDFATYYCQHSRDLPLTFGGG |
| | | SHWYFDVWGQGTLVTVSSAST | | IKRTVAAPSVFIFPPSDEQLK | | TKVEIKSSASTKGPSVEPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLMISRTPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSEVQLVQSGAEVKK | | | | |
| | | PGASVKVSCKASGYTFTNYYM | | | | |
| | | YWVRQAPGQGLEWMGGINPSR | | | | |
| | | GGTNFNEKFKNRVTLTTDSST | | | | |
| | | STAYMELSSLRSEDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |
| PAL054- | 713 | EVQLVESGGGLVQPGGSLRLS | 725 | DIQMTQSPSSLSASVGDRVTI | 737 | DIQLTQSPSSLSASVGDRVTI |
| 0406.1La | | CAASGYTFTNYGMNWVRQAPG | | TCSASQDISNYLNWYQQKPGK | | TCRASKGVSTSGYSYLHWYQQ |
| | | KGLEWVGWINTYTGEPTYAAD | | APKVLIYFTSSLHSGVPSRES | | KPGKAPKLLIYLASYLESGVP |
| | | FKRRFTFSLDTSKSTAYLQMN | | GSGSGTDFTLTISSLQPEDFA | | SRFSGSGSGTDFTLTISSLQP |
| | | SLRAEDTAVYYCAKYPHYYGS | | TYYCQQYSTVPWTFGQGTKVE | | EDFATYYCQHSRDLPLTFGGG |
| | | SHWYFDVWGQGTLVTVSSAST | | IKRTVAAPSVFIFPPSDEQLK | | TKVEIKSSASTKGPSVEPLAP |
| | | KGPSVFPLAPSSKSTSGGTAA | | SGTASVVCLLNNFYPREAKVQ | | SSKSTSGGTAALGCLVKDYFP |
| | | LGCLVKDYFPEPVTVSWNSGA | | WKVDNALQSGNSQESVTEQDS | | EPVTVSWNSGALTSGVHTFPA |
| | | LTSGVHTFPAVLQSSGLYSLS | | KDSTYSLSSTLTLSKADYEKH | | VLQSSGLYSLSSVVTVPSSSL |
| | | SVVTVPSSSLGTQTYICNVNH | | KVYACEVTHQGLSSPVTKSEN | | GTQTYICNVNHKPSNTKVDKK |
| | | KPSNTKVDKKVEPKSCDKTHT | | RGEC | | VEPKSC |
| | | CPPCPAPEAAGGPSVFLFPPK | | | | |
| | | PKDTLYITREPEVTCVVVDVS | | | | |
| | | HEDPEVKFNWYVDGVEVHNAK | | | | |
| | | TKPREEQYNSTYRVVSVLTVL | | | | |
| | | HQDWLNGKEYKCKVSNKALPA | | | | |
| | | PIEKTISKAKGQPREPQVYTL | | | | |
| | | PPSRDELTKNQVSLTCLVKGF | | | | |
| | | YPSDIAVEWESNGQPENNYKT | | | | |
| | | TPPVLDSDGSFFLYSKLTVDK | | | | |
| | | SRWQQGNVFSCSVMHEALHNH | | | | |
| | | YTQKSLSLSPGGGSGGGGSGG | | | | |
| | | GGSGGGGSEVQLVQSGAEVKK | | | | |
| | | PGASVKVSCKASGYTFTNYYM | | | | |
| | | YWVRQAPGQGLEWMGGINPSR | | | | |
| | | GGTNFNEKFKNRVTLTTDSST | | | | |
| | | STAYMELSSLRSEDTAVYYCA | | | | |
| | | RRDYRFDLGFDYWGQGTTVTV | | | | |
| | | SSASVAAPSVFIFPPSDEQLK | | | | |
| | | SGTASVVCLLNNFYPREAKVQ | | | | |
| | | WKVDNALQSGNSQESVTEQDS | | | | |
| | | KDSTYSLSSTLTLSKADYEKH | | | | |
| | | KVYACEVTHQGLSSPVTKSEN | | | | |
| | | RGEC | | | | |

### VH Domains

In some embodiments, a binding protein provided herein comprises a first VH sequence selected from SEQ ID NOs: 295-296, 301-334, and 738-742 and a second VH sequence selected from SEQ ID NOs: 295-296, 301-334 and 738-742. In some embodiments, a binding protein provided herein comprises a first VH sequence selected from SEQ ID NOs: 295-296 and a second VH sequence selected from SEQ ID NOs: 301-334. In some embodiments, a binding protein provided herein comprises a first VH sequence comprising SEQ ID NO: 3 and a second VH sequence selected from SEQ ID NOs: 301-334.

In some embodiments, a binding protein provided herein comprises a first VH sequence having at least about 80%, 90%, 91%, 92%, 93%, 94%, 95%, 96%, 97%, 98% or 99% identity to a VH sequence selected from SEQ ID NOs: 295-296 and a second VH sequence selected from SEQ ID NOs: 301-334. In some embodiments, a binding protein provided herein comprises a first VH sequence having at least about 80%, 90%, 91%, 92%, 93%, 94%, 95%, 96%, 97%, 98% or 99% identity to a VH sequence comprising SEQ ID NO: 3 and a second VH sequence selected from SEQ ID NOs: 301-334. In some embodiments, a binding protein provided herein comprises a first VH sequence selected from SEQ ID NOs: 295-296 and a second VH sequence selected from SEQ ID NOs: 301-334, with up to 1, 2, 3, 4, 5, 6, 7, 8, 9, 10, 11, 12, 13, 14, 15, 16, 17, 18, 19, or 20 amino acid substitutions. In some embodiments, a binding protein provided herein comprises a first VH sequence comprising SEQ ID NO: 3 and a second VH sequence selected from SEQ ID NOs: 301-334, with up to 1, 2, 3, 4, 5, 6, 7, 8, 9, 10, 11, 12, 13, 14, 15, 16, 17, 18, 19, or 20 amino acid substitutions. In some embodiments, the amino acid substitutions are conservative amino acid substitutions. In some embodiments, the bispecific binding proteins described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining bispecific binding proteins.

TABLE 5A
Sequences of VEGF Heavy Chain
Variable Regions (VH)

| SEQ ID | |
|---|---|
| NO | VH |
| 295 | EVQLVESGGGLVQPGGSLRLSCAASGYTFT |
| | NYGMNWVRQAPGKGLEWVGWINTYTGEPTY |
| | AADFKRRFTFSLDTSKSTAYLQMNSLRAED |
| | TAVYYCAKYPHYYGSSHWYFDVWGQGTLVT |
| | VSS |
| 296 | EVQLVESGGGLVQPGGSLRLSCAASGYTFT |
| | NYGMNWVRQAPGKCLEWVGWINTYTGEPTY |
| | AADFKRRFTFSLDTSKSTAYLQMNSLRAED |
| | TAVYYCAKYPHYYGSSHWYFDVWGQGTLVT |
| | VSS |

TABLE 5B
Sequences of PD-1 Heavy Chain Variable Regions (VH)

| SEQ ID | |
|---|---|
| NO | VH |
| 301 | QVQLVQSGVEVKKPGASVKVSCKASGYTFTNYYMYWVRQAPGQGLEWMGGINPS |
| | NGGTNFNEKFKNRVTLTTDSSTTTAYMELKSLQFDDTAVYYCARRDYRFDMGED |
| | YWGQGTTVTVSS |
| 302 | QVQLVQSGVEVKKPGASVKVSCKASGYTFTNYYMYWVRQAPGQGLEWMGGINPS |
| | QGGTNFNEKFKNRVTLTTDSSTTTAYMELKSLQFDDTAVYYCARRDYRFDLGED |
| | YWGQGTTVTVSS |
| 303 | QVQLVQSGVEVKKPGASVKVSCKASGYTFTNYYMYWVRQAPGQGLEWMGGINPS |
| | RGGTNFNEKFKNRVTLTTDSSTTTAYMELKSLQFDDTAVYYCARRDYREDLGED |
| | YWGQGTTVTVSS |
| 304 | QVQLVQSGVEVKKPGASVKVSCKASGYTFTNYYMYWVRQAPGQGLEWMGGINPS |
| | NGGTNFNEKFKNRVTLTTDSSTTTAYMELKSLQFDDTAVYYCARRDYRFDMGED |
| | YWGQGTTVTVSS |
| 305 | QVQLVQSGVEVKKPGASVKVSCKASGYTFTNYYMYWVRQAPGQCLEWMGGINPS |
| | NGGTNFNEKFKNRVTLTTDSSTTTAYMELKSLQFDDTAVYYCARRDYRFDMGED |
| | YWGQGTTVTVSS |
| 306 | QVQLVQSGVEVKKPGASVKVSCKASGYTFTNYYMYWVRQAPGQCLEWMGGINPS |
| | QGGTNFNEKFKNRVTLTTDSSTTTAYMELKSLQFDDTAVYYCARRDYREDLGED |
| | YWGQGTTVTVSS |
| 307 | QVQLVQSGVEVKKPGASVKVSCKASGYTFTNYYMYWVRQAPGQCLEWMGGINPS |
| | RGGTNFNEKFKNRVTLTTDSSTTTAYMELKSLQFDDTAVYYCARRDYRFDLGED |
| | YWGQGTTVTVSS |
| 308 | EVQLVESGGGLVQPGGSLRLSCAASGYTFTNYYMYWVRQAPGKGLEWMGGINPS |
| | QGGTNFNEKFKNRFTISRDNSKNTLYLQMNSLRAEDTAVYYCARRDYRFDLGED |
| | YWGQGTLVTVSS |
| 309 | EVQLVESGGGLVQPGGSLRLSCAASGYTFTNYYMYWVRQAPGKCLEWMGGINPS |
| | QGGTNFNEKFKNRFTISRDNSKNTLYLQMNSLRAEDTAVYYCARRDYREDLGED |
| | YWGQGTLVTVSS |
| 310 | QVQLVQSGVEVKKPGASVKVSCKASGYTFTNYYMYWVRQAPGQGLEWMGGINPS |
| | QGGTNFNEKFKNRVTLTTDSSTTTAYMELKSLQFDDTAVYYCARRDYRFDLGED |
| | YWGQGTGVRVSS |
| 311 | QVQLVQSGVEVKKPGASVKVSCKASGYTFTNYYMYWVRQAPGQCLEWMGGINPS |
| | QGGTNFNEKFKNRVTLTTDSSTTTAYMELKSLQFDDTAVYYCARRDYREDLGED |
| | YWGQGTGVRVSS |
| 312 | QVQLVQSGVEVKKPGASVKVSCKASGYTFTNYYMYWVRQAPGQGLEWMGGINPS |
| | NGGTNFNEKFKNRVTLTTDSSTTTAYMELKSLRFDDTAVYYCARRDYRFDMGED |
| | YWGQGTTVTVSS |
| 313 | QVQLVQSGVEVKKPGASVKVSCKASGYTFTNYYMYWVRQAPGQGLEWMGGINPS |
| | QGGTNFNEKFKNRVTLTTDSSTTTAYMELKSLRFDDTAVYYCARRDYRFDLGED |
| | YWGQGTTVTVSS |
| 314 | QVQLVQSGVEVKKPGASVKVSCKASGYTFTNYYMYWVRQAPGQGLEWMGGINPS |
| | RGGTNFNEKFKNRVTLTTDSSTTTAYMELKSLRFDDTAVYYCARRDYRFDLGED |
| | YWGQGTTVTVSS |
| 315 | QVQLVQSGVEVKKPGASVKVSCKASGYTFTNYYMYWVRQAPGQCLEWMGGINPS |
| | NGGTNFNEKFKNRVTLTTDSSTTTAYMELKSLRFDDTAVYYCARRDYRFDMGED |
| | YWGQGTTVTVSS |
| 316 | QVQLVQSGVEVKKPGASVKVSCKASGYTFTNYYMYWVRQAPGQCLEWMGGINPS |
| | QGGTNFNEKFKNRVTLTTDSSTTTAYMELKSLRFDDTAVYYCARRDYREDLGED |
| | YWGQGTTVTVSS |
| 317 | QVQLVQSGVEVKKPGASVKVSCKASGYTFTNYYMYWVRQAPGQCLEWMGGINPS |
| | RGGTNFNEKFKNRVTLTTDSSTTTAYMELKSLRFDDTAVYYCARRDYREDLGED |
| | YWGQGTTVTVSS |
| 318 | EVQLVQSGAEVKKPGASVKVSCKASGYTFTNYYMYWVRQAPGQGLEWMGGINPS |
| | QGGTNFNEKFKNRVTLTTDSSTSTAYMELSSLRSEDTAVYYCARRDYREDLGFD |
| | YWGQGTTVTVSS |
| 319 | EVQLLESGGGLVQPGGSLRLSCKASGYTFTNYYMYWVRQAPGKGLEWMGGINPS |
| | QGGTNFNEKFKNRVTLSTDSSKNTAYLQMNSLRAEDTAVYYCARRDYREDLGED |
| | YWGQGTTVTVSS |
| 320 | EVQLVQSGAEVKKPGASVKVSCKASGYTFTNYYMYWVRQAPGQCLEWMGGINPS |
| | QGGTNFNEKFKNRVTLTTDSSTSTAYMELSSLRSEDTAVYYCARRDYRFDLGFD |
| | YWGQGTTVTVSS |
| 321 | EVQLLESGGGLVQPGGSLRLSCKASGYTFTNYYMYWVRQAPGKCLEWMGGINPS |
| | QGGTNFNEKFKNRVTLSTDSSKNTAYLQMNSLRAEDTAVYYCARRDYREDLGED |
| | YWGQGTTVTVSS |
| 322 | EVQLVQSGAEVKKPGASVKVSCKASGYTFTNYYMYWVRQAPGQGLEWMGGINPS |
| | NGGTNFNEKFKNRVTLTTDSSTSTAYMELSSLRSEDTAVYYCARRDYRFDMGED |
| | YWGQGTTVTVSS |
| 323 | EVQLVQSGAEVKKPGASVKVSCKASGYTFTNYYMYWVRQAPGQCLEWMGGINPS |
| | NGGTNFNEKFKNRVTLTTDSSTSTAYMELSSLRSEDTAVYYCARRDYRFDMGED |
| | YWGQGTTVTVSS |
| 324 | EVQLVQSGAEVKKPGASVKVSCKASGYTFTNYYMYWVRQAPGQCLEWMGGINPS |
| | RGGTNFNEKFKNRVTLTTDSSTSTAYMELSSLRSEDTAVYYCARRDYRFDLGED |
| | YWGQGTTVTVSS |
| 325 | EVQLVQSGAEVKKPGASVKVSCKASGYTFTNYYMYWVRQAPGQCLEWMGGINPS |
| | QGGTNFNEKFKNRVTLTTDSSTSTAYMELSSLRSEDTAVYYCARRDYRFDMGED |
| | YWGQGTTVTVSS |
| 326 | EVQLVQSGAEVKKPGASVKVSCKASGYTFTNYYMYWVRQAPGQCLEWMGGINPS |
| | RGGTNFNEKFKNRVTLTTDSSTSTAYMELSSLRSEDTAVYYCARRDYRFDMGED |
| | YWGQGTTVTVSS |
| 327 | EVQLVQSGAEVKKPGASVKVSCKASGYTFTNYYMYWVRQAPGQCLEWMGGINPS |
| | NGGTNFNEKFKNRVTLTTDSSTSTAYMELSSLRSEDTAVYYCARRDYREDLGED |
| | YWGQGTTVTVSS |
| 328 | EVQLVESGGGLVQPGGSLRLSCAASGSIASIHAMGWVRQAPGKEREWVSVITWS |
| | GGITYYADSVKGRFTISRDNSKNTLYLQMNSLRAEDTAVYYCAGDKHQSSWYDY |
| | WGQGTLVTVSS |
| 329 | EVQLVESGGGLVQPGGSLRLSCAASGSIASIHAMGWVRQAPGKEREWVSVITWS |
| | GGITYYADSVKGRFTISRDNSKNTVYLQMNSLRAEDTAVYYCAGDKHQSSWYDY |
| | WGQGTLVTVSS |
| 330 | EVQLVESGGGLVQPGGSLRLSCAASGSIASIHAMGWVRQAPGKEREFVSVITWS |
| | GGITYYADSVKGRFTISRDNSKNTLYLQMNSLRAEDTAVYYCAGDKHQSSWYDY |
| | WGQGTLVTVSS |
| 331 | EVQLVESGGGLVQPGGSLRLSCAASGSIASIHAMGWVRQAPGKEREFVSVITWS |
| | GGITYYADSVKGRFTISRDNSKNTVYLQMNSLRAEDTAVYYCAGDKHQSSWYDY |
| | WGQGTLVTVSS |
| 332 | EVQLVESGGGLVQPGGSLRLSCAASGSIASIHAMGWVRQAPGKEREFVAVITWS |
| | GGITYYADSVKGRFTISRDNSKNIVYLQMNSLRAEDTAVYYCAGDKHQSSWYDY |
| | WGQGTLVTVSS |
| 333 | EVQLVESGGGLVQPGGSLRLSCAASGSIASIHAMGWVRQAPGKEREWVAVITWS |
| | GGITYYADSVKGRFTISRDNSKNTVYLQMNSLRAEDTAVYYCAGDKHQSSWYDY |
| | WGQGTLVTVSS |
| 334 | EVQLVESGGGLVQPGGSLRLSCAASGFAFSSYDMSWVRQAPGKGLDWVATISGG |
| | GRYTYYPDSVKGRFTISRDNSKNNLYLQMNSLRAEDTALYYCANRYGEAWFAYW |
| | GQGTLVTVSS |
| 738 | EVQLVESGGGLVQPGGSLRLSCAASGSIASIHAMGWVRQAPGKEREFVAVITWS |
| | GGITYYADSVKGRFTISRDNSKNTVYLQMNSLRPEDTAVYYCAGDKHQSSWYDY |
| | WGQGTLVTVSS |
| 739 | EVQLVESGGGLVQPGGSLRLSCAASGSIASIHAMGWVRQAPGKEREFVAVITWS |
| | GGITYYADSVKGRFTISRDNSKNTVYLQMNSLRPEDTALYYCAGDKHQSSWYDY |
| | WGQGTLVTVSS |
| 740 | EVQLVESGGGLVQPGGSLRLSCAASGSIASIHAMGWFRQAPGKEREFVAVITWS |
| | GGITYYADSVKGRFTISRDNSKNTVYLQMNSLRPEDTAVYYCAGDKHQSSWYDY |
| | WGQGTLVTVSS |
| 741 | EVQLVESGGGLVQPGGSLRLSCAASGSIASIHAMGWFRQAPGKEREFVAVITWS |
| | GGITYYADSVKGRFTISRDNSKNIVYLQMNSLRPEDTALYYCAGDKHQSSWYDY |
| | WGQGTLVTVSS |
| 742 | EVQLVQSGAEVKKPGASVKVSCKASGYTFTNYYMYWVRQAPGQGLEWMGGINPS |
| | RGGTNFNEKFKNRVTLTTDSSTSTAYMELSSLRSEDTAVYYCARRDYRFDLGED |
| | YWGQGTTVTVSS |

### VL Domains

In some embodiments, a binding protein provided herein comprises a first VL sequence selected from SEQ ID NOs: 298-300, 335-349, and 743-744 and a second VL sequence selected from SEQ ID NOs: 298-300, 335-349, and 743-744. In some embodiments, a binding protein provided herein comprises a first VL sequence selected from SEQ ID NOs: 298-300 and a second VL sequence selected from SEQ ID NOs: 335-349 and 743-744. In some embodiments, a binding protein provided herein comprises a first VL sequence comprising SEQ ID NO: 298 and a second VL sequence selected from SEQ ID NOs: 335-349 and 743-744. In some embodiments, a binding protein provided herein comprises a first VL sequence having at least about 80%, 90%, 91%, 92%, 93%, 94%, 95%, 96%, 97%, 98% or 99% identity to a VL sequence comprising SEQ ID NO: 298 and a second VL sequence selected from SEQ ID NOs: 335-349 and 743-744. In some embodiments, a binding protein provided herein comprises a first VL sequence comprising SEQ ID NO: 299 and a second VL sequence selected from SEQ ID NOs: 335-349 and 743-744. In some embodiments, a binding protein provided herein comprises a first VL sequence having at least about 80%, 90%, 91%, 92%, 93%, 94%, 95%, 96%, 97%, 98% or 99% identity to a VL sequence comprising SEQ ID NO: 299 and a second VL sequence selected from SEQ ID NOs: 335-349 and 743-744. In some embodiments, a binding protein provided herein comprises a first VL sequence comprising SEQ ID NO: 300 and a second VL sequence selected from SEQ ID NOs: 335-349 and 743-744. In some embodiments, a binding protein provided herein comprises a first VL sequence having at least about 80%, 90%, 91%, 92%, 93%, 94%, 95%, 96%, 97%, 98% or 99% identity to a VL sequence comprising SEQ ID NO: 300 and a second VL sequence selected from SEQ ID NOs: 335-349 and 743-744.

In some embodiments, a binding protein provided herein comprises a first VL sequence selected from SEQ ID NOs: 298-300, and a second VL sequence selected from SEQ ID NOs: 335-349 and 743-744, with up to 1, 2, 3, 4, 5, 6, 7, 8, 9, 10, 11, 12, 13, 14, 15, 16, 17, 18, 19, or 20 amino acid substitutions. In some embodiments, a binding protein provided herein comprises a first VL sequence comprising SEQ ID NO: 298 and a second VL sequence selected from SEQ ID NOs: 335-349 and 743-744, with up to 1, 2, 3, 4, 5, 6, 7, 8, 9, 10, 11, 12, 13, 14, 15, 16, 17, 18, 19, or 20 amino acid substitutions.

TABLE 6A
Sequences of VEGF Light Chain
Variable Regions (VL)

| SEQ ID | |
|---|---|
| NO | VL |
| 298 | DIQMTQSPSSLSASVGDRVTITCSASQDIS |
| | NYLNWYQQKPGKAPKVLIYFTSSLHSGVPS |
| | RFSGSGSGTDFTLTISSLQPEDFATYYCQQ |
| | YSTVPWTFGQGTKVEIK |
| 299 | DIQMTQSPSSLSASVGDRVTITCSASQDIS |
| | NYLNWYQQKPGKAPKVLIYFTSSLHSGVPS |
| | RFSGSGSGTDFTLTISSLQPEDFATYYCQQ |
| | YSTVPWTFGQGTKVEIKR |
| 300 | DIQMTQSPSSLSASVGDRVTITCSASQDIS |
| | NYLNWYQQKPGKAPKVLIYFTSSLHSGVPS |
| | RFSGSGSGTDFTLTISSLQPEDFATYYCQQ |
| | YSTVPWTFGCGIKVEIKR |

TABLE 6B
Sequences of PD-1 Light Chain Variable Regions (VL)

| SEQ ID | |
|---|---|
| NO | VL |
| 335 | EIVLTQSPATLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGGGTKVEI |
| | K |
| 336 | EIVLTQSPATLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGGGTKVEI |
| | KR |
| 337 | EIVLTQSPATLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGCGTKVEI |
| | KR |
| 338 | EIVLTQSPATLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGGGTKVEI |
| | KSS |
| 339 | EIVLTQSPATLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGCGTKVEI |
| | K |
| 340 | EIVLTQSPATLSVSPGERATLSCRASKGVSTSGYSYLHWYQQKPGQAPRLLIYLA |
| | SYLESGIPARFSGSGSGTEFTLTISSLQSEDFAVYYCQHSRDLPLTFGGGTKVEI |
| | KR |
| 341 | EIVLTQSPATLSVSPGERATLSCRASKGVSTSGYSYLHWYQQKPGQAPRLLIYLA |
| | SYLESGIPARFSGSGSGTEFTLTISSLQSEDFAVYYCQHSRDLPLTFGCGTKVEI |
| | KR |
| 342 | EIVLTQSPSTLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGGGTKVEI |
| | KR |
| 343 | EIVLTQSPSTLSLSPGERATLSCRASKGVSTSGYSYLHWYQQKPGQAPRLLIYLA |
| | SYLESGVPARFSGSGSGTDFTLTISGLEPEDFAVYYCQHSRDLPLTFGGGTKVEI |
| | KR |
| 344 | EIVLTQSPSTLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGCGTKVEI |
| | KR |
| 345 | EIVLTQSPSTLSLSPGERATLSCRASKGVSTSGYSYLHWYQQKPGQAPRLLIYLA |
| | SYLESGVPARFSGSGSGTDFTLTISGLEPEDFAVYYCQHSRDLPLTFGCGTKVEI |
| | KR |
| 346 | DIQLTQSPSSLSASVGDRVTITCRASKGVSTSGYSYLHWYQQKPGKAPKLLIYLA |
| | SYLESGVPSRFSGSGSGTDFTLTISSLQPEDFATYYCQHSRDLPLTFGGGTKVEI |
| | KR |
| 347 | DIQLTQSPSSLSASVGDRVTITCRASKGVSTSGYSYLHWYQQKPGKAPKLLIYLA |
| | SYLESGVPSRFSGSGSGTDFTLTISSLQPEDFATYYCQHSRDLPLTFGCGTKVEI |
| | KR |
| 348 | EIVLTQSPSTLSLSPGERATLSCRASKGVSTSGYSYLHWYQQKPGQAPRLLIYLA |
| | SYLESGVPARFSGSGSGTDFTLTISSLEPEDFAVYYCQHSRDLPLTFGGGTKVEI |
| | K |
| 349 | DIQMTQSPSSMSASVGDRVTFTCRASQDINTYLSWFQQKPGKSPKTLIYRANRLV |
| | SGVPSRFSGSGSGQDYTLTISSLQPEDMATYYCLQYDEFPLTFGAGTKLELKR |
| 743 | DIQLTQSPSSLSASVGDRVTITCRASKGVSTSGYSYLHWYQQKPGKAPKLLIYLA |
| | SYLESGVPSRFSGSGSGTDFTLTISSLQPEDFATYYCQHSRDLPLTFGCGTKVEI |
| | K |
| 744 | DIQLTQSPSSLSASVGDRVTITCRASKGVSTSGYSYLHWYQQKPGKAPKLLIYLA |
| | SYLESGVPSRFSGSGSGTDFTLTISSLQPEDFATYYCQHSRDLPLTFGGGTKVEI |
| | K |

### VH-VL Combinations

In some embodiments the CDR-H1, CDR-H2, and CDR-H3 of the VH within a PD-1 binding region (anti-PD1 VH) comprise the amino acid sequences of (a) SEQ ID NOs: 445, 456, and 457, respectively; (b) SEQ ID NOs: 451, 458, and 459, respectively; or (c) SEQ ID NOs: 443, 460, and 457, respectively, and the CDR-L1, CDR-L2, and CDR-HL of the VL within a PD-1 binding region (anti-PD1 VL) comprise the amino acid sequences of (a) SEQ ID NOs: 448, 449, and 450, respectively; (b) SEQ ID NO: 454, SAS, and SEQ ID NO: 450, respectively; or (c) SEQ ID NOs: 448, 449, or 450, respectively, and the anti-PD1 VH and anti-PD1 VL have amino acid sequences that are at least 85% identical to that of: (a) SEQ ID NOs: 302 and 335, respectively; (b) SEQ ID NOs: 302 and 336, respectively; (c) SEQ ID NOs: 302 and 340, respectively; (d) SEQ ID NOs: 302 and 342, respectively; (e) SEQ ID NOs: 302 and 343, respectively; (f) SEQ ID NOs: 302 and 346, respectively; (g) SEQ ID NOs: 306 and 337, respectively; (h) SEQ ID NOs: 306 and 339, respectively; (i) SEQ ID NOs: 306 and 341, respectively; (j) SEQ ID NOs: 306 and 344, respectively; (k) SEQ ID NOs: 306 and 345, respectively; (l) SEQ ID NOs: 306 and 347, respectively; (m) SEQ ID NOs: 308 and 340, respectively; (n) SEQ ID NOs: 309 and 341, respectively; (o) SEQ ID NOs: 310 and 336, respectively; (p) SEQ ID NOs: 311 and 337, respectively; (q) SEQ ID NOs: 313 and 336, respectively; (r) SEQ ID NOs: 313 and 342, respectively; (s) SEQ ID NOs: 313 and 343, respectively; (t) SEQ ID NOs: 316 and 337, respectively; (u) SEQ ID NOs: 316 and 344, respectively; (v) SEQ ID NOs: 316 and 345, respectively; (w) SEQ ID NOs: 318 and 336, respectively; (x) SEQ ID NOs: 318 and 340, respectively; (y) SEQ ID NOs: 318 and 346, respectively; (z) SEQ ID NOs: 319 and 336, respectively; (aa) SEQ ID NOs: 319 and 340, respectively; (bb) SEQ ID NOs: 319 and 346, respectively; (cc) SEQ ID NOs: 320 and 337, respectively; (dd) SEQ ID NOs: 320 and 341, respectively; (ee) SEQ ID NOs: 320 and 347, respectively; (ff) SEQ ID NOs: 320 and 743, respectively; (gg) SEQ ID NOs: 321 and 337, respectively; (hh) SEQ ID NOs: 321 and 341, respectively; or (ii) SEQ ID NOs: 321 and 347, respectively. In some embodiments, the anti-PD1 VH and anti-PD1 VL have amino acid sequences of: (a) SEQ ID NOs: 302 and 335, respectively; (b) SEQ ID NOs: 302 and 336, respectively; (c) SEQ ID NOs: 302 and 340, respectively; (d) SEQ ID NOs: 302 and 342, respectively; (e) SEQ ID NOs: 302 and 343, respectively; (f) SEQ ID NOs: 302 and 346, respectively; (g) SEQ ID NOs: 306 and 337, respectively; (h) SEQ ID NOs: 306 and 339, respectively; (i) SEQ ID NOs: 306 and 341, respectively; (j) SEQ ID NOs: 306 and 344, respectively; (k) SEQ ID NOs: 306 and 345, respectively; (l) SEQ ID NOs: 306 and 347, respectively; (m) SEQ ID NOs: 308 and 340, respectively; (n) SEQ ID NOs: 309 and 341, respectively; (o) SEQ ID NOs: 310 and 336, respectively; (p) SEQ ID NOs: 311 and 337, respectively; (q) SEQ ID NOs: 313 and 336, respectively; (r) SEQ ID NOs: 313 and 342, respectively; (s) SEQ ID NOs: 313 and 343, respectively; (t) SEQ ID NOs: 316 and 337, respectively; (u) SEQ ID NOs: 316 and 344, respectively; (v) SEQ ID NOs: 316 and 345, respectively; (w) SEQ ID NOs: 318 and 336, respectively; (x) SEQ ID NOs: 318 and 340, respectively; (y) SEQ ID NOs: 318 and 346, respectively; (z) SEQ ID NOs: 319 and 336, respectively; (aa) SEQ ID NOs: 319 and 340, respectively; (bb) SEQ ID NOs: 319 and 346, respectively; (cc) SEQ ID NOs: 320 and 337, respectively; (dd) SEQ ID NOs: 320 and 341, respectively; (ee) SEQ ID NOs: 320 and 347, respectively; (ff) SEQ ID NOs: 320 and 743, respectively; (gg) SEQ ID NOs: 321 and 337, respectively; (hh) SEQ ID NOs: 321 and 341, respectively; or (ii) SEQ ID NOs: 321 and 347, respectively.

In some embodiments the CDR-H1, CDR-H2, and CDR-H3 of the VH within a PD-1 binding region (anti-PD1 VH) comprise the amino acid sequences of (a) SEQ ID NOs: 445, 456, and 447, respectively; (b) SEQ ID NOs: 451, 458, and 453, respectively; or (c) SEQ ID NOs: 443, 460, and 447, respectively, and the CDR-L1, CDR-L2, and CDR-HL of the VL within a PD-1 binding region (anti-PD1 VL) comprise the amino acid sequences of (a) SEQ ID NOs: 448, 449, and 450, respectively; (b) SEQ ID NO: 454, SAS, and SEQ ID NO: 450, respectively; or (c) SEQ ID NOs: 448, 449, or 450, respectively, and the anti-PD1 VH and anti-PD1 VL have amino acid sequences that are at least 85% identical to that of: (a) SEQ ID NOs: 303 and 336, respectively; (b) SEQ ID NOs: 325 and 347, respectively; or (c) SEQ ID NOs: 325 and 743, respectively. In some embodiments, the anti-PD1 VH and anti-PD1 VL have amino acid sequences of: (a) SEQ ID NOs: 303 and 336, respectively; (b) SEQ ID NOs: 325 and 347, respectively; or (c) SEQ ID NOs: 325 and 743, respectively.

In some embodiments the CDR-H1, CDR-H2, and CDR-H3 of the VH within a PD-1 binding region (anti-PD1 VH) comprise the amino acid sequences of (a) SEQ ID NOs: 445, 461, and 447, respectively; (b) SEQ ID NOs: 451, 462, and 453, respectively; or (c) SEQ ID NOs: 443, 463, and 447, respectively, and the CDR-L1, CDR-L2, and CDR-HL of the VL within a PD-1 binding region (anti-PD1 VL) comprise the amino acid sequences of (a) SEQ ID NOs: 448, 449, and 450, respectively; (b) SEQ ID NO: 454, SAS, and SEQ ID NO: 450, respectively; or (c) SEQ ID NOs: 448, 449, or 450, respectively, and the anti-PD1 VH and anti-PD1 VL have amino acid sequences that are at least 85% identical to that of: SEQ ID NOs: 326 and 347, respectively; or SEQ ID NOs: 326 and 743, respectively. In some embodiments, the anti-PD1 VH and anti-PD1 VL have amino acid sequences of: SEQ ID NOs: 326 and 347, respectively; or SEQ ID NOs: 326 and 743, respectively.

In some embodiments the CDR-H1, CDR-H2, and CDR-H3 of the VH within a PD-1 binding region (anti-PD1 VH) comprise the amino acid sequences of (a) SEQ ID NOs: 445, 446, and 457, respectively; (b) SEQ ID NOs: 451, 452, and 459, respectively; or (c) SEQ ID NOs: 443, 455, and 457, respectively, and the CDR-L1, CDR-L2, and CDR-HL of the VL within a PD-1 binding region (anti-PD1 VL) comprise the amino acid sequences of (a) SEQ ID NOs: 448, 449, and 450, respectively; (b) SEQ ID NO: 454, SAS, and SEQ ID NO: 450, respectively; or (c) SEQ ID NOs: 448, 449, or 450, respectively, and the anti-PD1 VH and anti-PD1 VL have amino acid sequences that are at least 85% identical to that of: SEQ ID NOs: 327 and 347, respectively; or SEQ ID NOs: 327 and 743, respectively. In some embodiments, the anti-PD1 VH and anti-PD1 VL have amino acid sequences of: SEQ ID NOs: 327 and 347, respectively; or SEQ ID NOs: 327 and 743, respectively.

In some embodiments the CDR-H1, CDR-H2, and CDR-H3 of the VH within a PD-1 binding region (anti-PD1 VH) comprise the amino acid sequences of (a) SEQ ID NOs: 445, 461, and 457, respectively; (b) SEQ ID NOs: 451, 462, and 459, respectively; or (c) SEQ ID NOs: 443, 463, and 457, respectively, and the CDR-L1, CDR-L2, and CDR-HL of the VL within a PD-1 binding region (anti-PD1 VL) comprise the amino acid sequences of (a) SEQ ID NOs: 448, 449, and 450, respectively; (b) SEQ ID NO: 454, SAS, and SEQ ID NO: 450, respectively; or (c) SEQ ID NOs: 448, 449, or 450, respectively, and the anti-PD1 VH and anti-PD1 VL have amino acid sequences that are at least 85% identical to that of: (a) SEQ ID NOs: 303 and 335, respectively; (b) SEQ ID NOs: 303 and 342, respectively; (c) SEQ ID NOs: 303 and 343, respectively; (d) SEQ ID NOs: 307 and 337, respectively; (e) SEQ ID NOs: 307 and 344, respectively; (f) SEQ ID NOs: 307 and 345, respectively; (g) SEQ ID NOs: 314 and 336, respectively; (h) SEQ ID NOs: 314 and 342, respectively; (i) SEQ ID NOs: 314 and 343, respectively; (j) SEQ ID NOs: 317 and 337, respectively; (k) SEQ ID NOs: 317 and 344, respectively; (l) SEQ ID NOs: 317 and 345, respectively; or (m) SEQ ID NOs: 324 and 347, respectively. In some embodiments, the anti-PD1 VH and anti-PD1 VL have amino acid sequences of: (a) SEQ ID NOs: 303 and 335, respectively; (b) SEQ ID NOs: 303 and 342, respectively; (c) SEQ ID NOs: 303 and 343, respectively; (d) SEQ ID NOs: 307 and 337, respectively; (e) SEQ ID NOs: 307 and 344, respectively; (f) SEQ ID NOs: 307 and 345, respectively; (g) SEQ ID NOs: 314 and 336, respectively; (h) SEQ ID NOs: 314 and 342, respectively; (i) SEQ ID NOs: 314 and 343, respectively; (j) SEQ ID NOs: 317 and 337, respectively; (k) SEQ ID NOs: 317 and 344, respectively; (l) SEQ ID NOs: 317 and 345, respectively; or (m) SEQ ID NOs: 324 and 347, respectively.

In some embodiments the CDR-H1, CDR-H2, and CDR-H3 of the VH within a PD-1 binding region (anti-PD1 VH) comprise the amino acid sequences of (a) SEQ ID NOs: 445, 446, and 447, respectively; (b) SEQ ID NOs: 451, 452, and 453, respectively; or (c) SEQ ID NOs: 443, 455, and 447, respectively, and the CDR-L1, CDR-L2, and CDR-HL of the VL within a PD-1 binding region (anti-PD1 VL) comprise the amino acid sequences of (a) SEQ ID NOs: 448, 449, and 450, respectively; (b) SEQ ID NO: 454, SAS, and SEQ ID NO: 450, respectively; or (c) SEQ ID NOs: 448, 449, or 450, respectively, and the anti-PD1 VH and anti-PD1 VL have amino acid sequences that are at least 85% identical to that of: (a) SEQ ID NOs: 301 and 335, respectively; (b) SEQ ID NOs: 301 and 336, respectively; (c) SEQ ID NOs: 301 and 342, respectively; (d) SEQ ID NOs: 301 and 343, respectively; (e) SEQ ID NOs: 301 and 348, respectively; (f) SEQ ID NOs: 305 and 337, respectively; (g) SEQ ID NOs: 305 and 344, respectively; (h) SEQ ID NOs: 305 and 345, respectively; (i) SEQ ID NOs: 312 and 335, respectively; (j) SEQ ID NOs: 312 and 336, respectively; (k) SEQ ID NOs: 312 and 342, respectively; (l) SEQ ID NOs: 312 and 343, respectively; (m) SEQ ID NOs: 315 and 337, respectively; (n) SEQ ID NOs: 315 and 344, respectively; (o) SEQ ID NOs: 315 and 345, respectively; (p) SEQ ID NOs: 322 and 346, respectively; (q) SEQ ID NOs: 323 and 347, respectively; or (r) SEQ ID NOs: 323 and 743, respectively. In some embodiments, the anti-PD1 VH and anti-PD1 VL have amino acid sequences of: (a) SEQ ID NOs: 301 and 335, respectively; (b) SEQ ID NOs: 301 and 336, respectively; (c) SEQ ID NOs: 301 and 342, respectively; (d) SEQ ID NOs: 301 and 343, respectively; (e) SEQ ID NOs: 301 and 348, respectively; (f) SEQ ID NOs: 305 and 337, respectively; (g) SEQ ID NOs: 305 and 344, respectively; (h) SEQ ID NOs: 305 and 345, respectively; (i) SEQ ID NOs: 312 and 335, respectively; (j) SEQ ID NOs: 312 and 336, respectively; (k) SEQ ID NOs: 312 and 342, respectively; (l) SEQ ID NOs: 312 and 343, respectively; (m) SEQ ID NOs: 315 and 337, respectively; (n) SEQ ID NOs: 315 and 344, respectively; (o) SEQ ID NOs: 315 and 345, respectively; (p) SEQ ID NOs: 322 and 346, respectively; (q) SEQ ID NOs: 323 and 347, respectively; or (r) SEQ ID NOs: 323 and 743, respectively.

In some embodiments, a binding protein provided herein comprises a first VH sequence selected from SEQ ID NO: 295 or SEQ ID NO: 296 and a second VH sequence selected from SEQ ID NOs: 301-334 and 738-742; and a first VL sequence selected from SEQ ID NOs: 298-300, and a second VL sequence selected from SEQ ID NOs: 335-349 and 743-744. In some embodiments, a binding protein provided herein comprises a first VH sequence comprising SEQ ID NO: 295 and a second VH sequence selected from SEQ ID NOs: 301-334 and 738-742; and a first VL sequence comprising SEQ ID NOs: 298 and a second VL sequence selected from SEQ ID NOs: 335-349 and 743-744.

In certain aspects, any of SEQ ID NO: 295 or SEQ ID NO: 296 can be combined with any of SEQ ID NOs: 298-300, and any of SEQ ID NOs: 301-334 and 738-742 can be combined with any of SEQ ID NOs: 335-349 and 743-744.

In some embodiments, a binding protein provided herein comprises a first VH sequence having at least about 80%, 90%, 91%, 92%, 93%, 94%, 95%, 96%, 97%, 98% or 99% identity to a VH sequence provided in selected from SEQ ID NO: 295 or SEQ ID NO: 296 and a second VH sequence selected from SEQ ID NOs: 301-334 and 738-742; and a first VL sequence having at least about 80%, 90%, 91%, 92%, 93%, 94%, 95%, 96%, 97%, 98% or 99% identity to a VL sequence provided in SEQ ID NOs: 298-300 and a second VL sequence selected from SEQ ID NOs: 335-349 and 743-744. In some embodiments, a binding protein provided herein comprises a first VH sequence having at least about 80%, 90%, 91%, 92%, 93%, 94%, 95%, 96%, 97%, 98% or 99% identity to a VH sequence comprising SEQ ID NO: 295 and a second VH sequence selected from SEQ ID NOs 301-334 and 738-742; and a first VL sequence having at least about 80%, 90%, 91%, 92%, 93%, 94%, 95%, 96%, 97%, 98% or 99% identity to a VL sequence comprising SEQ ID NO: 298 and a second VL sequence selected from SEQ ID NOs: 335-349 and 743-744.

In some embodiments, a binding protein provided herein comprises a first VH sequence provided in SEQ ID NO: 295 or SEQ ID NO: 296 and a second VH sequence selected from SEQ ID NOs: 301-334 and 738-742, with up to 1, 2, 3, 4, 5, 6, 7, 8, 9, 10, 11, 12, 13, 14, 15, 16, 17, 18, 19, or 20 amino acid substitutions; and a first VL sequence provided in SEQ ID NOs: 298-300 and a second VL sequence selected from SEQ ID NOs: 335-349 and 743-744, with up to 1, 2, 3, 4, 5, 6, 7, 8, 9, 10, 11, 12, 13, 14, 15, 16, 17, 18, 19, or 20 amino acid substitutions. In some embodiments, a binding protein provided herein comprises a first VH sequence comprising SEQ ID NO: 295 and a second VH sequence selected from SEQ ID NOs: 301-334 and 738-742, with up to 1, 2, 3, 4, 5, 6, 7, 8, 9, 10, 11, 12, 13, 14, 15, 16, 17, 18, 19, or 20 amino acid substitutions; and a first VL sequence comprising SEQ ID NO: 298 and a second VL sequence selected from SEQ ID NOs: 335-349 and 743-744, with up to 1, 2, 3, 4, 5, 6, 7, 8, 9, 10, 11, 12, 13, 14, 15, 16, 17, 18, 19, or 20 amino acid substitutions. In some embodiments, the amino acid substitutions are conservative amino acid substitutions. In some embodiments, the binding proteins described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining binding proteins.

### Bispecific Formats

In some embodiments, the format of the bispecific binding proteins disclosed herein have any of the bispecific antibody formats described herein. In some embodiments, the format of the bispecific binding proteins disclosed herein is selected from the group consisting of (a) single chain Fv (scFv), (b) tandem scFv format of bispecific T cell engager (BiTE), (c) disulfide-linked diabody format of dual affinity retargeting (DART) bsAb, (d) tandem diabody (TandAb), (e) single-domain antibody (VHH), (f) conventional immunoglobulin G (IgG), (g) IgGs with additional binding units such as scFv, (h) IgGs with additional binding units such as VHH, (i) dual variable domain immunoglobulin (DVD-Ig), (j) quadromab bsAb, (k) knobs-into-holes (KiH) bsAb with a common light chain, (l) KiH-CrossMabCH1-CL, and (m) bsAb by controlled Fab arm exchange (cFAE). (see Shim et al. (2020) Biomolecules 26; 10(3):360.) In some embodiments, the format of the bispecific binding proteins disclosed herein is selected from the group consisting of IgG-scFv, IgG-VHH, and DVD-Ig. In some embodiments, the format of the bispecific binding proteins disclosed herein is IgG-scFv. In some embodiments, the format of the bispecific binding proteins disclosed herein is IgG-VHH. In some embodiments, the format of the bispecific binding proteins disclosed herein is DVD-Ig. In some embodiments, the format of the bispecific binding proteins disclosed herein is kiH-CrossMabCH1-CL.

In some embodiments, bispecific binding proteins comprise an scFv which is characterized one or more stabilizing features, e.g., particular amino acid residues or sequences. In some embodiments, the one or more stabilizing features in the scFv are an engineered disulfide bond between the contact surface of the VH and VL domains. In some embodiments, the disulfide bond is at the position of H44-L100 (Kabat numbering).

In some embodiments, the binding protein comprises a heavy chain as disclosed in Table 4A or Table 4B and a light chain disclosed in Table 4A or Table 4B. In some embodiments, a binding protein provided herein comprises a first VH sequence as listed in Table 5A and second VH sequence as listed in Table 5B. In some embodiments, a binding protein provided herein comprises a first VL sequence as listed in Table 6A and second VL sequence as listed in Table 6B. In some embodiments, a binding protein provided herein comprises a heavy chain constant region as listed in Table 7. In some embodiments, a binding protein provided herein comprises a light chain constant region as listed in Table 8. In some embodiments, a binding protein provided herein comprises scFv or VHH sequence as listed in Table 9. In some embodiments, a binding protein provided herein comprises a first VH sequence as listed in Table 5A and second VH sequence as listed in Table 5B; a first VL sequence as listed in Table 6A and second VL sequence as listed in Table 6B; a heavy chain constant region as listed in Table 7; a light chain constant region as listed in Table 8; and a scFv or VHH sequence as listed in Table 9.

In some embodiments, the format of the bispecific binding proteins disclosed herein is IgG-scFv. In some embodiments, the bispecific format comprises a first VH sequence (VH1) comprising any one of SEQ ID NO: 295 or SEQ ID NO: 296, a heavy chain constant region (CH(1-3)) comprising any one of SEQ ID NOs: 350-352, and a first linker comprising at least 90% sequence identity to GGGGSGGGGSGGGGSGGGGS (SEQ ID NO: 1030), a first VL sequence (VL1) comprising any one of SEQ ID NOs: 298-300, a second linker comprising at least 90% sequence identity to GGGGSGGGGSGGGGSGGGGS (SEQ ID NO: 1030), a second VH (VH2) sequence comprising any one of SEQ ID NOs: 301-334 and 738-742, a second VL sequence (VL2) comprising any one of SEQ ID NOs: 335-349 and 743-744, and a light chain constant domain (CL) comprising SEQ ID NO: 353.

In some embodiments, the bispecific format comprises a first VH sequence (VH1) comprising any one of SEQ ID NOs: 301-334 and 738-742, a heavy chain constant region (CH(1-3)) comprising any one of SEQ ID NOs: 350-352, and a first linker comprising at least 90% sequence identity to GGGGSGGGGSGGGGSGGGGS (SEQ ID NO: 1030), a first VL sequence (VL1) comprising any one of SEQ ID NOs: 298-300, a second linker comprising at least 90% sequence identity to GGGGSGGGGSGGGGSGGGGS (SEQ ID NO: 1030), a second VH (VH2) sequence comprising any one of SEQ ID NO: 295 or SEQ ID NO: 296, a second VL sequence (VL2) comprising any one of SEQ ID NOs: 335-349 and 743-744, and a light chain constant domain (CL) comprising SEQ ID NO: 353.

In some embodiments, the bispecific format comprises a first VH sequence (VHT) comprising any one of SEQ ID NOs: 295-296, 301-334, and 738-742, a heavy chain constant region (CH(1-3)) comprising any one of SEQ ID NOs: 350-352, and a first linker comprising at least 90% sequence identity to GGGGSGGGGSGGGGSGGGGS (SEQ ID NO: 1030), a scFv or VHH comprising any one of SEQ ID NOs: 354-430, a first VL sequence comprising any one of SEQ ID Nos: 298-300, and a light chain constant domain (CL) comprising SEQ ID NO: 353.

TABLE 7
Sequences of Exemplary Heavy Chain
Constant Regions 1-3

| SEQ ID | |
|---|---|
| NO | CH1-3 |
| 350 | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGG |
| | PSVFLFPPKPKDTLMISRTPEVTCVVVDVS |
| | HEDPEVKENWYVDGVEVHNAKIKPREEQYN |
| | STYRVVSVLIVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGK |
| 351 | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGG |
| | PSVFLFPPKPKDTLYITREPEVTCVVVDVS |
| | HEDPEVKENWYVDGVEVHNAKIKPREEQYN |
| | STYRVVSVLIVLHQDWLNGKEYKCKVSNKA |
| | LPAPIEKTISKAKGQPREPQVYTLPPSRDE |
| | LIKNQVSLICLVKGFYPSDIAVEWESNGQP |
| | ENNYKTTPPVLDSDGSFFLYSKLTVDKSRW |
| | QQGNVFSCSVMHEALHNHYTQKSLSLSPGK |
| 352 | ASTKGPSVFPLAPSSKSTSGGTAALGCLVK |
| | DYFPEPVTVSWNSGALTSGVHTFPAVLQSS |
| | GLYSLSSVVTVPSSSLGTQTYICNVNHKPS |
| | NTKVDKKVEPKSCDKTHTCPPCPAPEAAGG |
| | PSVFLFPPKPKDTLMISRTPEVTCVVVDVS |
| | HEDPEVKENWYVDGVEVHNAKIKPREEQYN |
| | STYRVVSVLIVLHQDWLNGKEYKCKVSNKA |
| | LPAPIEKTISKAKGQPREPQVYTLPPSRDE |
| | LTKNQVSLTCLVKGFYPSDIAVEWESNGQP |
| | ENNYKTTPPVLDSDGSFFLYSKLTVDKSRW |
| | QQGNVFSCSVLHEALHSHYTQKSLSLSPGK |

TABLE 8
Sequence of Exemplary Light Constant Region

| SEQ ID | |
|---|---|
| NO | CL |
| 353 | RTVAAPSVFIFPPSDEQLKSGTASVVCLLNNFY |
| | PREAKVQWKVDNALQSGNSQESVTEQDSKDSTY |
| | SLSSTLILSKADYEKHKVYACEVTHQGLSSPVT |
| | KSENRGEC |

TABLE 9
Exemplary scFv and VHH Sequences

| SEQ | |
|---|---|
| ID NO | scFV |
| 354 | QVQLVQSGVEVKKPGASVKVSCKASGYTFTNYYMYWVRQAPGQGLEWMGGINPSNGG |
| | TNFNEKFKNRVTLTIDSSTTTAYMELKSLQFDDTAVYYCARRDYRFDMGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 355 | QVQLVQSGVEVKKPGASVKVSCKASGYTFTNYYMYWVRQAPGQGLEWMGGINPSQGG |
| | TNFNEKFKNRVTLTTDSSTTTAYMELKSLQFDDTAVYYCARRDYRFDLGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 356 | QVQLVQSGVEVKKPGASVKVSCKASGYTFTNYYMYWVRQAPGQGLEWMGGINPSRGG |
| | TNFNEKFKNRVTLTTDSSTTTAYMELKSLQFDDTAVYYCARRDYREDLGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 357 | QVQLVQSGVEVKKPGASVKVSCKASGYTFTNYYMYWVRQAPGQCLEWMGGINPSNGG |
| | TNFNEKFKNRVTLTTDSSTTTAYMELKSLQFDDTAVYYCARRDYRFDMGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 358 | QVQLVQSGVEVKKPGASVKVSCKASGYTFTNYYMYWVRQAPGQCLEWMGGINPSQGG |
| | TNFNEKFKNRVTLTTDSSTTTAYMELKSLQFDDTAVYYCARRDYRFDLGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 359 | QVQLVQSGVEVKKPGASVKVSCKASGYTFTNYYMYWVRQAPGQCLEWMGGINPSRGG |
| | TNFNEKFKNRVTLTTDSSTTTAYMELKSLQFDDTAVYYCARRDYRFDLGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 360 | EIVLTQSPATLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGGGTKVEIKSSG |
| | GGGSGGGGSGGGGSGGGGSQVQLVQSGVEVKKPGASVKVSCKASGYTFTNYYMYWVR |
| | QAPGQGLEWMGGINPSQGGTNFNEKFKNRVTLTTDSSTTTAYMELKSLQFDDTAVYY |
| | CARRDYRFDLGFDYWGQGTTVTVSS |
| 361 | EIVLTQSPATLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGCGTKVEIKGGG |
| | GSGGGGSGGGGSGGGGSQVQLVQSGVEVKKPGASVKVSCKASGYTFTNYYMYWVRQA |
| | PGQCLEWMGGINPSQGGTNFNEKFKNRVTLTTDSSTTTAYMELKSLQFDDTAVYYCA |
| | RRDYRFDLGFDYWGQGTTVTVSS |
| 362 | EVQLVESGGGLVQPGGSLRLSCAASGYTFTNYYMYWVRQAPGKGLEWMGGINPSQGG |
| | TNFNEKFKNRFTISRDNSKNTLYLQMNSLRAEDTAVYYCARRDYREDLGFDYWGQGT |
| | LVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGGGTKVEIKR |
| 363 | EVQLVESGGGLVQPGGSLRLSCAASGYTFTNYYMYWVRQAPGKCLEWMGGINPSQGG |
| | TNFNEKFKNRFTISRDNSKNTLYLQMNSLRAEDTAVYYCARRDYRFDLGEDYWGQGT |
| | LVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGCGTKVEIKR |
| 364 | QVQLVQSGVEVKKPGASVKVSCKASGYTFTNYYMYWVRQAPGQGLEWMGGINPSQGG |
| | TNFNEKFKNRVTLTTDSSTTTAYMELKSLQFDDTAVYYCARRDYRFDLGEDYWGQGT |
| | GVRVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 365 | QVQLVQSGVEVKKPGASVKVSCKASGYTFTNYYMYWVRQAPGQCLEWMGGINPSQGG |
| | TNFNEKFKNRVTLTTDSSTTTAYMELKSLQFDDTAVYYCARRDYRFDLGEDYWGQGT |
| | GVRVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 366 | QVQLVQSGVEVKKPGASVKVSCKASGYTFTNYYMYWVRQAPGQGLEWMGGINPSNGG |
| | TNFNEKFKNRVTLTTDSSTTTAYMELKSLRFDDTAVYYCARRDYRFDMGEDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 367 | QVQLVQSGVEVKKPGASVKVSCKASGYTFTNYYMYWVRQAPGQGLEWMGGINPSNGG |
| | TNFNEKFKNRVTLTTDSSTTTAYMELKSLRFDDTAVYYCARRDYRFDMGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 368 | QVQLVQSGVEVKKPGASVKVSCKASGYTFTNYYMYWVRQAPGQGLEWMGGINPSNGG |
| | TNFNEKFKNRVTLTTDSSTTTAYMELKSLRFDDTAVYYCARRDYRFDMGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGGGTKVEIKR |
| 369 | QVQLVQSGVEVKKPGASVKVSCKASGYTFTNYYMYWVRQAPGQGLEWMGGINPSNGG |
| | TNFNEKFKNRVTLTTDSSTTTAYMELKSLQFDDTAVYYCARRDYRFDMGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 370 | QVQLVQSGVEVKKPGASVKVSCKASGYTFTNYYMYWVRQAPGQGLEWMGGINPSNGG |
| | TNFNEKFKNRVTLITDSSTTTAYMELKSLQFDDTAVYYCARRDYRFDMGEDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGGGTKVEIKR |
| 371 | QVQLVQSGVEVKKPGASVKVSCKASGYTFTNYYMYWVRQAPGQGLEWMGGINPSQGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 372 | QVQLVQSGVEVKKPGASVKVSCKASGYTFTNYYMYWVRQAPGQGLEWMGGINPSQGG |
| | TNFNEKFKNRVTLITDSSTTTAYMELKSLREDDTAVYYCARRDYRFDLGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 373 | QVQLVQSGVEVKKPGASVKVSCKASGYTFTNYYMYWVRQAPGQGLEWMGGINPSQGG |
| | TNFNEKFKNRVTLTTDSSTTTAYMELKSLRFDDTAVYYCARRDYREDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGGGTKVEIKR |
| 374 | QVQLVQSGVEVKKPGASVKVSCKASGYTFTNYYMYWVRQAPGQGLEWMGGINPSQGG |
| | TNFNEKFKNRVTLTTDSSTTTAYMELKSLQFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 375 | QVQLVQSGVEVKKPGASVKVSCKASGYTFTNYYMYWVRQAPGQGLEWMGGINPSQGG |
| | TNFNEKFKNRVTLTTDSSTTTAYMELKSLQFDDTAVYYCARRDYRFDLGEDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGGGTKVEIKR |
| 376 | QVQLVQSGVEVKKPGASVKVSCKASGYTFTNYYMYWVRQAPGQGLEWMGGINPSRGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 377 | QVQLVQSGVEVKKPGASVKVSCKASGYTFTNYYMYWVRQAPGQGLEWMGGINPSRGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 378 | QVQLVQSGVEVKKPGASVKVSCKASGYTFTNYYMYWVRQAPGQGLEWMGGINPSRGG |
| | TNFNEKFKNRVTLTTDSSTTTAYMELKSLRFDDTAVYYCARRDYREDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGGGTKVEIKR |
| 379 | QVQLVQSGVEVKKPGASVKVSCKASGYTFTNYYMYWVRQAPGQGLEWMGGINPSRGG |
| | TNFNEKFKNRVTLITDSSTTTAYMELKSLQFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 380 | QVQLVQSGVEVKKPGASVKVSCKASGYTFTNYYMYWVRQAPGQGLEWMGGINPSRGG |
| | TNFNEKFKNRVTLTTDSSTTTAYMELKSLQFDDTAVYYCARRDYREDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGGGTKVEIKR |
| 381 | QVQLVQSGVEVKKPGASVKVSCKASGYTFTNYYMYWVRQAPGQCLEWMGGINPSNGG |
| | TNFNEKFKNRVTLTTDSSTTTAYMELKSLRFDDTAVYYCARRDYRFDMGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 382 | QVQLVQSGVEVKKPGASVKVSCKASGYTFTNYYMYWVRQAPGQCLEWMGGINPSNGG |
| | TNFNEKFKNRVTLTTDSSTTTAYMELKSLRFDDTAVYYCARRDYRFDMGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 383 | QVQLVQSGVEVKKPGASVKVSCKASGYTFTNYYMYWVRQAPGQCLEWMGGINPSNGG |
| | TNFNEKFKNRVTLTTDSSTTTAYMELKSLREDDTAVYYCARRDYRFDMGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGCGTKVEIKR |
| 384 | QVQLVQSGVEVKKPGASVKVSCKASGYTFTNYYMYWVRQAPGQCLEWMGGINPSNGG |
| | TNFNEKFKNRVTLTTDSSTTTAYMELKSLQFDDTAVYYCARRDYRFDMGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 385 | QVQLVQSGVEVKKPGASVKVSCKASGYTFTNYYMYWVRQAPGQCLEWMGGINPSNGG |
| | TNFNEKFKNRVTLTIDSSTTTAYMELKSLQFDDTAVYYCARRDYRFDMGEDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGCGTKVEIKR |
| 386 | QVQLVQSGVEVKKPGASVKVSCKASGYTFTNYYMYWVRQAPGQCLEWMGGINPSQGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 387 | QVQLVQSGVEVKKPGASVKVSCKASGYTFTNYYMYWVRQAPGQCLEWMGGINPSQGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 388 | QVQLVQSGVEVKKPGASVKVSCKASGYTFTNYYMYWVRQAPGQCLEWMGGINPSQGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGCGTKVEIKR |
| 389 | QVQLVQSGVEVKKPGASVKVSCKASGYTFTNYYMYWVRQAPGQCLEWMGGINPSQGG |
| | TNFNEKFKNRVTLTTDSSTTTAYMELKSLQFDDTAVYYCARRDYREDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 390 | QVQLVQSGVEVKKPGASVKVSCKASGYTFTNYYMYWVRQAPGQCLEWMGGINPSQGG |
| | TNFNEKFKNRVTLTTDSSTTTAYMELKSLQFDDTAVYYCARRDYREDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGCGTKVEIKR |
| 391 | QVQLVQSGVEVKKPGASVKVSCKASGYTFTNYYMYWVRQAPGQCLEWMGGINPSRGG |
| | TNFNEKFKNRVTLTTDSSTTTAYMELKSLRFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 392 | QVQLVQSGVEVKKPGASVKVSCKASGYTFTNYYMYWVRQAPGQCLEWMGGINPSRGG |
| | TNFNEKFKNRVTLTTDSSTTTAYMELKSLREDDTAVYYCARRDYREDLGFDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 393 | QVQLVQSGVEVKKPGASVKVSCKASGYTFTNYYMYWVRQAPGQCLEWMGGINPSRGG |
| | TNFNEKFKNRVTLTTDSSTTTAYMELKSLRFDDTAVYYCARRDYRFDLGEDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLTFGCGTKVEIKR |
| 394 | QVQLVQSGVEVKKPGASVKVSCKASGYTFTNYYMYWVRQAPGQCLEWMGGINPSRGG |
| | TNFNEKFKNRVTLTTDSSTTTAYMELKSLQFDDTAVYYCARRDYRFDLGFDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGIKVEIKR |
| 395 | QVQLVQSGVEVKKPGASVKVSCKASGYTFTNYYMYWVRQAPGQCLEWMGGINPSRGG |
| | TNFNEKFKNRVTLTTDSSTTTAYMELKSLQFDDTAVYYCARRDYRFDLGEDYWGQGT |
| | TVTVSSGGGGSGGGGSGGGGSGGGGSEIVLTQSPSTLSLSPGERATLSCRASKGVST |
| | SGYSYLHWYQQKPGQAPRLLIYLASYLESGVPARFSGSGSGTDFTLTISGLEPEDFA |
| | VYYCQHSRDLPLIFGCGTKVEIKR |
| 396 | QVQLVQSGVEVKKPGASVKVSCKASGYTFTNYYMYWVRQAPGQGLEWMGGINPSQGG |
| | TNFNEKFKNRVTLTTDSSTTTAYMELKSLQFDDTAVYYCARRDYRFDLGFDYWGQGT |
| | TVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLIFGGGTKVEIKR |
| 397 | QVQLVQSGVEVKKPGASVKVSCKASGYTFTNYYMYWVRQAPGQGLEWMGGINPSQGG |
| | TNFNEKFKNRVTLTTDSSTTTAYMELKSLQFDDTAVYYCARRDYRFDLGFDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGGGTKVEIKR |
| 398 | EVQLVQSGAEVKKPGASVKVSCKASGYTFTNYYMYWVRQAPGQGLEWMGGINPSQGG |
| | TNFNEKFKNRVTLTTDSSTSTAYMELSSLRSEDTAVYYCARRDYRFDLGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 399 | EVQLVQSGAEVKKPGASVKVSCKASGYTFTNYYMYWVRQAPGQGLEWMGGINPSQGG |
| | TNFNEKFKNRVTLTTDSSTSTAYMELSSLRSEDTAVYYCARRDYREDLGFDYWGQGT |
| | TVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGGGTKVEIKR |
| 400 | EVQLVQSGAEVKKPGASVKVSCKASGYTFTNYYMYWVRQAPGQGLEWMGGINPSQGG |
| | TNFNEKFKNRVTLTTDSSTSTAYMELSSLRSEDTAVYYCARRDYREDLGFDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGGGTKVEIKR |
| 401 | EVQLLESGGGLVQPGGSLRLSCKASGYTFTNYYMYWVRQAPGKGLEWMGGINPSQGG |
| | TNFNEKFKNRVTLSTDSSKNTAYLQMNSLRAEDTAVYYCARRDYRFDLGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGGGTKVEIKR |
| 402 | EVQLLESGGGLVQPGGSLRLSCKASGYTFTNYYMYWVRQAPGKGLEWMGGINPSQGG |
| | TNFNEKFKNRVTLSTDSSKNTAYLQMNSLRAEDTAVYYCARRDYRFDLGEDYWGQGT |
| | TVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGGGTKVEIKR |
| 403 | EVQLLESGGGLVQPGGSLRLSCKASGYTFTNYYMYWVRQAPGKGLEWMGGINPSQGG |
| | TNFNEKFKNRVTLSTDSSKNTAYLQMNSLRAEDTAVYYCARRDYRFDLGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGGGTKVEIKR |
| 404 | QVQLVQSGVEVKKPGASVKVSCKASGYTFTNYYMYWVRQAPGQCLEWMGGINPSQGG |
| | TNFNEKFKNRVTLTTDSSTTTAYMELKSLQFDDTAVYYCARRDYRFDLGFDYWGQGT |
| | TVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGCGTKVEIKR |
| 405 | QVQLVQSGVEVKKPGASVKVSCKASGYTFTNYYMYWVRQAPGQCLEWMGGINPSQGG |
| | TNFNEKFKNRVTLTTDSSTTTAYMELKSLQFDDTAVYYCARRDYRFDLGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 406 | EVQLVQSGAEVKKPGASVKVSCKASGYTFTNYYMYWVRQAPGQCLEWMGGINPSQGG |
| | TNFNEKFKNRVTLITDSSTSTAYMELSSLRSEDTAVYYCARRDYRFDLGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 407 | EVQLVQSGAEVKKPGASVKVSCKASGYTFTNYYMYWVRQAPGQCLEWMGGINPSQGG |
| | TNFNEKFKNRVTLTTDSSTSTAYMELSSLRSEDTAVYYCARRDYREDLGFDYWGQGT |
| | TVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGCGTKVEIKR |
| 408 | EVQLVQSGAEVKKPGASVKVSCKASGYTFTNYYMYWVRQAPGQCLEWMGGINPSQGG |
| | TNFNEKFKNRVTLTTDSSTSTAYMELSSLRSEDTAVYYCARRDYRFDLGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 409 | EVQLLESGGGLVQPGGSLRLSCKASGYTFTNYYMYWVRQAPGKCLEWMGGINPSQGG |
| | TNFNEKFKNRVTLSTDSSKNTAYLQMNSLRAEDTAVYYCARRDYRFDLGEDYWGQGT |
| | TVTVSSGGGGSGGGGSGGGGSGGGGSEIVLTQSPATLSLSPGERATLSCRASKGVST |
| | SGYSYLHWYQQKPGQAPRLLIYLASYLESGVPARFSGSGSGTDFTLTISSLEPEDFA |
| | VYYCQHSRDLPLTFGCGTKVEIKR |
| 410 | EVQLLESGGGLVQPGGSLRLSCKASGYTFTNYYMYWVRQAPGKCLEWMGGINPSQGG |
| | TNFNEKFKNRVTLSTDSSKNTAYLQMNSLRAEDTAVYYCARRDYRFDLGFDYWGQGT |
| | TVTVSSGGGGSGGGGSGGGGSGGGGSEIVLTQSPATLSVSPGERATLSCRASKGVST |
| | SGYSYLHWYQQKPGQAPRLLIYLASYLESGIPARFSGSGSGTEFTLTISSLQSEDFA |
| | VYYCQHSRDLPLTFGCGTKVEIKR |
| 411 | EVQLLESGGGLVQPGGSLRLSCKASGYTFTNYYMYWVRQAPGKCLEWMGGINPSQGG |
| | TNFNEKFKNRVTLSTDSSKNTAYLQMNSLRAEDTAVYYCARRDYRFDLGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 412 | EIVLTQSPATLSLSPGERATLSCRASKGVSTSGYSYLHWYQQKPGQAPRLLIYLASY |
| | LESGVPARFSGSGSGIDFTLTISSLEPEDFAVYYCQHSRDLPLTFGGGTKVEIKGGG |
| | GSGGGGSGGGGSGGGGSQVQLVQSGVEVKKPGASVKVSCKASGYTFTNYYMYWVRQA |
| | PGQGLEWMGGINPSNGGTNFNEKFKNRVTLTIDSSTTTAYMELKSLQFDDTAVYYCA |
| | RRDYRFDMGFDYWGQGTTVTVSS |
| 413 | EIVLTQSPATLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGGGTKVEIKGGG |
| | GSGGGGSGGGGSGGGGSQVQLVQSGVEVKKPGASVKVSCKASGYTFTNYYMYWVRQA |
| | PGQGLEWMGGINPSRGGTNFNEKFKNRVTLTTDSSTTTAYMELKSLQFDDTAVYYCA |
| | RRDYRFDLGFDYWGQGTTVTVSS |
| 414 | EIVLTQSPATLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGGGTKVEIKGGG |
| | GSGGGGSGGGGSGGGGSQVQLVQSGVEVKKPGASVKVSCKASGYTFTNYYMYWVRQA |
| | PGQGLEWMGGINPSNGGTNFNEKFKNRVTLTTDSSTTTAYMELKSLRFDDTAVYYCA |
| | RRDYRFDMGFDYWGQGTTVTVSS |
| 415 | EIVLTQSPSTLSLSPGERATLSCRASKGVSTSGYSYLHWYQQKPGQAPRLLIYLASY |
| | LESGVPARFSGSGSGTDFTLTISSLEPEDFAVYYCQHSRDLPLTFGGGTKVEIKGGG |
| | GSGGGGSGGGGSGGGGSQVQLVQSGVEVKKPGASVKVSCKASGYTFTNYYMYWVRQA |
| | PGQGLEWMGGINPSNGGTNFNEKFKNRVTLTTDSSTTTAYMELKSLQFDDTAVYYCA |
| | RRDYRFDMGFDYWGQGTTVTVSS |
| 416 | EVQLVQSGAEVKKPGASVKVSCKASGYTFTNYYMYWVRQAPGQGLEWMGGINPSNGG |
| | TNFNEKFKNRVTLTTDSSTSTAYMELSSLRSEDTAVYYCARRDYRFDMGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGGGTKVEIKR |
| 417 | EVQLVQSGAEVKKPGASVKVSCKASGYTFTNYYMYWVRQAPGQCLEWMGGINPSNGG |
| | TNFNEKFKNRVTLTTDSSTSTAYMELSSLRSEDTAVYYCARRDYRFDMGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLIFGCGTKVEIKR |
| 418 | EVQLVQSGAEVKKPGASVKVSCKASGYTFTNYYMYWVRQAPGQCLEWMGGINPSRGG |
| | TNFNEKFKNRVTLTTDSSTSTAYMELSSLRSEDTAVYYCARRDYRFDLGEDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 419 | EVQLVQSGAEVKKPGASVKVSCKASGYTFTNYYMYWVRQAPGQCLEWMGGINPSQGG |
| | TNFNEKFKNRVTLTTDSSTSTAYMELSSLRSEDTAVYYCARRDYRFDMGFDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 420 | EVQLVQSGAEVKKPGASVKVSCKASGYTFTNYYMYWVRQAPGQCLEWMGGINPSRGG |
| | TNFNEKFKNRVTLTTDSSTSTAYMELSSLRSEDTAVYYCARRDYRFDMGFDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 421 | EVQLVQSGAEVKKPGASVKVSCKASGYTFTNYYMYWVRQAPGQCLEWMGGINPSNGG |
| | TNFNEKFKNRVTLTTDSSTSTAYMELSSLRSEDTAVYYCARRDYRFDLGFDYWGQGT |
| | TVTVSSGGGGSGGGGSGGGGSGGGGSDIQLTQSPSSLSASVGDRVTITCRASKGVST |
| | SGYSYLHWYQQKPGKAPKLLIYLASYLESGVPSRFSGSGSGTDFTLTISSLQPEDFA |
| | TYYCQHSRDLPLTFGCGTKVEIKR |
| 422 | EVQLVESGGGLVQPGGSLRLSCAASGYTFTNYGMNWVRQAPGKGLEWVGWINTYTGE |
| | PTYAADFKRRFTFSLDISKSTAYLQMNSLRAEDTAVYYCAKYPHYYGSSHWYFDVWG |
| | QGTLVTVSSGGGGSGGGGSGGGGSGGGGSDIQMTQSPSSLSASVGDRVTITCSASQD |
| | ISNYLNWYQQKPGKAPKVLIYFTSSLHSGVPSRESGSGSGTDFTLTISSLQPEDFAT |
| | YYCQQYSTVPWTFGQGTKVEIKR |
| 423 | EVQLVESGGGLVQPGGSLRLSCAASGYTFTNYGMNWVRQAPGKCLEWVGWINTYTGE |
| | PTYAADFKRRFTFSLDTSKSTAYLQMNSLRAEDTAVYYCAKYPHYYGSSHWYFDVWG |
| | QGTLVTVSSGGGGSGGGGSGGGGSGGGGSDIQMTQSPSSLSASVGDRVTITCSASQD |
| | ISNYLNWYQQKPGKAPKVLIYFTSSLHSGVPSRFSGSGSGTDFTLTISSLQPEDFAT |
| | YYCQQYSTVPWTFGCGTKVEIKR |
| 424 | EVQLVESGGGLVQPGGSLRLSCAASGSIASIHAMGWVRQAPGKEREWVSVITWSGGI |
| | TYYADSVKGRFTISRDNSKNTLYLQMNSLRAEDTAVYYCAGDKHQSSWYDYWGQGTL |
| | VTVSS |
| 425 | EVQLVESGGGLVQPGGSLRLSCAASGSIASIHAMGWVRQAPGKEREWVSVITWSGGI |
| | TYYADSVKGRFTISRDNSKNIVYLQMNSLRAEDTAVYYCAGDKHQSSWYDYWGQGTL |
| | VTVSS |
| 426 | EVQLVESGGGLVQPGGSLRLSCAASGSIASIHAMGWVRQAPGKEREFVSVITWSGGI |
| | TYYADSVKGRFTISRDNSKNTLYLQMNSLRAEDTAVYYCAGDKHQSSWYDYWGQGTL |
| | VTVSS |
| 427 | EVQLVESGGGLVQPGGSLRLSCAASGSIASIHAMGWVRQAPGKEREFVSVITWSGGI |
| | TYYADSVKGRFTISRDNSKNTVYLQMNSLRAEDTAVYYCAGDKHQSSWYDYWGQGTL |
| | VTVSS |
| 428 | EVQLVESGGGLVQPGGSLRLSCAASGSIASIHAMGWVRQAPGKEREFVAVITWSGGI |
| | TYYADSVKGRFTISRDNSKNTVYLQMNSLRAEDTAVYYCAGDKHQSSWYDYWGQGTL |
| | VTVSS |
| 429 | EVQLVESGGGLVQPGGSLRLSCAASGSIASIHAMGWVRQAPGKEREWVAVITWSGGI |
| | TYYADSVKGRFTISRDNSKNIVYLQMNSLRAEDTAVYYCAGDKHQSSWYDYWGQGTL |
| | VTVSS |
| 430 | EVQLVESGGGLVQPGGSLRLSCAASGFAFSSYDMSWVRQAPGKGLDWVATISGGGRY |
| | TYYPDSVKGRFTISRDNSKNNLYLQMNSLRAEDTALYYCANRYGEAWFAYWGQGTLV |
| | TVSSGGGGSGGGGSGGGGSGGGGSDIQMTQSPSSMSASVGDRVTFTCRASQDINTYL |
| | SWFQQKPGKSPKTLIYRANRLVSGVPSRESGSGSGQDYTLTISSLQPEDMATYYCLQ |
| | YDEFPLTFGAGTKLELKR |

### Fc Modifications

Binding proteins (e.g., PD-1 binding proteins or bispecific PD-1/VEGF binding proteins) described herein typically comprise an immunoglobulin Fc region. Fc regions typically comprise one or more Fe polypeptides, such as a first Fc polypeptide and a second Fc polypeptide. An IgG Fc polypeptide typically contains two constant heavy domains (CH2 and CH-3) and a hinge region connected to the CH2 domain. Typical Fc regions comprise two Fc polypeptides which dimerize with one another; however, an Fc region may have a single Fc polypeptide or more than two Fc polypeptides, e.g., as may be present in some antibody formats.

In some embodiments, the binding proteins described herein comprise an IgG1 Fc region (e.g., human IgG1 Fc region), that is, except for having particular residue(s) at certain positions as noted herein, the Fc region has an amino acid sequence that is substantially similar to that of the Fc region within a wild type IgG1 Fc. In some embodiments, the wild type IgG1 Fc is a human IgG1 Fc, in which each Fc polypeptide comprises an amino acid sequence of SEQ ID NO: 792, 911, or 1022. In some embodiments, the binding proteins described herein comprise an Fc region, each Fc polypeptide of which has an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc polypeptide within a wild-type IgG1 Fc.

In certain embodiments, human IgG1 Fc regions include an SRDEL (SEQ ID NO: 1031) allotype or an SREEM (SEQ ID NO: 1032) allotype.

In some embodiments, the binding proteins described herein comprise an IgG2 Fc region (e.g., human IgG2 Fc region), that is, except for having particular residue(s) at certain positions as noted herein, the Fc region has an amino acid sequence that is substantially similar to that of the Fc region within a wild type IgG2 Fc. In some embodiments, the wild type IgG2 Fc is a human IgG2 Fc, in which each Fc polypeptide comprises an amino acid sequence of SEQ ID NO: 793 or 912. In some embodiments, the binding proteins described herein comprise an Fc region, each Fc polypeptide of which has an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc polypeptide within a wild-type IgG2 Fc.

In some embodiments, the binding proteins described herein comprise an IgG4 Fc region (e.g., human IgG4 Fc region), that is, except for having particular residue(s) at certain positions as noted herein, the Fc region has an amino acid sequence that is substantially similar to that of the Fc region within a wild type IgG4 Fc. In some embodiments, the wild type IgG4 Fc is a human IgG4 Fc, in which each Fc polypeptide comprises an amino acid sequence of SEQ ID NO: 794 or 913. In some embodiments, the binding proteins described herein comprise an Fc region, each Fc polypeptide of which has an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc polypeptide within a wild-type IgG4 Fc.

In some embodiments, the binding protein comprises a modified Fc comprising one or more modifications. In some embodiments, the one or more modifications are located in a Fc from IgG1 (e.g., human IgG1 (hIgG1). In some embodiments, the one or more modifications are located in a Fc from IgG4 (e.g., human IgG4 (hIgG4). In some embodiments, the one or more modifications are located in a Fc from IgG2. In some embodiments, the one or more modifications promote selective binding of Fc-gamma receptors. In any embodiment, a constant heavy chain region can include a C-terminal lysine.

Amino acid sequences of exemplary Fc regions are provided in Tables 10A and 10B. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

TABLE 10A
Exemplary Fc Polypeptide Sequences

| | SEQ ID | |
|---|---|---|
| Name | NO | Fc polypeptide sequence |
| hIgG1 | 792 | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG2 | 793 | ASTKGPSVFPLAPCSRSTSESTAALGCLVKDYFPEPVTVSWNSGAL |
| | | TSGVHTFPAVLQSSGLYSLSSVVTVPSSNFGTQTYTCNVDHKPSNT |
| | | KVDKTVERKCCVECPPCPAPPVAGPSVFLFPPKPKDTLMISRTPEV |
| | | TCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVMHEALHNHYTQKSLSL |
| | | SPG |
| hIgG4 | 794 | ASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNT |
| | | KVDKRVESKYGPPCPSCPAPEFLGGPSVFLFPPKPKDTLMISRTPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLG |
| IgG4-SP | 795 | ASTKGPSVFPLAPCSRSTSESTAALGCLVKDYFPEPVTVSWNSGAL |
| (S228P) | | TSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRIPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLG |
| IgG4-SPLE | 796 | ASTKGPSVFPLAPCSRSTSESTAALGCLVKDYFPEPVTVSWNSGAL |
| (S228P/ | | TSGVHTFPAVLQSSGLYSLSSVVTVPSSSLGTKTYTCNVDHKPSNT |
| L235E) | | KVDKRVESKYGPPCPPCPAPEELGGPSVFLFPPKPKDTLMISRTPE |
| | | VTCVVVDVSQEDPEVQFNWYVDGVEVHNAKIKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKITPP |
| | | VLDSDGSFFLYSRLTVDKSRWQEGNVFSCSVMHEALHNHYTQKSLS |
| | | LSLG |
| hIgG1- | 797 | ASTKGPSVFPLAPSSKSTSGGTAALGCLVKDYFPEPVTVSWNSGAL |
| N297A | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYASTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 798 | ASTKGPSVFPLAPSSKSTSGGTAALGCLVKDYFPEPVTVSWNSGAL |
| D265A | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVAVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 799 | ASTKGPSVFPLAPSSKSTSGGTAALGCLVKDYFPEPVTVSWNSGAL |
| LALA | | TSGVHTFPAVLQSSGLYSLSSVVTVPSSSLGTQTYICNVNHKPSNT |
| (L234A/ | | KVDKKVEPKSCDKTHTCPPCPAPEAAGGPSVFLFPPKPKDTLMISR |
| L235A) | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 800 | ASTKGPSVFPLAPSSKSTSGGTAALGCLVKDYFPEPVTVSWNSGAL |
| LAGA | | TSGVHTFPAVLQSSGLYSLSSVVTVPSSSLGTQTYICNVNHKPSNT |
| (L235A/ | | KVDKKVEPKSCDKTHTCPPCPAPELAGAPSVFLFPPKPKDTLMISR |
| G237A) | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 801 | ASTKGPSVFPLAPSSKSTSGGTAALGCLVKDYFPEPVTVSWNSGAL |
| LALAGA | | TSGVHTFPAVLQSSGLYSLSSVVTVPSSSLGTQTYICNVNHKPSNT |
| (L234A/ | | KVDKKVEPKSCDKTHTCPPCPAPEAAGAPSVFLFPPKPKDTLMISR |
| L235A/ | | TPEVTCVVVDVSHEDPEVKFNWYVDGVEVHNAKTKPREEQYNSTYR |
| G237A) | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 802 | ASTKGPSVFPLAPSSKSTSGGTAALGCLVKDYFPEPVTVSWNSGAL |
| LALAPG | | TSGVHTFPAVLQSSGLYSLSSVVTVPSSSLGTQTYICNVNHKPSNT |
| (L234A/ | | KVDKKVEPKSCDKTHTCPPCPAPEAAGGPSVFLFPPKPKDTLMISR |
| L235A/ | | TPEVTCVVVDVSHEDPEVKFNWYVDGVEVHNAKIKPREEQYNSTYR |
| P329G) | | VVSVLTVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1-YTE | 803 | ASTKGPSVFPLAPSSKSTSGGTAALGCLVKDYFPEPVTVSWNSGAL |
| (M252Y/ | | TSGVHTFPAVLQSSGLYSLSSVVTVPSSSLGTQTYICNVNHKPSNT |
| S254T/ | | KVDKKVEPKSCDKTHTCPPCPAPELLGGPSVFLFPPKPKDTLYITR |
| T256E) | | EPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 804 | ASTKGPSVFPLAPSSKSTSGGTAALGCLVKDYFPEPVTVSWNSGAL |
| N297A/YTE | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLYITR |
| | | EPEVTCVVVDVSHEDPEVKFNWYVDGVEVHNAKTKPREEQYASTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 805 | ASTKGPSVFPLAPSSKSTSGGTAALGCLVKDYFPEPVTVSWNSGAL |
| D265A/YTE | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLYITR |
| | | EPEVTCVVVAVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 806 | ASTKGPSVFPLAPSSKSTSGGTAALGCLVKDYFPEPVTVSWNSGAL |
| LALA/YTE | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLYITR |
| | | EPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 807 | ASTKGPSVFPLAPSSKSTSGGTAALGCLVKDYFPEPVTVSWNSGAL |
| LAGA/YTE | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLYITR |
| | | EPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 808 | ASTKGPSVFPLAPSSKSTSGGTAALGCLVKDYFPEPVTVSWNSGAL |
| LALAGA/YTE | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLYITR |
| | | EPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 809 | ASTKGPSVFPLAPSSKSTSGGTAALGCLVKDYFPEPVTVSWNSGAL |
| LALAPG/YTE | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLYITR |
| | | EPEVTCVVVDVSHEDPEVKFNWYVDGVEVHNAKTKPREEQYNSTYR |
| | | VVSVLTVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1-LS | 810 | ASTKGPSVFPLAPSSKSTSGGTAALGCLVKDYFPEPVTVSWNSGAL |
| (M428L/ | | TSGVHTFPAVLQSSGLYSLSSVVTVPSSSLGTQTYICNVNHKPSNT |
| N434S) | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1- | 811 | ASTKGPSVFPLAPSSKSTSGGTAALGCLVKDYFPEPVTVSWNSGAL |
| N297A/LS | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKIKPREEQYASTYR |
| | | VVSVLIVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1- | 812 | ASTKGPSVFPLAPSSKSTSGGTAALGCLVKDYFPEPVTVSWNSGAL |
| D265A/LS | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVAVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1- | 813 | ASTKGPSVFPLAPSSKSTSGGTAALGCLVKDYFPEPVTVSWNSGAL |
| LALA/LS | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1- | 814 | ASTKGPSVFPLAPSSKSTSGGTAALGCLVKDYFPEPVTVSWNSGAL |
| LAGA/LS | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1- | 815 | ASTKGPSVFPLAPSSKSTSGGTAALGCLVKDYFPEPVTVSWNSGAL |
| LALAGA/LS | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1- | 816 | ASTKGPSVFPLAPSSKSTSGGTAALGCLVKDYFPEPVTVSWNSGAL |
| LALAPG/LS | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKIKPREEQYNSTYR |
| | | VVSVLTVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1-DHS | 817 | ASTKGPSVFPLAPSSKSTSGGTAALGCLVKDYFPEPVTVSWNSGAL |
| (L309D/ | | TSGVHTFPAVLQSSGLYSLSSVVTVPSSSLGTQTYICNVNHKPSNT |
| Q311H/ | | KVDKKVEPKSCDKTHTCPPCPAPELLGGPSVFLFPPKPKDTLMISR |
| N434S) | | TPEVTCVVVDVSHEDPEVKFNWYVDGVEVHNAKIKPREEQYNSTYR |
| | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1- | 818 | ASTKGPSVFPLAPSSKSTSGGTAALGCLVKDYFPEPVTVSWNSGAL |
| N297A/DHS | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYASTYR |
| | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1- | 819 | ASTKGPSVFPLAPSSKSTSGGTAALGCLVKDYFPEPVTVSWNSGAL |
| D265A/DHS | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVAVSHEDPEVKFNWYVDGVEVHNAKTKPREEQYNSTYR |
| | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1- | 820 | ASTKGPSVFPLAPSSKSTSGGTAALGCLVKDYFPEPVTVSWNSGAL |
| LALA/DHS | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1- | 821 | ASTKGPSVFPLAPSSKSTSGGTAALGCLVKDYFPEPVTVSWNSGAL |
| LAGA/DHS | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | TPEVTCVVVDVSHEDPEVKFNWYVDGVEVHNAKTKPREEQYNSTYR |
| | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1- | 822 | ASTKGPSVFPLAPSSKSTSGGTAALGCLVKDYFPEPVTVSWNSGAL |
| LALAGA/DHS | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | TPEVTCVVVDVSHEDPEVKFNWYVDGVEVHNAKIKPREEQYNSTYR |
| | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1- | 823 | ASTKGPSVFPLAPSSKSTSGGTAALGCLVKDYFPEPVTVSWNSGAL |
| LALAPG/DHS | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVDHHDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG4-YTE | 824 | ASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNT |
| | | KVDKRVESKYGPPCPSCPAPEFLGGPSVFLFPPKPKDTLYITREPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLG |
| hIgG4- | 825 | ASTKGPSVFPLAPCSRSTSESTAALGCLVKDYFPEPVTVSWNSGAL |
| SP/YTE | | TSGVHTFPAVLQSSGLYSLSSVVTVPSSSLGTKTYTCNVDHKPSNT |
| | | KVDKRVESKYGPPCPPCPAPEFLGGPSVFLFPPKPKDTLYITREPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLG |
| hIgG4- | 826 | ASTKGPSVFPLAPCSRSTSESTAALGCLVKDYFPEPVTVSWNSGAL |
| SPLE/YTE | | TSGVHTFPAVLQSSGLYSLSSVVTVPSSSLGTKTYTCNVDHKPSNT |
| | | KVDKRVESKYGPPCPPCPAPEELGGPSVFLFPPKPKDTLYITREPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLG |
| hIgG4-LS | 827 | ASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNT |
| | | KVDKRVESKYGPPCPSCPAPEFLGGPSVFLFPPKPKDTLMISRTPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHSHYTQKSLS |
| | | LSLG |
| hIgG4- | 828 | ASTKGPSVFPLAPCSRSTSESTAALGCLVKDYFPEPVTVSWNSGAL |
| SP/LS | | TSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHSHYTQKSLS |
| | | LSLG |
| hIgG4- | 829 | ASTKGPSVFPLAPCSRSTSESTAALGCLVKDYFPEPVTVSWNSGAL |
| SPLE/LS | | TSGVHTFPAVLQSSGLYSLSSVVTVPSSSLGTKTYTCNVDHKPSNT |
| | | KVDKRVESKYGPPCPPCPAPEELGGPSVFLFPPKPKDTLMISRTPE |
| | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHSHYTQKSLS |
| | | LSLG |
| hIgG4-DHS | 830 | ASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNT |
| | | KVDKRVESKYGPPCPSCPAPEFLGGPSVFLFPPKPKDTLMISRTPE |
| | | VTCVVVDVSQEDPEVQFNWYVDGVEVHNAKTKPREEQFNSTYRVVS |
| | | VLTVDHHDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHSHYTQKSLS |
| | | LSLG |
| hIgG4- | 831 | ASTKGPSVFPLAPCSRSTSESTAALGCLVKDYFPEPVTVSWNSGAL |
| SP/DHS | | TSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPE |
| | | VTCVVVDVSQEDPEVQFNWYVDGVEVHNAKTKPREEQFNSTYRVVS |
| | | VLTVDHHDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHSHYTQKSLS |
| | | LSLG |
| hIgG4- | 832 | ASTKGPSVFPLAPCSRSTSESTAALGCLVKDYFPEPVTVSWNSGAL |
| SPLE/DHS | | TSGVHTFPAVLQSSGLYSLSSVVTVPSSSLGTKTYTCNVDHKPSNT |
| | | KVDKRVESKYGPPCPPCPAPEELGGPSVFLFPPKPKDTLMISRTPE |
| | | VTCVVVDVSQEDPEVQFNWYVDGVEVHNAKTKPREEQFNSTYRVVS |
| | | VLTVDHHDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHSHYTQKSLS |
| | | LSLG |
| hIgG2-YTE | 833 | ASTKGPSVFPLAPCSRSTSESTAALGCLVKDYFPEPVTVSWNSGAL |
| | | TSGVHTFPAVLQSSGLYSLSSVVTVPSSNFGTQTYTCNVDHKPSNT |
| | | KVDKTVERKCCVECPPCPAPPVAGPSVFLFPPKPKDTLYITREPEV |
| | | TCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVMHEALHNHYTQKSLSL |
| | | SPG |
| hIgG2-LS | 834 | ASTKGPSVFPLAPCSRSTSESTAALGCLVKDYFPEPVTVSWNSGAL |
| | | TSGVHTFPAVLQSSGLYSLSSVVTVPSSNFGTQTYTCNVDHKPSNT |
| | | KVDKTVERKCCVECPPCPAPPVAGPSVFLFPPKPKDTLMISRTPEV |
| | | TCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVLHEALHSHYTQKSLSL |
| | | SPG |
| hIgG2-DHS | 835 | ASTKGPSVFPLAPCSRSTSESTAALGCLVKDYFPEPVTVSWNSGAL |
| | | TSGVHTFPAVLQSSGLYSLSSVVTVPSSNFGTQTYTCNVDHKPSNT |
| | | KVDKTVERKCCVECPPCPAPPVAGPSVFLFPPKPKDTLMISRTPEV |
| | | TCVVVDVSHEDPEVQFNWYVDGVEVHNAKTKPREEQFNSTERVVSV |
| | | LTVDHHDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVMHEALHSHYTQKSLSL |
| | | SPG |
| hIgG1-LA | 836 | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 837 | ASTKGPSVFPLAPSSKSTSGGTAALGCLVKDYFPEPVTVSWNSGAL |
| N297A/LA | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKIKPREEQYASTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 838 | ASTKGPSVFPLAPSSKSTSGGTAALGCLVKDYFPEPVTVSWNSGAL |
| D265A/LA | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVAVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 839 | ASTKGPSVFPLAPSSKSTSGGTAALGCLVKDYFPEPVTVSWNSGAL |
| LALA/LA | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 840 | ASTKGPSVFPLAPSSKSTSGGTAALGCLVKDYFPEPVTVSWNSGAL |
| LAGA/LA | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | TPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 841 | ASTKGPSVFPLAPSSKSTSGGTAALGCLVKDYFPEPVTVSWNSGAL |
| LALAGA/LA | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 842 | ASTKGPSVFPLAPSSKSTSGGTAALGCLVKDYFPEPVTVSWNSGAL |
| LALAPG/LA | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKIKPREEQYNSTYR |
| | | VVSVLTVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPG |
| hIgG1- | 843 | ASTKGPSVFPLAPSSKSTSGGTAALGCLVKDYFPEPVTVSWNSGAL |
| N434A | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 844 | ASTKGPSVFPLAPSSKSTSGGTAALGCLVKDYFPEPVTVSWNSGAL |
| N297A/ | | TSGVHTFPAVLQSSGLYSLSSVVTVPSSSLGTQTYICNVNHKPSNT |
| N434A | | KVDKKVEPKSCDKTHTCPPCPAPELLGGPSVFLFPPKPKDTLMISR |
| | | TPEVTCVVVDVSHEDPEVKFNWYVDGVEVHNAKIKPREEQYASTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 845 | ASTKGPSVFPLAPSSKSTSGGTAALGCLVKDYFPEPVTVSWNSGAL |
| D265A/ | | TSGVHTFPAVLQSSGLYSLSSVVTVPSSSLGTQTYICNVNHKPSNT |
| N434A | | KVDKKVEPKSCDKTHTCPPCPAPELLGGPSVFLFPPKPKDTLMISR |
| | | TPEVTCVVVAVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 846 | ASTKGPSVFPLAPSSKSTSGGTAALGCLVKDYFPEPVTVSWNSGAL |
| LALA/ | | TSGVHTFPAVLQSSGLYSLSSVVTVPSSSLGTQTYICNVNHKPSNT |
| N434A | | KVDKKVEPKSCDKTHTCPPCPAPEAAGGPSVFLFPPKPKDTLMISR |
| | | TPEVTCVVVDVSHEDPEVKFNWYVDGVEVHNAKIKPREEQYNSTYR |
| | | VVSVLIVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 847 | ASTKGPSVFPLAPSSKSTSGGTAALGCLVKDYFPEPVTVSWNSGAL |
| LAGA/ | | TSGVHTFPAVLQSSGLYSLSSVVTVPSSSLGTQTYICNVNHKPSNT |
| N434A | | KVDKKVEPKSCDKTHTCPPCPAPELAGAPSVFLFPPKPKDTLMISR |
| | | TPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 848 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| N434A | | KVDKKVEPKSCDKTHTCPPCPAPEAAGAPSVFLFPPKPKDTLMISR |
| | | TPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 849 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| N434A | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLIVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPG |
| hIgG1- | 850 | ASTKGPSVFPLAPSSKSTSGGTAALGCLVKDYFPEPVTVSWNSGAL |
| N434W | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPG |
| hIgG1- | 851 | ASTKGPSVFPLAPSSKSTSGGTAALGCLVKDYFPEPVTVSWNSGAL |
| N297A/ | | TSGVHTFPAVLQSSGLYSLSSVVTVPSSSLGTQTYICNVNHKPSNT |
| N434W | | KVDKKVEPKSCDKTHTCPPCPAPELLGGPSVFLFPPKPKDTLMISR |
| | | TPEVTCVVVDVSHEDPEVKFNWYVDGVEVHNAKIKPREEQYASTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPG |
| hIgG1- | 852 | ASTKGPSVFPLAPSSKSTSGGTAALGCLVKDYFPEPVTVSWNSGAL |
| D265A/ | | TSGVHTFPAVLQSSGLYSLSSVVTVPSSSLGTQTYICNVNHKPSNT |
| N434W | | KVDKKVEPKSCDKTHTCPPCPAPELLGGPSVFLFPPKPKDTLMISR |
| | | TPEVTCVVVAVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPG |
| hIgG1- | 853 | ASTKGPSVFPLAPSSKSTSGGTAALGCLVKDYFPEPVTVSWNSGAL |
| LALA/ | | TSGVHTFPAVLQSSGLYSLSSVVTVPSSSLGTQTYICNVNHKPSNT |
| N434W | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | DPEVTCVVVAVSHEDPEVKFNWYVDGVEVHNAKTKPREEQYNSTYR |
| | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 860 | ASTKGPSVFPLAPSSKSTSGGTAALGCLVKDYFPEPVTVSWNSGAL |
| LALA/DQ | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | DPEVTCVVVDVSHEDPEVKFNWYVDGVEVHNAKTKPREEQYNSTYR |
| | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 861 | ASTKGPSVFPLAPSSKSTSGGTAALGCLVKDYFPEPVTVSWNSGAL |
| LAGA/DQ | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | DPEVTCVVVDVSHEDPEVKFNWYVDGVEVHNAKTKPREEQYNSTYR |
| | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 862 | ASTKGPSVFPLAPSSKSTSGGTAALGCLVKDYFPEPVTVSWNSGAL |
| LALAGA/DQ | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | DPEVTCVVVDVSHEDPEVKFNWYVDGVEVHNAKTKPREEQYNSTYR |
| | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 863 | ASTKGPSVFPLAPSSKSTSGGTAALGCLVKDYFPEPVTVSWNSGAL |
| LALAPG/DQ | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | DPEVTCVVVDVSHEDPEVKFNWYVDGVEVHNAKTKPREEQYNSTYR |
| | | VVSVLQVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1/DW | 864 | ASTKGPSVFPLAPSSKSTSGGTAALGCLVKDYFPEPVTVSWNSGAL |
| (T256D/ | | TSGVHTFPAVLQSSGLYSLSSVVTVPSSSLGTQTYICNVNHKPSNT |
| T307W) | | KVDKKVEPKSCDKTHTCPPCPAPELLGGPSVFLFPPKPKDTLMISR |
| | | DPEVTCVVVDVSHEDPEVKFNWYVDGVEVHNAKTKPREEQYNSTYR |
| | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 865 | ASTKGPSVFPLAPSSKSTSGGTAALGCLVKDYFPEPVTVSWNSGAL |
| N297A/DW | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | DPEVTCVVVDVSHEDPEVKFNWYVDGVEVHNAKTKPREEQYASTYR |
| | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 866 | ASTKGPSVFPLAPSSKSTSGGTAALGCLVKDYFPEPVTVSWNSGAL |
| D265A/DW | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | DPEVTCVVVAVSHEDPEVKFNWYVDGVEVHNAKTKPREEQYNSTYR |
| | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 867 | ASTKGPSVFPLAPSSKSTSGGTAALGCLVKDYFPEPVTVSWNSGAL |
| LALA/DW | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | DPEVTCVVVDVSHEDPEVKFNWYVDGVEVHNAKIKPREEQYNSTYR |
| | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 868 | ASTKGPSVFPLAPSSKSTSGGTAALGCLVKDYFPEPVTVSWNSGAL |
| LAGA/DW | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | DPEVTCVVVDVSHEDPEVKFNWYVDGVEVHNAKTKPREEQYNSTYR |
| | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 869 | ASTKGPSVFPLAPSSKSTSGGTAALGCLVKDYFPEPVTVSWNSGAL |
| LALAGA/DW | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | DPEVTCVVVDVSHEDPEVKFNWYVDGVEVHNAKTKPREEQYNSTYR |
| | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 870 | ASTKGPSVFPLAPSSKSTSGGTAALGCLVKDYFPEPVTVSWNSGAL |
| LALAPG/DW | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | DPEVTCVVVDVSHEDPEVKFNWYVDGVEVHNAKTKPREEQYNSTYR |
| | | VVSVLWVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1/YD | 871 | ASTKGPSVFPLAPSSKSTSGGTAALGCLVKDYFPEPVTVSWNSGAL |
| (M252Y/T256D) | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLYISR |
| | | DPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 872 | ASTKGPSVFPLAPSSKSTSGGTAALGCLVKDYFPEPVTVSWNSGAL |
| N297A/YD | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLYISR |
| | | DPEVTCVVVDVSHEDPEVKFNWYVDGVEVHNAKTKPREEQYASTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 873 | ASTKGPSVFPLAPSSKSTSGGTAALGCLVKDYFPEPVTVSWNSGAL |
| D265A/YD | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLYISR |
| | | DPEVTCVVVAVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 874 | ASTKGPSVFPLAPSSKSTSGGTAALGCLVKDYFPEPVTVSWNSGAL |
| LALA/YD | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLYISR |
| | | DPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 875 | ASTKGPSVFPLAPSSKSTSGGTAALGCLVKDYFPEPVTVSWNSGAL |
| LAGA/YD | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLYISR |
| | | DPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 876 | ASTKGPSVFPLAPSSKSTSGGTAALGCLVKDYFPEPVTVSWNSGAL |
| LALAGA/YD | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLYISR |
| | | DPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1- | 877 | ASTKGPSVFPLAPSSKSTSGGTAALGCLVKDYFPEPVTVSWNSGAL |
| LALAPG/YD | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLYISR |
| | | DPEVTCVVVDVSHEDPEVKFNWYVDGVEVHNAKTKPREEQYNSTYR |
| | | VVSVLTVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1/QVV | 878 | ASTKGPSVFPLAPSSKSTSGGTAALGCLVKDYFPEPVTVSWNSGAL |
| (T307Q/ | | TSGVHTFPAVLQSSGLYSLSSVVTVPSSSLGTQTYICNVNHKPSNT |
| Q311V/ | | KVDKKVEPKSCDKTHTCPPCPAPELLGGPSVFLFPPKPKDTLMISR |
| A378V) | | TPEVTCVVVDVSHEDPEVKFNWYVDGVEVHNAKTKPREEQYNSTYR |
| | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 879 | ASTKGPSVFPLAPSSKSTSGGTAALGCLVKDYFPEPVTVSWNSGAL |
| N297A/QVV | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYASTYR |
| | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 880 | ASTKGPSVFPLAPSSKSTSGGTAALGCLVKDYFPEPVTVSWNSGAL |
| D265A/QVV | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVAVSHEDPEVKFNWYVDGVEVHNAKIKPREEQYNSTYR |
| | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 88 | ASTKGPSVFPLAPSSKSTSGGTAALGCLVKDYFPEPVTVSWNSGAL |
| LALA/QVV | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKIKPREEQYNSTYR |
| | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 882 | ASTKGPSVFPLAPSSKSTSGGTAALGCLVKDYFPEPVTVSWNSGAL |
| LAGA/QVV | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | TPEVTCVVVDVSHEDPEVKFNWYVDGVEVHNAKTKPREEQYNSTYR |
| | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 883 | ASTKGPSVFPLAPSSKSTSGGTAALGCLVKDYFPEPVTVSWNSGAL |
| LALAGA/QVV | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYR |
| | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 884 | ASTKGPSVFPLAPSSKSTSGGTAALGCLVKDYFPEPVTVSWNSGAL |
| LALAPG/QVV | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLQVLHVDWLNGKEYKCKVSNKALGAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1/DDRVV | 885 | ASTKGPSVFPLAPSSKSTSGGTAALGCLVKDYFPEPVTVSWNSGAL |
| (T256D/ | | TSGVHTFPAVLQSSGLYSLSSVVTVPSSSLGTQTYICNVNHKPSNT |
| N286D/ | | KVDKKVEPKSCDKTHTCPPCPAPELLGGPSVFLFPPKPKDTLMISR |
| T307R/ | | DPEVTCVVVDVSHEDPEVKFNWYVDGVEVDNAKTKPREEQYNSTYR |
| Q311V/ | | VVSVLRVLHVDWLNGKEYKCKVSNKALPAPIEKTISKAKGQPREPQ |
| A378V) | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 886 | ASTKGPSVFPLAPSSKSTSGGTAALGCLVKDYFPEPVTVSWNSGAL |
| N297A/DDRVV | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | DPEVTCVVVDVSHEDPEVKFNWYVDGVEVDNAKTKPREEQYASTYR |
| | | VVSVLRVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 887 | ASTKGPSVFPLAPSSKSTSGGTAALGCLVKDYFPEPVTVSWNSGAL |
| D265A/DDRVV | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | DPEVTCVVVAVSHEDPEVKFNWYVDGVEVDNAKTKPREEQYNSTYR |
| | | VVSVLRVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 888 | ASTKGPSVFPLAPSSKSTSGGTAALGCLVKDYFPEPVTVSWNSGAL |
| LALA/DDRVV | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | DPEVTCVVVDVSHEDPEVKFNWYVDGVEVDNAKTKPREEQYNSTYR |
| | | VVSVLRVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 889 | ASTKGPSVFPLAPSSKSTSGGTAALGCLVKDYFPEPVTVSWNSGAL |
| LAGA/DDRVV | | TSGVHTFPAVLQSSGLYSLSSVVTVPSSSLGTQTYICNVNHKPSNT |
| | | KVDKKVEPKSCDKTHTCPPCPAPELAGAPSVFLFPPKPKDTLMISR |
| | | DPEVTCVVVDVSHEDPEVKFNWYVDGVEVDNAKTKPREEQYNSTYR |
| | | VVSVLRVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 890 | ASTKGPSVFPLAPSSKSTSGGTAALGCLVKDYFPEPVTVSWNSGAL |
| LALAGA/DDRVV | | TSGVHTFPAVLQSSGLYSLSSVVTVPSSSLGTQTYICNVNHKPSNT |
| | | KVDKKVEPKSCDKTHTCPPCPAPEAAGAPSVFLFPPKPKDTLMISR |
| | | DPEVTCVVVDVSHEDPEVKFNWYVDGVEVDNAKTKPREEQYNSTYR |
| | | VVSVLRVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 891 | ASTKGPSVFPLAPSSKSTSGGTAALGCLVKDYFPEPVTVSWNSGAL |
| LALAPG/DDRVV | | TSGVHTFPAVLQSSGLYSLSSVVTVPSSSLGTQTYICNVNHKPSNT |
| | | KVDKKVEPKSCDKTHTCPPCPAPEAAGGPSVFLFPPKPKDTLMISR |
| | | DPEVTCVVVDVSHEDPEVKFNWYVDGVEVDNAKTKPREEQYNSTYR |
| | | VVSVLRVLHVDWLNGKEYKCKVSNKALGAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPG |
| hIgG1- | 892 | ASTKGPSVFPLAPSSKSTSGGTAALGCLVKDYFPEPVTVSWNSGAL |
| Q311R/M428L | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHRDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG4- | 893 | ASTKGPSVFPLAPCSRSTSESTAALGCLVKDYFPEPVTVSWNSGAL |
| Q311R/M428L | | TSGVHTFPAVLQSSGLYSLSSVVTVPSSSLGTKTYTCNVDHKPSNT |
| | | KVDKRVESKYGPPCPSCPAPEFLGGPSVFLFPPKPKDTLMISRTPE |
| | | VTCVVVDVSQEDPEVQFNWYVDGVEVHNAKTKPREEQFNSTYRVVS |
| | | VLTVLHRDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHNHYTQKSLS |
| | | LSLG |
| IgG4- | 894 | ASTKGPSVFPLAPCSRSTSESTAALGCLVKDYFPEPVTVSWNSGAL |
| SP/Q311R/ | | TSGVHTFPAVLQSSGLYSLSSVVTVPSSSLGTKTYTCNVDHKPSNT |
| M428L | | KVDKRVESKYGPPCPPCPAPEFLGGPSVFLFPPKPKDTLMISRTPE |
| | | VTCVVVDVSQEDPEVQFNWYVDGVEVHNAKTKPREEQFNSTYRVVS |
| | | VLTVLHRDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHNHYTQKSLS |
| | | LSLG |
| IgG4- | 895 | ASTKGPSVFPLAPCSRSTSESTAALGCLVKDYFPEPVTVSWNSGAL |
| SPLE/Q311R/ | | TSGVHTFPAVLQSSGLYSLSSVVTVPSSSLGTKTYTCNVDHKPSNT |
| M428L | | KVDKRVESKYGPPCPPCPAPEELGGPSVFLFPPKPKDTLMISRIPE |
| | | VTCVVVDVSQEDPEVQFNWYVDGVEVHNAKTKPREEQFNSTYRVVS |
| | | VLTVLHRDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHNHYTQKSLS |
| | | LSLG |
| IgG2- | 896 | ASTKGPSVFPLAPCSRSTSESTAALGCLVKDYFPEPVTVSWNSGAL |
| Q311R/M428L | | TSGVHTFPAVLQSSGLYSLSSVVTVPSSNFGTQTYTCNVDHKPSNT |
| | | KVDKTVERKCCVECPPCPAPPVAGPSVFLFPPKPKDTLMISRTPEV |
| | | TCVVVDVSHEDPEVQFNWYVDGVEVHNAKTKPREEQFNSTERVVSV |
| | | LTVVHRDWLNGKEYKCKVSNKGLPAPIEKTISKTKGQPREPQVYTL |
| | | PPSREEMTKNQVSLTCLVKGFYPSDIAVEWESNGQPENNYKITPPM |
| | | LDSDGSFFLYSKLTVDKSRWQQGNVFSCSVLHEALHNHYTQKSLSL |
| | | SPG |
| hIgG1- | 897 | ASTKGPSVFPLAPSSKSTSGGTAALGCLVKDYFPEPVTVSWNSGAL |
| N297A/Q311R/ | | TSGVHTFPAVLQSSGLYSLSSVVTVPSSSLGTQTYICNVNHKPSNT |
| M428L | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYASTYR |
| | | VVSVLTVLHRDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG1- | 898 | ASTKGPSVFPLAPSSKSTSGGTAALGCLVKDYFPEPVTVSWNSGAL |
| D265A/Q311R/ | | TSGVHTFPAVLQSSGLYSLSSVVTVPSSSLGTQTYICNVNHKPSNT |
| M428L | | KVDKKVEPKSCDKTHTCPPCPAPELLGGPSVFLFPPKPKDTLMISR |
| | | TPEVTCVVVAVSHEDPEVKFNWYVDGVEVHNAKTKPREEQYNSTYR |
| | | VVSVLTVLHRDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG1- | 899 | ASTKGPSVFPLAPSSKSTSGGTAALGCLVKDYFPEPVTVSWNSGAL |
| LALA/Q311R/ | | TSGVHTFPAVLQSSGLYSLSSVVTVPSSSLGTQTYICNVNHKPSNT |
| M428L | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHRDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG1- | 900 | ASTKGPSVFPLAPSSKSTSGGTAALGCLVKDYFPEPVTVSWNSGAL |
| LAGA/Q311R/ | | TSGVHTFPAVLQSSGLYSLSSVVTVPSSSLGTQTYICNVNHKPSNT |
| M428L | | KVDKKVEPKSCDKTHTCPPCPAPELAGAPSVFLFPPKPKDTLMISR |
| | | TPEVTCVVVDVSHEDPEVKFNWYVDGVEVHNAKTKPREEQYNSTYR |
| | | VVSVLTVLHRDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG1- | 901 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| Q311R/M428L | | KVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYR |
| | | VVSVLTVLHRDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG1- | 902 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| Q311R/M428L | | KVDKKVEPKSCDKTHTCPPCPAPEAAGGPSVFLFPPKPKDTLMISR |
| | | TPEVTCVVVDVSHEDPEVKFNWYVDGVEVHNAKTKPREEQYNSTYR |
| | | VVSVLTVLHRDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPG |
| hIgG1- | 903 | ASTKGPSVFPLAPSSKSTSGGTAALGCLVKDYFPEPVTVSWNSGAL |
| variant | | TSGVHTFPAVLQSSGLYSLSSVVTVPSSSLGTQTYICNVNHKPSNT |
| | | KVDKRVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1 | 904 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| (M428L/ | | KVDKRVEPKSCDKTHTCPPCPAPELLGGPSVFLFPPKPKDTLMISR |
| N434S) LS | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPG |
| hIgG1 | 905 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| M252Y/S254T/ | | KVDKRVEPKSCDKTHTCPPCPAPELLGGPSVFLFPPKPKDTLYITR |
| T256E | | EPEVTCVVVDVSHEDPEVKFNWYVDGVEVHNAKTKPREEQYNSTYR |
| (YTE) | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1 | 906 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| M428L/N434A | | KVDKRVEPKSCDKTHTCPPCPAPELLGGPSVFLFPPKPKDTLMISR |
| (LA) | | TPEVTCVVVDVSHEDPEVKFNWYVDGVEVHNAKIKPREEQYNSTYR |
| | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVESCSVLHEALHAHYTQK |
| | | SLSLSPG |
| hIgG1 | 907 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| H433K/N434F | | KVDKRVEPKSCDKTHTCPPCPAPELLGGPSVFLFPPKPKDTLMISR |
| (KF) | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALKFHYTQK |
| | | SLSLSPG |
| hIgG1 | 908 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| L309D/Q311H/ | | KVDKRVEPKSCDKTHTCPPCPAPELLGGPSVFLFPPKPKDTLMISR |
| N434S | | TPEVTCVVVDVSHEDPEVKFNWYVDGVEVHNAKTKPREEQYNSTYR |
| (DHS) | | VVSVLTVDHHDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPG |
| hIgG1 | 909 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| N297A | | KVDKRVEPKSCDKTHTCPPCPAPELLGGPSVFLFPPKPKDTLMISR |
| | | TPEVTCVVVDVSHEDPEVKFNWYVDGVEVHNAKIKPREEQYASTYR |
| | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |
| hIgG1 | 910 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| LALAGA/YTE/ | | KVDKKVEPKSCDKTHTCPPCPAPEAAGAPSVFLFPPKPKDTLYITR |
| D356E/L358M | | EPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPG |

TABLE 10B
Exemplary Fc polypeptide Sequences

| | SEQ | |
|---|---|---|
| | ID | |
| Name | NO | Fc polypeptide sequence |
| hIgG1 | 911 | ASTKGPSVFPLAPSSKSTSGGTAALGCLVKDYFPEPVTVSWNSGAL |
| with C- | | TSGVHTFPAVLQSSGLYSLSSVVTVPSSSLGTQTYICNVNHKPSNT |
| terminal | | KVDKKVEPKSCDKTHTCPPCPAPELLGGPSVFLFPPKPKDTLMISR |
| lysine | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG2 | 912 | ASTKGPSVFPLAPCSRSTSESTAALGCLVKDYFPEPVTVSWNSGAL |
| with C- | | TSGVHTFPAVLQSSGLYSLSSVVTVPSSNFGTQTYTCNVDHKPSNT |
| terminal | | KVDKTVERKCCVECPPCPAPPVAGPSVFLFPPKPKDTLMISRTPEV |
| lysine | | TCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVMHEALHNHYTQKSLSL |
| | | SPGK |
| hIgG4 | 913 | ASTKGPSVFPLAPCSRSTSESTAALGCLVKDYFPEPVTVSWNSGAL |
| with C- | | TSGVHTFPAVLQSSGLYSLSSVVTVPSSSLGTKTYTCNVDHKPSNT |
| terminal | | KVDKRVESKYGPPCPSCPAPEFLGGPSVFLFPPKPKDTLMISRTPE |
| lysine | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLGK |
| IgG4-SP | 914 | ASTKGPSVFPLAPCSRSTSESTAALGCLVKDYFPEPVTVSWNSGAL |
| (S228P) | | TSGVHTFPAVLQSSGLYSLSSVVTVPSSSLGTKTYTCNVDHKPSNT |
| with C- | | KVDKRVESKYGPPCPPCPAPEFLGGPSVFLFPPKPKDTLMISRTPE |
| terminal | | VTCVVVDVSQEDPEVQFNWYVDGVEVHNAKTKPREEQFNSTYRVVS |
| lysine | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKITPP |
| | | VLDSDGSFFLYSRLTVDKSRWQEGNVFSCSVMHEALHNHYTQKSLS |
| | | LSLGK |
| IgG4-SPLE | 915 | ASTKGPSVFPLAPCSRSTSESTAALGCLVKDYFPEPVTVSWNSGAL |
| (S228P/ | | TSGVHTFPAVLQSSGLYSLSSVVTVPSSSLGTKTYTCNVDHKPSNT |
| L235E) | | KVDKRVESKYGPPCPPCPAPEELGGPSVFLFPPKPKDTLMISRTPE |
| with C- | | VTCVVVDVSQEDPEVQFNWYVDGVEVHNAKTKPREEQFNSTYRVVS |
| terminal | | VLTVLHQDWLNGKEYKCKVSNKGLPSSIEKTISKAKGQPREPQVYT |
| lysine | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLGK |
| hIgG1- | 916 | ASTKGPSVFPLAPSSKSTSGGTAALGCLVKDYFPEPVTVSWNSGAL |
| N297A | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVDVSHEDPEVKFNWYVDGVEVHNAKTKPREEQYASTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 917 | ASTKGPSVFPLAPSSKSTSGGTAALGCLVKDYFPEPVTVSWNSGAL |
| D265A | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVAVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 918 | ASTKGPSVFPLAPSSKSTSGGTAALGCLVKDYFPEPVTVSWNSGAL |
| LALA | | TSGVHTFPAVLQSSGLYSLSSVVTVPSSSLGTQTYICNVNHKPSNT |
| (L234A/ | | KVDKKVEPKSCDKTHTCPPCPAPEAAGGPSVFLFPPKPKDTLMISR |
| L235A) | | TPEVTCVVVDVSHEDPEVKFNWYVDGVEVHNAKTKPREEQYNSTYR |
| with C- | | VVSVLTVLHQDWLNGKEYKCKVSNKALPAPIEKTISKAKGQPREPQ |
| terminal | | VYTLPPSRDELTKNQVSLTCLVKGFYPSDIAVEWESNGQPENNYKT |
| lysine | | TPPVLDSDGSFFLYSKLTVDKSRWQQGNVFSCSVMHEALHNHYTQK |
| | | SLSLSPGK |
| hIgG1- | 919 | ASTKGPSVFPLAPSSKSTSGGTAALGCLVKDYFPEPVTVSWNSGAL |
| LAGA | | TSGVHTFPAVLQSSGLYSLSSVVTVPSSSLGTQTYICNVNHKPSNT |
| (L235A/ | | KVDKKVEPKSCDKTHTCPPCPAPELAGAPSVFLFPPKPKDTLMISR |
| G237A) | | TPEVTCVVVDVSHEDPEVKFNWYVDGVEVHNAKTKPREEQYNSTYR |
| with C- | | VVSVLTVLHQDWLNGKEYKCKVSNKALPAPIEKTISKAKGQPREPQ |
| terminal | | VYTLPPSRDELTKNQVSLTCLVKGFYPSDIAVEWESNGQPENNYKT |
| lysine | | TPPVLDSDGSFFLYSKLTVDKSRWQQGNVFSCSVMHEALHNHYTQK |
| | | SLSLSPGK |
| hIgG1- | 920 | ASTKGPSVFPLAPSSKSTSGGTAALGCLVKDYFPEPVTVSWNSGAL |
| LALAGA | | TSGVHTFPAVLQSSGLYSLSSVVTVPSSSLGTQTYICNVNHKPSNT |
| (L234A/ | | KVDKKVEPKSCDKTHTCPPCPAPEAAGAPSVFLFPPKPKDILMISR |
| L235A/ | | TPEVTCVVVDVSHEDPEVKFNWYVDGVEVHNAKTKPREEQYNSTYR |
| G237A) | | VVSVLTVLHQDWLNGKEYKCKVSNKALPAPIEKTISKAKGQPREPQ |
| with C- | | VYTLPPSRDELTKNQVSLTCLVKGFYPSDIAVEWESNGQPENNYKT |
| terminal | | TPPVLDSDGSFFLYSKLTVDKSRWQQGNVFSCSVMHEALHNHYTQK |
| lysine | | SLSLSPGK |
| hIgG1- | 921 | ASTKGPSVFPLAPSSKSTSGGTAALGCLVKDYFPEPVTVSWNSGAL |
| LALAPG | | TSGVHTFPAVLQSSGLYSLSSVVTVPSSSLGTQTYICNVNHKPSNT |
| (L234A/ | | KVDKKVEPKSCDKTHTCPPCPAPEAAGGPSVFLFPPKPKDTLMISR |
| L235A/ | | TPEVTCVVVDVSHEDPEVKFNWYVDGVEVHNAKTKPREEQYNSTYR |
| P329G) | | VVSVLTVLHQDWLNGKEYKCKVSNKALGAPIEKTISKAKGQPREPQ |
| with C- | | VYTLPPSRDELTKNQVSLTCLVKGFYPSDIAVEWESNGQPENNYKT |
| terminal | | TPPVLDSDGSFFLYSKLTVDKSRWQQGNVFSCSVMHEALHNHYTQK |
| lysine | | SLSLSPGK |
| hIgG1-YTE | 922 | ASTKGPSVFPLAPSSKSTSGGTAALGCLVKDYFPEPVTVSWNSGAL |
| (M252Y/ | | TSGVHTFPAVLQSSGLYSLSSVVTVPSSSLGTQTYICNVNHKPSNT |
| S254T/ | | KVDKKVEPKSCDKTHTCPPCPAPELLGGPSVFLFPPKPKDTLYITR |
| T256E) | | EPEVTCVVVDVSHEDPEVKFNWYVDGVEVHNAKTKPREEQYNSTYR |
| with C- | | VVSVLTVLHQDWLNGKEYKCKVSNKALPAPIEKTISKAKGQPREPQ |
| terminal | | VYTLPPSRDELTKNQVSLTCLVKGFYPSDIAVEWESNGQPENNYKT |
| lysine | | TPPVLDSDGSFFLYSKLTVDKSRWQQGNVFSCSVMHEALHNHYTQK |
| | | SLSLSPGK |
| hIgG1- | 923 | ASTKGPSVFPLAPSSKSTSGGTAALGCLVKDYFPEPVTVSWNSGAL |
| N297A/YTE | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLYITR |
| terminal | | EPEVTCVVVDVSHEDPEVKFNWYVDGVEVHNAKTKPREEQYASTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 924 | ASTKGPSVFPLAPSSKSTSGGTAALGCLVKDYFPEPVTVSWNSGAL |
| D265A/YTE | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLYITR |
| terminal | | EPEVTCVVVAVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 925 | ASTKGPSVFPLAPSSKSTSGGTAALGCLVKDYFPEPVTVSWNSGAL |
| LALA/YTE | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLYITR |
| terminal | | EPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 926 | ASTKGPSVFPLAPSSKSTSGGTAALGCLVKDYFPEPVTVSWNSGAL |
| LAGA/YTE | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLYITR |
| terminal | | EPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 927 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| YTE | | KVDKKVEPKSCDKTHTCPPCPAPEAAGAPSVFLFPPKPKDTLYITR |
| with C- | | EPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 928 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| YTE | | KVDKKVEPKSCDKTHTCPPCPAPEAAGGPSVFLFPPKPKDTLYITR |
| with C- | | EPEVTCVVVDVSHEDPEVKFNWYVDGVEVHNAKTKPREEQYNSTYR |
| terminal | | VVSVLTVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1-LS | 929 | ASTKGPSVFPLAPSSKSTSGGTAALGCLVKDYFPEPVTVSWNSGAL |
| (M428L/ | | TSGVHTFPAVLQSSGLYSLSSVVTVPSSSLGTQTYICNVNHKPSNT |
| N434S) | | KVDKKVEPKSCDKTHTCPPCPAPELLGGPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1- | 930 | ASTKGPSVFPLAPSSKSTSGGTAALGCLVKDYFPEPVTVSWNSGAL |
| N297A/LS | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVDVSHEDPEVKFNWYVDGVEVHNAKTKPREEQYASTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1- | 931 | ASTKGPSVFPLAPSSKSTSGGTAALGCLVKDYFPEPVTVSWNSGAL |
| D265A/LS | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVAVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1- | 932 | ASTKGPSVFPLAPSSKSTSGGTAALGCLVKDYFPEPVTVSWNSGAL |
| LALA/LS | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1- | 933 | ASTKGPSVFPLAPSSKSTSGGTAALGCLVKDYFPEPVTVSWNSGAL |
| LAGA/LS | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1- | 934 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| LS | | KVDKKVEPKSCDKTHTCPPCPAPEAAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1- | 935 | ASTKGPSVFPLAPSSKSTSGGTAALGCLVKDYFPEPVTVSWNSGAL |
| LALAPG/LS | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALGAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQK |
| | | SLSLSPGK |
| hIgG1-DHS | 936 | ASTKGPSVFPLAPSSKSTSGGTAALGCLVKDYFPEPVTVSWNSGAL |
| (L309D/ | | TSGVHTFPAVLQSSGLYSLSSVVTVPSSSLGTQTYICNVNHKPSNT |
| Q311H/ | | KVDKKVEPKSCDKTHTCPPCPAPELLGGPSVFLFPPKPKDTLMISR |
| N434S) | | TPEVTCVVVDVSHEDPEVKFNWYVDGVEVHNAKTKPREEQYNSTYR |
| with C- | | VVSVLTVDHHDWLNGKEYKCKVSNKALPAPIEKTISKAKGQPREPQ |
| terminal | | VYTLPPSRDELTKNQVSLTCLVKGFYPSDIAVEWESNGQPENNYKT |
| lysine | | TPPVLDSDGSFFLYSKLTVDKSRWQQGNVFSCSVMHEALHSHYTQK |
| | | SLSLSPGK |
| hIgG1- | 937 | ASTKGPSVFPLAPSSKSTSGGTAALGCLVKDYFPEPVTVSWNSGAL |
| N297A/DHS | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVDVSHEDPEVKFNWYVDGVEVHNAKTKPREEQYASTYR |
| lysine | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPGK |
| hIgG1- | 938 | ASTKGPSVFPLAPSSKSTSGGTAALGCLVKDYFPEPVTVSWNSGAL |
| D265A/DHS | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVAVSHEDPEVKFNWYVDGVEVHNAKTKPREEQYNSTYR |
| lysine | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPGK |
| hIgG1- | 939 | ASTKGPSVFPLAPSSKSTSGGTAALGCLVKDYFPEPVTVSWNSGAL |
| LALA/DHS | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLMISR |
| terminal | | TPEVTCVVVDVSHEDPEVKFNWYVDGVEVHNAKTKPREEQYNSTYR |
| lysine | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPGK |
| hIgG1- | 940 | ASTKGPSVFPLAPSSKSTSGGTAALGCLVKDYFPEPVTVSWNSGAL |
| LAGA/DHS | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLMISR |
| terminal | | TPEVTCVVVDVSHEDPEVKFNWYVDGVEVHNAKTKPREEQYNSTYR |
| lysine | | VVSVLTVDHHDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPGK |
| hIgG1- | 941 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| DHS | | KVDKKVEPKSCDKTHTCPPCPAPEAAGAPSVFLFPPKPKDTLMISR |
| with C- | | TPEVTCVVVDVSHEDPEVKFNWYVDGVEVHNAKTKPREEQYNSTYR |
| terminal | | VVSVLTVDHHDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPGK |
| hIgG1- | 942 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| DHS | | KVDKKVEPKSCDKTHTCPPCPAPEAAGGPSVFLFPPKPKDTLMISR |
| with C- | | TPEVTCVVVDVSHEDPEVKFNWYVDGVEVHNAKTKPREEQYNSTYR |
| terminal | | VVSVLTVDHHDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHSHYTQK |
| | | SLSLSPGK |
| hIgG4-YTE | 943 | ASTKGPSVFPLAPCSRSTSESTAALGCLVKDYFPEPVTVSWNSGAL |
| with C- | | TSGVHTFPAVLQSSGLYSLSSVVTVPSSSLGTKTYTCNVDHKPSNT |
| terminal | | KVDKRVESKYGPPCPSCPAPEFLGGPSVFLFPPKPKDTLYITREPE |
| lysine | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLGK |
| hIgG4- | 944 | ASTKGPSVFPLAPCSRSTSESTAALGCLVKDYFPEPVTVSWNSGAL |
| SP/YTE | | TSGVHTFPAVLQSSGLYSLSSVVTVPSSSLGTKTYTCNVDHKPSNT |
| with C- | | KVDKRVESKYGPPCPPCPAPEFLGGPSVFLFPPKPKDTLYITREPE |
| terminal | | VTCVVVDVSQEDPEVQFNWYVDGVEVHNAKTKPREEQFNSTYRVVS |
| lysine | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLGK |
| hIgG4- | 945 | ASTKGPSVFPLAPCSRSTSESTAALGCLVKDYFPEPVTVSWNSGAL |
| SPLE/YTE | | TSGVHTFPAVLQSSGLYSLSSVVTVPSSSLGTKTYTCNVDHKPSNT |
| with C- | | KVDKRVESKYGPPCPPCPAPEELGGPSVFLFPPKPKDTLYITREPE |
| terminal | | VTCVVVDVSQEDPEVQFNWYVDGVEVHNAKTKPREEQFNSTYRVVS |
| lysine | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLGK |
| hIgG4-LS | 946 | ASTKGPSVFPLAPCSRSTSESTAALGCLVKDYFPEPVTVSWNSGAL |
| with C- | | TSGVHTFPAVLQSSGLYSLSSVVTVPSSSLGTKTYTCNVDHKPSNT |
| terminal | | KVDKRVESKYGPPCPSCPAPEFLGGPSVFLFPPKPKDTLMISRTPE |
| lysine | | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHSHYTQKSLS |
| | | LSLGK |
| hIgG4- | 947 | ASTKGPSVFPLAPCSRSTSESTAALGCLVKDYFPEPVTVSWNSGAL |
| SP/LS | | TSGVHTFPAVLQSSGLYSLSSVVTVPSSSLGTKTYTCNVDHKPSNT |
| with C- | | KVDKRVESKYGPPCPPCPAPEFLGGPSVFLFPPKPKDTLMISRTPE |
| terminal | | VTCVVVDVSQEDPEVQFNWYVDGVEVHNAKTKPREEQFNSTYRVVS |
| lysine | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHSHYTQKSLS |
| | | LSLGK |
| hIgG4- | 948 | ASTKGPSVFPLAPCSRSTSESTAALGCLVKDYFPEPVTVSWNSGAL |
| SPLE/LS | | TSGVHTFPAVLQSSGLYSLSSVVTVPSSSLGTKTYTCNVDHKPSNT |
| with C- | | KVDKRVESKYGPPCPPCPAPEELGGPSVFLFPPKPKDTLMISRTPE |
| terminal | | VTCVVVDVSQEDPEVQFNWYVDGVEVHNAKTKPREEQFNSTYRVVS |
| lysine | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVLHEALHSHYTQKSLS |
| | | LSLGK |
| hIgG4-DHS | 949 | ASTKGPSVFPLAPCSRSTSESTAALGCLVKDYFPEPVTVSWNSGAL |
| with C- | | TSGVHTFPAVLQSSGLYSLSSVVTVPSSSLGTKTYTCNVDHKPSNT |
| terminal | | KVDKRVESKYGPPCPSCPAPEFLGGPSVFLFPPKPKDTLMISRTPE |
| lysine | | VTCVVVDVSQEDPEVQFNWYVDGVEVHNAKTKPREEQFNSTYRVVS |
| | | VLTVDHHDWLNGKEYKCKVSNKGLPSSIEKTISKAKGQPREPQVYT |
| | | LPPSQEEMTKNQVSLTCLVKGFYPSDIAVEWESNGQPENNYKITPP |
| | | VLDSDGSFFLYSRLTVDKSRWQEGNVFSCSVMHEALHSHYTQKSLS |
| | | LSLGK |
| hIgG4- | 950 | ASTKGPSVFPLAPCSRSTSESTAALGCLVKDYFPEPVTVSWNSGAL |
| SP/DHS | | TSGVHTFPAVLQSSGLYSLSSVVTVPSSSLGTKTYTCNVDHKPSNT |
| with C- | | KVDKRVESKYGPPCPPCPAPEFLGGPSVFLFPPKPKDTLMISRTPE |
| terminal | | VTCVVVDVSQEDPEVQFNWYVDGVEVHNAKTKPREEQFNSTYRVVS |
| lysine | | VLTVDHHDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHSHYTQKSLS |
| | | LSLGK |
| hIgG4- | 951 | ASTKGPSVFPLAPCSRSTSESTAALGCLVKDYFPEPVTVSWNSGAL |
| SPLE/DHS | | TSGVHTFPAVLQSSGLYSLSSVVTVPSSSLGTKTYTCNVDHKPSNT |
| with C- | | KVDKRVESKYGPPCPPCPAPEELGGPSVFLFPPKPKDTLMISRTPE |
| terminal | | VTCVVVDVSQEDPEVQFNWYVDGVEVHNAKTKPREEQFNSTYRVVS |
| lysine | | VLTVDHHDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHSHYTQKSLS |
| | | LSLGK |
| hIgG2-YTE | 952 | ASTKGPSVFPLAPCSRSTSESTAALGCLVKDYFPEPVTVSWNSGAL |
| with C- | | TSGVHTFPAVLQSSGLYSLSSVVTVPSSNFGTQTYTCNVDHKPSNT |
| terminal | | KVDKTVERKCCVECPPCPAPPVAGPSVFLFPPKPKDTLYITREPEV |
| lysine | | TCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVMHEALHNHYTQKSLSL |
| | | SPGK |
| hIgG2-LS | 953 | ASTKGPSVFPLAPCSRSTSESTAALGCLVKDYFPEPVTVSWNSGAL |
| with C- | | TSGVHTFPAVLQSSGLYSLSSVVTVPSSNFGTQTYTCNVDHKPSNT |
| terminal | | KVDKTVERKCCVECPPCPAPPVAGPSVFLFPPKPKDTLMISRTPEV |
| lysine | | TCVVVDVSHEDPEVQFNWYVDGVEVHNAKTKPREEQFNSTFRVVSV |
| | | LTVVHQDWLNGKEYKCKVSNKGLPAPIEKTISKTKGQPREPQVYTL |
| | | PPSREEMTKNQVSLTCLVKGFYPSDIAVEWESNGQPENNYKITPPM |
| | | LDSDGSFFLYSKLTVDKSRWQQGNVFSCSVLHEALHSHYTQKSLSL |
| | | SPGK |
| hIgG2-DHS | 954 | ASTKGPSVFPLAPCSRSTSESTAALGCLVKDYFPEPVTVSWNSGAL |
| with C- | | TSGVHTFPAVLQSSGLYSLSSVVTVPSSNFGTQTYTCNVDHKPSNT |
| terminal | | KVDKTVERKCCVECPPCPAPPVAGPSVFLFPPKPKDTLMISRTPEV |
| lysine | | TCVVVDVSHEDPEVQFNWYVDGVEVHNAKTKPREEQFNSTFRVVSV |
| | | LTVDHHDWLNGKEYKCKVSNKGLPAPIEKTISKTKGQPREPQVYTL |
| | | PPSREEMTKNQVSLTCLVKGFYPSDIAVEWESNGQPENNYKTTPPM |
| | | LDSDGSFFLYSKLTVDKSRWQQGNVFSCSVMHEALHSHYTQKSLSL |
| | | SPGK |
| hIgG1-LA | 955 | ASTKGPSVFPLAPSSKSTSGGTAALGCLVKDYFPEPVTVSWNSGAL |
| with C- | | TSGVHTFPAVLQSSGLYSLSSVVTVPSSSLGTQTYICNVNHKPSNT |
| terminal | | KVDKKVEPKSCDKTHTCPPCPAPELLGGPSVFLFPPKPKDTLMISR |
| lysine | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 956 | ASTKGPSVFPLAPSSKSTSGGTAALGCLVKDYFPEPVTVSWNSGAL |
| N297A/LA | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVDVSHEDPEVKFNWYVDGVEVHNAKTKPREEQYASTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 957 | ASTKGPSVFPLAPSSKSTSGGTAALGCLVKDYFPEPVTVSWNSGAL |
| D265A/LA | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVAVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 958 | ASTKGPSVFPLAPSSKSTSGGTAALGCLVKDYFPEPVTVSWNSGAL |
| LALA/LA | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 959 | ASTKGPSVFPLAPSSKSTSGGTAALGCLVKDYFPEPVTVSWNSGAL |
| LAGA/LA | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 960 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| LA | | KVDKKVEPKSCDKTHTCPPCPAPEAAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 961 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| LA | | KVDKKVEPKSCDKTHTCPPCPAPEAAGGPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQK |
| | | SLSLSPGK |
| hIgG1- | 962 | ASTKGPSVFPLAPSSKSTSGGTAALGCLVKDYFPEPVTVSWNSGAL |
| N434A | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 963 | ASTKGPSVFPLAPSSKSTSGGTAALGCLVKDYFPEPVTVSWNSGAL |
| N297A/ | | TSGVHTFPAVLQSSGLYSLSSVVTVPSSSLGTQTYICNVNHKPSNT |
| N434A | | KVDKKVEPKSCDKTHTCPPCPAPELLGGPSVFLFPPKPKDTLMISR |
| with C- | | TPEVTCVVVDVSHEDPEVKFNWYVDGVEVHNAKTKPREEQYASTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 964 | ASTKGPSVFPLAPSSKSTSGGTAALGCLVKDYFPEPVTVSWNSGAL |
| D265A/ | | TSGVHTFPAVLQSSGLYSLSSVVTVPSSSLGTQTYICNVNHKPSNT |
| N434A | | KVDKKVEPKSCDKTHTCPPCPAPELLGGPSVFLFPPKPKDTLMISR |
| with C- | | TPEVTCVVVAVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 965 | ASTKGPSVFPLAPSSKSTSGGTAALGCLVKDYFPEPVTVSWNSGAL |
| LALA/ | | TSGVHTFPAVLQSSGLYSLSSVVTVPSSSLGTQTYICNVNHKPSNT |
| N434A | | KVDKKVEPKSCDKTHTCPPCPAPEAAGGPSVFLFPPKPKDILMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 966 | ASTKGPSVFPLAPSSKSTSGGTAALGCLVKDYFPEPVTVSWNSGAL |
| LAGA/ | | TSGVHTFPAVLQSSGLYSLSSVVTVPSSSLGTQTYICNVNHKPSNT |
| N434A | | KVDKKVEPKSCDKTHTCPPCPAPELAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 967 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| N434A | | KVDKKVEPKSCDKTHTCPPCPAPEAAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 968 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| N434A | | KVDKKVEPKSCDKTHTCPPCPAPEAAGGPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHAHYTQK |
| | | SLSLSPGK |
| hIgG1- | 969 | ASTKGPSVFPLAPSSKSTSGGTAALGCLVKDYFPEPVTVSWNSGAL |
| N434W | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDILMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1- | 970 | ASTKGPSVFPLAPSSKSTSGGTAALGCLVKDYFPEPVTVSWNSGAL |
| N297A/ | | TSGVHTFPAVLQSSGLYSLSSVVTVPSSSLGTQTYICNVNHKPSNT |
| N434W | | KVDKKVEPKSCDKTHTCPPCPAPELLGGPSVFLFPPKPKDTLMISR |
| with C- | | TPEVTCVVVDVSHEDPEVKFNWYVDGVEVHNAKTKPREEQYASTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1- | 971 | ASTKGPSVFPLAPSSKSTSGGTAALGCLVKDYFPEPVTVSWNSGAL |
| D265A/ | | TSGVHTFPAVLQSSGLYSLSSVVTVPSSSLGTQTYICNVNHKPSNT |
| N434W | | KVDKKVEPKSCDKTHTCPPCPAPELLGGPSVFLFPPKPKDTLMISR |
| with C- | | TPEVTCVVVAVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1- | 972 | ASTKGPSVFPLAPSSKSTSGGTAALGCLVKDYFPEPVTVSWNSGAL |
| LALA/ | | TSGVHTFPAVLQSSGLYSLSSVVTVPSSSLGTQTYICNVNHKPSNT |
| N434W | | KVDKKVEPKSCDKTHTCPPCPAPEAAGGPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1- | 973 | ASTKGPSVFPLAPSSKSTSGGTAALGCLVKDYFPEPVTVSWNSGAL |
| LAGA/ | | TSGVHTFPAVLQSSGLYSLSSVVTVPSSSLGTQTYICNVNHKPSNT |
| N434W | | KVDKKVEPKSCDKTHTCPPCPAPELAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1- | 974 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| N434W | | KVDKKVEPKSCDKTHTCPPCPAPEAAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1- | 975 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| N434W | | KVDKKVEPKSCDKTHTCPPCPAPEAAGGPSVFLFPPKPKDTLMISR |
| with C- | | TPEVTCVVVDVSHEDPEVKFNWYVDGVEVHNAKTKPREEQYNSTYR |
| terminal | | VVSVLTVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHWHYTQK |
| | | SLSLSPGK |
| hIgG1/DQ | 976 | ASTKGPSVFPLAPSSKSTSGGTAALGCLVKDYFPEPVTVSWNSGAL |
| (T256D/ | | TSGVHTFPAVLQSSGLYSLSSVVTVPSSSLGTQTYICNVNHKPSNT |
| T307Q) | | KVDKKVEPKSCDKTHTCPPCPAPELLGGPSVFLFPPKPKDTLMISR |
| with C- | | DPEVTCVVVDVSHEDPEVKFNWYVDGVEVHNAKTKPREEQYNSTYR |
| terminal | | VVSVLQVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 977 | ASTKGPSVFPLAPSSKSTSGGTAALGCLVKDYFPEPVTVSWNSGAL |
| N297A/DQ | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | DPEVTCVVVDVSHEDPEVKFNWYVDGVEVHNAKTKPREEQYASTYR |
| lysine | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 978 | ASTKGPSVFPLAPSSKSTSGGTAALGCLVKDYFPEPVTVSWNSGAL |
| D265A/DQ | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | DPEVTCVVVAVSHEDPEVKFNWYVDGVEVHNAKTKPREEQYNSTYR |
| lysine | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 979 | ASTKGPSVFPLAPSSKSTSGGTAALGCLVKDYFPEPVTVSWNSGAL |
| LALA/DQ | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLMISR |
| terminal | | DPEVTCVVVDVSHEDPEVKFNWYVDGVEVHNAKTKPREEQYNSTYR |
| lysine | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 980 | ASTKGPSVFPLAPSSKSTSGGTAALGCLVKDYFPEPVTVSWNSGAL |
| LAGA/DQ | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLMISR |
| terminal | | DPEVTCVVVDVSHEDPEVKFNWYVDGVEVHNAKTKPREEQYNSTYR |
| lysine | | VVSVLQVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 981 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| DQ | | KVDKKVEPKSCDKTHTCPPCPAPEAAGAPSVFLFPPKPKDILMISR |
| with C- | | DPEVTCVVVDVSHEDPEVKFNWYVDGVEVHNAKTKPREEQYNSTYR |
| terminal | | VVSVLQVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 982 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| DQ | | KVDKKVEPKSCDKTHTCPPCPAPEAAGGPSVFLFPPKPKDTLMISR |
| with C- | | DPEVTCVVVDVSHEDPEVKFNWYVDGVEVHNAKTKPREEQYNSTYR |
| terminal | | VVSVLQVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1/DW | 983 | ASTKGPSVFPLAPSSKSTSGGTAALGCLVKDYFPEPVTVSWNSGAL |
| (T256D/ | | TSGVHTFPAVLQSSGLYSLSSVVTVPSSSLGTQTYICNVNHKPSNT |
| T307W) | | KVDKKVEPKSCDKTHTCPPCPAPELLGGPSVFLFPPKPKDILMISR |
| with C- | | DPEVTCVVVDVSHEDPEVKFNWYVDGVEVHNAKTKPREEQYNSTYR |
| terminal | | VVSVLWVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 984 | ASTKGPSVFPLAPSSKSTSGGTAALGCLVKDYFPEPVTVSWNSGAL |
| N297A/DW | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | DPEVTCVVVDVSHEDPEVKFNWYVDGVEVHNAKTKPREEQYASTYR |
| lysine | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 985 | ASTKGPSVFPLAPSSKSTSGGTAALGCLVKDYFPEPVTVSWNSGAL |
| D265A/DW | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | DPEVTCVVVAVSHEDPEVKFNWYVDGVEVHNAKTKPREEQYNSTYR |
| lysine | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 986 | ASTKGPSVFPLAPSSKSTSGGTAALGCLVKDYFPEPVTVSWNSGAL |
| LALA/DW | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLMISR |
| terminal | | DPEVTCVVVDVSHEDPEVKFNWYVDGVEVHNAKTKPREEQYNSTYR |
| lysine | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 987 | ASTKGPSVFPLAPSSKSTSGGTAALGCLVKDYFPEPVTVSWNSGAL |
| LAGA/DW | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLMISR |
| terminal | | DPEVTCVVVDVSHEDPEVKFNWYVDGVEVHNAKTKPREEQYNSTYR |
| lysine | | VVSVLWVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 988 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| DW | | KVDKKVEPKSCDKTHTCPPCPAPEAAGAPSVFLFPPKPKDTLMISR |
| with C- | | DPEVTCVVVDVSHEDPEVKFNWYVDGVEVHNAKTKPREEQYNSTYR |
| terminal | | VVSVLWVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 989 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| DW | | KVDKKVEPKSCDKTHTCPPCPAPEAAGGPSVFLFPPKPKDILMISR |
| with C- | | DPEVTCVVVDVSHEDPEVKFNWYVDGVEVHNAKTKPREEQYNSTYR |
| terminal | | VVSVLWVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1/YD | 990 | ASTKGPSVFPLAPSSKSTSGGTAALGCLVKDYFPEPVTVSWNSGAL |
| (M252Y/T25 | | TSGVHTFPAVLQSSGLYSLSSVVTVPSSSLGTQTYICNVNHKPSNT |
| 6D) | | KVDKKVEPKSCDKTHTCPPCPAPELLGGPSVFLFPPKPKDTLYISR |
| with C- | | DPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 991 | ASTKGPSVFPLAPSSKSTSGGTAALGCLVKDYFPEPVTVSWNSGAL |
| N297A/YD | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLYISR |
| terminal | | DPEVTCVVVDVSHEDPEVKFNWYVDGVEVHNAKTKPREEQYASTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 992 | ASTKGPSVFPLAPSSKSTSGGTAALGCLVKDYFPEPVTVSWNSGAL |
| D265A/YD | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLYISR |
| terminal | | DPEVTCVVVAVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 993 | ASTKGPSVFPLAPSSKSTSGGTAALGCLVKDYFPEPVTVSWNSGAL |
| LALA/YD | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDTLYISR |
| terminal | | DPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 994 | ASTKGPSVFPLAPSSKSTSGGTAALGCLVKDYFPEPVTVSWNSGAL |
| LAGA/YD | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLYISR |
| terminal | | DPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 995 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| YD | | KVDKKVEPKSCDKTHTCPPCPAPEAAGAPSVFLFPPKPKDTLYISR |
| with C- | | DPEVTCVVVDVSHEDPEVKFNWYVDGVEVHNAKTKPREEQYNSTYR |
| terminal | | VVSVLTVLHQDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1- | 996 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| YD | | KVDKKVEPKSCDKTHTCPPCPAPEAAGGPSVFLFPPKPKDTLYISR |
| with C- | | DPEVTCVVVDVSHEDPEVKFNWYVDGVEVHNAKTKPREEQYNSTYR |
| terminal | | VVSVLTVLHQDWLNGKEYKCKVSNKALGAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1/QVV | 997 | ASTKGPSVFPLAPSSKSTSGGTAALGCLVKDYFPEPVTVSWNSGAL |
| (T307Q/ | | TSGVHTFPAVLQSSGLYSLSSVVTVPSSSLGTQTYICNVNHKPSNT |
| Q311V/ | | KVDKKVEPKSCDKTHTCPPCPAPELLGGPSVFLFPPKPKDTLMISR |
| A378V) | | TPEVTCVVVDVSHEDPEVKFNWYVDGVEVHNAKTKPREEQYNSTYR |
| with C- | | VVSVLQVLHVDWLNGKEYKCKVSNKALPAPIEKTISKAKGQPREPQ |
| terminal | | VYTLPPSRDELTKNQVSLTCLVKGFYPSDIVVEWESNGQPENNYKT |
| lysine | | TPPVLDSDGSFFLYSKLTVDKSRWQQGNVFSCSVMHEALHNHYTQK |
| | | SLSLSPGK |
| hIgG1- | 998 | ASTKGPSVFPLAPSSKSTSGGTAALGCLVKDYFPEPVTVSWNSGAL |
| N297A/QVV | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVDVSHEDPEVKFNWYVDGVEVHNAKTKPREEQYASTYR |
| lysine | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 999 | ASTKGPSVFPLAPSSKSTSGGTAALGCLVKDYFPEPVTVSWNSGAL |
| D265A/QVV | | TSGVHTFPAVLQSSGLYSLSSVVTVPSSSLGTQTYICNVNHKPSNT |
| with C- | | KVDKKVEPKSCDKTHTCPPCPAPELLGGPSVFLFPPKPKDTLMISR |
| terminal | | TPEVTCVVVAVSHEDPEVKFNWYVDGVEVHNAKTKPREEQYNSTYR |
| lysine | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1000 | ASTKGPSVFPLAPSSKSTSGGTAALGCLVKDYFPEPVTVSWNSGAL |
| LALA/QVV | | TSGVHTFPAVLQSSGLYSLSSVVTVPSSSLGTQTYICNVNHKPSNT |
| with C- | | KVDKKVEPKSCDKTHTCPPCPAPEAAGGPSVFLFPPKPKDILMISR |
| terminal | | TPEVTCVVVDVSHEDPEVKFNWYVDGVEVHNAKTKPREEQYNSTYR |
| lysine | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1001 | ASTKGPSVFPLAPSSKSTSGGTAALGCLVKDYFPEPVTVSWNSGAL |
| LAGA/QVV | | TSGVHTFPAVLQSSGLYSLSSVVTVPSSSLGTQTYICNVNHKPSNT |
| with C- | | KVDKKVEPKSCDKTHTCPPCPAPELAGAPSVFLFPPKPKDTLMISR |
| terminal | | TPEVTCVVVDVSHEDPEVKFNWYVDGVEVHNAKTKPREEQYNSTYR |
| lysine | | VVSVLQVLHVDWLNGKEYKCKVSNKALPAPIEKTISKAKGQPREPQ |
| | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1002 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| QVV | | KVDKKVEPKSCDKTHTCPPCPAPEAAGAPSVFLFPPKPKDTLMISR |
| with C- | | TPEVTCVVVDVSHEDPEVKFNWYVDGVEVHNAKTKPREEQYNSTYR |
| terminal | | VVSVLQVLHVDWLNGKEYKCKVSNKALPAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1003 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| QVV | | KVDKKVEPKSCDKTHTCPPCPAPEAAGGPSVFLFPPKPKDILMISR |
| with C- | | TPEVTCVVVDVSHEDPEVKFNWYVDGVEVHNAKTKPREEQYNSTYR |
| terminal | | VVSVLQVLHVDWLNGKEYKCKVSNKALGAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1/ | 1004 | ASTKGPSVFPLAPSSKSTSGGTAALGCLVKDYFPEPVTVSWNSGAL |
| DDRVV | | TSGVHTFPAVLQSSGLYSLSSVVTVPSSSLGTQTYICNVNHKPSNT |
| (T256D/ | | KVDKKVEPKSCDKTHTCPPCPAPELLGGPSVFLFPPKPKDTLMISR |
| N286D/ | | DPEVTCVVVDVSHEDPEVKFNWYVDGVEVDNAKTKPREEQYNSTYR |
| T307R/ | | VVSVLRVLHVDWLNGKEYKCKVSNKALPAPIEKTISKAKGQPREPQ |
| Q311V/ | | VYTLPPSRDELTKNQVSLTCLVKGFYPSDIVVEWESNGQPENNYKT |
| A378V) | | TPPVLDSDGSFFLYSKLTVDKSRWQQGNVFSCSVMHEALHNHYTQK |
| with C- | | SLSLSPGK |
| terminal | | |
| lysine | | |
| hIgG1- | 1005 | ASTKGPSVFPLAPSSKSTSGGTAALGCLVKDYFPEPVTVSWNSGAL |
| N297A/ | | TSGVHTFPAVLQSSGLYSLSSVVTVPSSSLGTQTYICNVNHKPSNT |
| DDRVV | | KVDKKVEPKSCDKTHTCPPCPAPELLGGPSVFLFPPKPKDTLMISR |
| with C- | | DPEVTCVVVDVSHEDPEVKFNWYVDGVEVDNAKTKPREEQYASTYR |
| terminal | | VVSVLRVLHVDWLNGKEYKCKVSNKALPAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1006 | ASTKGPSVFPLAPSSKSTSGGTAALGCLVKDYFPEPVTVSWNSGAL |
| D265A/ | | TSGVHTFPAVLQSSGLYSLSSVVTVPSSSLGTQTYICNVNHKPSNT |
| DDRVV | | KVDKKVEPKSCDKTHTCPPCPAPELLGGPSVFLFPPKPKDTLMISR |
| with C- | | DPEVTCVVVAVSHEDPEVKFNWYVDGVEVDNAKTKPREEQYNSTYR |
| terminal | | VVSVLRVLHVDWLNGKEYKCKVSNKALPAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1007 | ASTKGPSVFPLAPSSKSTSGGTAALGCLVKDYFPEPVTVSWNSGAL |
| LALA/ | | TSGVHTFPAVLQSSGLYSLSSVVTVPSSSLGTQTYICNVNHKPSNT |
| DDRVV | | KVDKKVEPKSCDKTHTCPPCPAPEAAGGPSVFLFPPKPKDTLMISR |
| with C- | | DPEVTCVVVDVSHEDPEVKFNWYVDGVEVDNAKTKPREEQYNSTYR |
| terminal | | VVSVLRVLHVDWLNGKEYKCKVSNKALPAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1008 | ASTKGPSVFPLAPSSKSTSGGTAALGCLVKDYFPEPVTVSWNSGAL |
| LAGA/ | | TSGVHTFPAVLQSSGLYSLSSVVTVPSSSLGTQTYICNVNHKPSNT |
| DDRVV | | KVDKKVEPKSCDKTHTCPPCPAPELAGAPSVFLFPPKPKDTLMISR |
| with C- | | DPEVTCVVVDVSHEDPEVKFNWYVDGVEVDNAKTKPREEQYNSTYR |
| terminal | | VVSVLRVLHVDWLNGKEYKCKVSNKALPAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1009 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| DDRVV | | KVDKKVEPKSCDKTHTCPPCPAPEAAGAPSVFLFPPKPKDTLMISR |
| with C- | | DPEVTCVVVDVSHEDPEVKFNWYVDGVEVDNAKTKPREEQYNSTYR |
| terminal | | VVSVLRVLHVDWLNGKEYKCKVSNKALPAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1010 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| DDRVV | | KVDKKVEPKSCDKTHTCPPCPAPEAAGGPSVFLFPPKPKDTLMISR |
| with C- | | DPEVTCVVVDVSHEDPEVKFNWYVDGVEVDNAKTKPREEQYNSTYR |
| terminal | | VVSVLRVLHVDWLNGKEYKCKVSNKALGAPIEKTISKAKGQPREPQ |
| lysine | | VYTLPPSRDELTKNQVSLTCLVKGFYPSDIVVEWESNGQPENNYKT |
| | | TPPVLDSDGSFFLYSKLTVDKSRWQQGNVFSCSVMHEALHNHYTQK |
| | | SLSLSPGK |
| hIgG1- | 1011 | ASTKGPSVFPLAPSSKSTSGGTAALGCLVKDYFPEPVTVSWNSGAL |
| Q311R/ | | TSGVHTFPAVLQSSGLYSLSSVVTVPSSSLGTQTYICNVNHKPSNT |
| M428L | | KVDKKVEPKSCDKTHTCPPCPAPELLGGPSVFLFPPKPKDTLMISR |
| with C- | | TPEVTCVVVDVSHEDPEVKFNWYVDGVEVHNAKTKPREEQYNSTYR |
| terminal | | VVSVLTVLHRDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPGK |
| hIgG4- | 1012 | ASTKGPSVFPLAPCSRSTSESTAALGCLVKDYFPEPVTVSWNSGAL |
| Q311R/ | | TSGVHTFPAVLQSSGLYSLSSVVTVPSSSLGIKTYTCNVDHKPSNT |
| M428L | | KVDKRVESKYGPPCPSCPAPEFLGGPSVFLFPPKPKDTLMISRTPE |
| with C- | | VTCVVVDVSQEDPEVQFNWYVDGVEVHNAKTKPREEQFNSTYRVVS |
| terminal | | VLTVLHRDWLNGKEYKCKVSNKGLPSSIEKTISKAKGQPREPQVYT |
| lysine | | LPPSQEEMTKNQVSLTCLVKGFYPSDIAVEWESNGQPENNYKTTPP |
| | | VLDSDGSFFLYSRLTVDKSRWQEGNVFSCSVLHEALHNHYTQKSLS |
| | | LSLGK |
| IgG4- | 1013 | ASTKGPSVFPLAPCSRSTSESTAALGCLVKDYFPEPVTVSWNSGAL |
| SP/Q311R/ | | TSGVHTFPAVLQSSGLYSLSSVVTVPSSSLGTKTYTCNVDHKPSNT |
| M428L | | KVDKRVESKYGPPCPPCPAPEFLGGPSVFLFPPKPKDTLMISRTPE |
| with C- | | VTCVVVDVSQEDPEVQFNWYVDGVEVHNAKTKPREEQFNSTYRVVS |
| terminal | | VLTVLHRDWLNGKEYKCKVSNKGLPSSIEKTISKAKGQPREPQVYT |
| lysine | | LPPSQEEMTKNQVSLTCLVKGFYPSDIAVEWESNGQPENNYKTTPP |
| | | VLDSDGSFFLYSRLTVDKSRWQEGNVFSCSVLHEALHNHYTQKSLS |
| | | LSLGK |
| IgG4- | 1014 | ASTKGPSVFPLAPCSRSTSESTAALGCLVKDYFPEPVTVSWNSGAL |
| SPLE/Q311R/ | | TSGVHTFPAVLQSSGLYSLSSVVTVPSSSLGTKTYTCNVDHKPSNT |
| M428L | | KVDKRVESKYGPPCPPCPAPEELGGPSVFLFPPKPKDTLMISRTPE |
| with C- | | VTCVVVDVSQEDPEVQFNWYVDGVEVHNAKTKPREEQFNSTYRVVS |
| terminal | | VLTVLHRDWLNGKEYKCKVSNKGLPSSIEKTISKAKGQPREPQVYT |
| lysine | | LPPSQEEMTKNQVSLTCLVKGFYPSDIAVEWESNGQPENNYKTTPP |
| | | VLDSDGSFFLYSRLTVDKSRWQEGNVFSCSVLHEALHNHYTQKSLS |
| | | LSLGK |
| IgG2- | 1015 | ASTKGPSVFPLAPCSRSTSESTAALGCLVKDYFPEPVTVSWNSGAL |
| Q311R/ | | TSGVHTFPAVLQSSGLYSLSSVVTVPSSNFGTQTYTCNVDHKPSNT |
| M428L | | KVDKTVERKCCVECPPCPAPPVAGPSVFLFPPKPKDTLMISRTPEV |
| with C- | | TCVVVDVSHEDPEVQFNWYVDGVEVHNAKTKPREEQFNSTERVVSV |
| terminal | | LTVVHRDWLNGKEYKCKVSNKGLPAPIEKTISKTKGQPREPQVYTL |
| lysine | | PPSREEMTKNQVSLTCLVKGFYPSDIAVEWESNGQPENNYKTTPPM |
| | | LDSDGSFFLYSKLTVDKSRWQQGNVFSCSVLHEALHNHYTQKSLSL |
| | | SPGK |
| hIgG1- | 1016 | ASTKGPSVFPLAPSSKSTSGGTAALGCLVKDYFPEPVTVSWNSGAL |
| N297A/ | | TSGVHTFPAVLQSSGLYSLSSVVTVPSSSLGTQTYICNVNHKPSNT |
| Q311R/M428L | | KVDKKVEPKSCDKTHTCPPCPAPELLGGPSVFLFPPKPKDTLMISR |
| with C- | | TPEVTCVVVDVSHEDPEVKFNWYVDGVEVHNAKTKPREEQYASTYR |
| terminal | | VVSVLTVLHRDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPGK |
| hIgG1- | 1017 | ASTKGPSVFPLAPSSKSTSGGTAALGCLVKDYFPEPVTVSWNSGAL |
| D265A/ | | TSGVHTFPAVLQSSGLYSLSSVVTVPSSSLGTQTYICNVNHKPSNT |
| Q311R/M428L | | KVDKKVEPKSCDKTHTCPPCPAPELLGGPSVFLFPPKPKDTLMISR |
| with C- | | TPEVTCVVVAVSHEDPEVKFNWYVDGVEVHNAKTKPREEQYNSTYR |
| terminal | | VVSVLTVLHRDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPGK |
| hIgG1- | 1018 | ASTKGPSVFPLAPSSKSTSGGTAALGCLVKDYFPEPVTVSWNSGAL |
| LALA/ | | TSGVHTFPAVLQSSGLYSLSSVVTVPSSSLGTQTYICNVNHKPSNT |
| Q311R/M428L | | KVDKKVEPKSCDKTHTCPPCPAPEAAGGPSVFLFPPKPKDTLMISR |
| with C- | | TPEVTCVVVDVSHEDPEVKFNWYVDGVEVHNAKTKPREEQYNSTYR |
| terminal | | VVSVLTVLHRDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPGK |
| hIgG1- | 1019 | ASTKGPSVFPLAPSSKSTSGGTAALGCLVKDYFPEPVTVSWNSGAL |
| LAGA/ | | TSGVHTFPAVLQSSGLYSLSSVVTVPSSSLGTQTYICNVNHKPSNT |
| Q311R/M428L | | KVDKKVEPKSCDKTHTCPPCPAPELAGAPSVFLFPPKPKDTLMISR |
| with C- | | TPEVTCVVVDVSHEDPEVKFNWYVDGVEVHNAKTKPREEQYNSTYR |
| terminal | | VVSVLTVLHRDWLNGKEYKCKVSNKALPAPIEKTISKAKGQPREPQ |
| lysine | | VYTLPPSRDELTKNQVSLTCLVKGFYPSDIAVEWESNGQPENNYKT |
| | | TPPVLDSDGSFFLYSKLTVDKSRWQQGNVFSCSVLHEALHNHYTQK |
| | | SLSLSPGK |
| hIgG1- | 1020 | ASTKGPSVFPLAPSSKSTSGGTAALGCLVKDYFPEPVTVSWNSGAL |
| LALAGA/ | | TSGVHTFPAVLQSSGLYSLSSVVTVPSSSLGTQTYICNVNHKPSNT |
| Q311R/ | | KVDKKVEPKSCDKTHTCPPCPAPEAAGAPSVFLFPPKPKDTLMISR |
| M428L | | TPEVTCVVVDVSHEDPEVKFNWYVDGVEVHNAKTKPREEQYNSTYR |
| with C- | | VVSVLTVLHRDWLNGKEYKCKVSNKALPAPIEKTISKAKGQPREPQ |
| terminal | | VYTLPPSRDELTKNQVSLTCLVKGFYPSDIAVEWESNGQPENNYKT |
| lysine | | TPPVLDSDGSFFLYSKLTVDKSRWQQGNVFSCSVLHEALHNHYTQK |
| | | SLSLSPGK |
| hIgG1- | 1021 | ASTKGPSVFPLAPSSKSTSGGTAALGCLVKDYFPEPVTVSWNSGAL |
| LALAPG/ | | TSGVHTFPAVLQSSGLYSLSSVVTVPSSSLGTQTYICNVNHKPSNT |
| Q311R/ | | KVDKKVEPKSCDKTHTCPPCPAPEAAGGPSVFLFPPKPKDTLMISR |
| M428L | | TPEVTCVVVDVSHEDPEVKFNWYVDGVEVHNAKTKPREEQYNSTYR |
| with C- | | VVSVLTVLHRDWLNGKEYKCKVSNKALGAPIEKTISKAKGQPREPQ |
| terminal | | VYTLPPSRDELTKNQVSLTCLVKGFYPSDIAVEWESNGQPENNYKT |
| lysine | | TPPVLDSDGSFFLYSKLTVDKSRWQQGNVFSCSVLHEALHNHYTQK |
| | | SLSLSPGK |
| hIgG1 - | 1022 | ASTKGPSVFPLAPSSKSTSGGTAALGCLVKDYFPEPVTVSWNSGAL |
| variant | | TSGVHTFPAVLQSSGLYSLSSVVTVPSSSLGTQTYICNVNHKPSNT |
| with C- | | KVDKRVEPKSCDKTHTCPPCPAPELLGGPSVFLFPPKPKDTLMISR |
| terminal | | TPEVTCVVVDVSHEDPEVKFNWYVDGVEVHNAKTKPREEQYNSTYR |
| lysine | | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1 | 1023 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| (M428L/ | | KVDKRVEPKSCDKTHTCPPCPAPELLGGPSVFLFPPKPKDTLMISR |
| N434S) LS | | TPEVTCVVVDVSHEDPEVKFNWYVDGVEVHNAKTKPREEQYNSTYR |
| with C- | | VVSVLTVLHQDWLNGKEYKCKVSNKALPAPIEKTISKAKGQPREPQ |
| terminal | | VYTLPPSREEMTKNQVSLTCLVKGFYPSDIAVEWESNGQPENNYKT |
| lysine | | TPPVLDSDGSFFLYSKLTVDKSRWQQGNVFSCSVLHEALHSHYTQK |
| | | SLSLSPGK |
| hIgG1 | 1024 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| M252Y/S254T/ | | KVDKRVEPKSCDKTHTCPPCPAPELLGGPSVFLFPPKPKDILYITR |
| T256E | | EPEVTCVVVDVSHEDPEVKFNWYVDGVEVHNAKTKPREEQYNSTYR |
| (YTE) | | VVSVLTVLHQDWLNGKEYKCKVSNKALPAPIEKTISKAKGQPREPQ |
| with C- | | VYTLPPSREEMTKNQVSLTCLVKGFYPSDIAVEWESNGQPENNYKT |
| terminal | | TPPVLDSDGSFFLYSKLTVDKSRWQQGNVFSCSVMHEALHNHYTQK |
| lysine | | SLSLSPGK |
| hIgG1 | 1025 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| M428L/ | | KVDKRVEPKSCDKTHTCPPCPAPELLGGPSVFLFPPKPKDTLMISR |
| N434A (LA) | | TPEVTCVVVDVSHEDPEVKFNWYVDGVEVHNAKTKPREEQYNSTYR |
| with C- | | VVSVLTVLHQDWLNGKEYKCKVSNKALPAPIEKTISKAKGQPREPQ |
| terminal | | VYTLPPSREEMTKNQVSLTCLVKGFYPSDIAVEWESNGQPENNYKT |
| lysine | | TPPVLDSDGSFFLYSKLTVDKSRWQQGNVFSCSVLHEALHAHYTQK |
| | | SLSLSPGK |
| hIgG1 | 1026 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| H433K/ | | KVDKRVEPKSCDKTHTCPPCPAPELLGGPSVFLFPPKPKDTLMISR |
| N434F(KF) | | TPEVTCVVVDVSHEDPEVKFNWYVDGVEVHNAKTKPREEQYNSTYR |
| with C- | | VVSVLTVLHQDWLNGKEYKCKVSNKALPAPIEKTISKAKGQPREPQ |
| terminal | | VYTLPPSREEMTKNQVSLTCLVKGFYPSDIAVEWESNGQPENNYKT |
| lysine | | TPPVLDSDGSFFLYSKLTVDKSRWQQGNVFSCSVMHEALKFHYTQK |
| | | SLSLSPGK |
| hIgG1 | 1027 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| L309D/Q311H/ | | KVDKRVEPKSCDKTHTCPPCPAPELLGGPSVFLFPPKPKDTLMISR |
| N434S | | TPEVTCVVVDVSHEDPEVKFNWYVDGVEVHNAKTKPREEQYNSTYR |
| (DHS) | | VVSVLTVDHHDWLNGKEYKCKVSNKALPAPIEKTISKAKGQPREPQ |
| with C- | | VYTLPPSREEMTKNQVSLTCLVKGFYPSDIAVEWESNGQPENNYKT |
| terminal | | TPPVLDSDGSFFLYSKLTVDKSRWQQGNVFSCSVMHEALHSHYTQK |
| lysine | | SLSLSPGK |
| hIgG1 | 1028 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| N297A | | KVDKRVEPKSCDKTHTCPPCPAPELLGGPSVFLFPPKPKDTLMISR |
| with C- | | TPEVTCVVVDVSHEDPEVKFNWYVDGVEVHNAKTKPREEQYASTYR |
| terminal | | VVSVLTVLHQDWLNGKEYKCKVSNKALPAPIEKTISKAKGQPREPQ |
| lysine | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| hIgG1 | 1029 | ASTKGPSVFPLAPSSKSTSGGTAALGCLVKDYFPEPVTVSWNSGAL |
| variant with | | TSGVHTFPAVLQSSGLYSLSSVVTVPSSSLGTQTYICNVNHKPSNT |
| LALAGA/ | | KVDKKVEPKSCDKTHTCPPCPAPEAAGAPSVFLFPPKPKDTLYITR |
| YTE/ | | EPEVTCVVVDVSHEDPEVKFNWYVDGVEVHNAKTKPREEQYNSTYR |
| D356E/ | | VVSVLTVLHQDWLNGKEYKCKVSNKALPAPIEKTISKAKGQPREPQ |
| L358M | | VYTLPPSREEMTKNQVSLTCLVKGFYPSDIAVEWESNGQPENNYKT |
| with C- | | TPPVLDSDGSFFLYSKLTVDKSRWQQGNVFSCSVMHEALHNHYTQK |
| terminal | | SLSLSPGK |
| lysine | | |

In some embodiments, any binding protein described herein may comprise a Fc comprising one or more modifications with any one of the Fc modifications described herein. In some embodiments, any binding protein described herein can comprise any one of the Fc sequences in Tables 10A-10B (SEQ ID NOs: 792-1029). In some embodiments, the CH(1-3) domain of a binding protein in Table 7 is substituted with any one of the Fc sequences in Tables 10A-10B (SEQ ID NOs: 792-1029).

In some embodiments, one or more modifications in the modified Fc is selected from the group consisting of: S298A, E333A, K334A, K326A, F243L, R292P, Y300L, V305I, P396L, F243L, R292P, Y300L, L235V, P396L, F243L, S239D, I332E, A330L, S267E, L328F, D265S, S239E, K326A, A327H, G237F, K326E, G236A, D270L, H268D, S324T, L234F, N325L, V266L, and S267D. In some embodiments, one or more modifications in the modified Fc is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W.

In some embodiments, the modified Fc comprises a specific combination of amino acid substitutions selected from the group consisting of: L234A/L235A; V234A/G237A; L235A/G237A/E318A; S228P/L236E; H268Q/V309L/A330S/A331S; C220S/C226S/C229S/P238S; C226S/C229S/E3233P/L235V/L235A; L234F/L235E/P331S; C226S/P230S; L234A/G237A; L234A/L235A/G237A; and L234A/L235A/P329G.

In some embodiments, the modified Fc comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA); M428L/N434A (LA); L234A/G237A (LAGA); L234A/L235A/G237A (LALAGA); L234A/L235A/P329G (LALAPG); N297A/YTE; D265A/YTE; LALA/YTE; LAGA/YTE; LALAGA/YTE; LALAPG/YTE; N297A/LS; D265A/LS; LALA/LS; LAGA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS; SP/DHS; SPLE/DHS; N297A/LA; D265A/LA; LALA/LA; LAGA/LA; LALAGA/LA; LALAPG/LA; N297A/N434A; D265A/N434A; LALA/N434A; LAGA/N434A; LALAGA/N434A; LALAPG/N434A; N297A/N434W; D265A/N434W; LALA/N434W; LAGA/N434W; LALAGA/N434W; LALAPG/N434W; N297A/DQ; D265A/DQ; LALA/DQ; LAGA/DQ; LALAGA/DQ; LALAPG/DQ; N297A/DW; D265A/DW; LALA/DW; LAGA/DW; LALAGA/DW; LALAPG/DW; N297A/YD; D265A/YD; LALA/YD; LAGA/YD; LALAGA/YD; LALAPG/YD; N297A/QVV; D265A/QVV; LALA/QVV; LAGA/QVV, LALAGA/QVV; LALAPG/QVV; N297A/DDRVV; D265A/DDRVV; LALA/DDRVV; LAGA/DDRVV; LALAGA/DDRVV; and LALAPG/DDRVV. In some embodiments, the modified Fc comprises a specific combination of amino acid substitutions selected from the group consisting of M428L/N434S (LS) and M252Y/S254T/T256E (YTE). In some embodiments, the modified Fc comprises M428L/N434S (LS) (e.g., SEQ ID NO: 810, 827, 834, 904, 929, 946, 953, or 1023) modifications. In some embodiments, the modified Fc comprises M252Y/S254T/T256E (YTE) modifications (e.g., SEQ ID NO: 803, 824, 833, 905, 922, 952, or 1024).

In some embodiments, the bispecific binding proteins described herein includes modifications to improve its ability to mediate effector function. Such modifications are known in the art and include afucosylation, or engineering of the affinity of the Fc towards an activating receptor, mainly FCGR3a for antibody-dependent cellular cytotoxicity (ADCC), and towards C1q for complement-dependent cytotoxicity (CDC).

In some embodiments, modifications to Fc regions reduce or abrogate effector functions, e.g., Fcγ receptor-mediated effector functions. Non-limiting examples of effector-reducing mutations or sets of mutations include, e.g., aglycosylation mutations (e.g., N297A or N297Q or N297G), L234A/L235A (for IgG1 Fc regions), H268Q/V309L/A330S/P331S (for IgG2 Fc regions), and V234A/G237A/P238S/H268A/V309L/A330S/P331S (for IgG2 Fc regions). In some embodiments, effector function is reduced by modifying the attached sugar structures. Non-limiting examples of effector-reducing sugar modifications include increasing sialylation or galactosylation of the sugar chains.

In some aspects, the bispecific binding proteins provided herein comprises a Fc domain (e.g., IgG1) with reduced fucose content at position Asn 297 (EU numbering) compared to a naturally occurring Fc domain. Such Fc domains are known to have improved ADCC. In some aspects, such binding proteins do not comprise any fucose at position Asn 297.

In some embodiments, the bispecific binding proteins described herein comprises an Fc region with one or more amino acid substitutions which improve ADCC, such as a substitution at one or more of positions 298, 333, and 334 of the Fc region. In some embodiments, the bispecific binding proteins provided herein comprises an Fc region with one or more amino acid substitutions at positions 239, 332, and 330.

In some embodiments, the Fc comprises an amino acid sequence having at least 80% sequence identity with the amino acid sequence according to any one of SEQ ID NOs in Table 10A or 10B. In some embodiments, the Fc comprises an amino acid sequence having at least 85% sequence identity with the amino acid sequence according to any one of SEQ ID NOs in Table 10A or 10B. In some embodiments, the Fc comprises an amino acid sequence having at least 90% sequence identity with the amino acid sequence according to any one of SEQ ID NOs in Table 10A or 10B. In some embodiments, the Fc comprises an amino acid sequence having at least 95% sequence identity with the amino acid sequence according to any one of SEQ ID NOs in Table 10A or 10B. In some embodiments, the Fc comprises an amino acid sequence having at least 96% sequence identity with the amino acid sequence according to any one of SEQ ID NOs in Table 10A or 10B. In some embodiments, the Fc comprises an amino acid sequence having at least 97% sequence identity with the amino acid sequence according to any one of SEQ ID NOs in Table 10A or 10B. In some embodiments, the Fc comprises an amino acid sequence having at least 98% sequence identity with the amino acid sequence according to any one of SEQ ID NOs in Table 10A or 10B. In some embodiments, the Fc comprises an amino acid sequence having at least 99% sequence identity with the amino acid sequence according to any one of SEQ ID NOs in Table 10A or 10B. In some embodiments, the Fc comprises the amino acid sequence according to any one of SEQ ID NOs in Table 10A or 10B.

In some embodiments, the bispecific binding proteins described herein comprise an Fc region with at least one galactose residue in the oligosaccharide attached to the Fc region. Such variants may have improved CDC function.

In some embodiments, the bispecific binding proteins described herein comprise one or more alterations that improves or diminishes C1q binding and/or CDC.

In certain embodiments, the Fc region comprises one or more amino acid substitutions, wherein the one or more substitutions result in an increase in one or more of antibody half-life, ADCC activity, ADCP activity, or CDC activity compared with the Fc without the one or more substitutions. In certain embodiments, the one or more amino acid substitutions results in increased antibody half-life at pH 6.0 compared to an antibody comprising a wild-type Fc region. In certain embodiments, the binding protein has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region.

In certain embodiments, the Fc region comprises one or more amino acid substitutions, wherein the one or more substitutions result in a decrease in one or more of ADCC activity, ADCP activity, or CDC activity compared with the Fc without the one or more substitutions.

In certain embodiments, the Fc region binds an Fcγ Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

In some embodiments, the bispecific binding proteins described herein comprise an extended half-life (i.e., serum half-life). In some embodiments, the bispecific binding proteins described herein comprise a half-life of at least about 14, 28, 42, 56, 70, 84, 96, or more than 96 weeks. In some embodiments, the binding protein described herein comprises a half-life in a range of about 14 days to about 96 days, about 14 days to about 84 days, about 14 days to about 70 days, about 14 days to about 56 days, about 14 days to about 42 days, about 14 days to about 28 days, of about 28 days to about 96 days, about 28 days to about 84 days, about 28 days to about 70 days, about 28 days to about 56 days, about 28 days to about 42 days, of about 42 days to about 96 days, about 42 days to about 84 days, about 42 days to about 70 days, or about 42 days to about 56 days. In some embodiments, the bispecific binding proteins described herein comprise a half-life in a range of about 42 days to about 56 days. In some embodiments, the bispecific binding proteins described herein comprise a half-life of at least about 50 days. In some embodiments, the bispecific binding proteins described herein comprise a half-life of about 50 days. Methods of measuring half-life are known in the art. In some embodiments, the half-life is measured in a non-human primate. In some embodiments, the half-life is measured in a human. In some embodiments, the half-life is measured following intravenous administration. In some embodiments, the half-life is measured following subcutaneous administration.

In some embodiments, the bispecific binding proteins described herein have a half-life that is at least 20% longer than a comparator antibody. In some embodiments, the comparator antibody comprises the same complementarity determining regions and variable regions but different Fc regions. In some embodiments, the half-life of the bispecific binding proteins described herein is at least 30%, at least 40%, at least 50%, at least 60%, at least 70%, at least 80%, or at least 90% longer than the half-life of the comparator antibody. In some embodiments, the half-life of the bispecific binding proteins described herein is longer than the half-life of the comparator antibody by at least 2 fold, at least 3 fold, at least 4 fold, at least 5 fold, at least 6 fold, at least 7 fold, at least 8 fold, at least 9 fold, or at least 10 fold.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., VEGF or PD-1) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of a binding protein disclosed here in for a non-target molecule is less than about 50% of the affinity for VEGF or PD-1. In some embodiments, the affinity of a binding protein disclosed here in for a non-target molecule is less than about 40% of the affinity for VEGF or PD-1. In some embodiments, the affinity of a binding protein disclosed here in for a non-target molecule is less than about 40% of the affinity for VEGF or PD-1. In some embodiments, the affinity of a binding protein disclosed here in for a non-target molecule is less than about 20% of the affinity for VEGF or PD-1. In some embodiments, the affinity of a binding protein disclosed here in for a non-target molecule is less than about 10% of the affinity for VEGF or PD-1. In some embodiments, the affinity of a binding protein disclosed here in for a non-target molecule is less than about 1% of the affinity for VEGF or PD-1. In some embodiments, the affinity of a binding protein disclosed here in for a non-target molecule is less than about 0.1% of the affinity for VEGF or PD-1.

When used herein in the context of two or more binding proteins, the term “competes with” or “cross-competes with” indicates that the two or more binding proteins compete for binding to an antigen (e.g., VEGF). In one exemplary assay, VEGF is coated on a surface and contacted with a first anti-VEGF antibody, after which a second anti-VEGF antibody is added. In another exemplary assay, a first anti-VEGF antibody is coated on a surface and contacted with VEGF, and then a second anti-VEGF antibody is added. If the presence of the first anti-VEGF antibody reduces binding of the second anti-VEGF antibody, in either assay, then the binding proteins compete with each other. The term “competes with” also includes combinations of binding proteins where one antibody reduces binding of another antibody, but where no competition is observed when the binding proteins are added in the reverse order. However, in some embodiments, the first and second binding proteins inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the binding proteins used in the competition assays based on the affinities of the binding proteins for VEGF and the valency of the binding proteins. The assays described in this definition are illustrative, and a skilled artisan can utilize any suitable assay to determine if binding proteins compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Binding proteins identified by competition assay (competing antibody) include binding proteins binding to the same epitope as the reference antibody and binding proteins binding to an adjacent epitope sufficiently proximal to the epitope bound by the reference antibody for steric hindrance to occur. For example, a second, competing antibody can be identified that competes for binding to VEGF with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In certain embodiments, the antibody binds a human VEGF and PD-1.

In some embodiments, the antibody provided herein binds VEGF or PD-1 with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody provided herein binds VEGF or PD-1 with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Pharmaceutical Compositions

The present application provides compositions comprising the disclosed binding proteins (e.g., PD-1 binding proteins, or VEGF/PD-1 bispecific binding proteins) including pharmaceutical compositions with one or more pharmaceutically acceptable excipients. In some embodiments the composition is sterile. The pharmaceutical compositions generally comprise an effective amount of a binding protein.

These compositions can comprise, in addition to one or more of the binding proteins disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

The binding protein that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16^{th} edition, Osol, A. (ed), 1980.

A composition can be administered alone or in combination with other treatments, either simultaneously or sequentially dependent upon the condition to be treated.

### Methods

### Methods of Preparation

Methods for making binding proteins are known in the art. See Milstein and Cuello (1983) NATURE 305:537, International (PCT) Publication No. WO93/08829, and Traunecker et al. (1991) EMBO J., 10:3655. For further details of generating binding proteins see, for example, Suresh et al. (1986) METHODS ENZYMOL. 121:210. Binding proteins include cross-linked or “heteroconjugate” or “heterodimer” binding proteins. For example, one of the binding proteins in the heterodimer can be coupled to avidin, the other to biotin. Heterodimer binding proteins may be made using any convenient cross-linking method. Suitable cross-linking agents are well known in the art, and are disclosed in U.S. Pat. No. 4,676,980, along with a number of cross-linking techniques.

For example, binding proteins described herein can be produced using recombinant methods and compositions, e.g., as described in U.S. Pat. No. 4,816,567. In one embodiment, an isolated nucleic acid encoding a chain of a binding protein described herein is provided. Such a nucleic acid may encode an amino acid sequence comprising the VL sequence(s) and/or an amino acid sequence comprising the VH sequence(s) of the antibody (e.g., the light and/or heavy chains of the antibody). In a further embodiment, one or more vectors (e.g., expression vectors) comprising such nucleic acids are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL sequence(s) of the antibody and an amino acid sequence comprising the VH sequence(s) of the antigen-binding polypeptide construct, or (2) a first vector comprising a nucleic acid that encodes an amino acid sequence comprising the VL sequence(s) of the antigen-binding polypeptide construct and a second vector comprising a nucleic acid that encodes an amino acid sequence comprising the VH sequence(s) of the antigen-binding polypeptide construct. In one embodiment, the host cell is eukaryotic, e.g., a Chinese Hamster Ovary (CHO) cell, or human embryonic kidney (HEK) cell, or lymphoid cell (e.g., Y0, NS0, Sp20 cell). In one embodiment, a method of making a binding protein is provided, wherein the method comprises culturing a host cell comprising nucleic acids each encoding at least one chain of the binding protein, as provided above, under conditions suitable for expression of the binding protein, and optionally recovering the binding protein from the host cell (or host cell culture medium).

For recombinant production of the binding protein, nucleic acid encoding at least one chain of an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When a binding protein or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antibody or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” antibody produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per.C6, or BHK.

For example, antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated binding proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the binding proteins described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

If required, the binding proteins can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and binding proteins, and these proteins can find use in the present invention for purification of binding proteins. For example, the bacterial proteins A and G bind to the Fc region. Likewise, the bacterial protein L binds to the Fab region of some binding proteins. Purification can often be enabled by a particular fusion partner. For example, binding proteins may be purified using glutathione resin if a GST fusion is employed, Ni⁺² affinity chromatography if a His-tag is employed or immobilized anti-flag antibody if a flag-tag is used. For general guidance in suitable purification techniques, see, e.g., incorporated entirely by reference Protein Purification: Principles and Practice, 3^{rd} Ed., Scopes, Springer-Verlag, NY, 1994, incorporated entirely by reference. The degree of purification necessary will vary depending on the use of the binding proteins. In some instances, no purification is necessary.

In certain embodiments, the binding proteins are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, binding proteins described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

In certain embodiments, an antibody described herein has an aggregation temperature greater than about 69° C., greater than about 70° C., greater than about 71° C., greater than about 72° C., greater than about 73° C., greater than about 74° C., greater than about 75° C., or greater than about 76° C., for example, between about 69° C. and about 77° C., between about 70° C. and about 76° C., between about 71° C. and about 75° C. In certain embodiments, aggregation temperature is measured using DSF.

In certain embodiments, an antibody described herein has reduced hydrophobicity as compared to lebrikizumab as measured by hydrophobic interaction chromatography (HIC). In certain embodiments, the antibody exhibits an HIC retention time that is less than about 15.2 min. In certain embodiments, the antibody exhibits an HIC retention time that is between about 13 min and about 15 min.

### Methods of Use/Methods of Treatment

In an aspect, the present application provides methods of contacting VEGF and/or PD-1 with a binding protein described herein.

In an aspect, the present application provides methods of using the binding proteins described herein or a pharmaceutical compositions thereof for treatment of a disorder or disease in a subject. In certain aspects, described herein is a method for treating a subject in need thereof, the method comprising administering to a mammalian subject a therapeutically effective amount of a binding protein described herein or pharmaceutical composition thereof. In certain embodiments, the present application provides methods of treating a disorder or disease associated with elevated levels of VEGF and/or PD-1 in a subject.

In certain aspects, described herein are methods for treating a pathology associated with VEGF and/or PD-1 activity, the method comprising administering to a subject a therapeutically effective amount a binding protein described herein or a pharmaceutical composition thereof.

### Subjects

In certain embodiments, the subject is a mammal, such as a primate. In some embodiments, the subject is human.

The subject may be suffering from, exhibit at least one symptom of, diagnosed with, and/or identified as at risk of a disease or condition associated with aberrant VEGF and/or PD-1 expression and/or signaling. Non-limiting examples of such diseases or conditions include cancer (including solid tumors and liquid cancers), such as brain cancers (e.g., glioblastoma), chest and thoracic cancers (e.g., esophageal cancer, pleural mesothelioma, lung cancers (e.g., non-small cell lung cancer (NSCLC) including EGRm NSCLC, non-squamous NSCLC, and squamous NSCLC; and small cell lung cancer (SCLC)), gastrointestinal cancers (e.g., biliary cancer, colorectal cancer (all types, including MSI-H/dMMR and non-MSI-H/dMMR), gastric cancer, gastroesophageal junction (GEJ) cancer), hepatocellular cancer, or primary peritoneal cancel), head and neck cancers (e.g., head and neck squamous cell carcinoma, nasopharyngeal cancer, or thyroid cancer), hematological cancers (e.g., classical Hodgkin lymphoma or primary mediastinal large B-cell lymphoma (PMBCL)), kidney cancers (e.g., renal cell carcinoma), liver and biliary cancers (e.g., biliary tract cancer or hepatocellular carcinoma), reproductive cancers (e.g., breast cancer (including triple negative breast cancer), cervical cancer, endometrial cancer, fallopian tube cancer, ovarian cancer including epithelial ovarian cancer, and urothelial cancer), and soft tissue cancers (e.g., alveolar soft part carcinoma or soft tissue sarcoma).

### Methods of Administration

In some embodiments, the methods provided herein are useful for the treatment of a disease or disorder in an individual. In an embodiment, the individual is a human and a binding protein described herein is administered to the human.

In some embodiments, a binding protein is administered intravenously, intramuscularly, subcutaneously, topically, orally, transdermally, intraperitoneally, intraorbitally, by implantation, by inhalation, intrathecally, intraventricularly, or intranasally. An effective amount of a binding protein may be administered for the treatment of a disease or disorder. The appropriate dosage of a binding protein may be determined based on the type of disease or disorder to be treated, the type of the binding protein, the severity and course of the disease or disorder, the clinical condition of the individual, the individual's clinical history and response to the treatment, and the discretion of the attending physician.

In some embodiments, an antibody provided herein is administered with at least one additional therapeutic agent. Any suitable additional therapeutic or immunotherapeutic agent may be administered with an antibody provided herein. Additional therapeutic agents include agents that are used to treat or prevent a disease or disorder such as, but not limited to, cancer, e.g., a cancer associated with elevated levels of VEGF and/or PD-1, a PD-1 expressing cancer, non-small cell lung cancer, etc.

The additional therapeutic agent can be administered by any suitable means. In some embodiments, an antibody provided herein and the additional therapeutic agent are included in the same pharmaceutical composition. In some embodiments, an antibody provided herein and the additional therapeutic agent are included in different pharmaceutical compositions.

In embodiments where a binding protein provided herein and the additional therapeutic agent are included in different pharmaceutical compositions, administration of the binding protein can occur prior to, simultaneously, and/or following, administration of the additional therapeutic agent. In some embodiments, administration of an antibody provided herein and the additional therapeutic agent occur within about one month of each other. In some embodiments, administration of a binding protein provided herein and the additional therapeutic agent occur within about one week of each other. In some embodiments, administration of a binding protein provided herein and the additional therapeutic agent occur within about one day of each other. In some embodiments, administration of a binding protein provided herein and the additional therapeutic agent occur within about twelve hours of each other. In some embodiments, administration of a binding protein provided herein and the additional therapeutic agent occur within about one hour of each other.

### Kits and Articles of Manufacture

The present application provides kits comprising any one or more of the binding protein compositions described herein and instructions for use. In some embodiments, the kits further contain a component selected from any of secondary binding proteins, reagents for immunohistochemistry analysis, pharmaceutically acceptable excipient and instruction manual and any combination thereof. In one specific embodiment, the kit comprises a pharmaceutical composition comprising any one or more of the binding protein compositions described herein, with one or more pharmaceutically acceptable excipients.

The present application also provides articles of manufacture comprising any one of the binding protein compositions or kits described herein. Examples of an article of manufacture include vials (including sealed vials).

### EXAMPLES

Provided herein are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3^{rd} Ed. (Plenum Press) Vols A and B (1992).

### Methods

### Gene Synthesis and Plasmid Construction

The coding sequences for HCs and LCs of the bispecific antibody were generated by DNA synthesis and PCR, subsequently subcloned into a plasmid for protein expression in mammalian cell system. The gene sequences in the expression vectors were confirmed by DNA sequencing.

### Expression of Antibody Constructs

Bispecific constructs were engineered using methods known to those of ordinary skill in the art (e.g., transient expression of bispecific antibodies performed by co-transfection of paired HC and LC constructs into CHO cells using PEI method).

### SEC-HPLC Analysis of Antibody Construct

Standard analytical SEC-HPLC was performed using Shimadzu LC-10 HPLC instrument (Shimadzu Corp.).

### Measuring Antibody Binding Kinetics Using Surface Plasmon Resonance (SPR)

A Biacore 8K SPR system (GE HealthCare) equipped with Series S Sensor Chip Protein G was used to determine the binding kinetic rate and affinity constants using methods known to those of ordinary skill in the art. Samples were injected in a multi-cycle manner over freshly captured bispecific antibody, by regenerating the capture surfaces with injection of glycine. The data was processed and analyzed with Biacore Insight Evaluation Software Version 2.0.15.12933 (GE Healthcare). The responses from the two buffer blank injections were then subtracted from the reference subtracted data to yield double-referenced data, which were fit to a 1:1 binding model to determine the apparent association (kₐ) and dissociation rate constants (k_{d}). Their ratio provides the apparent equilibrium dissociation constant or affinity constant (K_{D}=k_{d}/kₐ).

For first-pass assays, SPR analysis was conducted on antibodies against PD-1-hFc and VEGFA for K_{D}, for example.

### Assessing Blockade by Cell-Line-Based Assays

The blockade of the full signalling complex of VEGF and/or PD-1 was assessed using cell-line based assays and/or ELISA. Methods for conducting those assays are known to those of ordinary skill in the art.

### PD-1 Cell Based Binding Assay

PD-1 binding was assessed in Jurkat-hPD-1 cells in the presence or absence of 2× conc. of VEGF (2×VEGF) by flow cytometry. Briefly, Jurkat-hPD-1 cells were resuspended in FACS buffer (2% FBS in IX PBS). Bispecific antibodies and 2×VEGFA were pre-incubated for 30 min at RT and added to the cells. Cells were incubated at 4° C. for 1 h and washed with FACS buffer. Cells were resuspended with 100 μL secondary antibody (1:500 dilution in FACS buffer) and incubated 4° C. for 1 h. After the incubation cells were washed with FACS buffer and analysed by FACS. The data was plotted and analyzed in GraphPad Prism 10.

### PD-1 Reporter Assay

PD-1 signal blocking was assessed by reporter assay coculturing Jurkat-NFAT-hPD-1 and Hep3B-OS8-hPD-L1 cells. Briefly, Hep3B-OS8-PD-L1 cells and Jurkat (6C8)-NFAT-PD-1 cells were co-cultured in the presence of the bispecific antibody (+/−VEGF) and incubated in a humidified 37° C., 5% CO2 incubator for 6 hours. After the incubation, ONE-Glo™ Luciferase Assay System were added to each well to allow complete cell lysis and luminescence was measured by a Thermo Fisher Varioskan LUX multimode microplate reader. The data was plotted and analyzed in GraphPad Prism 10.

### PD-1 Internalization Assay

PD1 internalization was assessed by measuring PD-1 level on PD-1-expressing Jurkat cells by flow cytometry. Briefly, Jurkat-hPD-1 cells were seeded into 96 well plate at 2E5 cells per well. Bispecific antibodies and 2× conc. of VEGF were pre-incubated for 30 min at RT. Cells were then treated with the antibodies (+/−VEGF) in warm culture medium at 37° C. for 0, 0.5, 2, 4 and 6 h. After incubation at 37° C., cells were kept on ice and fixed with PFA with a final concentration 2% for 15 min. Cells were then labeled with anti-human IgG Fc-Alexa 647 (1:500) at 4° C. for 1 h and analyzed by flow cytometry. The data was plotted and analyzed in GraphPad Prism 10.

### VEGF Reporter Assay

VEGFA induced signal blocking was assessed by reporter assay using human VEGF R2 (Luc) HEK293 reporter cell. Briefly, human VEGF R2 (Luc.) HEK293 reporter cells were resuspended cell in assay medium and plated (3E4/well) in 96-well one day before the assay. Bispecific antibodies and 2× conc. of VEGF were pre-incubated for 30 min at RT and added to the cells. Cells were incubated in a humidified 37° C., 5% CO2 incubator for 4 hours. After the incubation, ONE-Glo™ Luciferase Assay System were added to each well to allow complete cell lysis and luminescence was measured by a Thermo Fisher Varioskan LUX multimode microplate reader. The data was plotted and analyzed in GraphPad Prism 10.

### Human Umbilical Vein Endothelial Cells (HUVEC) Proliferation Assay

Inhibition of VEGF-mediated cell proliferation was assessed in HUVEC culture. Briefly, HUVEC were digested with 3 ml TrypLE at 37° C. for 3 min and washed. Cells were then resuspended in assay medium and seeded in assay plate, and incubated at 37° C. overnight. Bispecific antibodies and 2× conc. of VEGF were pre-incubated for 30 min at RT.

Antibodies (+/−VEGFA) were added to the cells and incubated at 37° C. for 3 days. After the incubation CellTiter-Glo Luminescent buffer was added to the plate, and incubated at RT for 10 min. Luminescence was measured by Thermo Varioskan LUX afterwards. The data is plotted and analyzed in GraphPad Prism 10.

### T Cell Activation in Human PBMC and Hepatocellular Carcinoma Cell Line Co-Culture

T cell activation was assessed in human PBMC and Hep3B-OS8-hPD-L1 co-culture assay in the presence or absence of 2×VEGFA by measuring IL-2 secretion. Briefly, Hep3B-OS8-PD-L1 cells were resuspended with mitomycin, 10 ug/ml (in 5-10 ml 10% FBS+RPMI 1640 medium) and incubated at 37° C. for 1.5 hr. Bispecific antibodies and 2× conc. of VEGFA were pre-incubated for 30 min at RT. During incubation, PBMCs from healthy volunteers were resuspended in 10% FBS+RPMI 1640 medium. PBMC cells (5E4/well), Hep3B-OS8-PD-L1 cells (5E3/well) and antibodies (+/−VEGF) were added to 96 well plate in a humidified 37° C., 5% CO2 incubator for 72 hours. After 72 h, cell culture supernatants were collected, and concentration of IL-2 and IFN is measured by human IL-2 ELISA kit. The data was plotted and analyzed in GraphPad Prism 10.

### T Cell Activation in Human Monocyte Derived Dendritic Cells and CD4+ T Cells Via Mixed Lymphocyte Reaction (MLR) Assay

T cell activation was assessed in human monocyte derived dendritic cell (moDC) and CD4+ T cell mixed lymphocyte reaction (MLR) by measuring IL-2 and IFNγ secretion. Briefly, CD14+ monocyte were isolated from human PBMCs using the CD14 microbeads isolation kit (MILTENYI-130-050-201) according to the manufacturer protocol. The isolated CD14+ monocytes were then added to a 6-well plate followed by 2 mL/well of hGM-CSF & hIL-4 with assay media to induce dendritic cell differentiation. The plate was then incubated at 37° C. in 5% CO2 for 6 days. After the incubation period, the cells were treated with rhGM-CSF (50 ng/mL), IL-4 (25 ng/ml) and LPS (1 μg/mL) to mature the dendritic cells (DCs). The DCs were then harvested and diluted into 0.2×106 cells/mL. CD4+ T cells were isolated from human PBMCs as well with the Human CD4+ T Cell Isolation Kit utilizing the manufacturer protocol. The isolated CD4+ T cells were resuspended at 2×10⁶ cells/mL using assay media followed by distribution to a 96 well plate at 50 μL/well (0.1×106). Bispecific antibodies and 2× conc. of VEGF were pre-incubated for 30 min at RT. The antibody mixtures (+/−VEGFA) were transferred to the T cell culture, mixed well and incubated at 37° C. for 30 minutes. After incubation, the monocyte derived dendritic cells were added to the plate at a volume of 50 μL/well (0.2×106 cells/mL, 0.01×106 cells/well) and were incubated at 37° C., 5% CO2. After the 48-hour incubation period, 50 μL of the supernatant was transferred to a new 96-well plate for IL-2 quantification using the cytometric bead array (CBA) and a 96-hour incubation period, 50 μL of the supernatant was transferred to a new 96-well plate for IFNγ quantification via the CBA. The data is plotted and analyzed in GraphPad Prism 10.

### Comparators

In some examples provided herein, comparators were used to evaluate bioactivity of the bispecific antibodies disclosed herein. In some cases, an ivonescimab (also referred to as PAL054-0001.1L or AK112 herein) antibody was used. AK112 is a PD-1/VEGF bispecific antibody comprising a heavy chain (SEQ ID NO: 128) and a light chain (SEQ ID NO: 275).

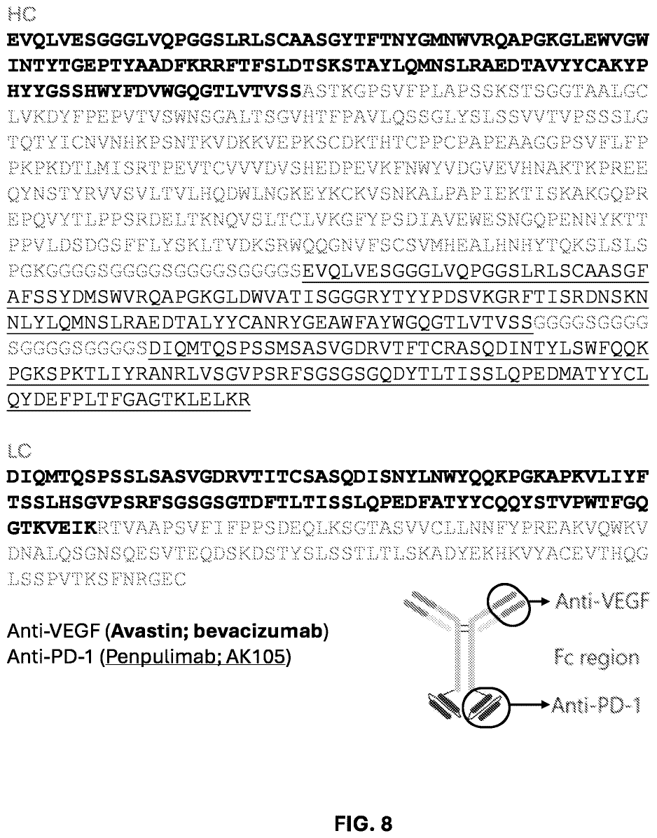

AK112 comprises VH and VL domains from bevacizumab and penpulimab (AK105) (FIG. 8).

Additional comparator sequences (controls and benchmarks are provided in Table 11).

TABLE 11

| | | | | |
|---|---|---|---|---|
| | | SEQ | | SEQ |
| Construct | | ID | | ID |
| Name | HC Protein | NO | LC Protein | NO |

Benchmarks

| | | | | |
|---|---|---|---|---|
| PAL054- | EVQLVESGGGLVQPGGSLRLSCAASGYTFTNYGMN | 128 | DIQMTQSPSSLSAS | 275 |
| 0001.1L | WVRQAPGKGLEWVGWINTYTGEPTYAADFKRRFTF | | VGDRVTITCSASQD | |
| (Ivonescimab; | SLDTSKSTAYLQMNSLRAEDTAVYYCAKYPHYYGS | | ISNYLNWYQQKPGK | |
| AK112) | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKIKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLILS | |
| | GGSGGGGSGGGGSGGGGSEVQLVESGGGLVQPGGS | | KADYEKHKVYACEV | |
| | LRLSCAASGFAFSSYDMSWVRQAPGKGLDWVATIS | | THQGLSSPVTKSEN | |
| | GGGRYTYYPDSVKGRFTISRDNSKNNLYLQMNSLR | | RGEC | |
| | AEDTALYYCANRYGEAWFAYWGQGTLVTVSSGGGG | | | |
| | SGGGGSGGGGSGGGGSDIQMTQSPSSMSASVGDRV | | | |
| | TFTCRASQDINTYLSWFQQKPGKSPKTLIYRANRL | | | |
| | VSGVPSRFSGSGSGQDYTLTISSLQPEDMATYYCL | | | |
| | QYDEFPLTFGAGTKLELKR | | | |
| PAL054- | EVQLVESGGGLVQPGGSLRLSCAASGYTFTNYGMN | 129 | DIQMTQSPSSLSAS | 276 |
| 0001.1La | WVRQAPGKGLEWVGWINTYTGEPTYAADFKRRFTE | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLYITREPEVTCVVVDVSHEDPEVK | | GQGTKVEIKRTVAA | |
| | FNWYVDGVEVHNAKTKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLIVDK | | QSGNSQESVTEQDS | |
| | SRWQQGNVFSCSVMHEALHNHYTQKSLSLSPGKGG | | KDSTYSLSSTLILS | |
| | GGSGGGGSGGGGSGGGGSEVQLVESGGGLVQPGGS | | KADYEKHKVYACEV | |
| | LRLSCAASGFAFSSYDMSWVRQAPGKGLDWVATIS | | THQGLSSPVTKSEN | |
| | GGGRYTYYPDSVKGRFTISRDNSKNNLYLQMNSLR | | RGEC | |
| | AEDTALYYCANRYGEAWFAYWGQGTLVTVSSGGGG | | | |
| | SGGGGSGGGGSGGGGSDIQMTQSPSSMSASVGDRV | | | |
| | TFTCRASQDINTYLSWFQQKPGKSPKTLIYRANRL | | | |
| | VSGVPSRFSGSGSGQDYTLTISSLQPEDMATYYCL | | | |
| | QYDEFPLTFGAGTKLELKR | | | |
| PAL054- | EVQLVESGGGLVQPGGSLRLSCAASGFAFSSYDMS | 745 | DIQMTQSPSSMSAS | 746 |
| 0002.1L | WVRQAPGKGLDWVATISGGGRYTYYPDSVKGRFTI | | VGDRVTFTCRASQD | |
| | SRDNSKNNLYLQMNSLRAEDTALYYCANRYGEAWF | | INTYLSWFQQKPGK | |
| | AYWGQGTLVTVSSASTKGPSVFPLAPSSKSTSGGT | | SPKTLIYRANRLVS | |
| | AALGCLVKDYFPEPVTVSWNSGALTSGVHTFPAVL | | GVPSRFSGSGSGQD | |
| | QSSGLYSLSSVVTVPSSSLGTQTYICNVNHKPSNT | | YTLTISSLQPEDMA | |
| | KVDKKVEPKSCDKTHTCPPCPAPEAAGAPSVFLFP | | TYYCLQYDEFPLTF | |
| | PKPKDTLMISRTPEVTCVVVDVSHEDPEVKENWYV | | GAGTKLELKRTVAA | |
| | DGVEVHNAKTKPREEQYNSTYRVVSVLIVLHQDWL | | PSVFIFPPSDEQLK | |
| | NGKEYKCKVSNKALPAPIEKTISKAKGQPREPQVY | | SGTASVVCLLNNFY | |
| | TLPPSRDELTKNQVSLTCLVKGFYPSDIAVEWESN | | PREAKVQWKVDNAL | |
| | GQPENNYKTTPPVLDSDGSFFLYSKLTVDKSRWQQ | | QSGNSQESVTEQDS | |
| | GNVFSCSVMHEALHNHYTQKSLSLSPGK | | KDSTYSLSSTLTLS | |
| | | | KADYEKHKVYACEV | |
| | | | THQGLSSPVTKSEN | |
| | | | RGEC | |
| PAL054- | EPKSCDKTHTCPPCPAPEAAGGPSVFLFPPKPKDT | 747 | | |
| 0003.1L | LMISRTPEVTCVVVDVSHEDPEVKFNWYVDGVEVH | | | |
| | NAKTKPREEQYNSTYRVVSVLTVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKTTPPVLDSDGSFFLYSKLTVDKSRWQQGNVFSC | | | |
| | SVMHEALHNHYTQKSLSLSPGKGGGGSGGGGSGGG | | | |
| | GSGGGGSEVQLVESGGGLVQPGGSLRLSCAASGFA | | | |
| | FSSYDMSWVRQAPGKGLDWVATISGGGRYTYYPDS | | | |
| | VKGRFTISRDNSKNNLYLQMNSLRAEDTALYYCAN | | | |
| | RYGEAWFAYWGQGTLVTVSSGGGGSGGGGSGGGGS | | | |
| | GGGGSDIQMTQSPSSMSASVGDRVTFTCRASQDIN | | | |
| | TYLSWFQQKPGKSPKTLIYRANRLVSGVPSRFSGS | | | |
| | GSGQDYTLTISSLQPEDMATYYCLQYDEFPLIEGA | | | |
| | GTKLELKR | | | |
| PAL054- | QVQLVQSGVEVKKPGASVKVSCKASGYTFTNYYMY | 748 | EIVLTQSPATLSLS | 749 |
| 0004.4 | WVRQAPGQGLEWMGGINPSNGGTNFNEKFKNRVTL | | PGERATLSCRASKG | |
| (Pembroli | TTDSSTTTAYMELKSLQFDDTAVYYCARRDYRFDM | | VSTSGYSYLHWYQQ | |
| zumab) | GFDYWGQGTTVTVSSASTKGPSVFPLAPCSRSTSE | | KPGQAPRLLIYLAS | |
| | STAALGCLVKDYFPEPVTVSWNSGALTSGVHTFPA | | YLESGVPARFSGSG | |
| | VLQSSGLYSLSSVVTVPSSSLGTKTYTCNVDHKPS | | SGTDFTLTISSLEP | |
| | NTKVDKRVESKYGPPCPPCPAPEFLGGPSVFLFPP | | EDFAVYYCQHSRDL | |
| | KPKDTLMISRTPEVTCVVVDVSQEDPEVQFNWYVD | | PLTFGGGTKVEIKR | |
| | GVEVHNAKTKPREEQFNSTYRVVSVLIVLHQDWLN | | TVAAPSVFIFPPSD | |
| | GKEYKCKVSNKGLPSSIEKTISKAKGQPREPQVYT | | EQLKSGTASVVCLL | |
| | LPPSQEEMTKNQVSLTCLVKGFYPSDIAVEWESNG | | NNFYPREAKVQWKV | |
| | QPENNYKTTPPVLDSDGSFFLYSRLTVDKSRWQEG | | DNALQSGNSQESVT | |
| | NVFSCSVMHEALHNHYTQKSLSLSLGK | | EQDSKDSTYSLSST | |
| | | | LTLSKADYEKHKVY | |
| | | | ACEVTHQGLSSPVT | |
| | | | KSFNRGEC | |
| PAL054- | EPKSCDKTHTCPPCPAPEAAGGPSVFLFPPKPKDT | 750 | | |
| 0005.1L | LMISRTPEVTCVVVDVSHEDPEVKENWYVDGVEVH | | | |
| | NAKTKPREEQYNSTYRVVSVLIVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKTTPPVLDSDGSFFLYSKLTVDKSRWQQGNVFSC | | | |
| | SVMHEALHNHYTQKSLSLSPGKGGGGSGGGGSGGG | | | |
| | GSGGGGSQVQLVQSGVEVKKPGASVKVSCKASGYT | | | |
| | FTNYYMYWVRQAPGQGLEWMGGINPSNGGTNFNEK | | | |
| | FKNRVTLTTDSSTTTAYMELKSLQFDDTAVYYCAR | | | |
| | RDYRFDMGFDYWGQGTTVTVSSGGGGSGGGGSGGG | | | |
| | GSGGGGSEIVLTQSPATLSLSPGERATLSCRASKG | | | |
| | VSTSGYSYLHWYQQKPGQAPRLLIYLASYLESGVP | | | |
| | ARFSGSGSGTDFTLTISSLEPEDFAVYYCQHSRDL | | | |
| | PLTFGGGTKVEIKR | | | |
| PAL054- | EPKSCDKTHTCPPCPAPEAAGGPSVFLFPPKPKDT | 751 | | |
| 0006.1L | LMISRTPEVTCVVVDVSHEDPEVKENWYVDGVEVH | | | |
| | NAKTKPREEQYNSTYRVVSVLTVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKTTPPVLDSDGSFFLYSKLTVDKSRWQQGNVFSC | | | |
| | SVMHEALHNHYTQKSLSLSPGKGGGGSGGGGSGGG | | | |
| | GSGGGGSQVQLVQSGVEVKKPGASVKVSCKASGYT | | | |
| | FTNYYMYWVRQAPGQCLEWMGGINPSNGGINENEK | | | |
| | FKNRVTLTTDSSTTTAYMELKSLQFDDTAVYYCAR | | | |
| | RDYRFDMGFDYWGQGTTVTVSSGGGGSGGGGSGGG | | | |
| | GSGGGGSEIVLTQSPATLSLSPGERATLSCRASKG | | | |
| | VSTSGYSYLHWYQQKPGQAPRLLIYLASYLESGVP | | | |
| | ARFSGSGSGTDFTLTISSLEPEDFAVYYCQHSRDL | | | |
| | PLTFGCGTKVEIKR | | | |
| PAL054- | EVQLVESGGGLVQPGGSLRLSCAASGYTFTNYGMN | 752 | DIQMTQSPSSLSAS | 753 |
| 0007.1 | WVRQAPGKGLEWVGWINTYTGEPTYAADFKRRFTF | | VGDRVTITCSASQD | |
| (Bevacizumab) | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPELLGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKTKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSREEMTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGK | | KDSTYSLSSTLTLS | |
| | | | KADYEKHKVYACEV | |
| | | | THQGLSSPVTKSEN | |
| | | | RGEC | |
| PAL054- | EVQLVESGGGLVQPGGSLRLSCAASGYTFTNYGMN | 754 | DIQMTQSPSSLSAS | 755 |
| 0008.1L | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKTKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLTLS | |
| | GGSGGGGSGGGGSGGGGSQVQLVESGGGVVQPGRS | | KADYEKHKVYACEV | |
| | LRLDCKASGITFSNSGMHWVRQAPGKGLEWVAVIW | | THQGLSSPVTKSEN | |
| | YDGSKRYYADSVKGRFTISRDNSKNILFLQMNSLR | | RGEC | |
| | AEDTAVYYCATNDDYWGQGTGVRVSSGGGGSGGGG | | | |
| | SGGGGSGGGGSEIVLTQSPATLSLSPGERATLSCR | | | |
| | ASQSVSSYLAWYQQKPGQAPRLLIYDASNRATGIP | | | |
| | ARFSGSGSGTDFTLTISSLEPEDFAVYYCQQSSNW | | | |
| | PRTFGQGTKVEIKR | | | |
| PAL054- | EVQLVESGGGLVQPGGSLRLSCAASGYTFTNYGMN | 756 | DIQMTQSPSSLSAS | 757 |
| 0008.1La | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLYITREPEVTCVVVDVSHEDPEVK | | GQGTKVEIKRTVAA | |
| | FNWYVDGVEVHNAKTKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLTLS | |
| | GGSGGGGSGGGGSGGGGSQVQLVESGGGVVQPGRS | | KADYEKHKVYACEV | |
| | LRLDCKASGITFSNSGMHWVRQAPGKGLEWVAVIW | | THQGLSSPVTKSEN | |
| | YDGSKRYYADSVKGRFTISRDNSKNTLFLQMNSLR | | RGEC | |
| | AEDTAVYYCATNDDYWGQGTGVRVSSGGGGSGGGG | | | |
| | SGGGGSGGGGSEIVLTQSPATLSLSPGERATLSCR | | | |
| | ASQSVSSYLAWYQQKPGQAPRLLIYDASNRATGIP | | | |
| | ARFSGSGSGTDFTLTISSLEPEDFAVYYCQQSSNW | | | |
| | PRTFGQGTKVEIKR | | | |
| PAL054- | EVQLVESGGGLVQPGGSLRLSCAASGYTFTNYGMN | 758 | DIQMTQSPSSLSAS | 759 |
| 0009.1L | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKIKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLILS | |
| | GGSGGGGSGGGGSGGGGSEVQLLESGGVLVQPGGS | | KADYEKHKVYACEV | |
| | LRLSCAASGFTFSNFGMTWVRQAPGKGLEWVSGIS | | THQGLSSPVTKSEN | |
| | GGGRDTYFADSVKGRFTISRDNSKNTLYLQMNSLK | | RGEC | |
| | GEDTAVYYCVKWGNIYFDYWGQGTLVTVSSGGGGS | | | |
| | GGGGGGGGSGGGGSDIQMTQSPSSLSASVGDSIT | | | |
| | ITCRASLSINTFLNWYQQKPGKAPNLLIYAASSLH | | | |
| | GGVPSRFSGSGSGTDFTLTIRTLQPEDFATYYCQQ | | | |
| | SSNTPFTFGPGTVVDFR | | | |
| PAL054- | EVQLVESGGGLVQPGGSLRLSCAASGYTFTNYGMN | 760 | DIQMTQSPSSLSAS | 761 |
| 0009.1La | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLYITREPEVTCVVVDVSHEDPEVK | | GQGTKVEIKRTVAA | |
| | FNWYVDGVEVHNAKIKPREEQYNSTYRVVSVLTVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLTLS | |
| | GGSGGGGSGGGGSGGGGSEVQLLESGGVLVQPGGS | | KADYEKHKVYACEV | |
| | LRLSCAASGFTFSNFGMTWVRQAPGKGLEWVSGIS | | THQGLSSPVTKSEN | |
| | GGGRDTYFADSVKGRFTISRDNSKNTLYLQMNSLK | | RGEC | |
| | GEDTAVYYCVKWGNIYFDYWGQGTLVTVSSGGGGS | | | |
| | GGGGSGGGGSGGGGSDIQMTQSPSSLSASVGDSIT | | | |
| | ITCRASLSINTFLNWYQQKPGKAPNLLIYAASSLH | | | |
| | GGVPSRFSGSGSGTDFTLTIRTLQPEDFATYYCQQ | | | |
| | SSNTPFTFGPGTVVDER | | | |
| PAL054- | EVQLVESGGGLVQPGGSLRLSCAASGYTFTNYGMN | 762 | DIQMTQSPSSLSAS | 763 |
| 0010.1La | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLYITREPEVTCVVVDVSHEDPEVK | | GQGTKVEIKRTVAA | |
| | FNWYVDGVEVHNAKTKPREEQYNSTYRVVSVLTVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLTLS | |
| | GGSGGGGSGGGGSGGGGSEVQLLESGGVLVQPGGS | | KADYEKHKVYACEV | |
| | LRLSCAASGFTFSNFGMTWVRQAPGKCLEWVSGIS | | THQGLSSPVTKSEN | |
| | GGGRDTYFADSVKGRFTISRDNSKNTLYLQMNSLK | | RGEC | |
| | GEDTAVYYCVKWGNIYFDYWGQGTLVTVSSGGGGS | | | |
| | GGGGSGGGGSGGGGSDIQMTQSPSSLSASVGDSIT | | | |
| | ITCRASLSINTFLNWYQQKPGKAPNLLIYAASSLH | | | |
| | GGVPSRFSGSGSGTDFTLTIRTLQPEDFATYYCQQ | | | |
| | SSNTPFTFGCGTVVDER | | | |
| PAL054- | EVQLVESGGGLVQPGGSLRLSCAASGYTFTNYGMN | 764 | DIQMTQSPSSLSAS | 765 |
| 0011.1L | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKTKPREEQYNSTYRVVSVLTVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLTLS | |
| | GGSGGGGSGGGGSGGGGSDVQLVESGGGVVQPGGS | | KADYEKHKVYACEV | |
| | LRLSCAASGSIASIHAMGWFRQAPGKEREFVAVIT | | THQGLSSPVTKSEN | |
| | WSGGITYYADSVKGRFTISRDNSKNTVYLQMNSLR | | RGEC | |
| | PEDTALYYCAGDKHQSSWYDYWGQGTLVTVSS | | | |
| PAL054- | EVQLVESGGGLVQPGGSLRLSCAASGYTFTNYGMN | 766 | DIQMTQSPSSLSAS | 767 |
| 0011.1La | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLYITREPEVTCVVVDVSHEDPEVK | | GQGTKVEIKRTVAA | |
| | FNWYVDGVEVHNAKTKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGKGG | | KDSTYSLSSTLTLS | |
| | GGSGGGGSGGGGSGGGGSDVQLVESGGGVVQPGGS | | KADYEKHKVYACEV | |
| | LRLSCAASGSIASIHAMGWFRQAPGKEREFVAVIT | | THQGLSSPVTKSEN | |
| | WSGGITYYADSVKGRFTISRDNSKNTVYLQMNSLR | | RGEC | |
| | PEDTALYYCAGDKHQSSWYDYWGQGTLVTVSS | | | |
| PAL054- | EVQLVESGGGLVQPGGSLRLSCAASGYTFTNYGMN | 768 | DIQMTQSPSSLSAS | 769 |
| 0012.1L | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKTKPREEQYNSTYRVVSVLTVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGGGG | | KDSTYSLSSTLILS | |
| | GSGGGGSGEVQLQESGGGLVQPGGSLRLSCAASGF | | KADYEKHKVYACEV | |
| | TFSSYWMYWLRQAPGKGLEWVSSINSDSSSTYYRD | | THQGLSSPVTKSEN | |
| | SVKGRFTISRDNAKNTLYLQMNSLKSEDTAVYYCA | | RGEC | |
| | KDPGGYAKGQGTQVTVSS | | | |
| PAL054- | SDTGRPFVEMYSEIPEIIHMTEGRELVIPCRVTSP | 770 | DIQMTQSPSSLSAS | 771 |
| 0013.1 | NITVTLKKFPLDTLIPDGKRIIWDSRKGFIISAAT | | VGDRVTITCRASQD | |
| | YKEIGLLTCEATVNGHLYKTNYLTHRQTNTGGGGS | | VSTAVAWYQQKPGK | |
| | GGGGSGGGGSEVQLVESGGGLVQPGGSLRLSCAAS | | APKLLIYSASFLYS | |
| | GFTFSDSWIHWVRQAPGKGLEWVAWISPYGGSTYY | | GVPSRFSGSGSGTD | |
| | ADSVKGRFTISADTSKNTAYLQMNSLRAEDTAVYY | | FTLTISSLQPEDFA | |
| | CARRHWPGGFDYWGQGTLVTVSSASTKGPSVFPLA | | TYYCQQYLYHPATF | |
| | PSSKSTSGGTAALGCLVKDYFPEPVTVSWNSGALT | | GQGTKVEIKRTVAA | |
| | SGVHTFPAVLQSSGLYSLSSVVTVPSSSLGTQTYI | | PSVFIFPPSDEQLK | |
| | CNVNHKPSNTKVDKRVEPKSCDKTHTCPPCPAPEL | | SGTASVVCLLNNFY | |
| | LGGPSVFLFPPKPKDTLMISRTPEVTCVVVDVSHE | | PREAKVQWKVDNAL | |
| | DPEVKFNWYVDGVEVHNAKTKPREEQYNATYRVVS | | QSGNSQESVTEQDS | |
| | VLTVLHQDWLNGKEYKCKVSNKALPAPIAATISKA | | KDSTYSLSSTLTLS | |
| | KGQPREPQVYTLPPSREEMTKNQVSLTCLVKGFYP | | KADYEKHKVYACEV | |
| | SDIAVEWESNGQPENNYKTTPPVLDSDGSFFLYSK | | THQGLSSPVTKSEN | |
| | LTVDKSRWQQGNVFSCSVMHEALHNHYTQKSLSLS | | RGEC | |
| | PG | | | |
| PAL054- | EVQLVESGGGLVQPGGSLRLSCAASGYTFTNYGMN | 772 | DIQMTQSPSSLSAS | 773 |
| 0014.1L | WVRQAPGKGLEWVGWINTYTGEPTYAADFKRRFTF | | VGDRVTITCSASQD | |
| | SLDTSKSTAYLQMNSLRAEDTAVYYCAKYPHYYGS | | ISNYLNWYQQKPGK | |
| | SHWYFDVWGQGTLVTVSSASTKGPSVFPLAPSSKS | | APKVLIYFTSSLHS | |
| | TSGGTAALGCLVKDYFPEPVTVSWNSGALTSGVHT | | GVPSRFSGSGSGTD | |
| | FPAVLQSSGLYSLSSVVTVPSSSLGTQTYICNVNH | | FTLTISSLQPEDFA | |
| | KPSNTKVDKKVEPKSCDKTHTCPPCPAPEAAGGPS | | TYYCQQYSTVPWTF | |
| | VFLFPPKPKDTLMISRTPEVTCVVVDVSHEDPEVK | | GQGTKVEIKRTVAA | |
| | FNWYVDGVEVHNAKTKPREEQYNSTYRVVSVLIVL | | PSVFIFPPSDEQLK | |
| | HQDWLNGKEYKCKVSNKALPAPIEKTISKAKGQPR | | SGTASVVCLLNNFY | |
| | EPQVYTLPPSRDELTKNQVSLTCLVKGFYPSDIAV | | PREAKVQWKVDNAL | |
| | EWESNGQPENNYKTTPPVLDSDGSFFLYSKLTVDK | | QSGNSQESVTEQDS | |
| | SRWQQGNVFSCSVMHEALHNHYTQKSLSLSPGGGG | | KDSTYSLSSTLTLS | |
| | GSGGGGSGGGGSGGGGSEVQLVESGGGLVQPGGSL | | KADYEKHKVYACEV | |
| | RLSCAVSGNIYNRNFMGWFRQAPGKGREGVSAIYT | | THQGLSSPVTKSEN | |
| | GTSRTYYADSVKGRFTISRDNAKNTVYLQMNSLRP | | RGEC | |
| | EDTAVYYCAADLRDGFWDTGVWNTWGQGTLVTVSS | | | |
| PAL054- | QVQLVQSGVEVKKPGASVKVSCKASGYTFTNYYMY | 774 | EIVLTQSPATLSLS | 775 |
| 0015.1a | WVRQAPGQGLEWMGGINPSNGGTNFNEKFKNRVTL | | PGERATLSCRASKG | |
| | TTDSSTTTAYMELKSLQFDDTAVYYCARRDYRFDM | | VSTSGYSYLHWYQQ | |
| | GFDYWGQGTTVTVSSASTKGPSVFPLAPSSKSTSG | | KPGQAPRLLIYLAS | |
| | GTAALGCLVKDYFPEPVTVSWNSGALTSGVHTFPA | | YLESGVPARFSGSG | |
| | VLQSSGLYSLSSVVTVPSSSLGTQTYICNVNHKPS | | SGTDFTLTISSLEP | |
| | NTKVDKKVEPKSCDKTHTCPPCPAPELLGGPSVEL | | EDFAVYYCQHSRDL | |
| | FPPKPKDTLMISRTPEVTCVVVDVSHEDPEVKENW | | PLTFGGGTKVEIKR | |
| | YVDGVEVHNAKTKPREEQYASTYRVVSVLIVLHQD | | TVAAPSVFIFPPSD | |
| | WLNGKEYKCKVSNKALPAPIEKTISKAKGQPREPQ | | EQLKSGTASVVCLL | |
| | VYTLPPSRDELTKNQVSLLCLVKGFYPSDIAVEWE | | NNFYPREAKVQWKV | |
| | SNGQPENNYKTTPPVLRSDGSFFLYSKLTVDKSRW | | DNALQSGNSQESVT | |
| | QQGNVFSCSVMHEALHNHYTQKSLSLSPGK | | EQDSKDSTYSLSST | |
| | | | LTLSKADYEKHKVY | |
| | | | ACEVTHQGLSSPVT | |
| | | | KSENRGEC | |
| PAL054- | EVQLVESGGGLVQPGGSLRLSCAASGFTISDYWIH | 776 | DIQMTQSPSSLSAS | 777 |
| 0015.1b | WVRQAPGKGLEWVAGITPAGGYTYYADSVKGRFTI | | VGDRVTITCRASQD | |
| | SADTSKNTAYLQMNSLRAEDTAVYYCARFVFFLPY | | VSTAVAWYQQKPGK | |
| | AMDYWGQGTLVTVSSASTKGPSVFPLAPSSKSTSG | | APKLLIYSASFLYS | |
| | GTAALGCLVKDYFPEPVTVSWNSGALTSGVHTFPA | | GVPSRFSGSGSGTD | |
| | VLQSSGLYSLSSVVTVPSSSLGTQTYICNVNHKPS | | FTLTISSLQPEDFA | |
| | NTKVDKKVEPKSCDKTHTCPPCPAPELLGGPSVFL | | TYYCQQGYGNPFTF | |
| | FPPKPKDTLMISRTPEVTCVVVDVSHEDPEVKENW | | GQGTKVEIKRTVAA | |
| | YVDGVEVHNAKTKPREEQYASTYRVVSVLIVLHQD | | PSVFIFPPSDEQLK | |
| | WLNGKEYKCKVSNKALPAPIEKTISKAKGQPREPQ | | SGTASVVCLLNNFY | |
| | VYTEPPSRDELTKNQVSLTCLVKGFYPSDIAVEWE | | PREAKVQWKVDNAL | |
| | SNGQPENNYKTTPPVLDSDGSFFLLSVLIVDKSRW | | QSGNSQESVTEQDS | |
| | QQGNVFSCSVMHEALHNHYTQKSLSLSPGK | | KDSTYSLSSTLTLS | |
| | | | KADYEKHKVYACEV | |
| | | | THQGLSSPVTKSEN | |
| | | | RGEC | |
| PAL054- | QVQLVESGGGLVQPGGSLRLSCAASGFAFSSYDMS | 778 | DIQMTQSPSSLSAS | 779 |
| 0016.1L | WVRQAPGKCLEWVATISGGGRYTYYPDSVKGRFTI | | VGDRVTITCSASQD | |
| | SRDNSKNNLYLQMNSLRAEDTAVYYCAVRYGETWE | | ISNYLNWYQQKPGK | |
| | AYWGQGTLVTVSSGGGGGGGGSGGGGSGGGGSDI | | APKVLIYFTSSLHS | |
| | QMTQSPSSLSASVGDRVTITCRASQDINTYLAWFQ | | GVPSRFSGSGSGTD | |
| | QKPGKAPKSLIYRANRLVSGVPSRFSGSGSGTDFT | | FTLTISSLQPEDFA | |
| | LTISSLQPEDMATYYCLQYDEFPLTFGCGTKLELK | | TYYCQQYSTVPWTF | |
| | GGGASGGGGSGGGGSEVQLVESGGGLVQPGGSLRL | | GQGTKVEIKRTVAA | |
| | SCAASGYTFTNYGMNWVRQAPGKGLEWVGWINTYT | | PSVFIFPPSDEQLK | |
| | GEPTYAADFKRRFTFSLDISKSTAYLQMNSLRAED | | SGTASVVCLLNNFY | |
| | TAVYYCAKYPHYYGSSHWYFDVWGQGTLVTVSSAS | | PREAKVQWKVDNAL | |
| | TKGPSVFPLAPSSKSTSGGTAALGCLVKDYFPEPV | | QSGNSQESVTEQDS | |
| | TVSWNSGALTSGVHTFPAVLQSSGLYSLSSVVTVP | | KDSTYSLSSTLTLS | |
| | SSSLGTQTYICNVNHKPSNTKVDKKVEPKSCDKTH | | KADYEKHKVYACEV | |
| | TCPPCPAPEAAGGPSVFLFPPKPKDTLMISRTPEV | | THQGLSSPVTKSEN | |
| | TCVVVDVSHEDPEVKFNWYVDGVEVHNAKTKPREE | | RGEC | |
| | QYNSTYRVVSVLTVLHQDWLNGKEYKCKVSNKALP | | | |
| | APIEKTISKAKGQPREPQVYTLPPSREEMTKNQVS | | | |
| | LTCLVKGFYPSDIAVEWESNGQPENNYKTTPPVLD | | | |
| | SDGSFFLYSKLTVDKSRWQQGNVFSCSVMHEALHN | | | |
| | HYTQKSLSLSPGK | | | |
| PAL054- | EVQLVESGGGLVQPGGSLRLSCAASGFVFSNYDMS | 780 | DIQMTQSPSSLSAS | 781 |
| 0017.4 | WVRQAPGKRLEWVATISGGGGYTYYSDSVKGRFTI | | VGDRVTITCSASQD | |
| | SRDNAKNSLYLQMNSLRAEDTAVYYCASPYGHYGF | | ISNYLNWYQQKPGK | |
| | EYWGQGTLVTVSSASTKGPSVFPLAPCSRSTSEST | | APKVLIYFTSSLHS | |
| | AALGCLVKDYFPEPVTVSWNSGALTSGVHTFPAVL | | GVPSRFSGSGSGTD | |
| | QSSGLYSLSSVVTVPSSSLGTKTYTCNVDHKPSNT | | FTLTISSLQPEDFA | |
| | KVDKRVGGGGSGGGGSGGGGSEVQLVESGGGLVQP | | TYYCQQYSTLPWTF | |
| | GGSLRLSCAASGYDFTHYGMNWVRQAPGKGLEWVG | | GQGTKVEIKRTVAA | |
| | WINTYTGEPTYAADFKRRFTFSLDISKSTAYLQMN | | PSVFIFPPSDEQLK | |
| | SLRAEDTAVYYCAKYPYYYGTSHWYFDVWGQGTLV | | SGTASVVCLLNNFY | |
| | TVSSASTKGPSVFPLAPCSRSTSESTAALGCLVKD | | PREAKVQWKVDNAL | |
| | YFPEPVTVSWNSGALTSGVHTFPAVLQSSGLYSLS | | QSGNSQESVTEQDS | |
| | SVVTVPSSSLGTKTYTCNVDHKPSNTKVDKRVESK | | KDSTYSLSSTLTLS | |
| | YGPPCPPCPAPEFLGGPSVFLFPPKPKDTLMISRT | | KADYEKHKVYACEV | |
| | PEVTCVVVDVSQEDPEVQFNWYVDGVEVHNAKTKP | | THQGLSSPVTKSEN | |
| | REEQFNSTYRVVSVLTVLHQDWLNGKEYKCKVSNK | | RGEC | |
| | GLPSSIEKTISKAKGQPREPQVYTLPPSQEEMTKN | | | |
| | QVSLTCLVKGFYPSDIAVEWESNGQPENNYKTTPP | | | |
| | VLDSDGSFFLYSRLTVDKSRWQEGNVFSCSVMHEA | | | |
| | LHNHYTQKSLSLSLGK | | | |
| PAL054- | QVQLVESGGGLVQPGGSLRLSCATSGSRRSIYAMG | 782 | DIQMTQSPSSLSAS | 783 |
| 0018.1L | WFRQAPGKGLEFVAGIGWAYATTYYADSVKGRETI | | VGDRVTITCSASQD | |
| | SRDNTKNTLYLQMNSLRAEDTAVYYCAADLDHSGF | | ISNYLNWYQQKPGK | |
| | DYWGQGTLVTVSSGGGGSGGGGSGGGGSEVQLVQS | | APKVLIYFTSSLHS | |
| | GGGVVQPGGSLRLSCAASGYTFTNYGMNWVRQAPG | | GVPSRFSGSGSGTD | |
| | KGLEWVGWINTYTGEPTYAADFKRRFTFSLDISKS | | FTLTISSLQPEDFA | |
| | TAYLQMNSLRAEDTAVYYCAKYPHYYGSSHWYFDV | | TYYCQQYSTVPWTF | |
| | WGQGTLVTVSSASTKGPSVFPLAPSSKSTSGGTAA | | GQGTKVEIKRTVAA | |
| | LGCLVKDYFPEPVTVSWNSGALTSGVHTFPAVLQS | | PSVFIFPPSDEQLK | |
| | SGLYSLSSVVTVPSSSLGTQTYICNVNHKPSNTKV | | SGTASVVCLLNNFY | |
| | DKKVEPKSCDKTHTCPPCPAPEAAGGPSVFLFPPK | | PREAKVQWKVDNAL | |
| | PKDTLMISRTPEVTCVVVDVSHEDPEVKENWYVDG | | QSGNSQESVTEQDS | |
| | VEVHNAKTKPREEQYNSTYRVVSVLTVLHQDWLNG | | KDSTYSLSSTLTLS | |
| | KEYKCKVSNKALPAPIEKTISKAKGQPREPQVYTL | | KADYEKHKVYACEV | |
| | PPSREEMTKNQVSLTCLVKGFYPSDIAVEWESNGQ | | THQGLSSPVTKSEN | |
| | PENNYKTTPPVLDSDGSFFLYSKLTVDKSRWQQGN | | RGEC | |
| | VFSCSVMHEALHNHYTQKSLSLSPGK | | | |

Controls

| | | | | |
|---|---|---|---|---|
| PAL054- | QVQLVQSGVEVKKPGASVKVSCKASGYTFTNYYMY | 784 | EIVLTQSPATLSLS | 785 |
| 0051.1La | WVRQAPGQGLEWMGGINPSQGGTNFNEKFKNRVTL | | PGERATLSCRASKG | |
| | TTDSSTTTAYMELKSLQFDDTAVYYCARRDYREDL | | VSTSGYSYLHWYQQ | |
| | GFDYWGQGTTVTVSSASTKGPSVFPLAPSSKSTSG | | KPGQAPRLLIYLAS | |
| | GTAALGCLVKDYFPEPVTVSWNSGALTSGVHTFPA | | YLESGVPARFSGSG | |
| | VLQSSGLYSLSSVVTVPSSSLGTQTYICNVNHKPS | | SGTDFTLTISSLEP | |
| | NTKVDKKVEPKSCDKTHTCPPCPAPEAAGGPSVFL | | EDFAVYYCQHSRDL | |
| | FPPKPKDTLYITREPEVTCVVVDVSHEDPEVKENW | | PLTFGGGTKVEIKR | |
| | YVDGVEVHNAKTKPREEQYNSTYRVVSVLTVLHQD | | TVAAPSVFIFPPSD | |
| | WLNGKEYKCKVSNKALPAPIEKTISKAKGQPREPQ | | EQLKSGTASVVCLL | |
| | VYTLPPSRDELTKNQVSLTCLVKGFYPSDIAVEWE | | NNFYPREAKVQWKV | |
| | SNGQPENNYKTTPPVLDSDGSFFLYSKLTVDKSRW | | DNALQSGNSQESVT | |
| | QQGNVFSCSVMHEALHNHYTQKSLSLSPGK | | EQDSKDSTYSLSST | |
| | | | LTLSKADYEKHKVY | |
| | | | ACEVTHQGLSSPVT | |
| | | | KSENRGEC | |
| PAL054- | QVQLVQSGVEVKKPGASVKVSCKASGYTFTNYYMY | 786 | EIVLTQSPATLSLS | 787 |
| 0052.1La | WVRQAPGQGLEWMGGINPSRGGTNFNEKFKNRVTL | | PGERATLSCRASKG | |
| | TTDSSTTTAYMELKSLQFDDTAVYYCARRDYRFDL | | VSTSGYSYLHWYQQ | |
| | GFDYWGQGTTVTVSSASTKGPSVFPLAPSSKSTSG | | KPGQAPRLLIYLAS | |
| | GTAALGCLVKDYFPEPVTVSWNSGALTSGVHTFPA | | YLESGVPARFSGSG | |
| | VLQSSGLYSLSSVVTVPSSSLGTQTYICNVNHKPS | | SGTDFTLTISSLEP | |
| | NTKVDKKVEPKSCDKTHTCPPCPAPEAAGGPSVFL | | EDFAVYYCQHSRDL | |
| | FPPKPKDTLYITREPEVTCVVVDVSHEDPEVKENW | | PLTFGGGTKVEIKR | |
| | YVDGVEVHNAKTKPREEQYNSTYRVVSVLTVLHQD | | TVAAPSVFIFPPSD | |
| | WLNGKEYKCKVSNKALPAPIEKTISKAKGQPREPQ | | EQLKSGTASVVCLL | |
| | VYTLPPSRDELTKNQVSLTCLVKGFYPSDIAVEWE | | NNFYPREAKVQWKV | |
| | SNGQPENNYKTTPPVLDSDGSFFLYSKLTVDKSRW | | DNALQSGNSQESVT | |
| | QQGNVFSCSVMHEALHNHYTQKSLSLSPGK | | EQDSKDSTYSLSST | |
| | | | LTLSKADYEKHKVY | |
| | | | ACEVTHQGLSSPVT | |
| | | | KSFNRGEC | |
| PAL054- | EPKSCDKTHTCPPCPAPEAAGGPSVFLFPPKPKDT | 788 | | |
| 0053.1La | LYITREPEVTCVVVDVSHEDPEVKFNWYVDGVEVH | | | |
| | NAKTKPREEQYNSTYRVVSVLTVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKTTPPVLDSDGSFFLYSKLTVDKSRWQQGNVFSC | | | |
| | SVMHEALHNHYTQKSLSLSPGKGGGGSGGGGSGGG | | | |
| | GSGGGGSQVQLVQSGVEVKKPGASVKVSCKASGYT | | | |
| | FTNYYMYWVRQAPGQGLEWMGGINPSQGGINENEK | | | |
| | FKNRVTLTTDSSTTTAYMELKSLQFDDTAVYYCAR | | | |
| | RDYRFDLGFDYWGQGTTVTVSSGGGGSGGGGSGGG | | | |
| | GSGGGGSEIVLTQSPATLSLSPGERATLSCRASKG | | | |
| | VSTSGYSYLHWYQQKPGQAPRLLIYLASYLESGVP | | | |
| | ARFSGSGSGTDFTLTISSLEPEDFAVYYCQHSRDL | | | |
| | PLTFGGGTKVEIKR | | | |
| PAL054- | EPKSCDKTHTCPPCPAPEAAGGPSVFLFPPKPKDT | 789 | | |
| 0054.1La | LYITREPEVTCVVVDVSHEDPEVKFNWYVDGVEVH | | | |
| | NAKTKPREEQYNSTYRVVSVLTVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKTTPPVLDSDGSFFLYSKLTVDKSRWQQGNVFSC | | | |
| | SVMHEALHNHYTQKSLSLSPGKGGGGSGGGGSGGG | | | |
| | GSGGGGSQVQLVQSGVEVKKPGASVKVSCKASGYT | | | |
| | FTNYYMYWVRQAPGQGLEWMGGINPSRGGINENEK | | | |
| | FKNRVTLTTDSSTTTAYMELKSLQFDDTAVYYCAR | | | |
| | RDYRFDLGFDYWGQGTTVTVSSGGGGSGGGGSGGG | | | |
| | GSGGGGSEIVLTQSPATLSLSPGERATLSCRASKG | | | |
| | VSTSGYSYLHWYQQKPGQAPRLLIYLASYLESGVP | | | |
| | ARFSGSGSGTDFTLTISSLEPEDFAVYYCQHSRDL | | | |
| | PLTFGGGTKVEIKR | | | |
| PAL054- | EPKSCDKTHTCPPCPAPEAAGGPSVFLFPPKPKDT | 790 | | |
| 0055.1La | LYITREPEVTCVVVDVSHEDPEVKENWYVDGVEVH | | | |
| | NAKTKPREEQYNSTYRVVSVLIVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKTTPPVLDSDGSFFLYSKLTVDKSRWQQGNVFSC | | | |
| | SVMHEALHNHYTQKSLSLSPGKGGGGSGGGGSGGG | | | |
| | GSGGGGSQVQLVQSGVEVKKPGASVKVSCKASGYT | | | |
| | FTNYYMYWVRQAPGQCLEWMGGINPSQGGINENEK | | | |
| | FKNRVTLTTDSSTTTAYMELKSLQFDDTAVYYCAR | | | |
| | RDYRFDLGFDYWGQGTTVTVSSGGGGSGGGGSGGG | | | |
| | GSGGGGSEIVLTQSPATLSLSPGERATLSCRASKG | | | |
| | VSTSGYSYLHWYQQKPGQAPRLLIYLASYLESGVP | | | |
| | ARFSGSGSGTDFTLTISSLEPEDFAVYYCQHSRDL | | | |
| | PLTFGCGTKVEIKR | | | |
| PAL054- | EPKSCDKTHTCPPCPAPEAAGGPSVFLFPPKPKDT | 791 | | |
| 0056.1La | LYITREPEVTCVVVDVSHEDPEVKFNWYVDGVEVH | | | |
| | NAKTKPREEQYNSTYRVVSVLTVLHQDWLNGKEYK | | | |
| | CKVSNKALPAPIEKTISKAKGQPREPQVYTLPPSR | | | |
| | DELTKNQVSLTCLVKGFYPSDIAVEWESNGQPENN | | | |
| | YKITPPVLDSDGSFFLYSKLTVDKSRWQQGNVFSC | | | |
| | SVMHEALHNHYTQKSLSLSPGKGGGGSGGGGSGGG | | | |
| | GSGGGGSQVQLVQSGVEVKKPGASVKVSCKASGYT | | | |
| | FTNYYMYWVRQAPGQCLEWMGGINPSRGGINENEK | | | |
| | FKNRVTLTTDSSTTTAYMELKSLQFDDTAVYYCAR | | | |
| | RDYRFDLGFDYWGQGTTVTVSSGGGGSGGGGSGGG | | | |
| | GSGGGGSEIVLTQSPATLSLSPGERATLSCRASKG | | | |
| | VSTSGYSYLHWYQQKPGQAPRLLIYLASYLESGVP | | | |
| | ARFSGSGSGTDFTLTISSLEPEDFAVYYCQHSRDL | | | |
| | PLTFGCGTKVEIKR | | | |

### Example 1: Determination of PD-1 Binding on PD-1 Expressing Cells

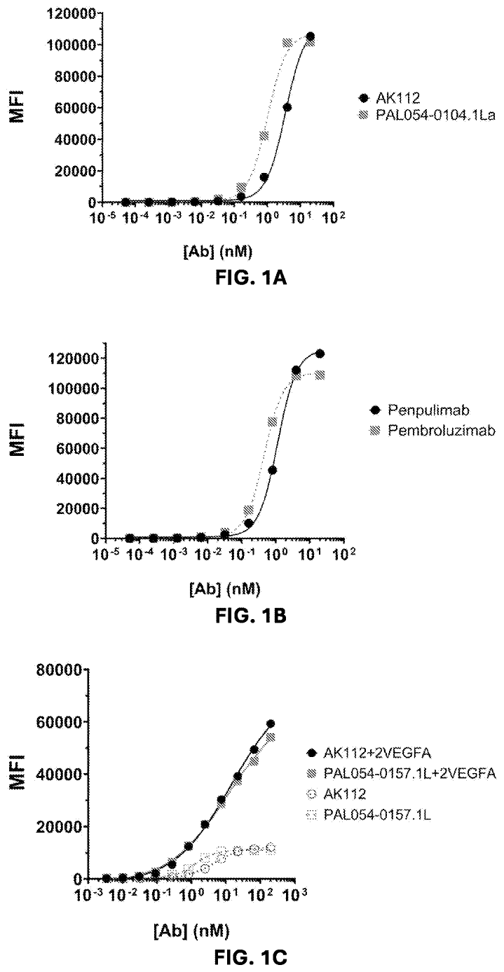

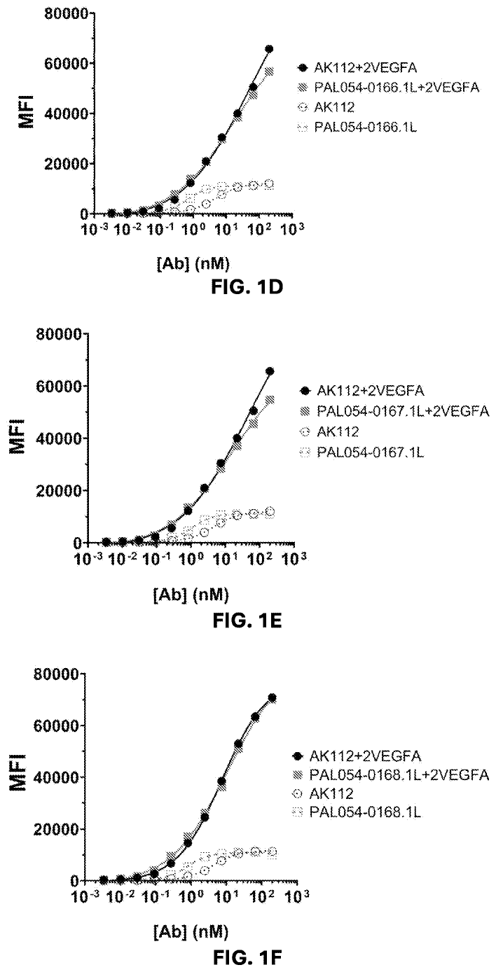

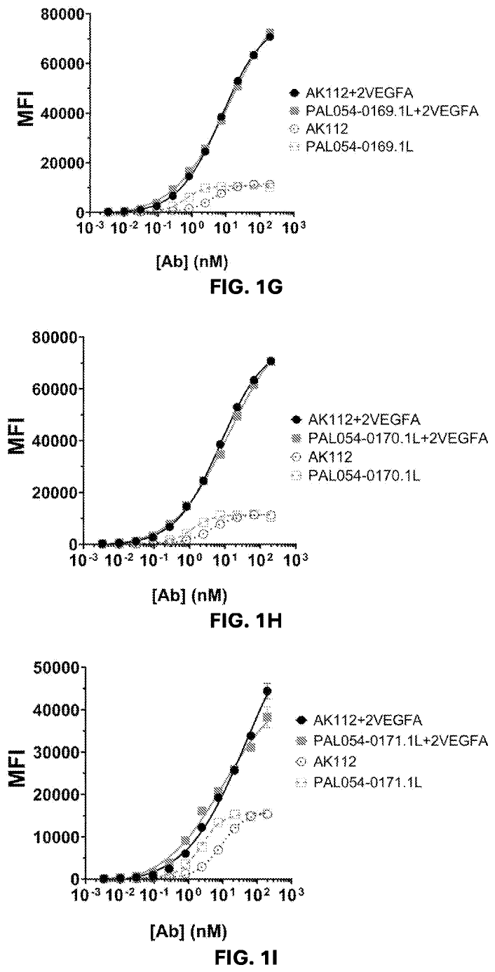

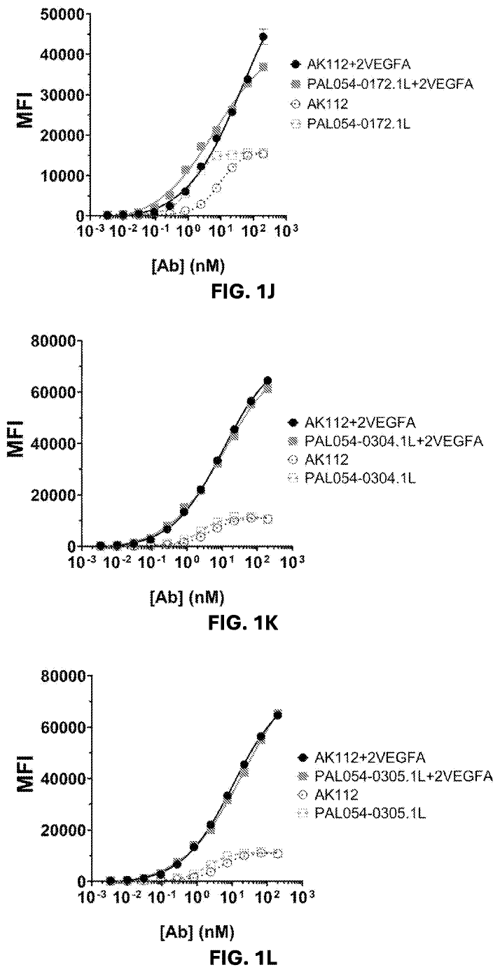

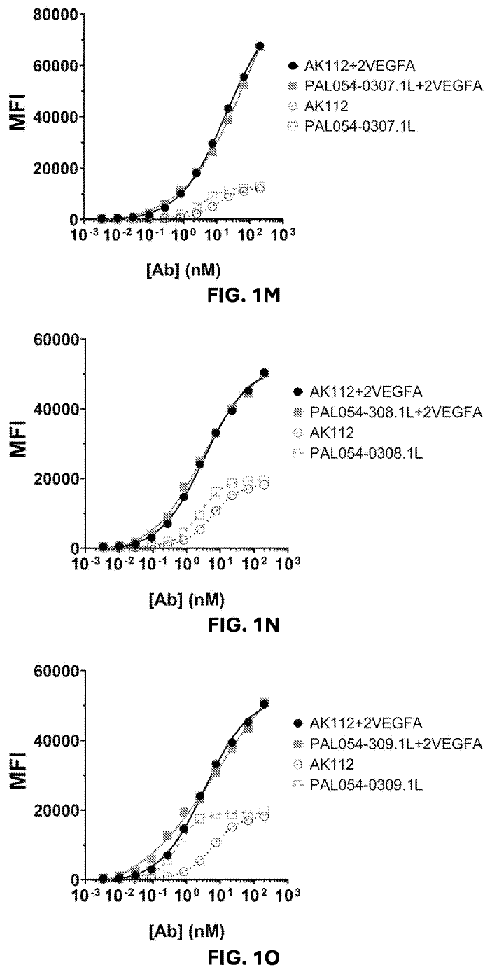

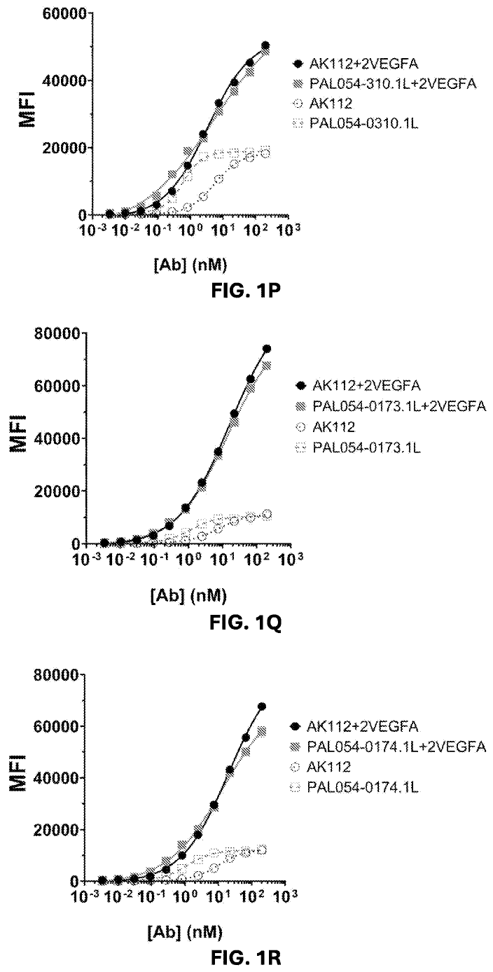

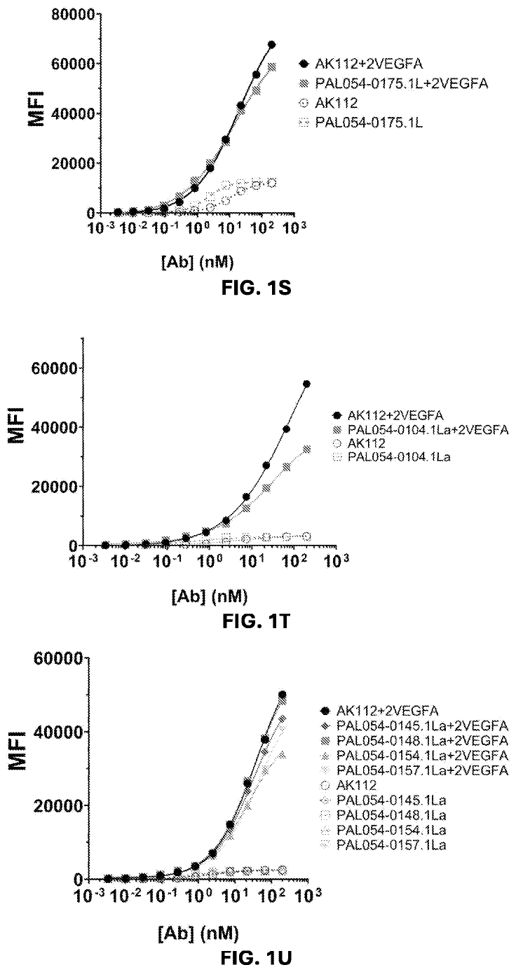

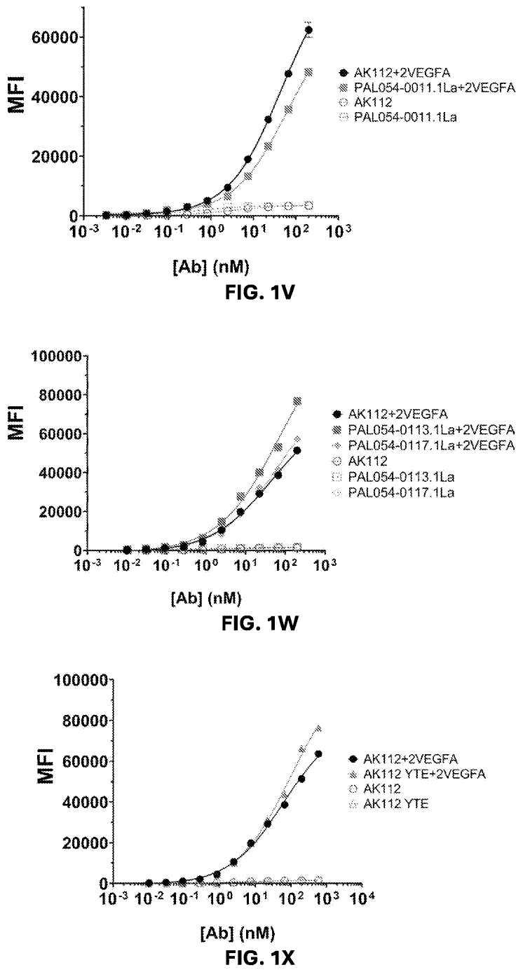

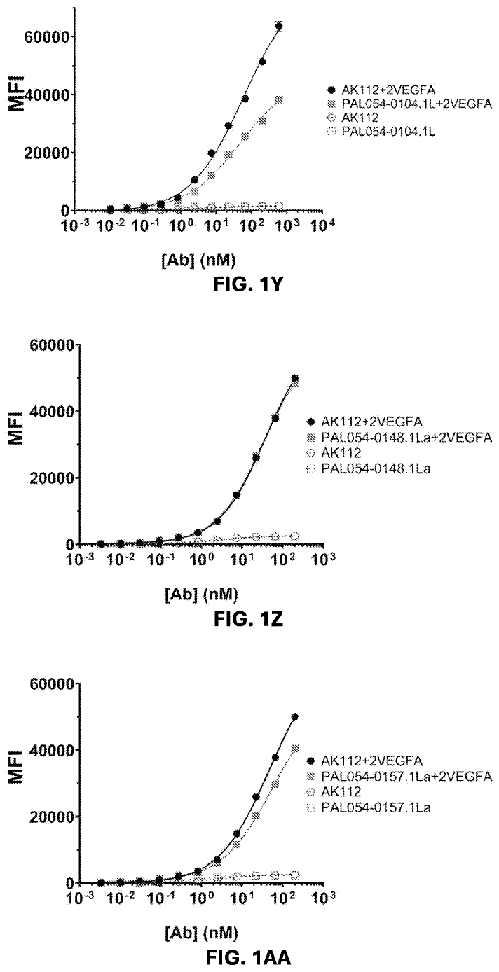

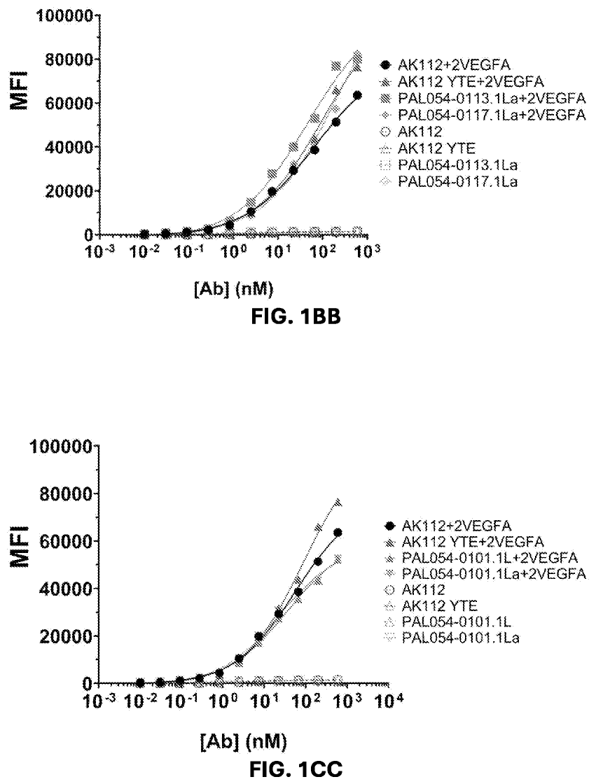

PD-1 binding was assessed in Jurkat-hPD-1 cells in the presence and absence of 2×VEGFA by flow cytometry as described above. Greater binding to PD-1 expressing cells was seen in the presence of VEGFA (referred to as VEGF or VEGFA interchangeably). The results are shown in FIGS. 1A-1CC and Table 12.

TABLE 12

| | Rel binding (MFI) at 200 |
|---|---|
| Bispecifics | nM (+VEGF) |
| PAL054-0001.1L (AK112) | 1 |
| PAL054-0145.1La | 0.9 |
| PAL054-0148.1La | 1 |
| PAL054-0154.1La | 0.8 |
| PAL054-0157.1La | 0.8 |
| PAL054-0011.1La | 0.8 |
| PAL054-0104.1La | 0.6 |
| PAL054-0104.1L | 0.6 |
| PAL054-0001.1La (AK112 YTE) | 1.3 |
| PAL054-0113.1La | 1.5 |
| PAL054-0117.1La | 1.1 |
| PAL054-0157.1L | 0.9 |
| PAL054-0166.1L | 0.9 |
| PAL054-0167.1L | 0.8 |
| PAL054-0168.1L | 1 |
| PAL054-0169.1L | 1 |
| PAL054-0170.1L | 1 |
| PAL054-0171.1L | 0.9 |
| PAL054-0172.1L | 0.8 |
| PAL054-0173.1L | 0.9 |
| PAL054-0174.1L | 0.9 |
| PAL054-0175.1L | 0.9 |
| PAL054-0304.1L | 1 |
| PAL054-0305.1L | 1 |
| PAL054-0307.1L | 1 |
| PAL054-0308.1L | 1 |
| PAL054-0309.1L | 1 |
| PAL054-0310.1L | 1 |

### Example 2: Determination of Antibody Affinity to PD-1

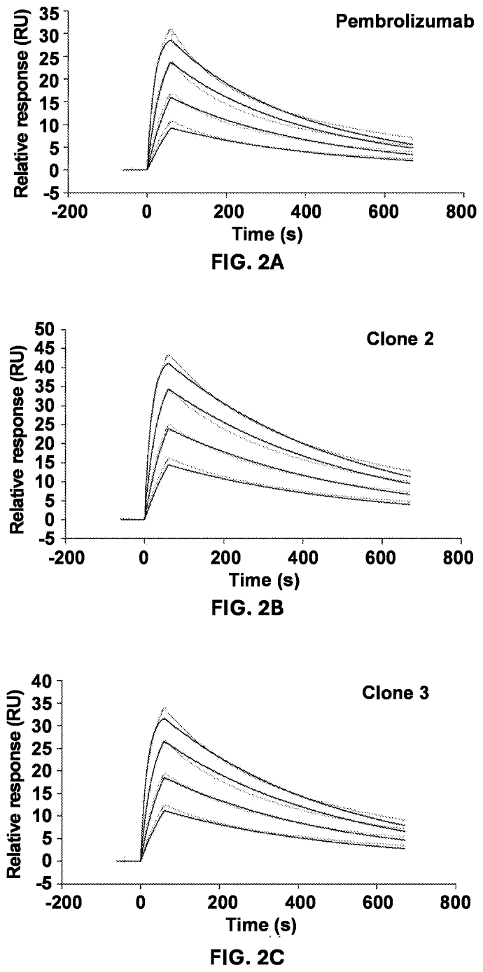

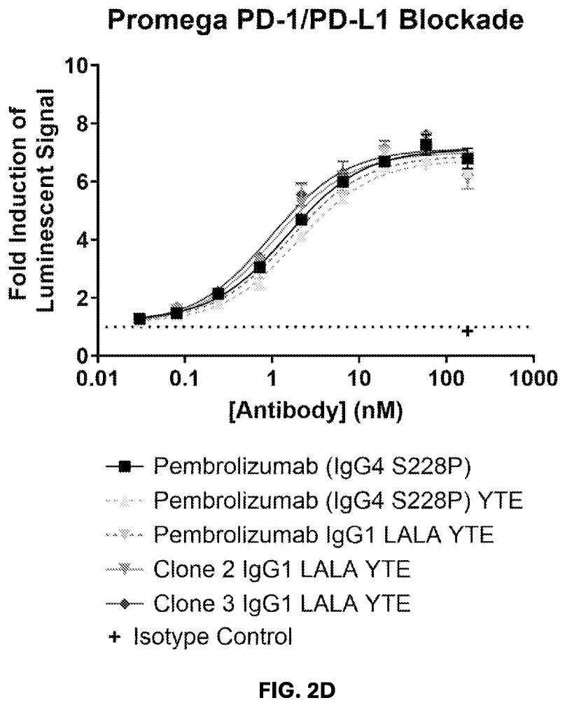

Binding affinity (K_{D}) of pembrolizumab and clone variants, with indicated Fc modifications, to PD-1 was determined by surface plasmon resonance (FIGS. 2A-2C and Table 13), and PD-1/PD-L1 blockade by the antibodies was analyzed by a PD-1 reporter assay (FIG. 2D).

TABLE 13

| Clone | ka (1/Ms) | kd (1/s) | KD (nM) |
|---|---|---|---|
| Pembrolizumab | 6.60E+05 | 3.10E−03 | 4.70 |
| Clone 2 (PAL054-0051.1La) | 5.73E+05 | 2.11E−03 | 3.69 |
| Clone 3 (PAL054-0052.1La) | 5.81E+05 | 2.27E−03 | 3.90 |

### Example 3: Binding of Bispecific Antibody to Human VEGF, Human PD-1 and Cynomolgus PD-1 Determined by ELISA

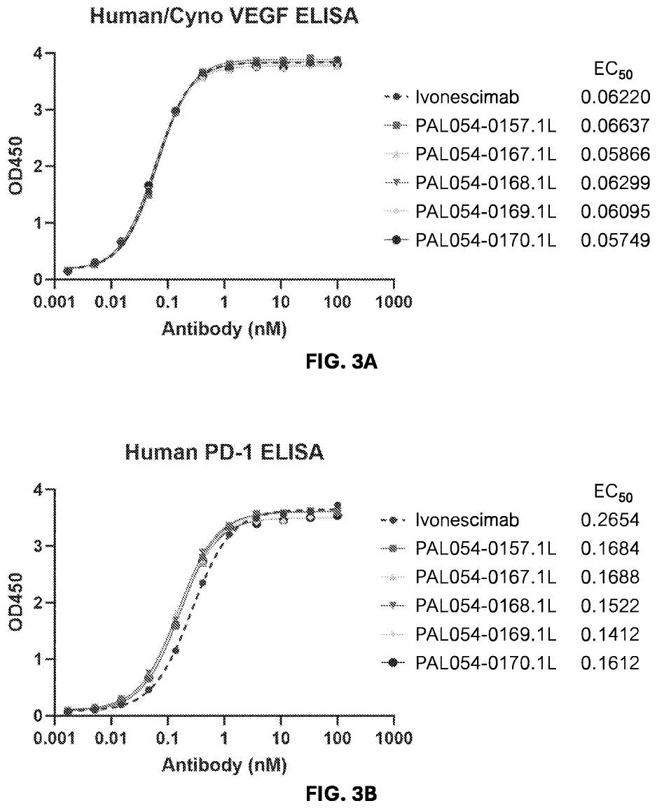

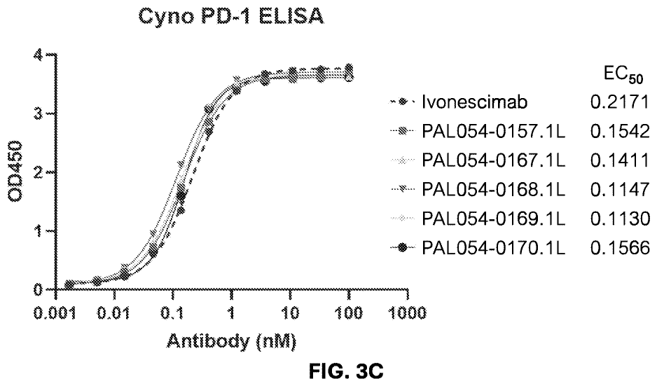

PD-1 binding assays were conducted to measure the affinity of AK112 and VEGF/PD-1 bispecific antibodies disclosed herein to PD-1 expressing cells. Half maximal effective concentration (EC₅₀) of binding of test articles to human VEGF, human PD-1, and cynomolgus PD-1 was determined by ELISA. Data is seen in FIGS. 3A-3C and Table 14.

TABLE 14

| | EC₅₀ (nM) | | |
|---|---|---|---|
| Test Article | Human VEGF | Human PD-1 | Cyno PD-1 |
| Ivonescimab | 0.0622 | 0.2654 | 0.2171 |
| PAL054-0157.1L | 0.06637 | 0.1684 | 0.1542 |
| PAL054-0167.1L | 0.05866 | 0.1688 | 0.1411 |
| PAL054-0168.1L | 0.06299 | 0.1522 | 0.1147 |
| PAL054-0169.1L | 0.06095 | 0.1412 | 0.1130 |
| PAL054-0170.1L | 0.05749 | 0.1612 | 0.1566 |

### Example 4: Determination of Bispecific Antibody Affinity to Human VEGF

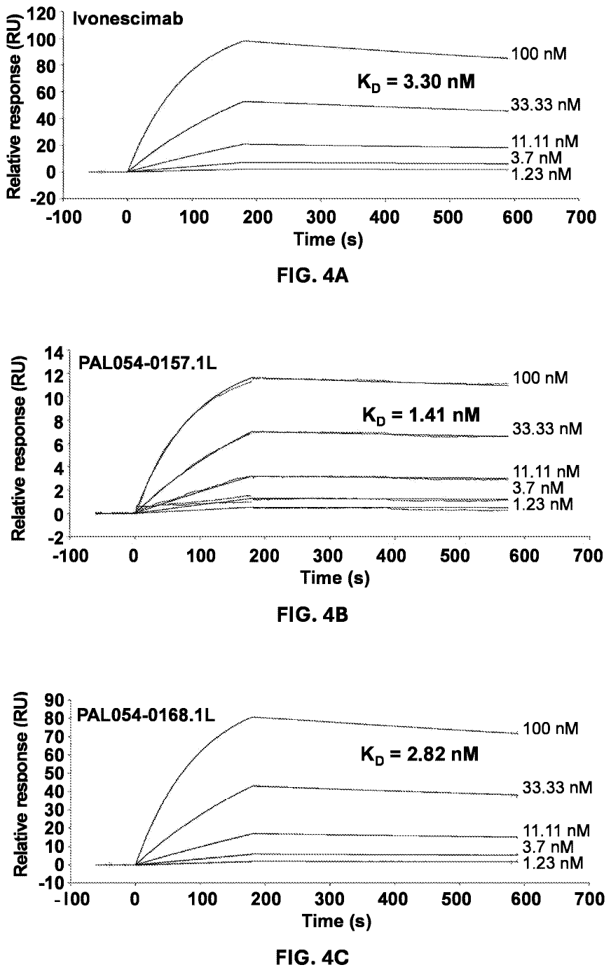

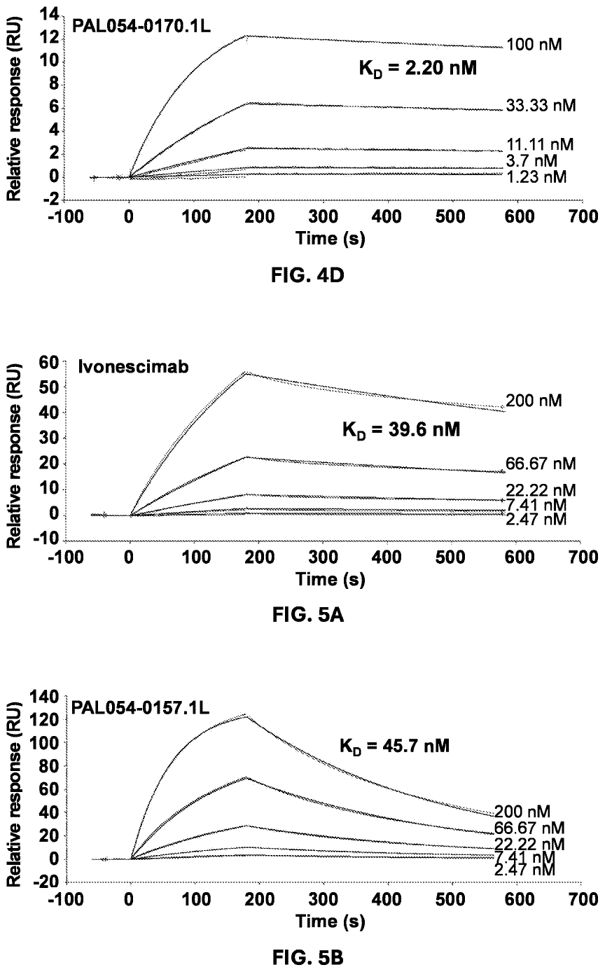

Binding affinity (K_{D}) of bispecific antibodies to human VEGF was determined by surface plasmon resonance by capturing test articles with Protein G chip and flowing human VEGF at 100, 33.33, 11.11, 3.7, and 1.23 nM (FIGS. 4A-4D and Table 15).

TABLE 15

| Test Article | Analyte | kₐ (1/Ms) | k_{d} (1/s) | K_{D} (M) |
|---|---|---|---|---|
| Ivonescimab | Human | 1.07E+05 | 3.55E−04 | 3.30E−09 |
| | VEGF165-His | | | |
| PAL054-0157.1L | Human | 1.08E+05 | 1.52E−04 | 1.41E−09 |
| | VEGF165-His | | | |
| PAL054-0166.1L | Human | 1.11E+05 | 2.83E−04 | 2.56E−09 |
| | VEGF165-His | | | |
| PAL054-0168.1L | Human | 1.04E+05 | 2.92E−04 | 2.82E−09 |
| | VEGF165-His | | | |
| PAL054-0170.1L | Human | 9.91E+04 | 2.18E−04 | 2.20E−09 |
| | VEGF165-His | | | |
| PAL054-0171.1L | Human | 1.11E+05 | 3.54E−04 | 3.19E−09 |
| | VEGF165-His | | | |
| PAL054-0172.1L | Human | 9.94E+04 | 3.02E−04 | 3.03E−09 |
| | VEGF165-His | | | |
| PAL054-0173.1L | Human | 1.06E+05 | 2.89E−04 | 2.73E−09 |
| | VEGF165-His | | | |
| PAL054-0174.1L | Human | 1.02E+05 | 2.97E−04 | 2.90E−09 |
| | VEGF165-His | | | |
| PAL054-0175.1L | Human | 1.07E+05 | 3.40E−04 | 3.18E−09 |
| | VEGF165-His | | | |
| PAL054-0304.1L | Human | 1.10E+05 | 2.87E−04 | 2.61E−09 |
| | VEGF165-His | | | |
| PAL054-0305.1L | Human | 9.72E+04 | 2.92E−04 | 3.01E−09 |
| | VEGF165-His | | | |
| PAL054-0307.1L | Human | 1.02E+05 | 2.73E−04 | 2.67E−09 |
| | VEGF165-His | | | |
| PAL054-0308.1L | Human | 1.05E+05 | 2.94E−04 | 2.79E−09 |
| | VEGF165-His | | | |
| PAL054-0309.1L | Human | 9.50E+04 | 2.97E−04 | 3.12E−09 |
| | VEGF165-His | | | |
| PAL054-0310.1L | Human | 1.04E+05 | 2.97E−04 | 2.87E−09 |
| | VEGF165-His | | | |

### Example 5: Determination of Bispecific Antibody Affinity to Human PD-1

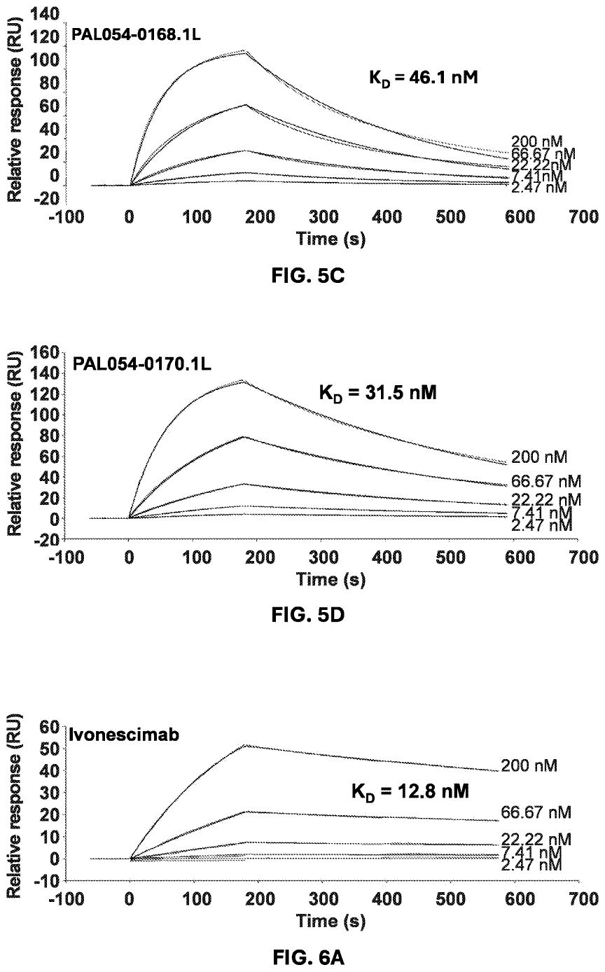

Binding affinity (K_{D}) of bispecific antibodies to human PD-1 was determined by surface plasmon resonance by capturing test articles with Protein G chip and flowing human PD-1 at 200, 66.67, 22.22, 7.41, and 2.47 nM (FIGS. 5A-5D and Table 16).

TABLE 16

| Test Article | Analyte | kₐ (1/Ms) | k_{d} (1/s) | K_{D} (M) |
|---|---|---|---|---|
| Ivonescimab | Human PD-1-His | 1.94E+04 | 7.69E−04 | 3.96E−08 |
| PAL054-0148.1L | Human PD-1-His | 5.39E+04 | 3.40E−03 | 6.31E−08 |
| PAL054-0157.1L | Human PD-1-His | 7.16E+04 | 3.27E−03 | 4.57E−08 |
| PAL054-0165.1L | Human PD-1-His | 9.17E+04 | 3.12E−03 | 3.40E−08 |
| PAL054-0166.1L | Human PD-1-His | 9.11E+04 | 2.96E−03 | 3.25E−08 |
| PAL054-0167.1L | Human PD-1-His | 6.71E+04 | 3.29E−03 | 4.91E−08 |
| PAL054-0168.1L | Human PD-1-His | 8.71E+04 | 4.01E−03 | 4.61E−08 |
| PAL054-0169.1L | Human PD-1-His | 8.54E+04 | 4.05E−03 | 4.74E−08 |
| PAL054-0170.1L | Human PD-1-His | 7.28E+04 | 2.29E−03 | 3.15E−08 |
| PAL054-0301.1L | Human PD-1-His | 4.96E+04 | 3.87E−03 | 7.80E−08 |
| PAL054-0302.1L | Human PD-1-His | 3.30E+04 | 2.59E−03 | 7.84E−08 |
| PAL054-0303.1L | Human PD-1-His | 2.77E+04 | 5.30E−04 | 1.91E−08 |
| PAL054-0304.1L | Human PD-1-His | 3.59E+04 | 5.36E−04 | 1.49E−08 |
| PAL054-0305.1L | Human PD-1-His | 3.73E+04 | 4.35E−04 | 1.16E−08 |
| PAL054-0306.1L | Human PD-1-His | 3.22E+04 | 2.69E−03 | 8.34E−08 |

### Example 6: Determination of Bispecific Antibody Affinity to Cynomolgus PD-1

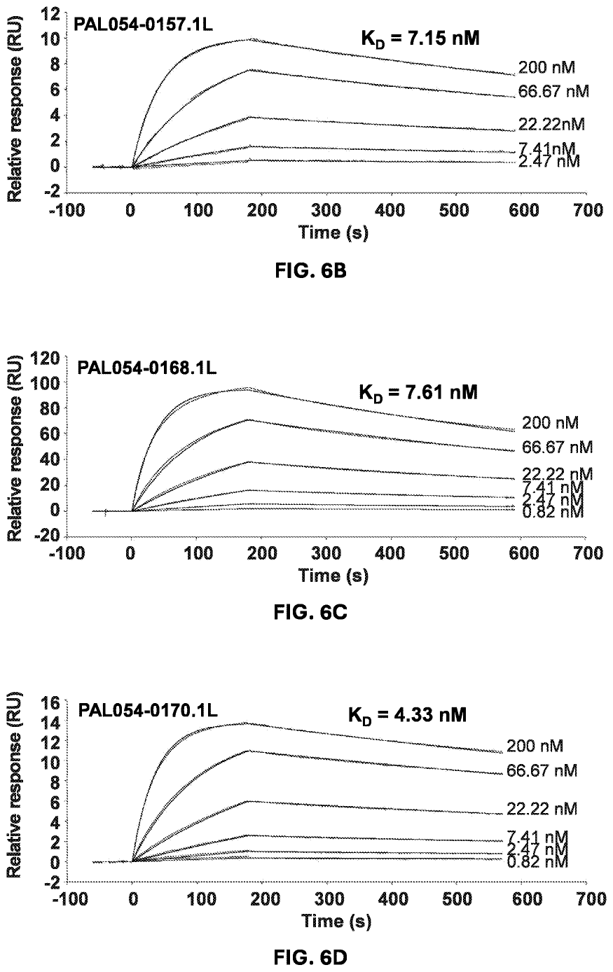

Binding affinity (K_{D}) of bispecific antibodies to cynomolgus PD-1 was determined by surface plasmon resonance by capturing test articles with Protein G chip and flowing cyno PD-1 at 200, 66.67, 22.22, 7.41, and 2.47 nM or at 200, 66.67, 22.22, 7.41, 2.47, and 0.82 nM (FIGS. 6A-6D and Table 17).

TABLE 17

| Test Article | Analyte | kₐ (1/Ms) | k_{d} (1/s) | K_{D} (M) |
|---|---|---|---|---|
| Ivonescimab | Cyno PD-1-His | 7.22E+04 | 9.01E−04 | 1.25E−08 |
| PAL054-0157.1L | Cyno PD-1-His | 1.11E+05 | 7.91E−04 | 7.15E−09 |
| PAL054-0166.1L | Cyno PD-1-His | 1.50E+05 | 7.70E−04 | 5.15E−09 |
| PAL054-0168.1L | Cyno PD-1-His | 1.35E+05 | 1.02E−03 | 7.61E−09 |
| PAL054-0170.1L | Cyno PD-1-His | 1.40E+05 | 6.08E−04 | 4.33E−09 |
| PAL054-0171.1L | Cyno PD-1-His | 9.93E+04 | 7.01E−04 | 7.06E−09 |
| PAL054-0172.1L | Cyno PD-1-His | 1.48E+05 | 7.49E−04 | 5.06E−09 |
| PAL054-0173.1L | Cyno PD-1-His | 1.29E+05 | 1.10E−03 | 8.52E−09 |
| PAL054-0174.1L | Cyno PD-1-His | 1.37E+05 | 1.11E−03 | 8.12E−09 |
| PAL054-0175.1L | Cyno PD-1-His | 1.15E+05 | 6.68E−04 | 5.82E−09 |
| PAL054-0304.1L | Cyno PD-1-His | 2.43E+04 | 3.51E−03 | 1.44E−07 |
| PAL054-0305.1L | Cyno PD-1-His | 3.06E+04 | 3.30E−03 | 1.08E−07 |
| PAL054-0307.1L | Cyno PD-1-His | 4.46E+04 | 4.39E−03 | 9.85E−08 |
| PAL054-0308.1L | Cyno PD-1-His | 3.20E+04 | 3.86E−03 | 1.20E−07 |
| PAL054-0309.1L | Cyno PD-1-His | 1.29E+05 | 6.00E−05 | 4.65E−10 |
| PAL054-0310.1L | Cyno PD-1-His | 1.18E+05 | 9.85E−05 | 8.35E−10 |

### Example 7: Size Exclusion Chromatography of Bispecific Variants, Controls, and Benchmarks

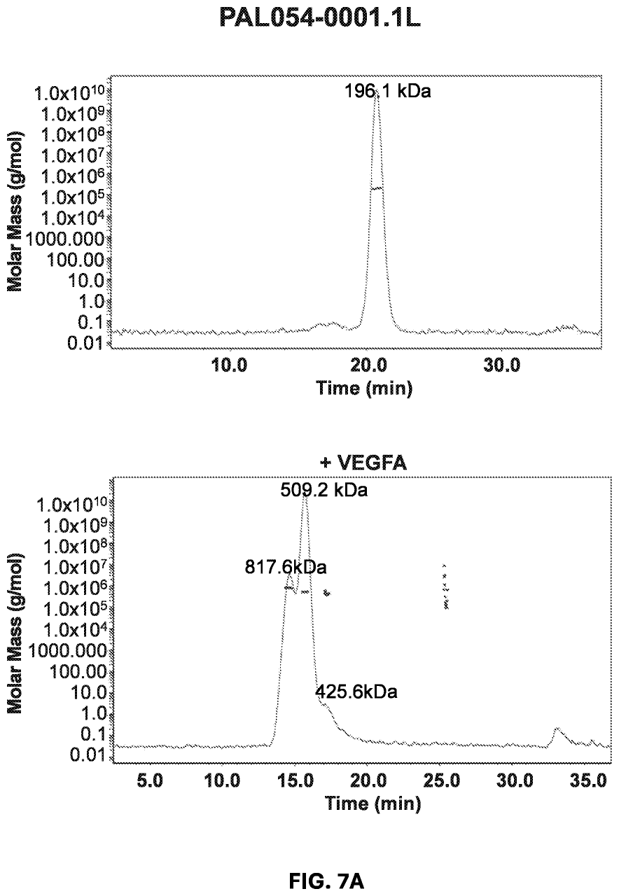

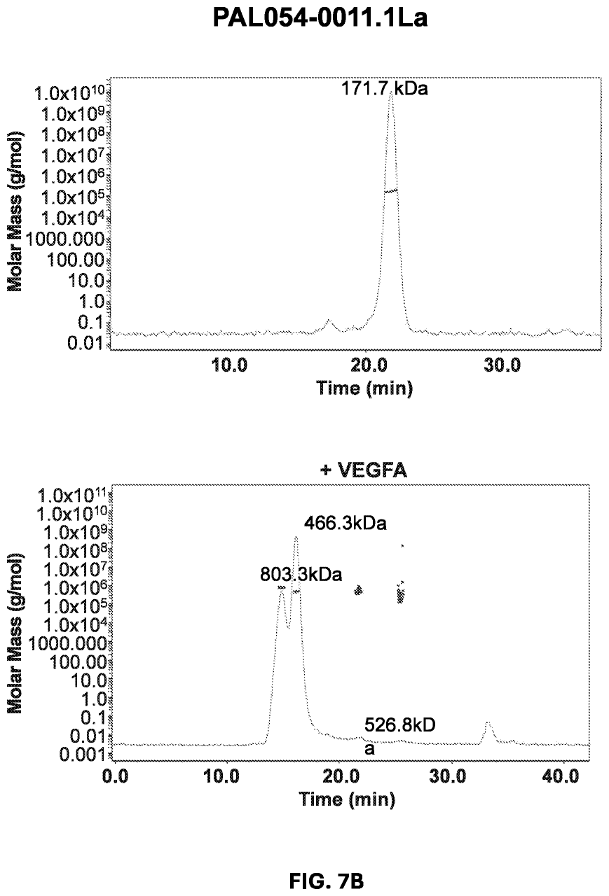

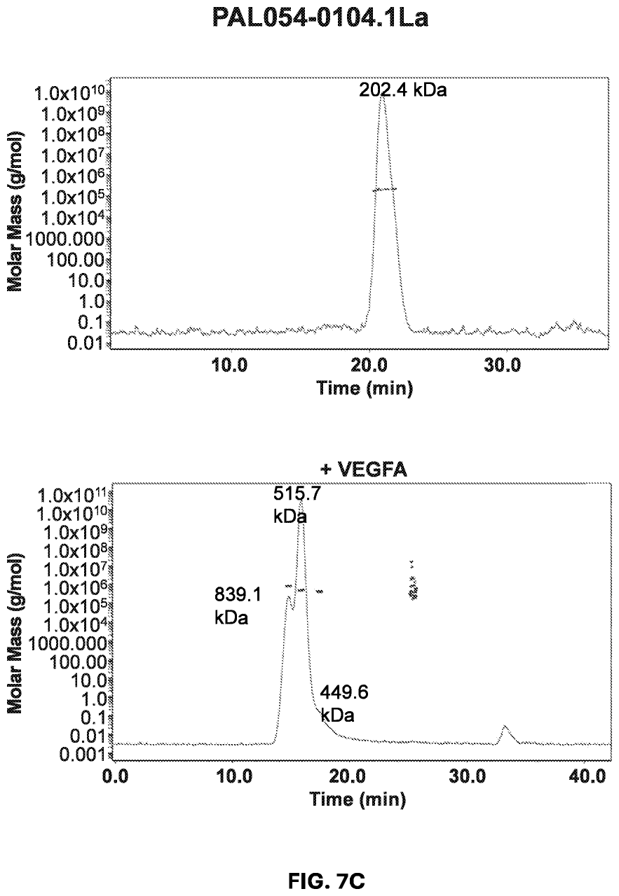

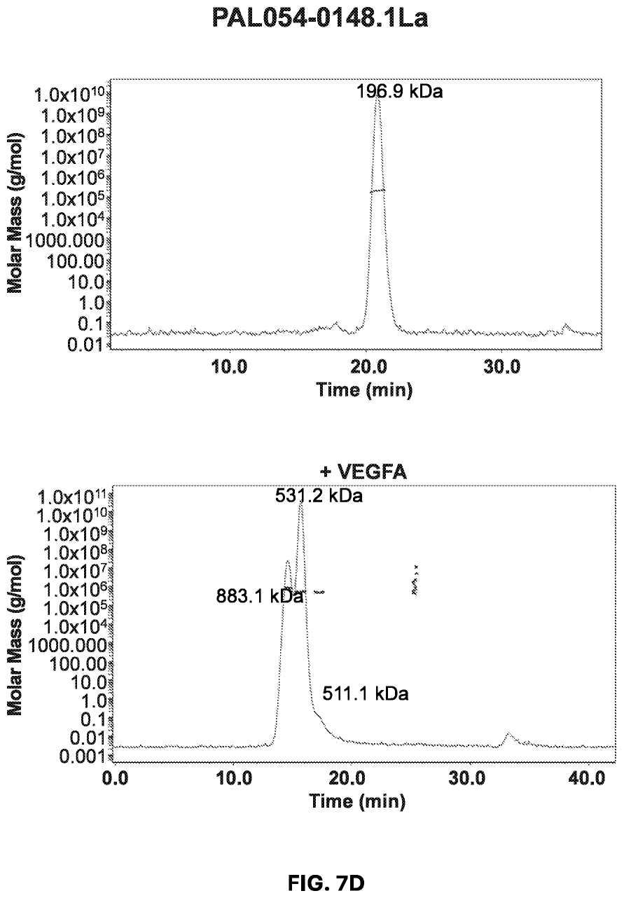

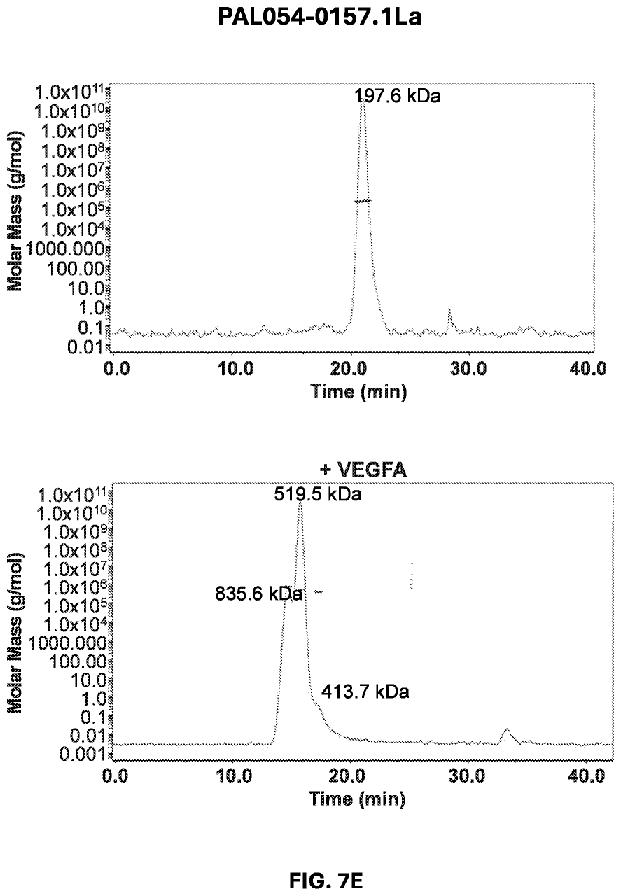

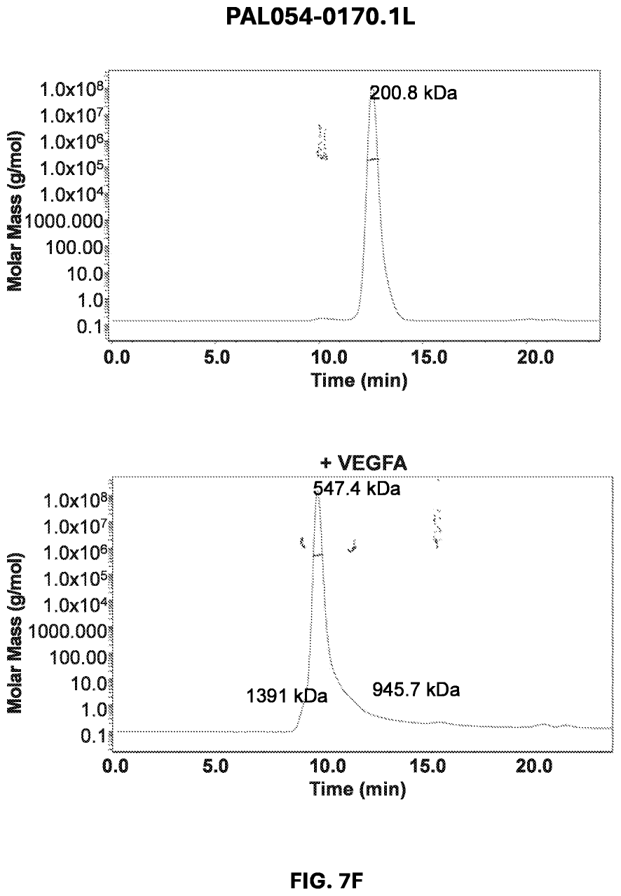

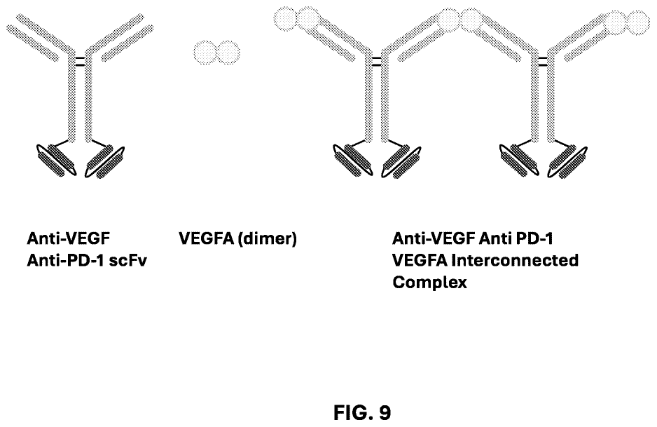

Size exclusion chromatography with multi-angle static light scattering (SEC-MALS) was used to characterize and compare benchmark bispecific, AK112, to controls and VEGF/PD-1 bispecific antibodies as disclosed herein. Exemplary clones disclosed herein exhibited similar SEC-MAL complex profiles to AK112 in the presence of VEGFA (FIGS. 7A-7F; Table 18). Without being bound by mechanism of action, the results strongly suggest that bispecific antibodies could enhance PD-1 binding through VEGFA daisy chaining (FIG. 9).

TABLE 18

| | | SEC-MALS | SEC-MALS | | |
|---|---|---|---|---|---|
| | Conc. | Loading | Loading | Theoretical | Measured |
| Sample name | (mg/ml) | (μl) | (nM) | Mw (kDa) | Mw (kDa) |
| VEGFA | 0.25 | 50 | 20 | 25 | / |
| PAL054-0001.1L | 2.88 | 50 | 5 | 201.1 | 196.1 |
| PAL054-0001.1La | 2.94 | 50 | 5 | 201.3 | 197.7 |
| PAL054-0011.1La | 4.43 | 50 | 5 | 174.9 | 171.7 |
| PAL054-0104.1La | 1.85 | 50 | 5 | 202.9 | 202.4 |
| PAL054-0148.1La | 2.38 | 50 | 5 | 202.5 | 196.9 |
| PAL054-0157.1La | 3.17 | 50 | 5 | 202.68 | 197.6 |
| PAL054-0170.1L | 4.72 | 50 | 2.5 | 202.50 | 200.8 |
| PAL054-0001.1L + VEGFA | / | 50 | 5 + 10 | / | 817.6, 509.2, 425.6 |
| PAL054-0001.1La + VEGFA | / | 50 | 5 + 10 | / | 882.1, 533.8, 595.7 |
| PAL054-0011.1La + VEGFA | / | 50 | 5 + 10 | / | 803.3, 466.3, 526.8 |
| PAL054-0104.1La + VEGFA | / | 50 | 5 + 10 | / | 839.1, 515.7, 449.6 |
| PAL054-0148.1La + VEGFA | / | 50 | 5 + 10 | / | 883.1, 531.2, 511.1 |
| PAL054-0157.1La + VEGFA | / | 50 | 5 + 10 | / | 835.6, 519.5, 413.7 |
| PAL054-0170.1L + VEGFA | / | 50 | 2.5 + 5 | / | 1391, 547.4, 945.7 |

### Example 8: Functional Activity of Bispecific Antibodies in Reporter Assay

### PD-1 Reporter Assays

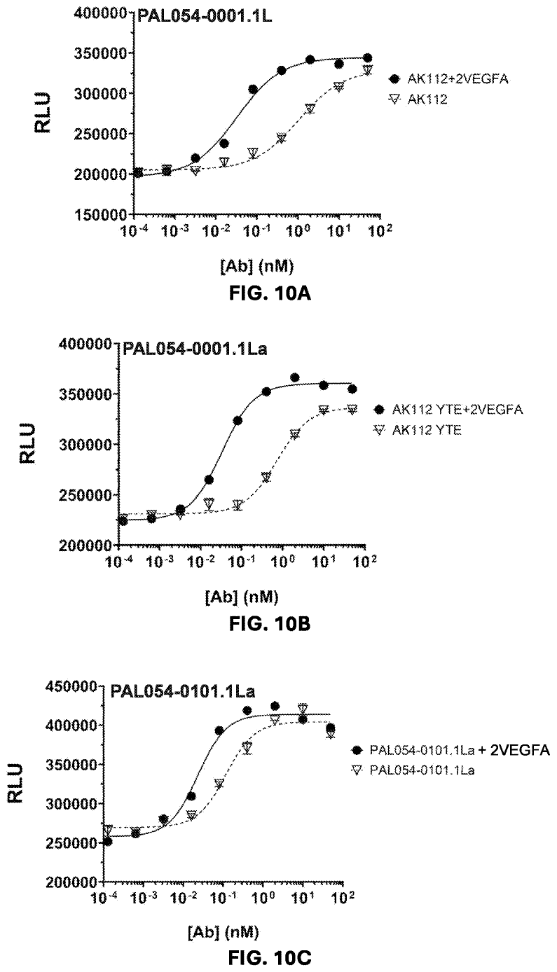

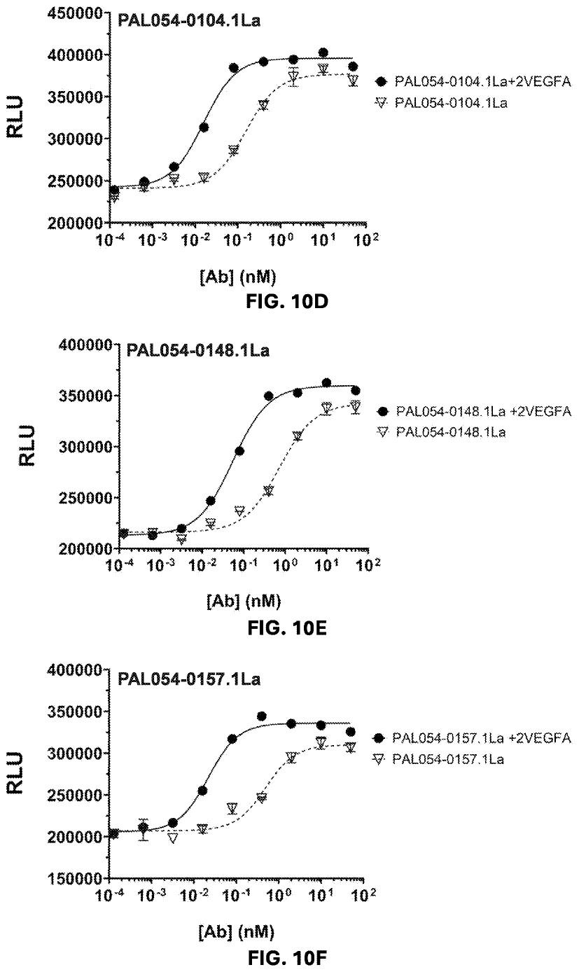

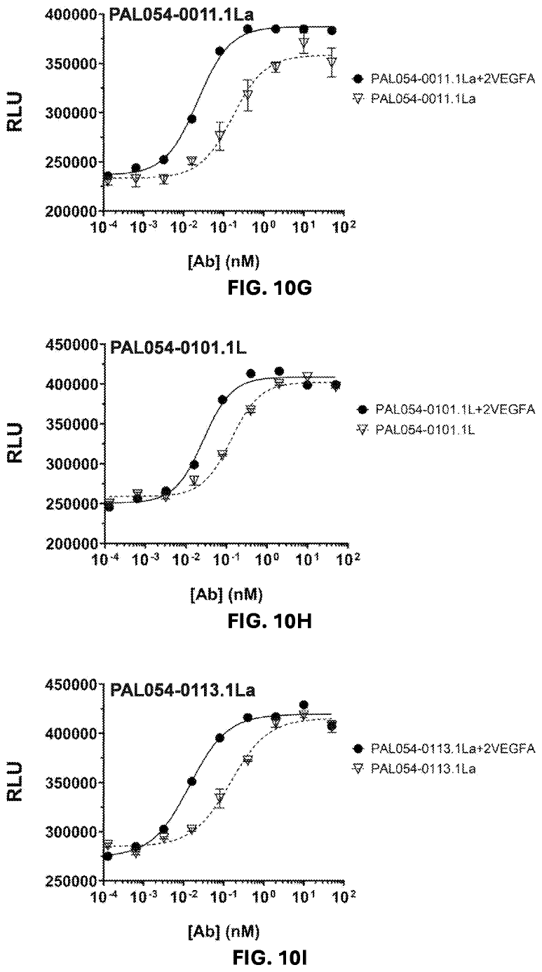

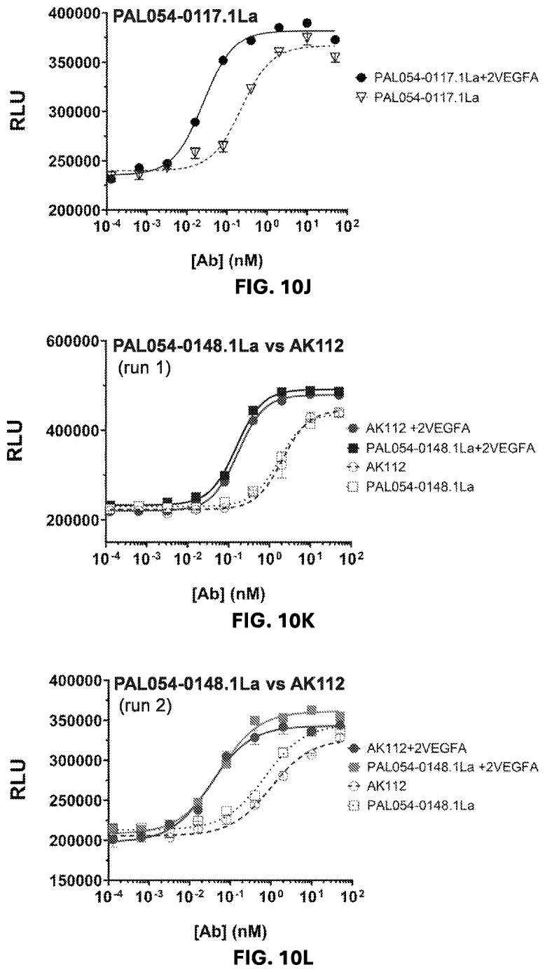

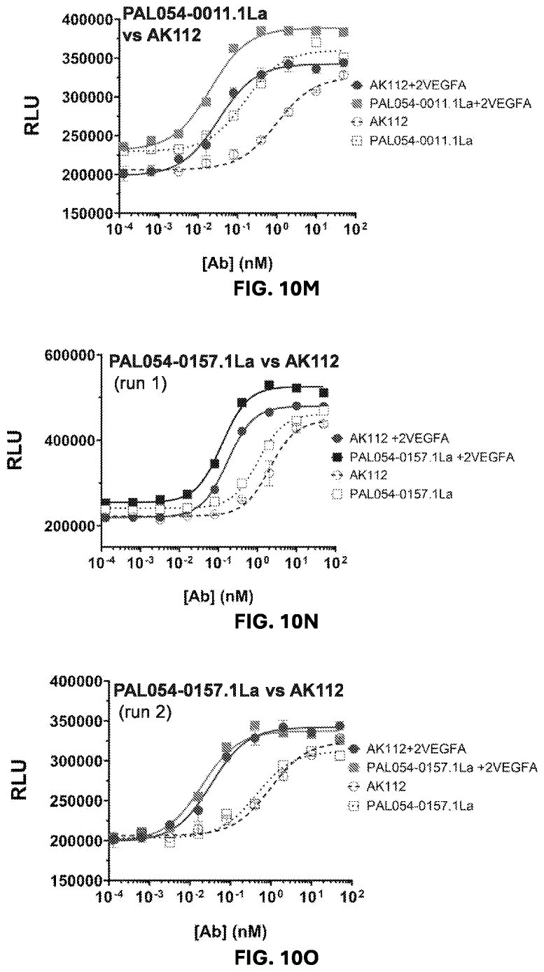

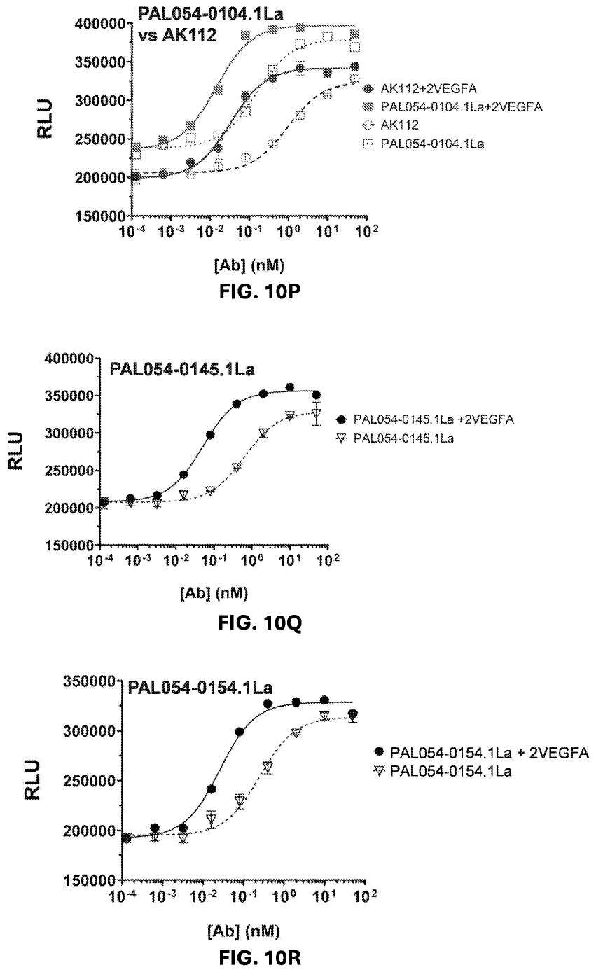

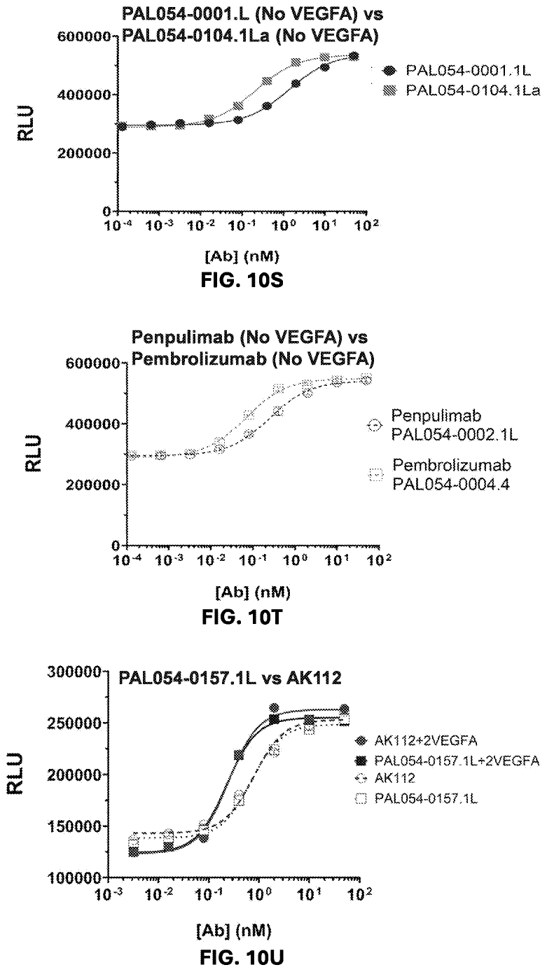

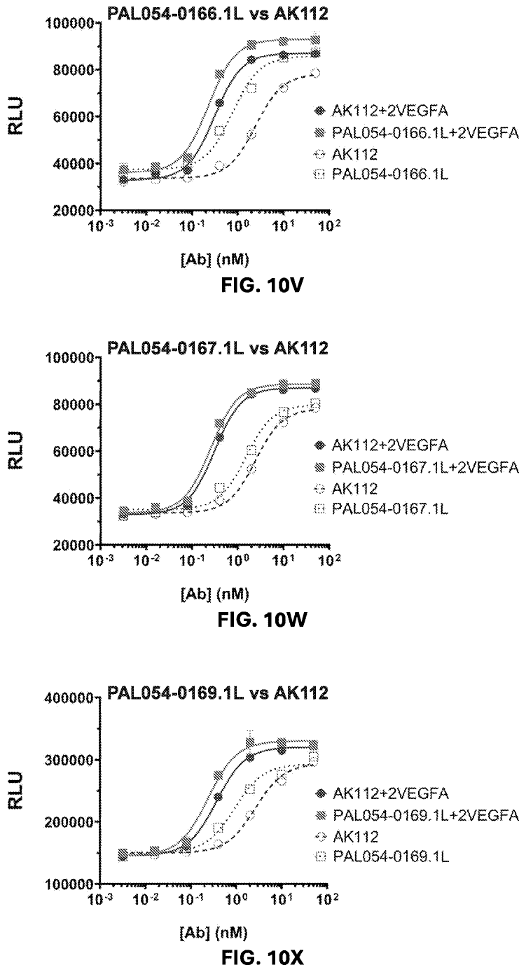

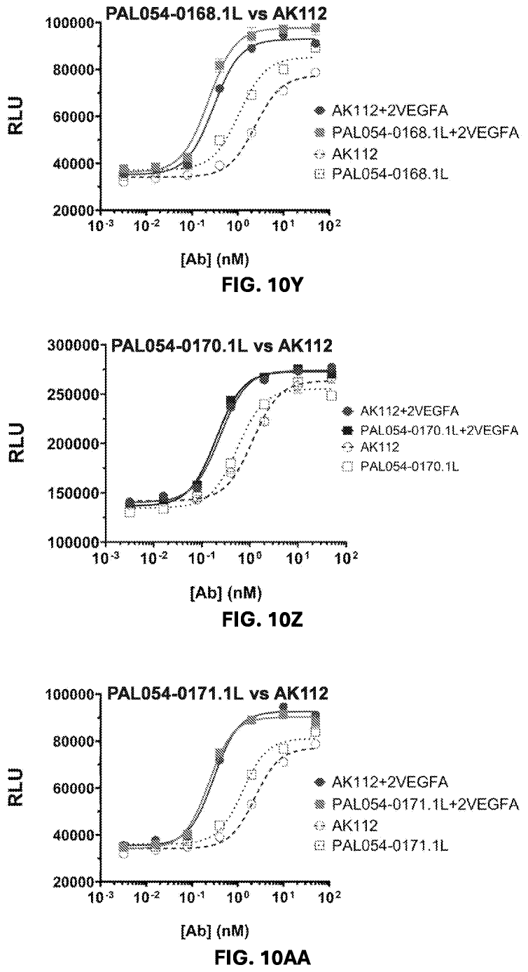

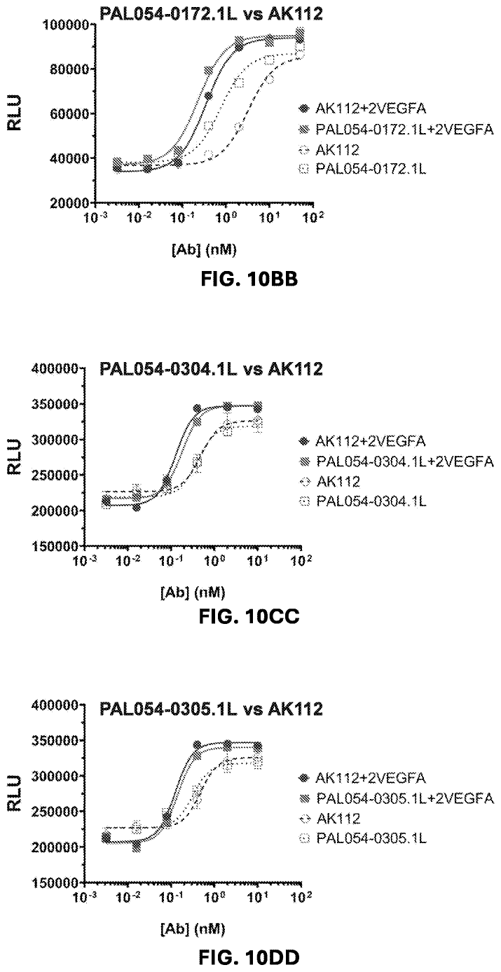

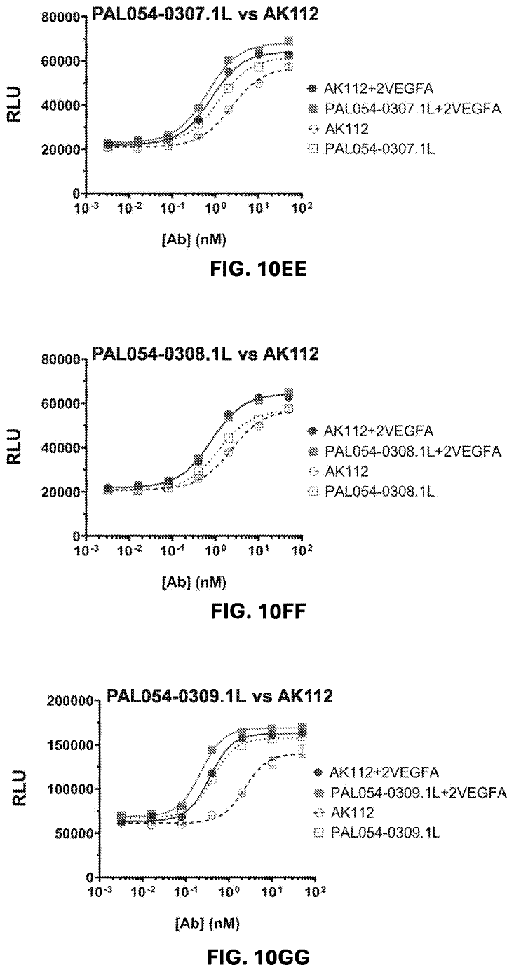

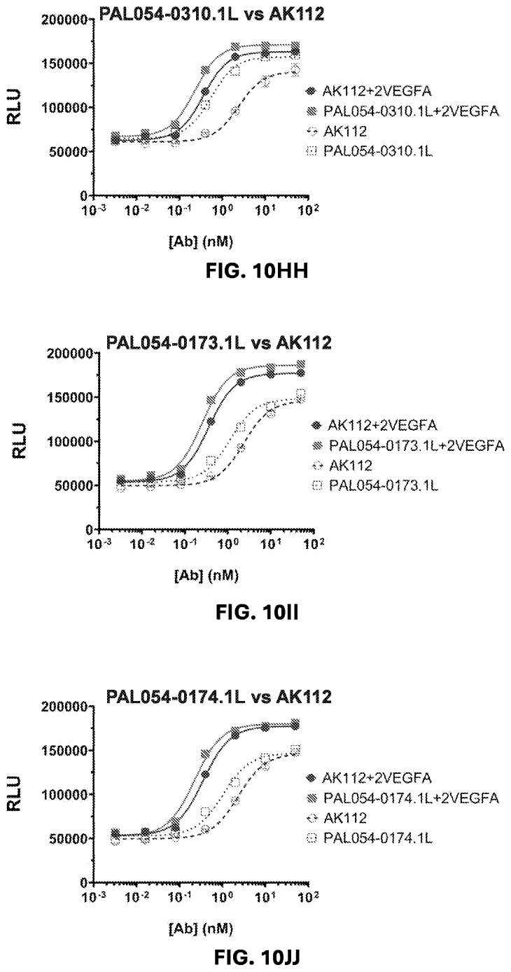

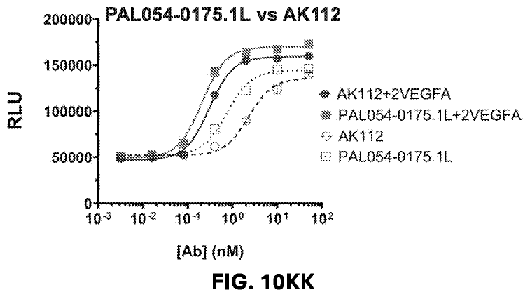

A PD-1 reporter assay was conducted on VEGF/PD-1 bispecific antibodies as disclosed herein and revealed enhanced functional potency of VEGF/PD-1 bispecific antibodies to block PD-1/PD-L1 signaling pathway in the presence of VEGFA (FIGS. 10A-10KK and Tables 19A-19C.

TABLE 19A
Run 1

| | PD-1 | PD-1 | Rel IC₅₀ |
|---|---|---|---|
| | Reporter IC₅₀ | Reporter IC₅₀ | PD-1 |
| Bispecifics | (nM) (−VEGF) | (nM) (+VEGF) | (+VEGF) |
| PAL054-0001.1L | 2.08 | 0.17 | 1.0 |
| (AK112) | | | |
| PAL054-0148.1La | 1.73 | 0.15 | 0.9 |
| PAL054-0157.1La | 1.04 | 0.12 | 0.7 |

TABLE 19B
Run 2

| | PD-1 | PD-1 | Rel IC₅₀ |
|---|---|---|---|
| | Reporter IC₅₀ | Reporter IC₅₀ | PD-1 |
| Bispecifics | (nM) (−VEGF) | (nM) (+VEGF) | (+VEGF) |
| PAL054-0001.1L | 1.05 | 0.033 | 1.0 |
| (AK112) | | | |
| PAL054-0148.1La | 0.73 | 0.056 | 1.7 |
| PAL054-0157.1La | 0.49 | 0.022 | 0.7 |

TABLE 19C
Run 3

| | PD-1 | PD-1 | Rel IC₅₀ |
|---|---|---|---|
| | Reporter IC₅₀ | Reporter IC₅₀ | PD-1 |
| Bispecifics | (nM) (−VEGF) | (nM) (+VEGF) | (+VEGF) |
| PAL054-0001.1L | 1 | 0.033 | 1 |
| PAL054-0145.1La | 0.64 | 0.052 | 1.7 |
| PAL054-0154.1La | 0.25 | 0.026 | 0.9 |
| PAL054-0011.1La | 0.17 | 0.022 | 0.7 |
| PAL054-0104.1La | 0.15 | 0.016 | 0.5 |
| PAL054-0001.1La | 0.73 | 0.033 | 1.1 |
| PAL054-0113.1La | 0.15 | 0.014 | 0.5 |
| PAL054-0117.1La | 0.23 | 0.025 | 0.8 |
| PAL054-0157.1L | 0.71 | 0.23 | 0.9 |
| PAL054-0166.1L | 0.77 | 0.23 | 0.7 |
| PAL054-0167.1L | 1.5 | 0.26 | 0.8 |
| PAL054-0168.1L | 1.1 | 0.23 | 0.8 |
| PAL054-0169.1L | 0.92 | 0.24 | 0.6 |
| PAL054-0170.1L | 0.55 | 0.2 | 0.8 |
| PAL054-0304.1L | 0.4 | 0.18 | 1.4 |
| PAL054-0305.1L | 0.31 | 0.14 | 1.1 |
| PAL054-0173.1L | 1.1 | 0.25 | 0.7 |
| PAL054-0174.1L | 1 | 0.23 | 0.6 |
| PAL054-0175.1L | 0.76 | 0.21 | 0.7 |
| PAL054-0307.1L | 1.2 | 0.65 | 0.8 |
| PAL054-0308.1L | 1.2 | 0.8 | 1.0 |
| PAL054-0309.1L | 0.42 | 0.22 | 0.6 |
| PAL054-0310.1L | 0.54 | 0.23 | 0.6 |
| PAL054-0171.1L | 1.3 | 0.23 | 0.8 |
| PAL054-0172.1L | 0.77 | 0.23 | 0.7 |

### VEGF Reporter Assays

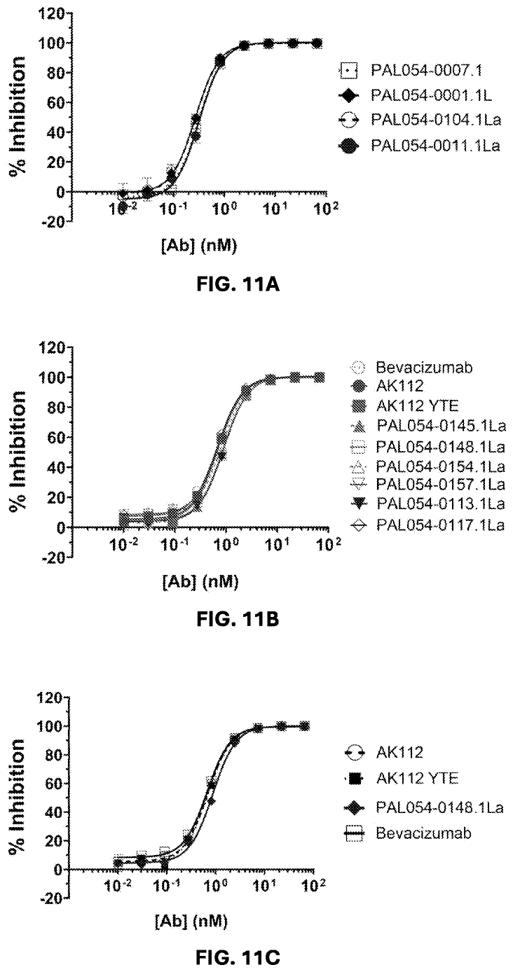

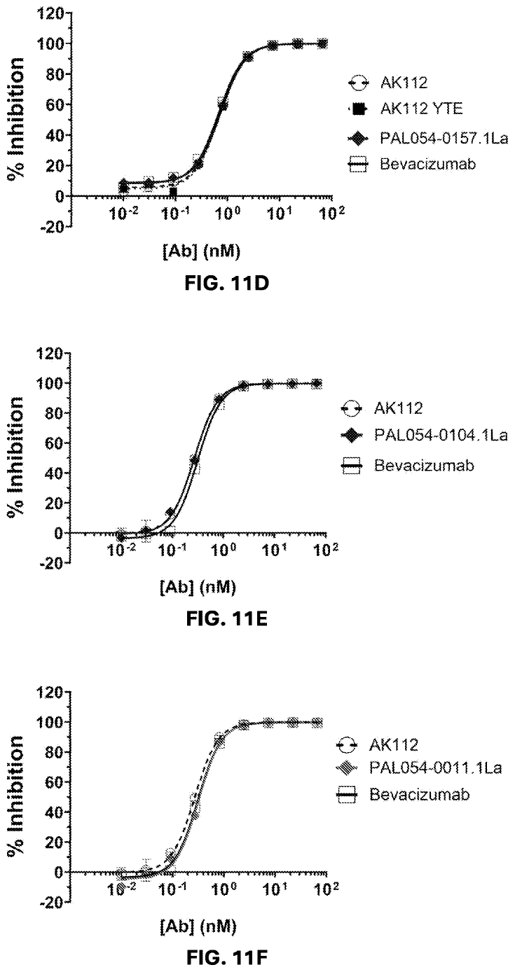

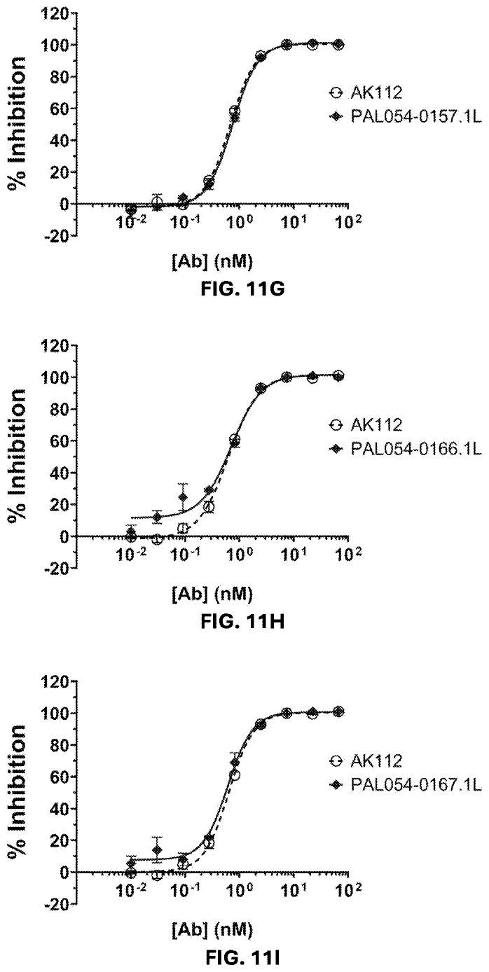

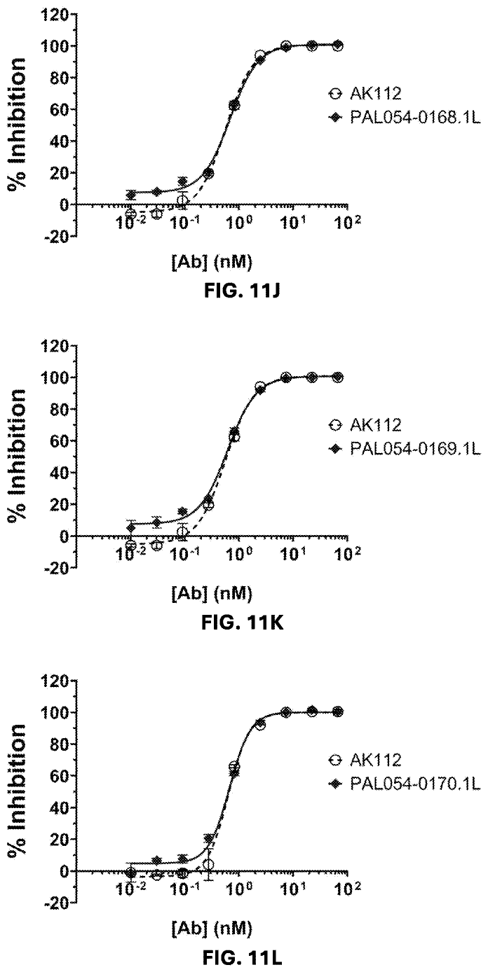

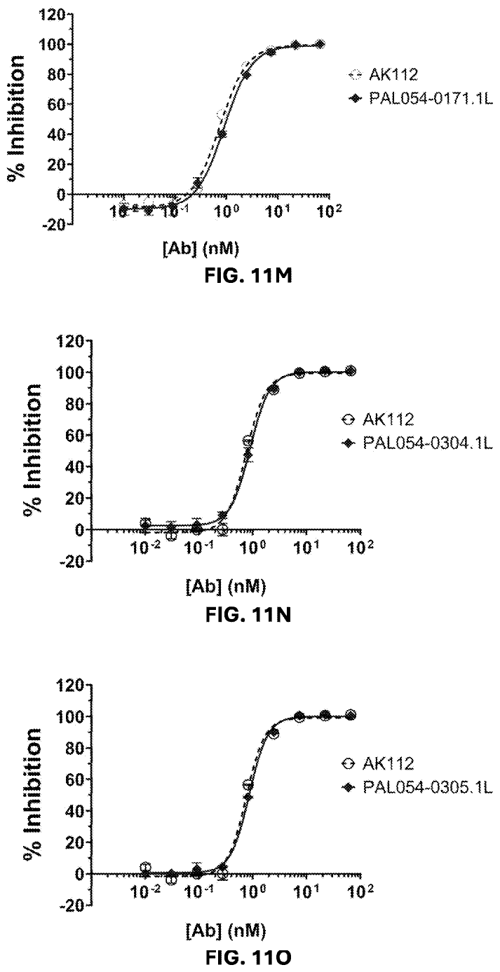

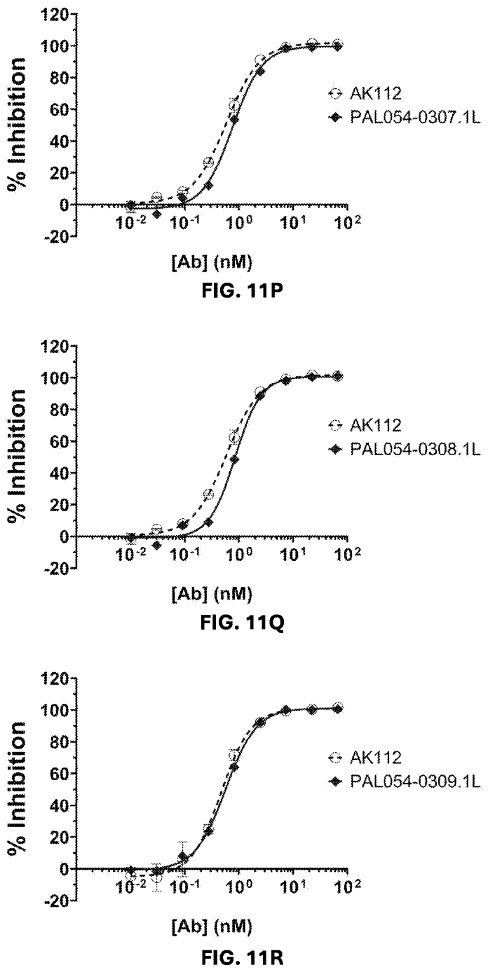

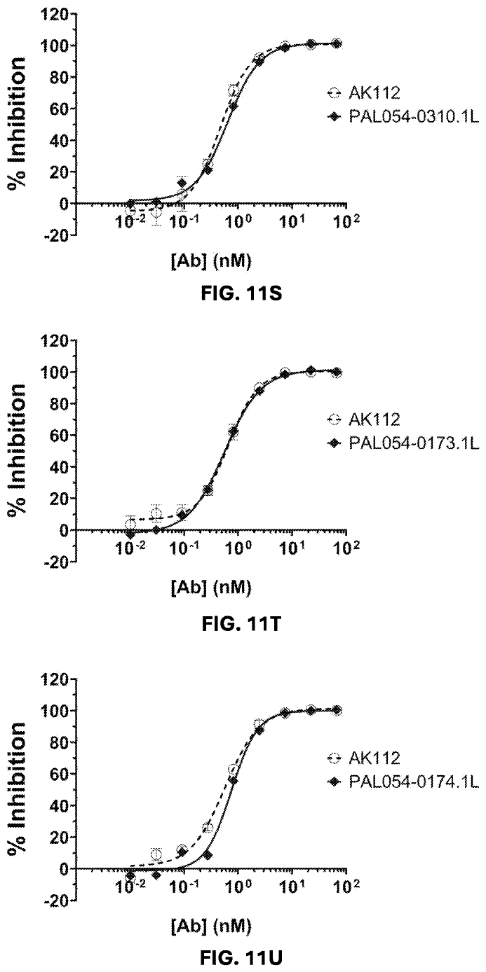

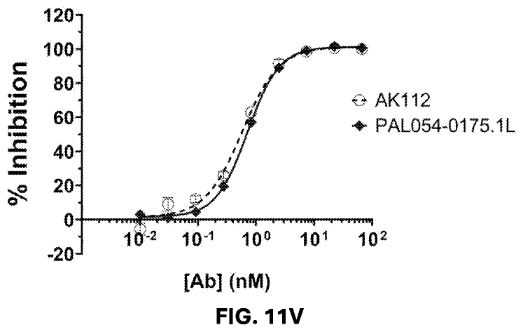

A VEGF reporter assay was conducted on VEGF/PD-1 bispecific antibodies as disclosed herein and the resultant IC₅₀ values are provided in FIGS. 11A-11V and Tables 20A and 20B.

TABLE 20A

| Bispecifics | Rel IC₅₀ (nM) |
|---|---|
| PAL054-0157.1L | 1.1 |
| Bevacizumab | 1.1 |
| PAL054-0166.1L | 1.2 |
| PAL054-0167.1L | 1.0 |
| PAL054-0168.1L | 1.2 |
| PAL054-0169.1L | 1.1 |
| PAL054-0170.1L | 1.0 |
| PAL054-0303.1L | 1.0 |
| PAL054-0304.1L | 1.2 |
| PAL054-0305.1L | 1.1 |
| PAL054-0173.1L | 0.9 |
| PAL054-0174.1L | 1.3 |
| PAL054-0175.1L | 1.2 |
| PAL054-0307.1L | 1.3 |
| PAL054-0308.1L | 1.4 |
| PAL054-0309.1L | 1.2 |
| PAL054-0310.1L | 1.4 |
| PAL054-0171.1L | 1.1 |

TABLE 20B

| Bispecifics | Rel IC₅₀ (nM) |
|---|---|
| PAL054-0001.1L (AK112) | 1.0 |
| PAL054-0001.1La (AK112 YTE) | 1.0 |
| PAL054-0145.1La | 1.3 |
| PAL054-0148.1La | 1.3 |
| PAL054-0154.1La | 1.2 |
| PAL054-0157.1La | 1.0 |
| PAL054-0011.1La | 1.2 |
| PAL054-0104.1La | 1.0 |
| PAL054-0001.1La | 1.0 |
| PAL054-0113.1La | 1.3 |
| PAL054-0117.1La | 1.0 |
| PAL054-0007.1 (Bevacizumab) | 1.2, 1.0 |

### Example 9: VEGF Effect on HUVEC Proliferation

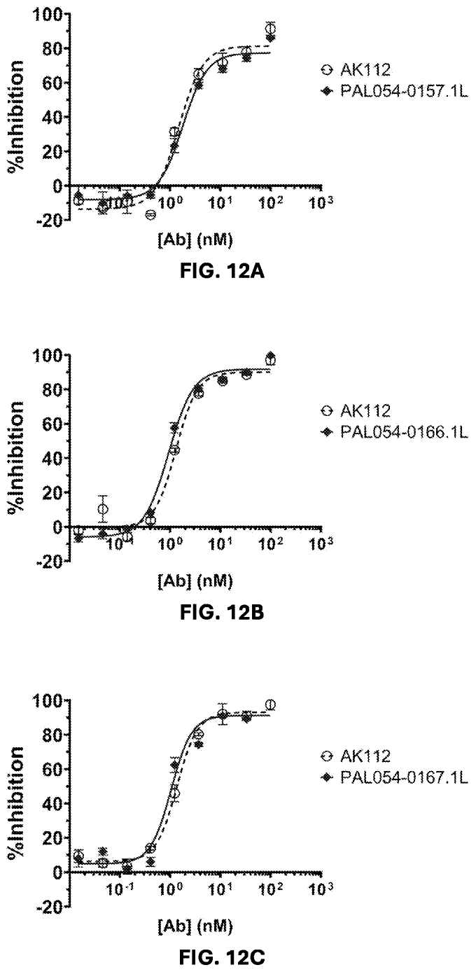

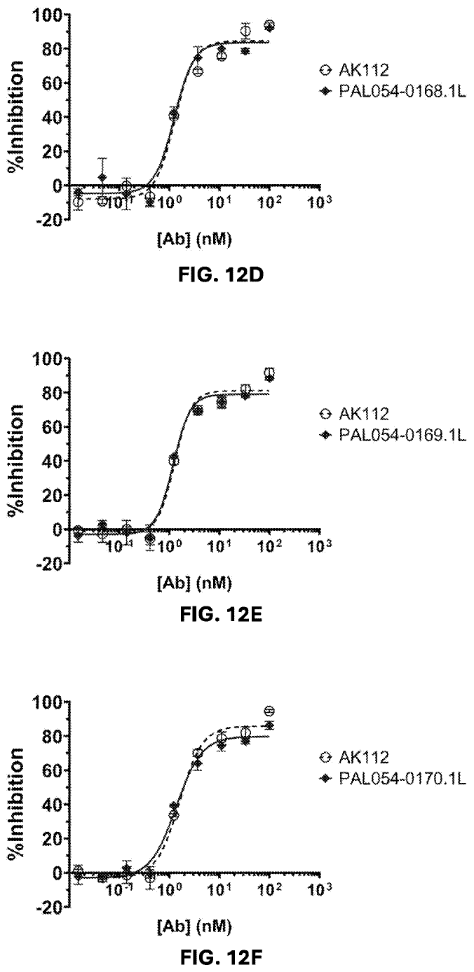

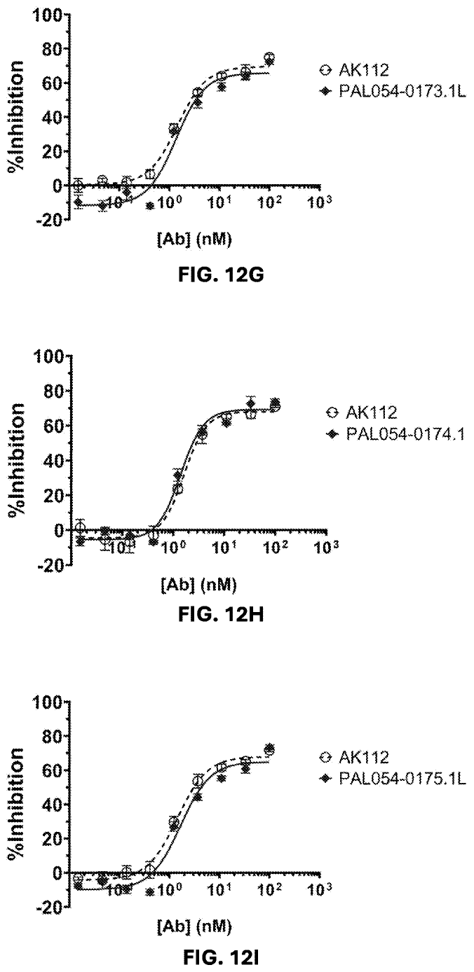

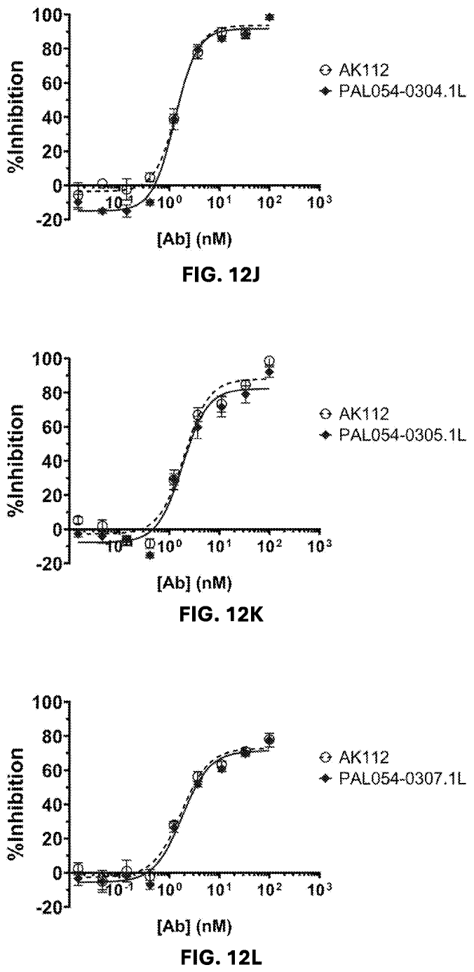

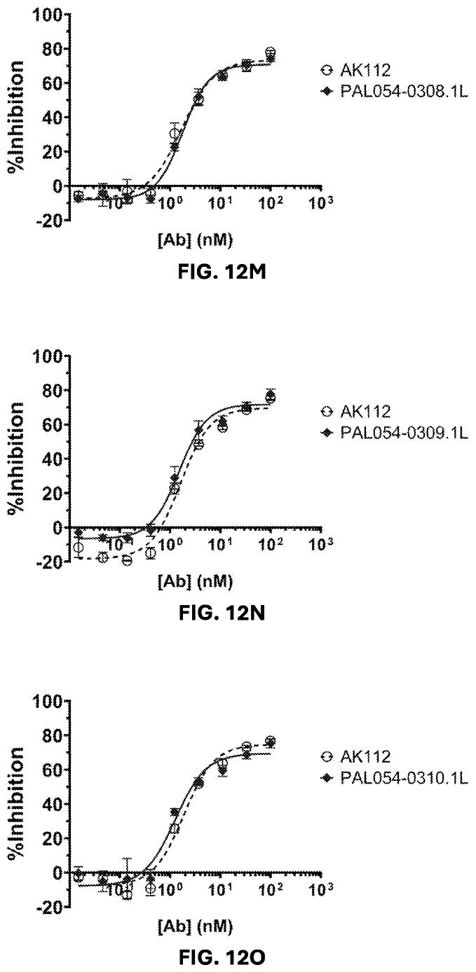

HUVEC proliferation assay was conducted on VEGF/PD-1 bispecific antibodies as disclosed herein and the resultant IC50 values are provided in FIGS. 12A-120 and Table 21.

TABLE 21
HUVEC Proliferation IC₅₀ Values

| Bispecifics | IC₅₀ (nM) | Positive control (AK112) IC₅₀ |
|---|---|---|
| PAL054-0157.1L | 1.8 | 1.4 |
| PAL054-0170.1L | 1.4 | 1.6 |
| PAL054-0304.1L | 1.3 | 1.4 |
| PAL054-0305.1L | 1.8 | 2 |
| PAL054-0173.1L | 1.4 | 1.5 |
| PAL054-0174.1L | 1.4 | 1.6 |
| PAL054-0175.1L | 1.7 | 1.4 |
| PAL054-0307.1L | 1.8 | 1.7 |
| PAL054-0308.1L | 1.8 | 1.7 |
| PAL054-0309.1L | 1.5 | 1.6 |
| PAL054-0310.1L | 1.3 | 1.9 |
| PAL054-0166.1L | 0.9 | 1.3 |
| PAL054-0167.1L | 1.1 | 1.4 |
| PAL054-0168.1L | 1.3 | 1.3 |
| PAL054-0169.1L | 1.2 | 1.3 |

### Example 10: VEGF Effect on PD-1 Internalization

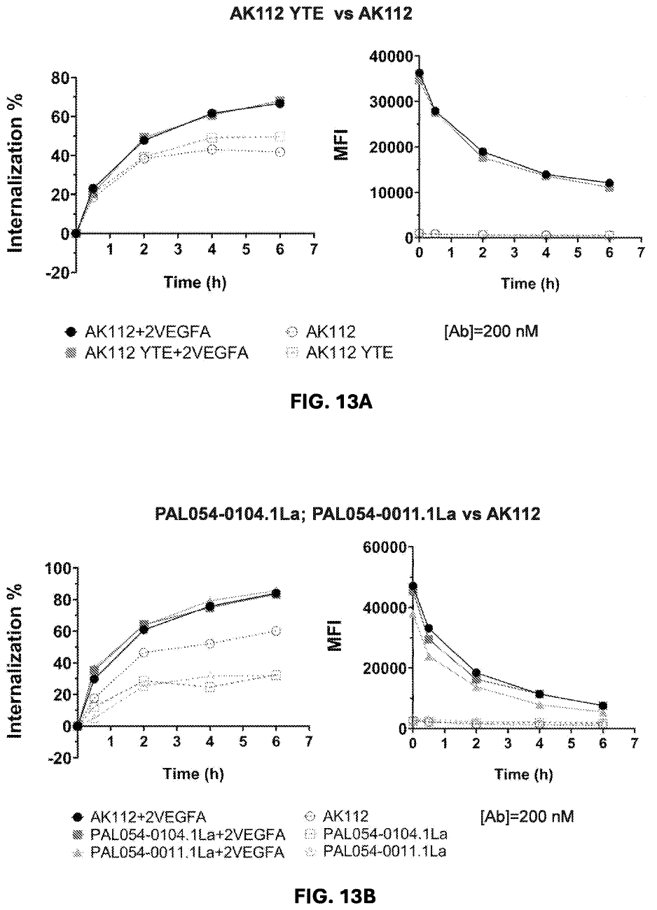

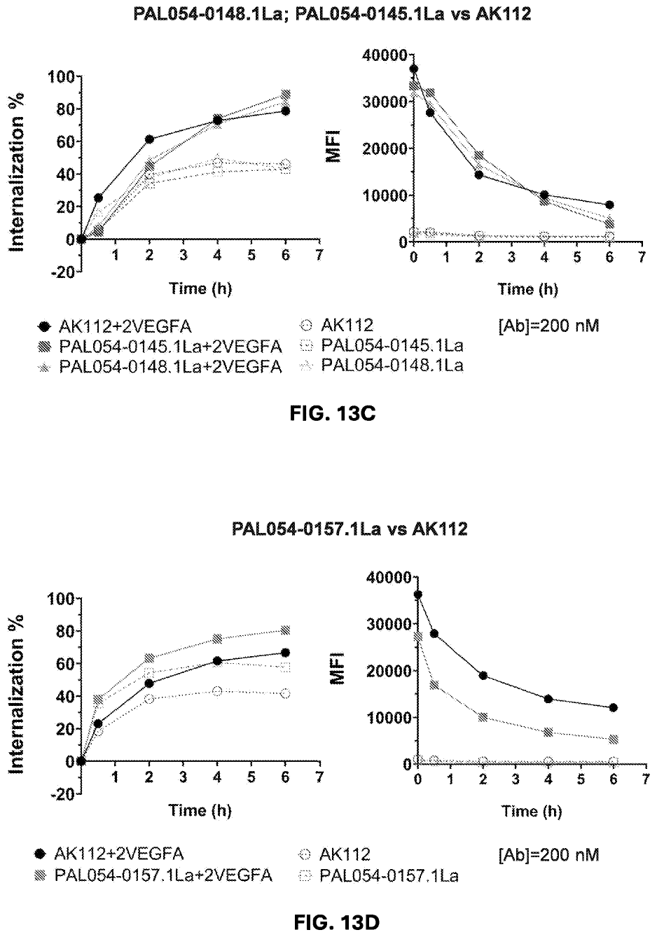

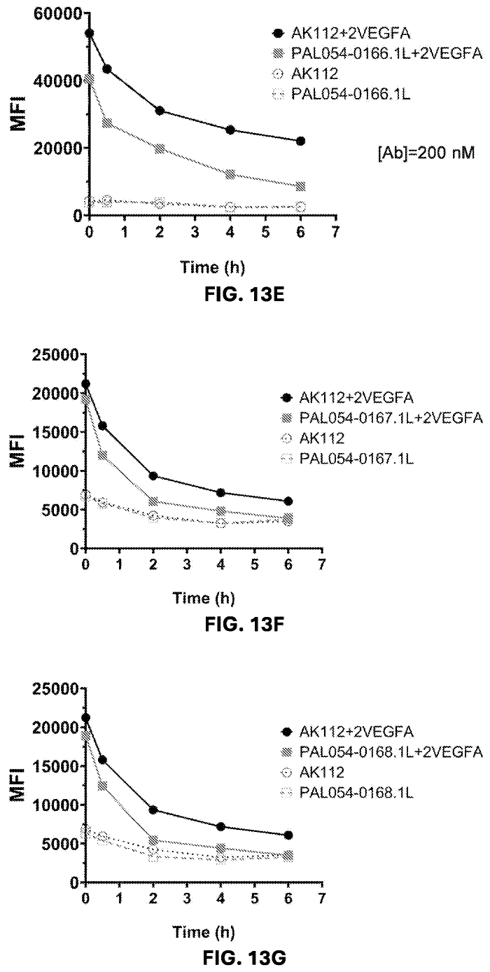

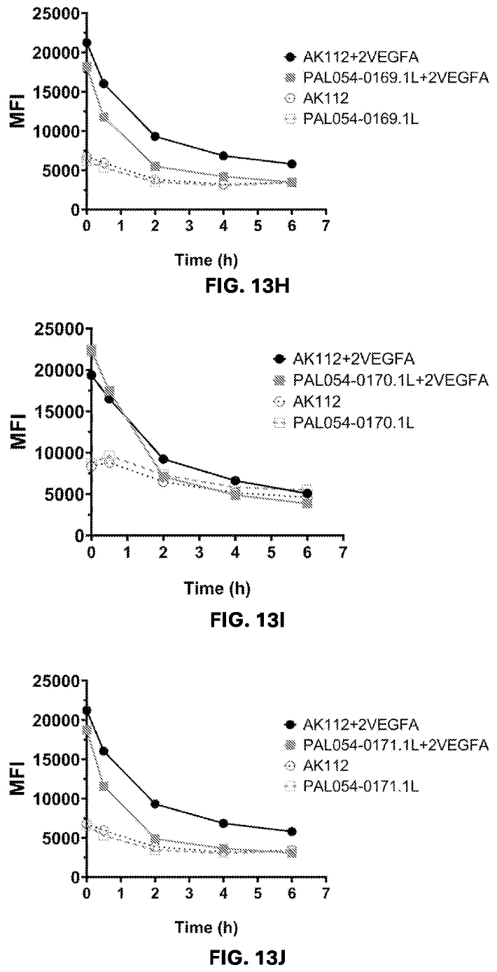

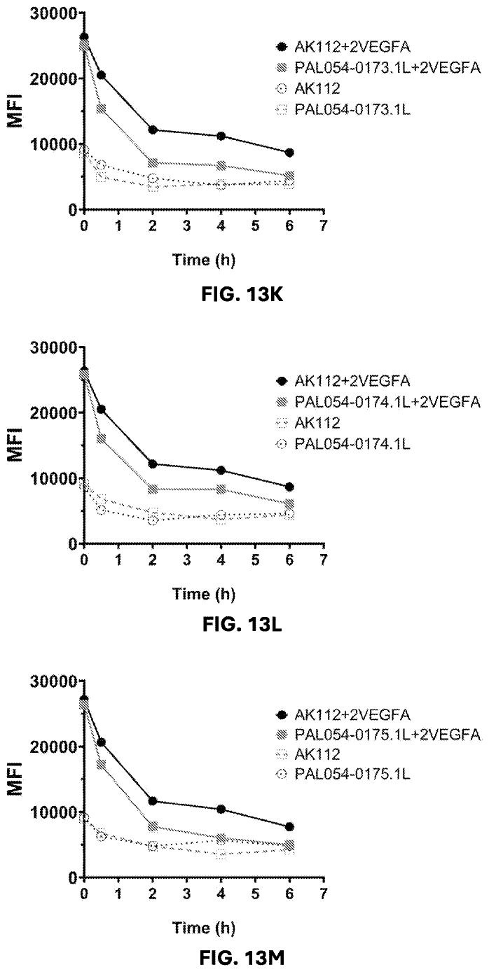

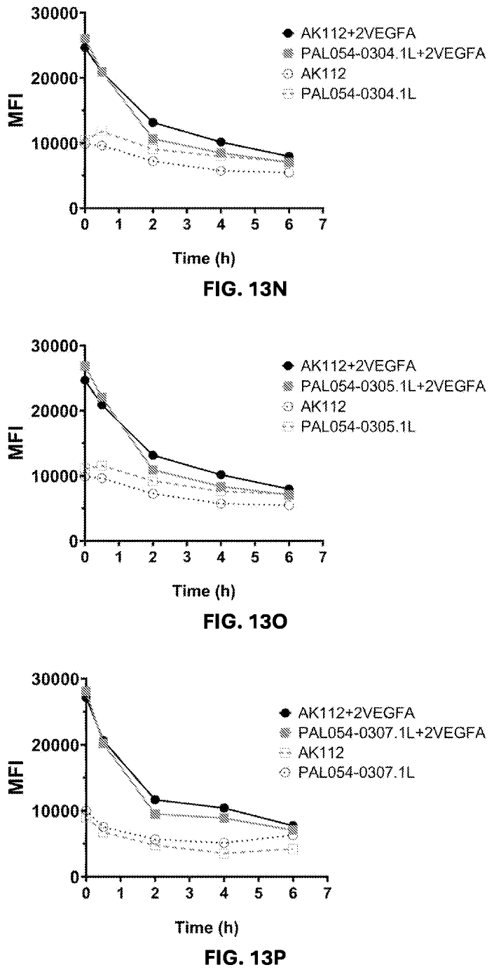

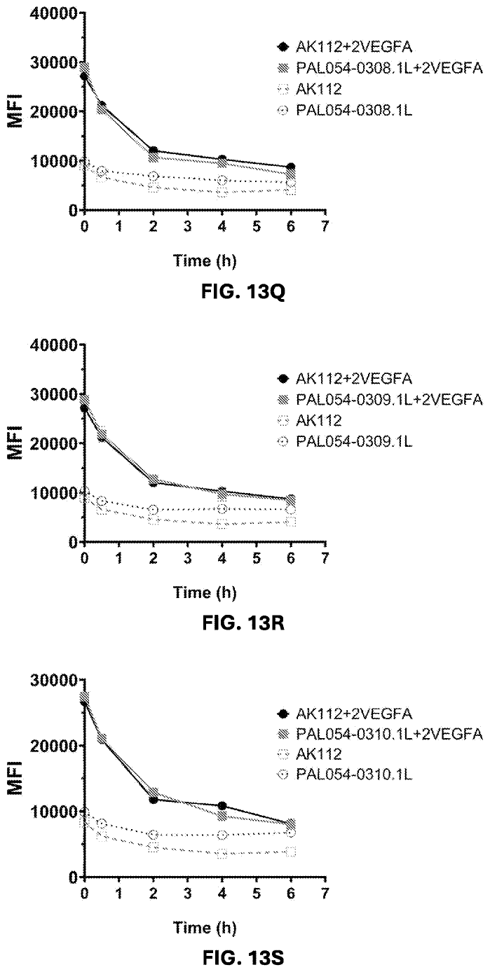

A PD-1 internalization assay was conducted on VEGF/PD-1 bispecific antibodies as disclosed herein and revealed enhanced internalization of PD-1 in the presence of VEGF. The results are shown in FIGS. 13A-13S.

### Example 11: Binding and Activity of Bispecific Reverse Constructs

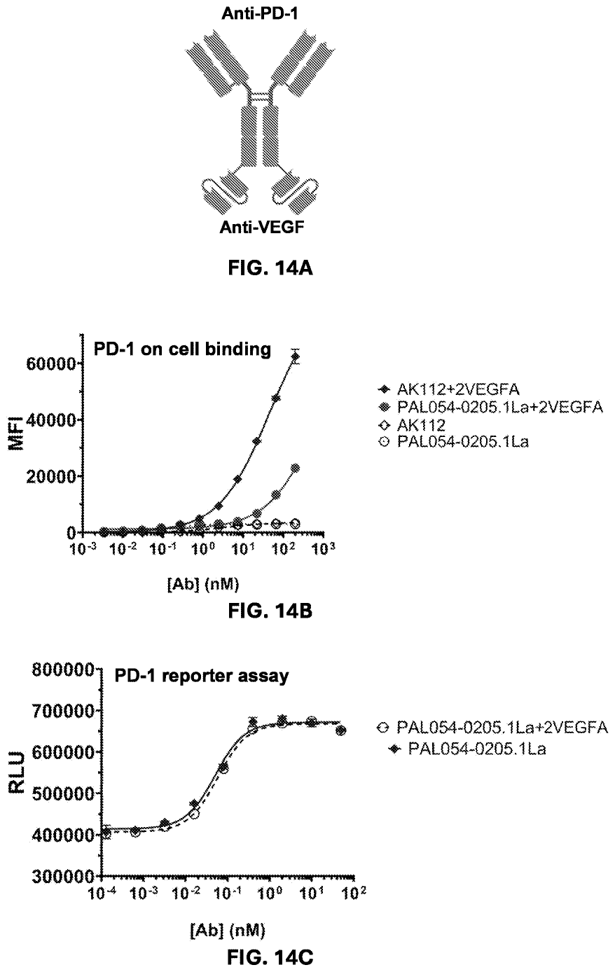

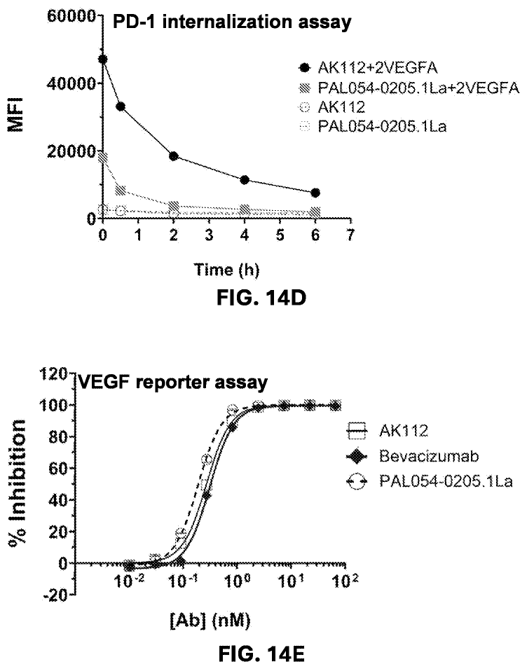

Cell binding, internalization, PD-1 reporter and VEGF reporter assays as known to those of ordinary skill in the art and described herein were conducted on PD-1-VEGF (scFv) reverse constructs (FIG. 14A), and the results are shown in FIGS. 14B-14E. The results did not show significant enhanced bioactivity of PD-1 in the presence of VEGFA.

### Example 12: T Cell Activation in Human PBMC and Hepatocellular Carcinoma Cell Line Co-Culture

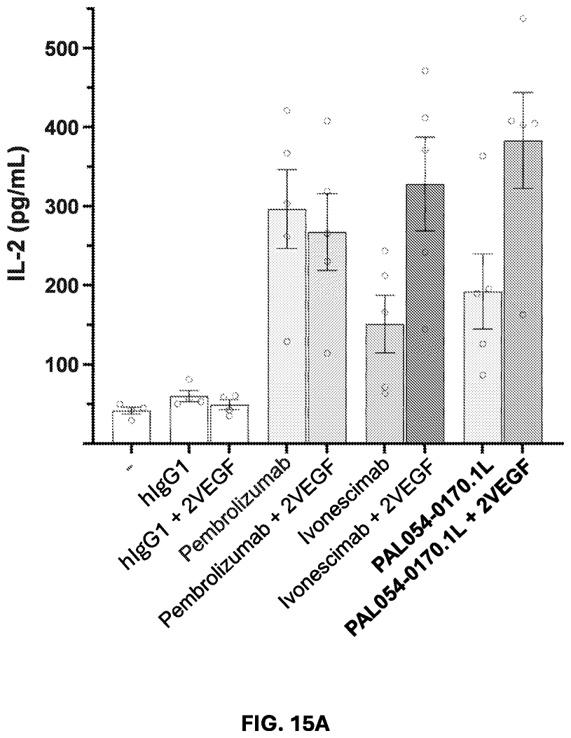

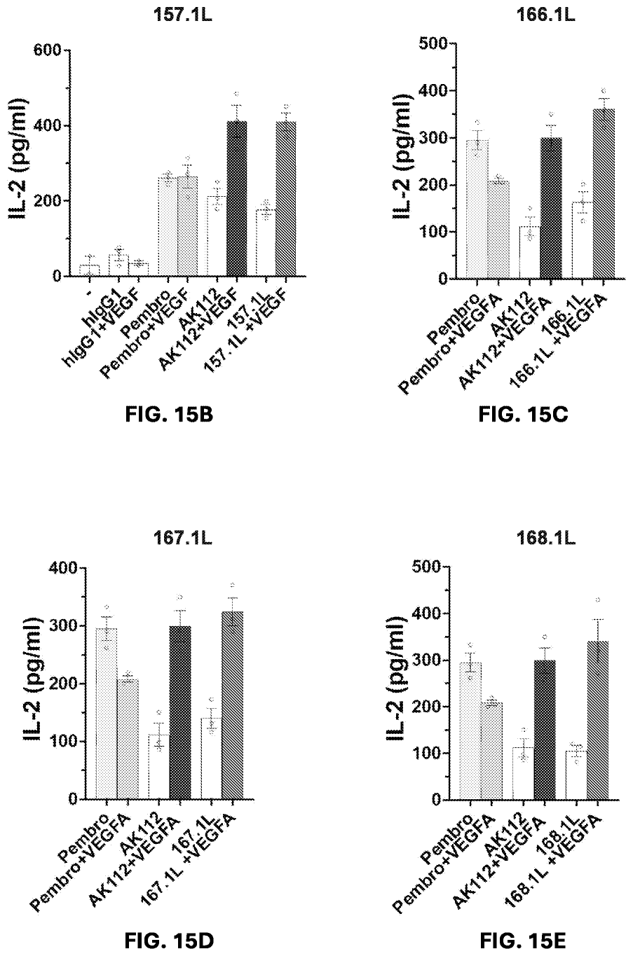

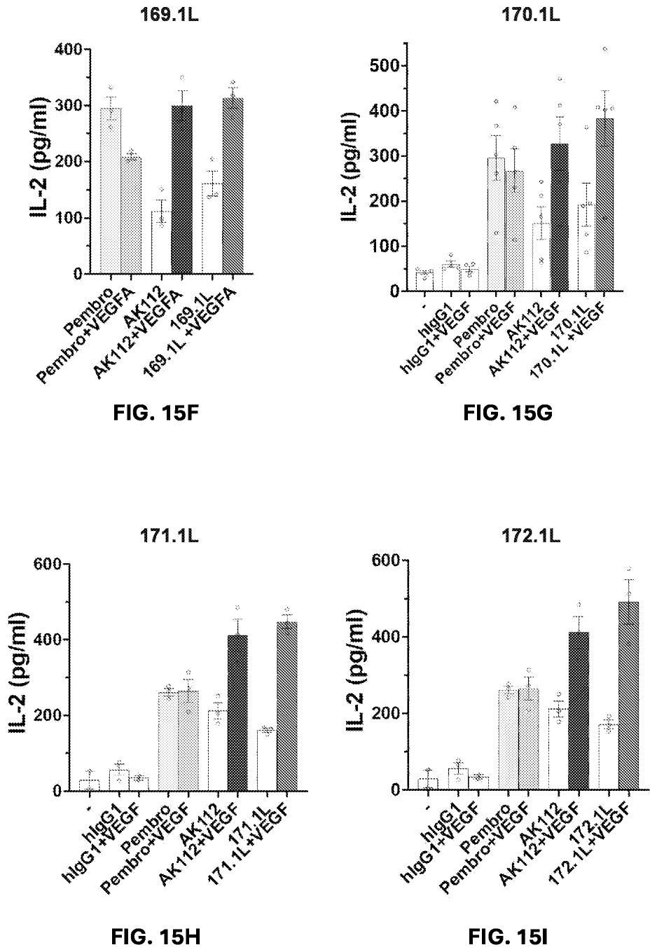

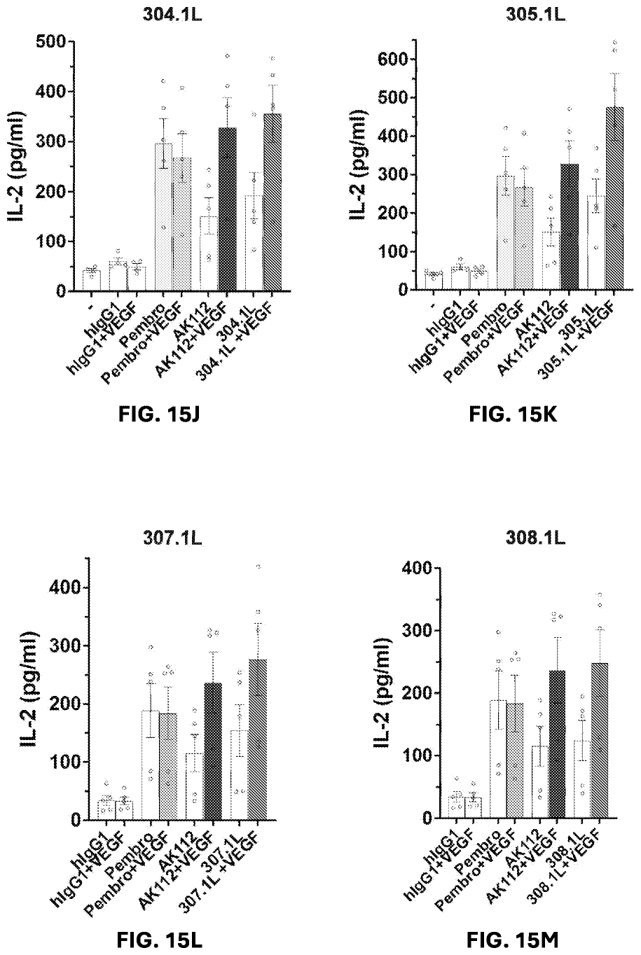

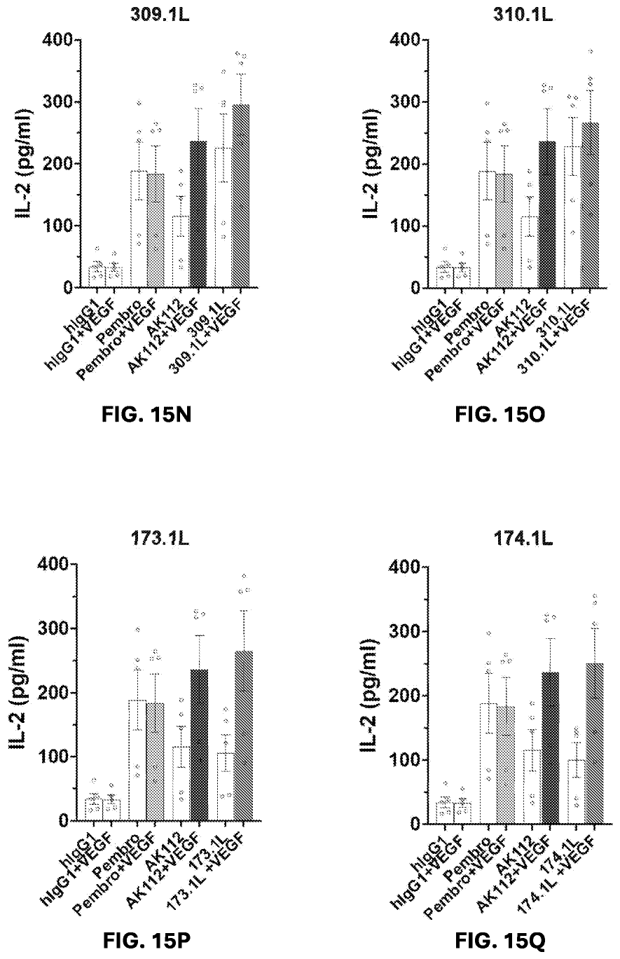

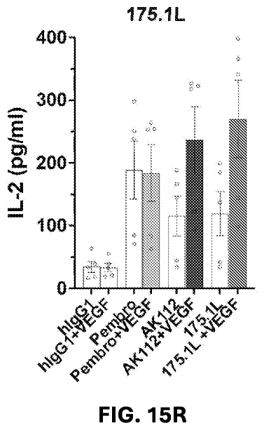

T cell activation was assessed in human PBMC and Hep3B-OS8-hPD-L1 co-culture assay in the presence or absence of 2×VEGFA (2VEGF) by measuring IL-2 secretion. For each bispecific clone, various controls were used including untreated (-), isotype control (hIgG1), PAL054-0004.4 (pembrolizumab), and/or PAL054-0001.1L (also referred to as ivonescimab or AKI 12) was tested. Data is seen in FIGS. 15A-15R. As seen in the data, bispecific-VEGF complex was found to enhance T cell activation in the co-culture assay.

### Example 13: T Cell Activation in Human Monocyte Derived Dendritic Cells and CD4+ T Cells Via Mixed Lymphocyte Reaction (MLR) Assay

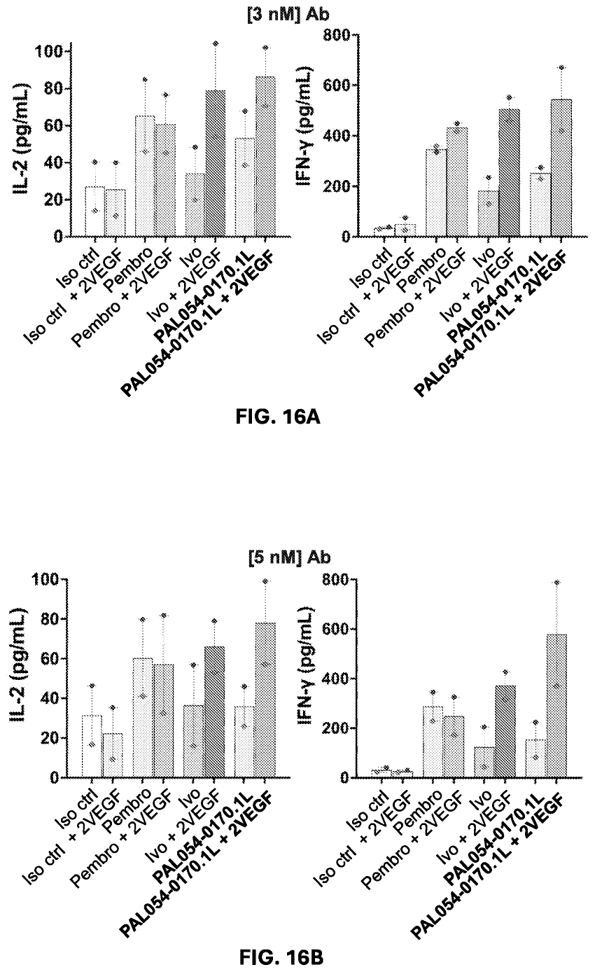

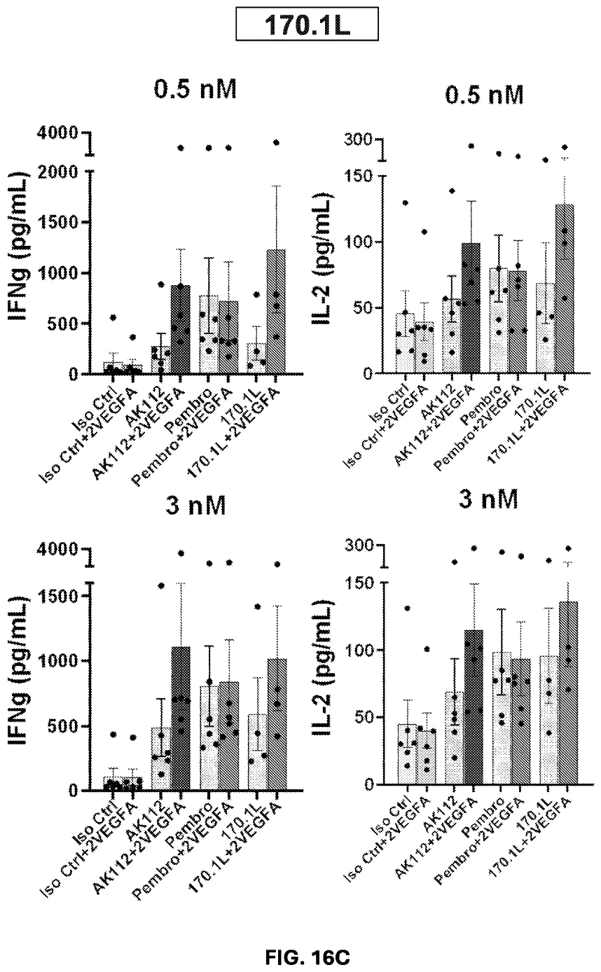

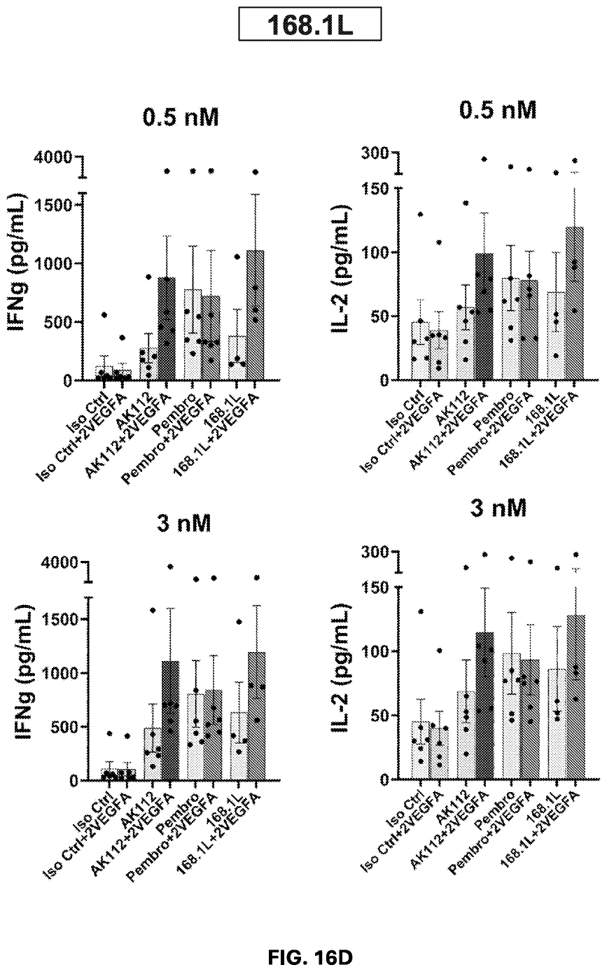

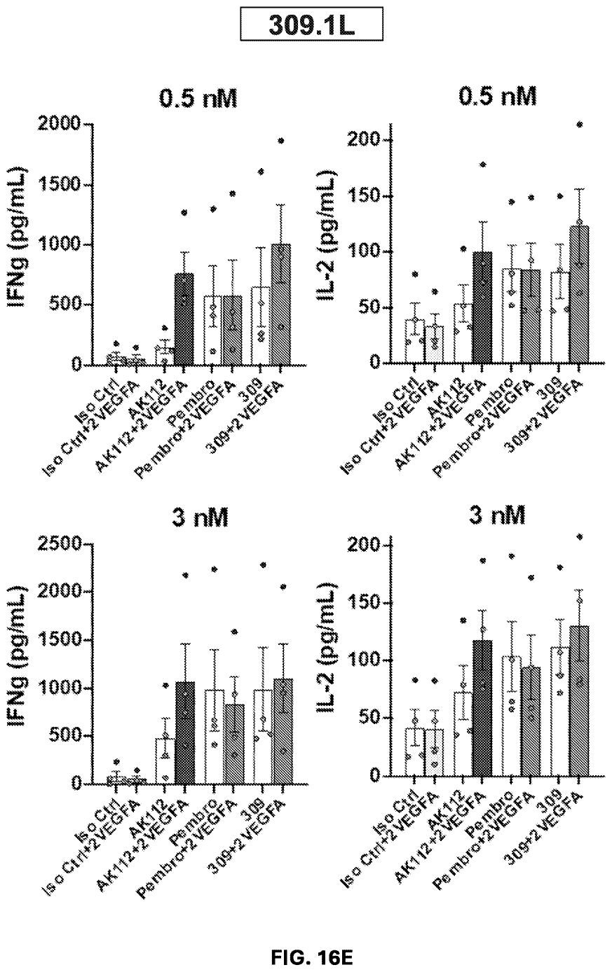

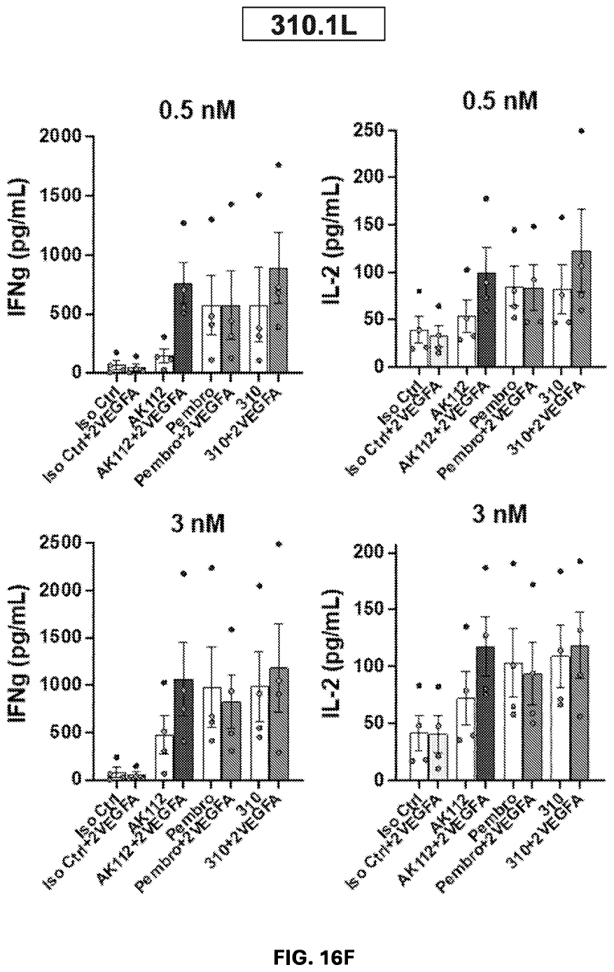

Additionally, T cell activation was assessed in human monocyte derived dendritic cell (moDC) and CD4+ T cell mixed lymphocyte reaction (MLR) by measuring IL-2 and IFNγ secretion. Data collected from 4 independent donor pairs and is seen in FIGS. 16A-16F. As seen in the data, bispecific VEGF complex enhanced T cell activation in DC-CD4+ T cell MLR assay.

### Example 14: Titer, Purity, and Thermal Stability Data for Benchmarks and Exemplary Bispecific Antibodies

Titer, purity, and thermal stability data for benchmarks and bispecific antibodies as disclosed herein were determined. Titer and purity of the antibody samples were determined using standard analytical techniques well known in the art.

Thermal stability was determined using two different methods.

### Method 1: Thermal Stability Determined Using UNCLE

To determine thermal stability, 9 microliters of each antibody sample were loaded into a Uni microcuvette designed specifically for use with the UNCLE instrument from Unchained Labs. Samples were subjected to a controlled thermal ramp from 20° C. to 95° C. at a rate of 0.5° C. per minute. Dynamic light scattering (DLS) measurements were performed both before and after the thermal ramp, with each measurement consisting of four acquisitions lasting five seconds each, to assess particle size and polydispersity. The melting temperature (Tm) of each sample was determined by the UNCLE software through analysis of the first derivative of the barycentric mean (BCM) of fluorescence intensity as a function of temperature. The aggregation onset temperature (Tagg) was calculated based on the intensity of scattered light measured at wavelengths of 266 nm or 473 nm. Size and polydispersity data were extracted from the DLS correlation function. This method provides a comprehensive assessment of antibody thermal stability and aggregation behavior. Data can be seen in Table 22.

TABLE 22

| | Titer | SEC-HPLC | Initial | Purity after | Tₘ1 | Tₘ2 | Tₘ3 | T_{agg}266 |
|---|---|---|---|---|---|---|---|---|
| Clone | (mg/L) | (%) | purity (%) | 3x F/T (%) | (° C.) | (° C.) | (° C.) | (° C.) |
| PAL054-0001.1L | 1830 | 95.51 | 98.1 | 98.1 | 62.1 | 71.5 | — | 58.0 |
| PAL054-0001.1La | 1780 | 96.55 | 98.1 | 98.1 | 61.9 | 69.4 | — | 57.0 |
| PAL054-0008.1La | 764.1 | 87.39 | N/A | N/A | N/A | N/A | N/A | N/A |
| PAL054-0009.1La | 92.7 | N/A | N/A | N/A | N/A | N/A | N/A | N/A |
| PAL054-0011.1La | 1152.8 | 98.13 | 99.3 | 99.2 | 53.2 | 64.9 | — | 55.5 |
| PAL054-0101.1La | 1131.7 | 91.49 | N/A | N/A | N/A | N/A | N/A | N/A |
| PAL054-0104.1La | 256 | 98.19 | 99.3 | 99.3 | 48.8 | 64.7 | — | 46.6 |
| PAL054-0145.1La | 578.5 | 91.22 | 92.8 | 92.8 | 42.7 | 68.4 | 73.8 | 41.8 |
| PAL054-0148.1La | 2009.1 | 93.85 | 94.6 | 95.4 | 47.2 | 64.5 | 69.9 | 46.3 |
| PAL054-0154.1La | 376.4 | 97.4 | 98.2 | 98.2 | 47.6 | 67.4 | 71.9 | 48.3 |
| PAL054-0157.1La | 1561.7 | 97.92 | 98.4 | 98.5 | 51.5 | 65.1 | 72.8 | 51.00 |
| PAL054-0170.1L | 1647.0 | 98.6 | 100 | 98.8 | 54.0 | 63.4 | — | 52.4 |

### Method 2: Thermal Stability Determined Using MicroCal™ VP DSC

To assess thermal stability and unfolding behavior, antibody samples were analyzed using the MicroCal™ VP-Capillary Differential Scanning Calorimeter (DSC) from Malvern. Prior to analysis, samples were diluted to a final concentration of 1 mg/mL using the respective reference buffer. A total of 400 microliters of reference buffer were dispensed into the odd-numbered wells of a 96-well plate, while 400 microliters of sample solution were added to the even-numbered wells.

The DSC instrument was programmed to execute a thermal scan from 10° C. to 110° C. at a constant scan rate of 200° C. per hour. Thermal transitions were monitored continuously throughout the scan. Data acquisition and analysis were performed using the MicroCal™ VP-Capillary DSC automated data analysis software. The thermal transition midpoint (Tm) was determined by identifying the peak of the heat capacity curve, while the onset of unfolding (T_onset) was calculated based on the deviation from baseline preceding the transition peak.

This method enables precise quantification of protein thermal stability and unfolding characteristics under controlled conditions. Data corresponding to these measurements are presented in Table 23.

TABLE 23

| | TₘOnset | Tₘ1 | Tₘ2 | Tₘ3 | Tₘ4 |
|---|---|---|---|---|---|
| Clone | (° C.) | (° C.) | (° C.) | (° C.) | (° C.) |
| PAL054-0148.1L | 51.86 | 57.3 | 75.28 | 81.99 | 88.17 |
| PAL054-0165.1L | 57.26 | 61.87 | 75.7 | 81.86 | 87.15 |
| PAL054-0157.1L | 57.54 | 62.88 | 75.66 | 81.59 | N/A |
| PAL054-0166.1L | 60.56 | 66.42 | 75.73 | 81.74 | N/A |
| PAL054-0001.1L | 59.06 | 66.46 | 75.25 | 81.9 | 88.31 |
| PAL054-0167.1L | 52.9 | 62.7 | 75.3 | 81.7 | 87.0 |
| PAL054-0168.1L | 55.5 | 63.3 | 75.8 | 81.4 | 85.8 |
| PAL054-0169.1L | 54.5 | 62.8 | 75.9 | 81.4 | 85.6 |
| PAL054-0170.1L | 58.8 | 65.8 | 75.5 | 81.6 | 86.4 |
| PAL054-0171.1L | 51.6 | 61.8 | 75.0 | 81.7 | 88.4 |
| PAL054-0172.1L | 55.2 | 64.8 | 75.1 | 81.6 | 88.0 |

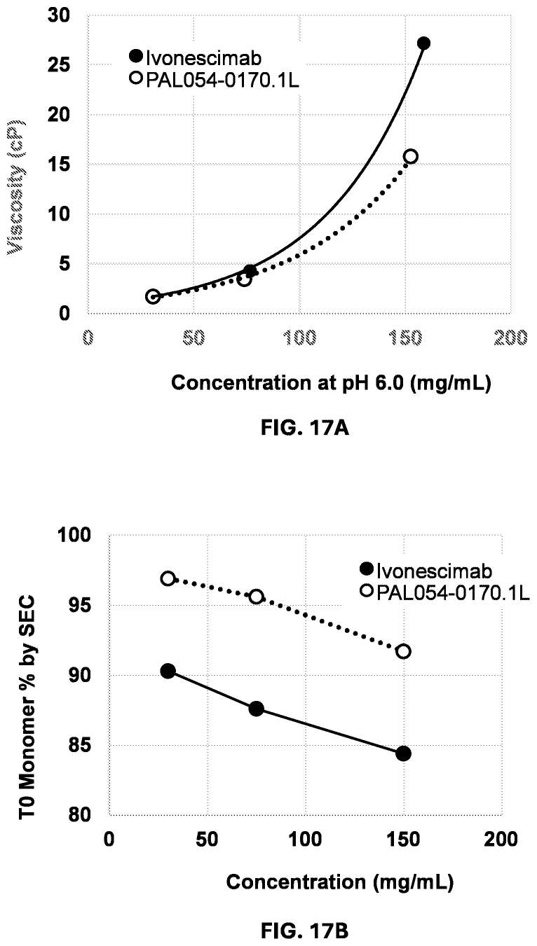

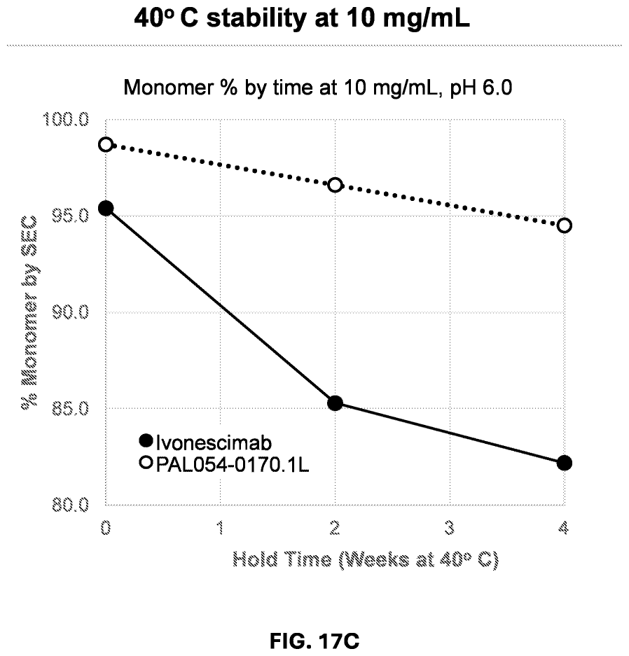

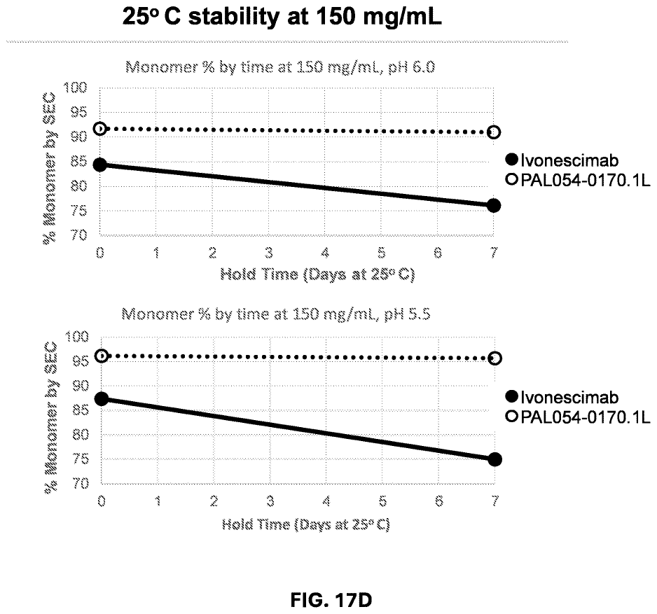

PAL054-0170.1L was further assayed for viscosity, aggregation, and stability. Data is seen in FIGS. 17A-17D that demonstrate PAL054-0170.1L has improved viscosity (FIG. 17A) and aggregation (FIG. 17B) as compared to ivonescimab as well as stability at 40° C. (FIG. 17C) and 25° C. (FIG. 17D).

### Example 15: Pharmacokinetics in Non-Human Primates (NHP)

In vivo pharmacokinetic (PK) studies were performed on bispecific antibodies described herein as compared to Ivonescimab (AK112). Studies were performed using cynomolgus monkey (Macaca fascicularis), and antibodies were administered by intravenous (IV) injection. All animals were males, ranging from 2.5 to 3.9 kg in weight (n=5/group). Animals were administered PAL054-0001.1L (AK112), PAL054-0104.1L (104), PAL054-0157.1L (157), PAL054-0167.1L (167), PAL054-0168.1L (168), PAL054-0169.1L (169), PAL054-0170.1L (170), or PAL054-0171.1L (171), respectively, by intravenous (IV) bolus injection on Day 0, as shown in Table 24, and serum samples were taken regularly throughout the study. PK parameters were determined from cynomolgus serum samples up to day 28.

TABLE 24

| Antibody | Half-Life (Days) |
|---|---|
| AK112 (PAL054-0001.1L) | 1.50 |
| PAL054-0104.1L | 2.21 |
| PAL054-0157.1L | 2.97 |
| PAL054-0167.1L | 2.09 |
| PAL054-0168.1L | 3.12 |
| PAL054-0169.1L | 2.40 |
| PAL054-0170.1L | 3.57 |
| PAL054-0171.1L | 3.15 |

Receptor occupancy (RO) was determined by resuspending cyno PBMCs with FACS buffer and seeded at 2×10⁵ cells/well into 96 well V bottom plate. Cells were then incubated with a saturating concentration (200 nM) of hIgG (isotype control) or test articles at 4° C. for 1 h. After the incubation, cells were washed and stained with Fixable Viability Stain 510, APC-Cy™7 Mouse Anti-Human CD3, Brilliant Violet 605™ anti-human CD4 Antibody, Alexa Fluor®700 anti-human CD8a Antibody, Brilliant Violet 421™ anti-human CD95 (Fas), goat F(ab′)2 anti-human IgG-Fc (PE) and incubated at 4° C. for 45 min. Cells were then washed and resuspend in FACS buffer for FACS analysis. PD-1 RO was calculated following the below equation,

RO (%)=[% CD3+CD95+(Control hIgG)/% CD3+CD95+(Bispecific)]×100

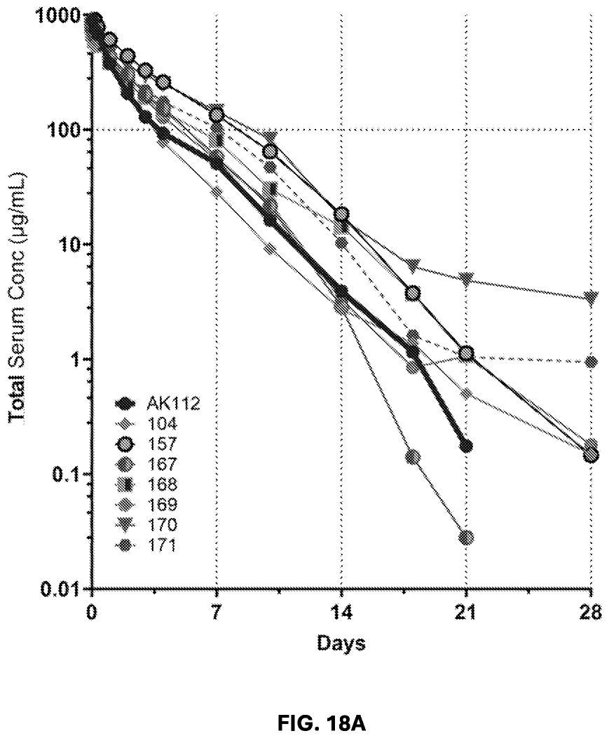

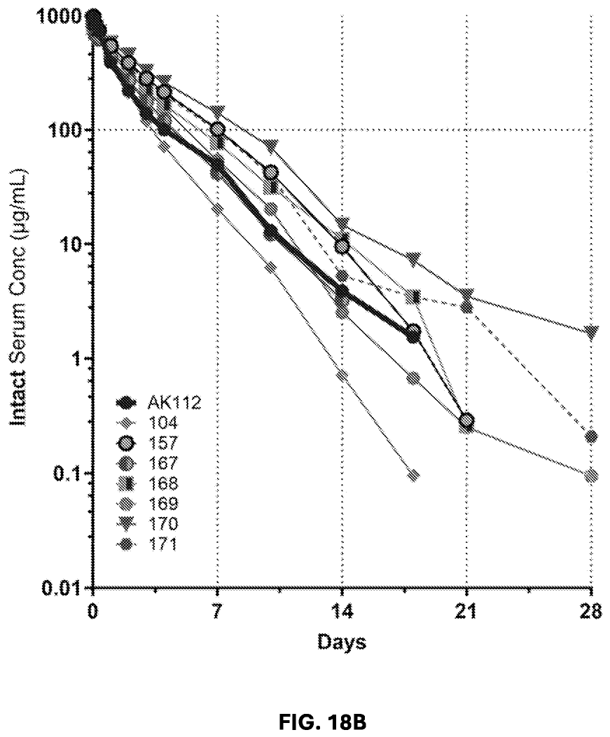

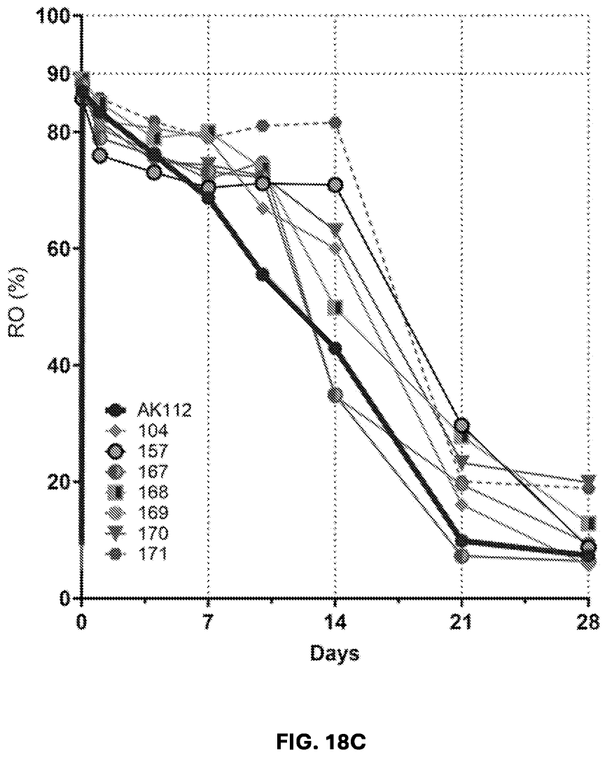

The results are summarized in FIGS. 18A-18C. The PK analysis demonstrated that PAL054-0170.1L had a half-life of 3.57 days, compared with 1.50 days for ivonescimab, and that ivonescimab and PAL054-0170.1L showed similar total and intact PK and receptor occupancy (RO) in NHP. The PK analysis demonstrated that the PAL054-0170.1L had improved half-life compared to those of ivonescimab (Table 24; FIGS. 18A-18B).

### Example 16: Efficacy Study with hPBMC Co-Administered with HCC827 Subcutaneously in Mouse Tumor Model

Seven to nine weeks old female SCID/Beige mice (Vital River Laboratory Animal Technology) divided into five groups with 8 mice per group were inoculated subcutaneously at the right hind flank with mixture of HCC827 tumor cells (4×10⁶), human PBMCs (8×10⁵) from healthy donor and indicated dose of antibody on day 0. Then the indicated antibodies (Table 25) were intravenously injected on day 7, 14, 21, 28, and 35. Tumor volume was measured twice a week up to day 68.

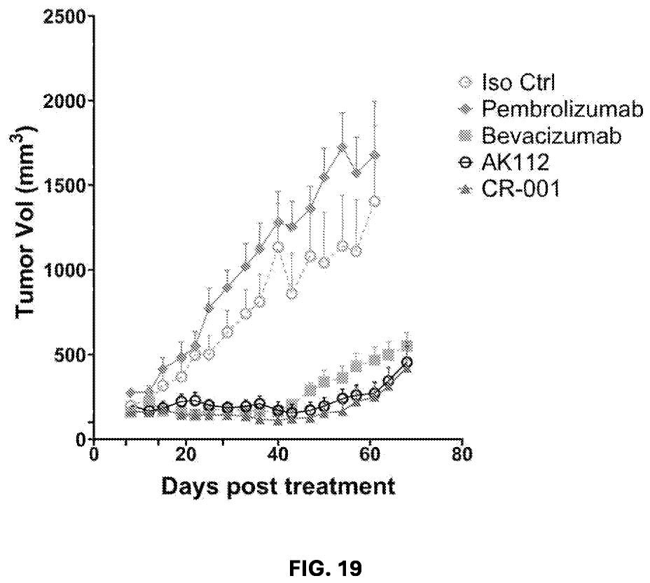

Anti-tumor activity of VEGF/PD-1 bispecific was evaluated and compared with that of bevacizumab in PBMC humanized SCID/Beige mice with subcutaneous HCC827 xenograft tumors. Treatment with PAL054-0170.1L resulted in strong tumor growth inhibition. The anti-tumor effect was greater than bevacizumab at the equivalent molarity doses of 10 mg/kg (FIG. 19).

TABLE 25
Mice cohorts for the in vivo study

| Treatment groups | Dose mg/kg |
|---|---|
| PAL054-0007.1 (Bevacizumab) | 10 |
| PAL054-0004.4 (Pembrolizumab) | 10 |
| PAL054-0001.1L (AK112) | 14 |
| PAL054-0170.1L | 14 |
| Isotype control | 10 |

### Numbered Embodiments

Embodiment 1. A bispecific antibody that binds vascular endothelial growth factor (VEGF) and programmed death receptor 1 (PD-1), comprising:
a) a heavy chain sequence comprising a sequence having at least 90% sequence identity to the amino acid sequence of any one of SEQ ID NOs: 22-23, 60, 62, 65-67, 70-72, 75-77, 142-143, 667-678, and 702-713; and
b) a light chain sequence comprising a sequence having at least 80% sequence identity to the amino acid sequence of any one of SEQ ID NOs: 148-194, 679-701, and 714-737.

Embodiment 2. The bispecific antibody of Embodiment 1, wherein the heavy chain sequence comprises a sequence having at least 90% sequence identity to the amino acid sequence of SEQ ID NO: 22; and the light chain sequence comprises a sequence having at least 80% sequence identity to the amino acid sequence of SEQ ID NO: 169.

Embodiment 3. The bispecific antibody of Embodiment 1, wherein the heavy chain sequence comprises a sequence having at least 90% sequence identity to the amino acid sequence of SEQ ID NO: 23; and the light chain sequence comprises a sequence having at least 80% sequence identity to the amino acid sequence of SEQ ID NO: 170.

Embodiment 4. The bispecific antibody of Embodiment 1, wherein the heavy chain sequence comprises a sequence having at least 90% sequence identity to the amino acid sequence of SEQ ID NO: 65; and the light chain sequence comprises a sequence having at least 80% sequence identity to the amino acid sequence of SEQ ID NO: 212.

Embodiment 5. The bispecific antibody of Embodiment 1, wherein the heavy chain sequence comprises a sequence having at least 90% sequence identity to the amino acid sequence of SEQ ID NO: 66; and the light chain sequence comprises a sequence having at least 80% sequence identity to the amino acid sequence of SEQ ID NO: 213.

Embodiment 6. The bispecific antibody of Embodiment 1, wherein the heavy chain sequence comprises a sequence having at least 90% sequence identity to the amino acid sequence of SEQ ID NO: 67; and the light chain sequence comprises a sequence having at least 80% sequence identity to the amino acid sequence of SEQ ID NO: 214.

Embodiment 7. The bispecific antibody of Embodiment 1, wherein the heavy chain sequence comprises a sequence having at least 90% sequence identity to the amino acid sequence of SEQ ID NO: 60; and the light chain sequence comprises a sequence having at least 80% sequence identity to the amino acid sequence of SEQ ID NO: 207.

Embodiment 8. The bispecific antibody of Embodiment 1, wherein the heavy chain sequence comprises a sequence having at least 90% sequence identity to the amino acid sequence of SEQ ID NO: 62; and the light chain sequence comprises a sequence having at least 80% sequence identity to the amino acid sequence of SEQ ID NO: 209.

Embodiment 9. The bispecific antibody of Embodiment 1, wherein the heavy chain sequence comprises a sequence having at least 90% sequence identity to the amino acid sequence of SEQ ID NO: 70; and the light chain sequence comprises a sequence having at least 80% sequence identity to the amino acid sequence of SEQ ID NO: 217.

Embodiment 10. The bispecific antibody of Embodiment 1, wherein the heavy chain sequence comprises a sequence having at least 90% sequence identity to the amino acid sequence of SEQ ID NO: 71; and the light chain sequence comprises a sequence having at least 80% sequence identity to the amino acid sequence of SEQ ID NO: 218.

Embodiment 11. The bispecific antibody of Embodiment 1, wherein the heavy chain sequence comprises a sequence having at least 90% sequence identity to the amino acid sequence of SEQ ID NO: 72; and the light chain sequence comprises a sequence having at least 80% sequence identity to the amino acid sequence of SEQ ID NO: 219.

Embodiment 12. The bispecific antibody of Embodiment 1, wherein the heavy chain sequence comprises a sequence having at least 90% sequence identity to the amino acid sequence of SEQ ID NO: 75; and the light chain sequence comprises a sequence having at least 80% sequence identity to the amino acid sequence of SEQ ID NO: 222.

Embodiment 13. The bispecific antibody of Embodiment 1, wherein the heavy chain sequence comprises a sequence having at least 90% sequence identity to the amino acid sequence of SEQ ID NO: 76; and the light chain sequence comprises a sequence having at least 80% sequence identity to the amino acid sequence of SEQ ID NO: 223.

Embodiment 14. The bispecific antibody of Embodiment 1, wherein the heavy chain sequence comprises a sequence having at least 90% sequence identity to the amino acid sequence of SEQ ID NO: 77; and the light chain sequence comprises a sequence having at least 80% sequence identity to the amino acid sequence of SEQ ID NO: 224.

Embodiment 15. The bispecific antibody of Embodiment 1, wherein the heavy chain sequence comprises a sequence having at least 90% sequence identity to the amino acid sequence of SEQ ID NO: 142; and the light chain sequence comprises a sequence having at least 80% sequence identity to the amino acid sequence of SEQ ID NO: 289.

Embodiment 16. The bispecific antibody of Embodiment 1, wherein the heavy chain sequence comprises a sequence having at least 90% sequence identity to the amino acid sequence of SEQ ID NO: 143; and the light chain sequence comprises a sequence having at least 80% sequence identity to the amino acid sequence of SEQ ID NO: 290.

Embodiment 17. The bispecific antibody of Embodiment 1, wherein the heavy chain sequence comprises a sequence having at least 90% sequence identity to the amino acid sequence of SEQ ID NO: 656; and the light chain sequence comprises a sequence having at least 80% sequence identity to the amino acid sequence of SEQ ID NO: 679.

Embodiment 18. The bispecific antibody of Embodiment 1, wherein the heavy chain sequence comprises a sequence having at least 90% sequence identity to the amino acid sequence of SEQ ID NO: 657; and the light chain sequence comprises a sequence having at least 80% sequence identity to the amino acid sequence of SEQ ID NO: 680.

Embodiment 19. The bispecific antibody of Embodiment 1, wherein the heavy chain sequence comprises a sequence having at least 90% sequence identity to the amino acid sequence of SEQ ID NO: 658; and the light chain sequence comprises a sequence having at least 80% sequence identity to the amino acid sequence of SEQ ID NO: 681.

Embodiment 20. The bispecific antibody of Embodiment 1, wherein the heavy chain sequence comprises a sequence having at least 90% sequence identity to the amino acid sequence of SEQ ID NO: 659; and the light chain sequence comprises a sequence having at least 80% sequence identity to the amino acid sequence of SEQ ID NO: 682.

Embodiment 21. The bispecific antibody of Embodiment 1, wherein the heavy chain sequence comprises a sequence having at least 90% sequence identity to the amino acid sequence of SEQ ID NO: 660; and the light chain sequence comprises a sequence having at least 80% sequence identity to the amino acid sequence of SEQ ID NO: 683.

Embodiment 22. The bispecific antibody of Embodiment 1, wherein the heavy chain sequence comprises a sequence having at least 90% sequence identity to the amino acid sequence of SEQ ID NO: 661; and the light chain sequence comprises a sequence having at least 80% sequence identity to the amino acid sequence of SEQ ID NO: 684.

Embodiment 23. The bispecific antibody of Embodiment 1, wherein the heavy chain sequence comprises a sequence having at least 90% sequence identity to the amino acid sequence of SEQ ID NO: 662; and the light chain sequence comprises a sequence having at least 80% sequence identity to the amino acid sequence of SEQ ID NO: 685.

Embodiment 24. The bispecific antibody of Embodiment 1, wherein the heavy chain sequence comprises a sequence having at least 90% sequence identity to the amino acid sequence of SEQ ID NO: 663; and the light chain sequence comprises a sequence having at least 80% sequence identity to the amino acid sequence of SEQ ID NO: 686.

Embodiment 25. The bispecific antibody of Embodiment 1, wherein the heavy chain sequence comprises a sequence having at least 90% sequence identity to the amino acid sequence of SEQ ID NO: 664; and the light chain sequence comprises a sequence having at least 80% sequence identity to the amino acid sequence of SEQ ID NO: 687.

Embodiment 26. The bispecific antibody of Embodiment 1, wherein the heavy chain sequence comprises a sequence having at least 90% sequence identity to the amino acid sequence of SEQ ID NO: 665; and the light chain sequence comprises a sequence having at least 80% sequence identity to the amino acid sequence of SEQ ID NO: 688.

Embodiment 27. The bispecific antibody of Embodiment 1, wherein the heavy chain sequence comprises a sequence having at least 90% sequence identity to the amino acid sequence of SEQ ID NO: 666; and the light chain sequence comprises a sequence having at least 80% sequence identity to the amino acid sequence of SEQ ID NO: 689.

Embodiment 28. The bispecific antibody of Embodiment 1, wherein the heavy chain sequence comprises a sequence having at least 90% sequence identity to the amino acid sequence of SEQ ID NO: 667; and the light chain sequence comprises a sequence having at least 80% sequence identity to the amino acid sequence of SEQ ID NO: 690.

Embodiment 29. The bispecific antibody of Embodiment 1, wherein the heavy chain sequence comprises a sequence having at least 90% sequence identity to the amino acid sequence of SEQ ID NO: 668; and the light chain sequence comprises a sequence having at least 80% sequence identity to the amino acid sequence of SEQ ID NO: 691.

Embodiment 30. The bispecific antibody of Embodiment 1, wherein the heavy chain sequence comprises a sequence having at least 90% sequence identity to the amino acid sequence of SEQ ID NO: 669; and the light chain sequence comprises a sequence having at least 80% sequence identity to the amino acid sequence of SEQ ID NO: 692.

Embodiment 31. The bispecific antibody of Embodiment 1, wherein the heavy chain sequence comprises a sequence having at least 90% sequence identity to the amino acid sequence of SEQ ID NO: 670; and the light chain sequence comprises a sequence having at least 80% sequence identity to the amino acid sequence of SEQ ID NO: 693.

Embodiment 32. The bispecific antibody of Embodiment 1, wherein the heavy chain sequence comprises a sequence having at least 90% sequence identity to the amino acid sequence of SEQ ID NO: 671; and the light chain sequence comprises a sequence having at least 80% sequence identity to the amino acid sequence of SEQ ID NO: 694.

Embodiment 33. The bispecific antibody of Embodiment 1, wherein the heavy chain sequence comprises a sequence having at least 90% sequence identity to the amino acid sequence of SEQ ID NO: 672; and the light chain sequence comprises a sequence having at least 80% sequence identity to the amino acid sequence of SEQ ID NO: 695.

Embodiment 34. The bispecific antibody of Embodiment 1, wherein the heavy chain sequence comprises a sequence having at least 90% sequence identity to the amino acid sequence of SEQ ID NO: 673; and the light chain sequence comprises a sequence having at least 80% sequence identity to the amino acid sequence of SEQ ID NO: 696.

Embodiment 35. The bispecific antibody of Embodiment 1, wherein the heavy chain sequence comprises a sequence having at least 90% sequence identity to the amino acid sequence of SEQ ID NO: 674; and the light chain sequence comprises a sequence having at least 80% sequence identity to the amino acid sequence of SEQ ID NO: 697.

Embodiment 36. The bispecific antibody of Embodiment 1, wherein the heavy chain sequence comprises a sequence having at least 90% sequence identity to the amino acid sequence of SEQ ID NO: 675; and the light chain sequence comprises a sequence having at least 80% sequence identity to the amino acid sequence of SEQ ID NO: 698.

Embodiment 37. The bispecific antibody of Embodiment 1, wherein the heavy chain sequence comprises a sequence having at least 90% sequence identity to the amino acid sequence of SEQ ID NO: 676; and the light chain sequence comprises a sequence having at least 80% sequence identity to the amino acid sequence of SEQ ID NO: 699.

Embodiment 38. The bispecific antibody of Embodiment 1, wherein the heavy chain sequence comprises a sequence having at least 90% sequence identity to the amino acid sequence of SEQ ID NO: 677; and the light chain sequence comprises a sequence having at least 80% sequence identity to the amino acid sequence of SEQ ID NO: 700.

Embodiment 39. The bispecific antibody of Embodiment 1, wherein the heavy chain sequence comprises a sequence having at least 90% sequence identity to the amino acid sequence of SEQ ID NO: 702; and a first light chain sequence comprises a sequence having at least 80% sequence identity to the amino acid sequence of SEQ ID NO: 714, and a second light chain sequence comprises a sequence having at least 80% sequence identity to the amino acid sequence of SEQ ID NO: 726.

Embodiment 40. The bispecific antibody of Embodiment 1, wherein the heavy chain sequence comprises a sequence having at least 90% sequence identity to the amino acid sequence of SEQ ID NO: 703; and a first light chain sequence comprises a sequence having at least 80% sequence identity to the amino acid sequence of SEQ ID NO: 715, and a second light chain sequence comprises a sequence having at least 80% sequence identity to the amino acid sequence of SEQ ID NO: 727.

Embodiment 41. The bispecific antibody of Embodiment 1, wherein the heavy chain sequence comprises a sequence having at least 90% sequence identity to the amino acid sequence of SEQ ID NO: 704; and a first light chain sequence comprises a sequence having at least 80% sequence identity to the amino acid sequence of SEQ ID NO: 716, and a second light chain sequence comprises a sequence having at least 80% sequence identity to the amino acid sequence of SEQ ID NO: 728.

Embodiment 42. The bispecific antibody of Embodiment 1, wherein the heavy chain sequence comprises a sequence having at least 90% sequence identity to the amino acid sequence of SEQ ID NO: 705; and a first light chain sequence comprises a sequence having at least 80% sequence identity to the amino acid sequence of SEQ ID NO: 717, and a second light chain sequence comprises a sequence having at least 80% sequence identity to the amino acid sequence of SEQ ID NO: 729.

Embodiment 43. The bispecific antibody of Embodiment 1, wherein the heavy chain sequence comprises a sequence having at least 90% sequence identity to the amino acid sequence of SEQ ID NO: 706; and a first light chain sequence comprises a sequence having at least 80% sequence identity to the amino acid sequence of SEQ ID NO: 718, and a second light chain sequence comprises a sequence having at least 80% sequence identity to the amino acid sequence of SEQ ID NO: 730.

Embodiment 44. The bispecific antibody of Embodiment 1, wherein the heavy chain sequence comprises a sequence having at least 90% sequence identity to the amino acid sequence of SEQ ID NO: 707; and a first light chain sequence comprises a sequence having at least 80% sequence identity to the amino acid sequence of SEQ ID NO: 719, and a second light chain sequence comprises a sequence having at least 80% sequence identity to the amino acid sequence of SEQ ID NO: 731.

Embodiment 45. The bispecific antibody of Embodiment 1, wherein the heavy chain sequence comprises a sequence having at least 90% sequence identity to the amino acid sequence of SEQ ID NO: 708; and a first light chain sequence comprises a sequence having at least 80% sequence identity to the amino acid sequence of SEQ ID NO: 720, and a second light chain sequence comprises a sequence having at least 80% sequence identity to the amino acid sequence of SEQ ID NO: 732.

Embodiment 46. The bispecific antibody of Embodiment 1, wherein the heavy chain sequence comprises a sequence having at least 90% sequence identity to the amino acid sequence of SEQ ID NO: 709; and a first light chain sequence comprises a sequence having at least 80% sequence identity to the amino acid sequence of SEQ ID NO: 721, and a second light chain sequence comprises a sequence having at least 80% sequence identity to the amino acid sequence of SEQ ID NO: 733.

Embodiment 47. The bispecific antibody of Embodiment 1, wherein the heavy chain sequence comprises a sequence having at least 90% sequence identity to the amino acid sequence of SEQ ID NO: 710; and a first light chain sequence comprises a sequence having at least 80% sequence identity to the amino acid sequence of SEQ ID NO: 722, and a second light chain sequence comprises a sequence having at least 80% sequence identity to the amino acid sequence of SEQ ID NO: 734.

Embodiment 48. The bispecific antibody of Embodiment 1, wherein the heavy chain sequence comprises a sequence having at least 90% sequence identity to the amino acid sequence of SEQ ID NO: 711; and a first light chain sequence comprises a sequence having at least 80% sequence identity to the amino acid sequence of SEQ ID NO: 723, and a second light chain sequence comprises a sequence having at least 80% sequence identity to the amino acid sequence of SEQ ID NO: 735.

Embodiment 49. The bispecific antibody of Embodiment 1, wherein the heavy chain sequence comprises a sequence having at least 90% sequence identity to the amino acid sequence of SEQ ID NO: 712; and a first light chain sequence comprises a sequence having at least 80% sequence identity to the amino acid sequence of SEQ ID NO: 724, and a second light chain sequence comprises a sequence having at least 80% sequence identity to the amino acid sequence of SEQ ID NO: 736.

Embodiment 50. The bispecific antibody of Embodiment 1, wherein the heavy chain sequence comprises a sequence having at least 90% sequence identity to the amino acid sequence of SEQ ID NO: 713; and a first light chain sequence comprises a sequence having at least 80% sequence identity to the amino acid sequence of SEQ ID NO: 725, and a second light chain sequence comprises a sequence having at least 80% sequence identity to the amino acid sequence of SEQ ID NO: 737.

Embodiment 51. The bispecific antibody of Embodiment 1, wherein the heavy chain sequence comprises a sequence having at least 90% sequence identity to the amino acid sequence of SEQ ID NO: 678; and the light chain sequence comprises a sequence having at least 80% sequence identity to the amino acid sequence of SEQ ID NO: 701.

Embodiment 52. A bispecific antibody that binds vascular endothelial growth factor (VEGF) and programmed death receptor 1 (PD-1), comprising:
a) a heavy chain sequence comprising a sequence having at least 95% sequence identity to the amino acid sequence of any one of SEQ ID NOs: 1-21, 24-57, 58, 59, 61, 63, 64, 68, 69, 73, 74, 78-97, 130-141, 144-147, and 656-666; and
b) a light chain sequence comprising a sequence having at least 80% sequence identity to the amino acid sequence of any one of SEQ ID NOs: 148-194, 679-701, and 714-737.

Embodiment 53. The bispecific antibody of Embodiment 52, wherein the heavy chain sequence comprises a sequence having at least 95% sequence identity to the amino acid sequence of SEQ ID NO: 1; and the light chain sequence comprises a sequence having at least 80% sequence identity to the amino acid sequence of SEQ ID NO: 148.

Embodiment 54. The bispecific antibody of Embodiment 52, wherein the heavy chain sequence comprises a sequence having at least 95% sequence identity to the amino acid sequence of SEQ ID NO: 2; and the light chain sequence comprises a sequence having at least 80% sequence identity to the amino acid sequence of SEQ ID NO: 149.

Embodiment 55. The bispecific antibody of Embodiment 52, wherein the heavy chain sequence comprises a sequence having at least 95% sequence identity to the amino acid sequence of SEQ ID NO: 3; and the light chain sequence comprises a sequence having at least 80% sequence identity to the amino acid sequence of SEQ ID NO: 150.

Embodiment 56. The bispecific antibody of Embodiment 52, wherein the heavy chain sequence comprises a sequence having at least 95% sequence identity to the amino acid sequence of SEQ ID NO: 4; and the light chain sequence comprises a sequence having at least 80% sequence identity to the amino acid sequence of SEQ ID NO: 151.

Embodiment 57. The bispecific antibody of Embodiment 52, wherein the heavy chain sequence comprises a sequence having at least 95% sequence identity to the amino acid sequence of SEQ ID NO: 5; and the light chain sequence comprises a sequence having at least 80% sequence identity to the amino acid sequence of SEQ ID NO: 152.

Embodiment 58. The bispecific antibody of Embodiment 52, wherein the heavy chain sequence comprises a sequence having at least 95% sequence identity to the amino acid sequence of SEQ ID NO: 6; and the light chain sequence comprises a sequence having at least 80% sequence identity to the amino acid sequence of SEQ ID NO: 153.

Embodiment 59. The bispecific antibody of Embodiment 52, wherein the heavy chain sequence comprises a sequence having at least 95% sequence identity to the amino acid sequence of SEQ ID NO: 7; and the light chain sequence comprises a sequence having at least 80% sequence identity to the amino acid sequence of SEQ ID NO: 154.

Embodiment 60. The bispecific antibody of Embodiment 52, wherein the heavy chain sequence comprises a sequence having at least 95% sequence identity to the amino acid sequence of SEQ ID NO: 8; and the light chain sequence comprises a sequence having at least 80% sequence identity to the amino acid sequence of SEQ ID NO: 155.

Embodiment 61. The bispecific antibody of Embodiment 52, wherein the heavy chain sequence comprises a sequence having at least 95% sequence identity to the amino acid sequence of SEQ ID NO: 9; and the light chain sequence comprises a sequence having at least 80% sequence identity to the amino acid sequence of SEQ ID NO: 156.

Embodiment 62. The bispecific antibody of Embodiment 52, wherein the heavy chain sequence comprises a sequence having at least 95% sequence identity to the amino acid sequence of SEQ ID NO: 10; and the light chain sequence comprises a sequence having at least 80% sequence identity to the amino acid sequence of SEQ ID NO: 157.

Embodiment 63. The bispecific antibody of Embodiment 52, wherein the heavy chain sequence comprises a sequence having at least 95% sequence identity to the amino acid sequence of SEQ ID NO: 11; and the light chain sequence comprises a sequence having at least 80% sequence identity to the amino acid sequence of SEQ ID NO: 158.

Embodiment 64. The bispecific antibody of Embodiment 52, wherein the heavy chain sequence comprises a sequence having at least 95% sequence identity to the amino acid sequence of SEQ ID NO: 12; and the light chain sequence comprises a sequence having at least 80% sequence identity to the amino acid sequence of SEQ ID NO: 159.

Embodiment 65. The bispecific antibody of Embodiment 52, wherein the heavy chain sequence comprises a sequence having at least 95% sequence identity to the amino acid sequence of SEQ ID NO: 13; and the light chain sequence comprises a sequence having at least 80% sequence identity to the amino acid sequence of SEQ ID NO: 160.

Embodiment 66. The bispecific antibody of Embodiment 52, wherein the heavy chain sequence comprises a sequence having at least 95% sequence identity to the amino acid sequence of SEQ ID NO: 14; and the light chain sequence comprises a sequence having at least 80% sequence identity to the amino acid sequence of SEQ ID NO: 161.

Embodiment 67. The bispecific antibody of Embodiment 52, wherein the heavy chain sequence comprises a sequence having at least 95% sequence identity to the amino acid sequence of SEQ ID NO: 15; and the light chain sequence comprises a sequence having at least 80% sequence identity to the amino acid sequence of SEQ ID NO: 162.

Embodiment 68. The bispecific antibody of Embodiment 52, wherein the heavy chain sequence comprises a sequence having at least 95% sequence identity to the amino acid sequence of SEQ ID NO: 16; and the light chain sequence comprises a sequence having at least 80% sequence identity to the amino acid sequence of SEQ ID NO: 163.

Embodiment 69. The bispecific antibody of Embodiment 52, wherein the heavy chain sequence comprises a sequence having at least 95% sequence identity to the amino acid sequence of SEQ ID NO: 17; and the light chain sequence comprises a sequence having at least 80% sequence identity to the amino acid sequence of SEQ ID NO: 164.

Embodiment 70. The bispecific antibody of Embodiment 52, wherein the heavy chain sequence comprises a sequence having at least 95% sequence identity to the amino acid sequence of SEQ ID NO: 18; and the light chain sequence comprises a sequence having at least 80% sequence identity to the amino acid sequence of SEQ ID NO: 165.

Embodiment 71. The bispecific antibody of Embodiment 52, wherein the heavy chain sequence comprises a sequence having at least 95% sequence identity to the amino acid sequence of SEQ ID NO: 19; and the light chain sequence comprises a sequence having at least 80% sequence identity to the amino acid sequence of SEQ ID NO: 166.

Embodiment 72. The bispecific antibody of Embodiment 52, wherein the heavy chain sequence comprises a sequence having at least 95% sequence identity to the amino acid sequence of SEQ ID NO: 20; and the light chain sequence comprises a sequence having at least 80% sequence identity to the amino acid sequence of SEQ ID NO: 167.

Embodiment 73. The bispecific antibody of Embodiment 52, wherein the heavy chain sequence comprises a sequence having at least 95% sequence identity to the amino acid sequence of SEQ ID NO: 21; and the light chain sequence comprises a sequence having at least 80% sequence identity to the amino acid sequence of SEQ ID NO: 168.

Embodiment 74. The bispecific antibody of Embodiment 52, wherein the heavy chain sequence comprises a sequence having at least 95% sequence identity to the amino acid sequence of SEQ ID NO: 24; and the light chain sequence comprises a sequence having at least 80% sequence identity to the amino acid sequence of SEQ ID NO: 171.

Embodiment 75. The bispecific antibody of Embodiment 52, wherein the heavy chain sequence comprises a sequence having at least 95% sequence identity to the amino acid sequence of SEQ ID NO: 25; and the light chain sequence comprises a sequence having at least 80% sequence identity to the amino acid sequence of SEQ ID NO: 172.

Embodiment 76. The bispecific antibody of Embodiment 52, wherein the heavy chain sequence comprises a sequence having at least 95% sequence identity to the amino acid sequence of SEQ ID NO: 26; and the light chain sequence comprises a sequence having at least 80% sequence identity to the amino acid sequence of SEQ ID NO: 173.

Embodiment 77. The bispecific antibody of Embodiment 52, wherein the heavy chain sequence comprises a sequence having at least 95% sequence identity to the amino acid sequence of SEQ ID NO: 27; and the light chain sequence comprises a sequence having at least 80% sequence identity to the amino acid sequence of SEQ ID NO: 174.

Embodiment 78. The bispecific antibody of Embodiment 52, wherein the heavy chain sequence comprises a sequence having at least 95% sequence identity to the amino acid sequence of SEQ ID NO: 28; and the light chain sequence comprises a sequence having at least 80% sequence identity to the amino acid sequence of SEQ ID NO: 175.

Embodiment 79. The bispecific antibody of Embodiment 52, wherein the heavy chain sequence comprises a sequence having at least 95% sequence identity to the amino acid sequence of SEQ ID NO: 29; and the light chain sequence comprises a sequence having at least 80% sequence identity to the amino acid sequence of SEQ ID NO: 176.

Embodiment 80. The bispecific antibody of Embodiment 52, wherein the heavy chain sequence comprises a sequence having at least 95% sequence identity to the amino acid sequence of SEQ ID NO: 30; and the light chain sequence comprises a sequence having at least 80% sequence identity to the amino acid sequence of SEQ ID NO: 177.

Embodiment 81. The bispecific antibody of Embodiment 52, wherein the heavy chain sequence comprises a sequence having at least 95% sequence identity to the amino acid sequence of SEQ ID NO: 31; and the light chain sequence comprises a sequence having at least 80% sequence identity to the amino acid sequence of SEQ ID NO: 178.

Embodiment 82. The bispecific antibody of Embodiment 52, wherein the heavy chain sequence comprises a sequence having at least 95% sequence identity to the amino acid sequence of SEQ ID NO: 32; and the light chain sequence comprises a sequence having at least 80% sequence identity to the amino acid sequence of SEQ ID NO: 179.

Embodiment 83. The bispecific antibody of Embodiment 52, wherein the heavy chain sequence comprises a sequence having at least 95% sequence identity to the amino acid sequence of SEQ ID NO: 33; and the light chain sequence comprises a sequence having at least 80% sequence identity to the amino acid sequence of SEQ ID NO: 180.

Embodiment 84. The bispecific antibody of Embodiment 52, wherein the heavy chain sequence comprises a sequence having at least 95% sequence identity to the amino acid sequence of SEQ ID NO: 34; and the light chain sequence comprises a sequence having at least 80% sequence identity to the amino acid sequence of SEQ ID NO: 181.

Embodiment 85. The bispecific antibody of Embodiment 52, wherein the heavy chain sequence comprises a sequence having at least 95% sequence identity to the amino acid sequence of SEQ ID NO: 35; and the light chain sequence comprises a sequence having at least 80% sequence identity to the amino acid sequence of SEQ ID NO: 182.

Embodiment 86. The bispecific antibody of Embodiment 52, wherein the heavy chain sequence comprises a sequence having at least 95% sequence identity to the amino acid sequence of SEQ ID NO: 36; and the light chain sequence comprises a sequence having at least 80% sequence identity to the amino acid sequence of SEQ ID NO: 183.

Embodiment 87. The bispecific antibody of Embodiment 52, wherein the heavy chain sequence comprises a sequence having at least 95% sequence identity to the amino acid sequence of SEQ ID NO: 37; and the light chain sequence comprises a sequence having at least 80% sequence identity to the amino acid sequence of SEQ ID NO: 184.

Embodiment 88. The bispecific antibody of Embodiment 52, wherein the heavy chain sequence comprises a sequence having at least 95% sequence identity to the amino acid sequence of SEQ ID NO: 38; and the light chain sequence comprises a sequence having at least 80% sequence identity to the amino acid sequence of SEQ ID NO: 185.

Embodiment 89. The bispecific antibody of Embodiment 52, wherein the heavy chain sequence comprises a sequence having at least 95% sequence identity to the amino acid sequence of SEQ ID NO: 39; and the light chain sequence comprises a sequence having at least 80% sequence identity to the amino acid sequence of SEQ ID NO: 186.

Embodiment 90. The bispecific antibody of Embodiment 52, wherein the heavy chain sequence comprises a sequence having at least 95% sequence identity to the amino acid sequence of SEQ ID NO: 40; and the light chain sequence comprises a sequence having at least 80% sequence identity to the amino acid sequence of SEQ ID NO: 187.

Embodiment 91. The bispecific antibody of Embodiment 52, wherein the heavy chain sequence comprises a sequence having at least 95% sequence identity to the amino acid sequence of SEQ ID NO: 41; and the light chain sequence comprises a sequence having at least 80% sequence identity to the amino acid sequence of SEQ ID NO: 188.

Embodiment 92. The bispecific antibody of Embodiment 52, wherein the heavy chain sequence comprises a sequence having at least 95% sequence identity to the amino acid sequence of SEQ ID NO: 42; and the light chain sequence comprises a sequence having at least 80% sequence identity to the amino acid sequence of SEQ ID NO: 189.

Embodiment 93. The bispecific antibody of Embodiment 52, wherein the heavy chain sequence comprises a sequence having at least 95% sequence identity to the amino acid sequence of SEQ ID NO: 43; and the light chain sequence comprises a sequence having at least 80% sequence identity to the amino acid sequence of SEQ ID NO: 190.

Embodiment 94. The bispecific antibody of Embodiment 52, wherein the heavy chain sequence comprises a sequence having at least 95% sequence identity to the amino acid sequence of SEQ ID NO: 44; and the light chain sequence comprises a sequence having at least 80% sequence identity to the amino acid sequence of SEQ ID NO: 191.

Embodiment 95. The bispecific antibody of Embodiment 52, wherein the heavy chain sequence comprises a sequence having at least 95% sequence identity to the amino acid sequence of SEQ ID NO: 45; and the light chain sequence comprises a sequence having at least 80% sequence identity to the amino acid sequence of SEQ ID NO: 192.

Embodiment 96. The bispecific antibody of Embodiment 52, wherein the heavy chain sequence comprises a sequence having at least 95% sequence identity to the amino acid sequence of SEQ ID NO: 46; and the light chain sequence comprises a sequence having at least 80% sequence identity to the amino acid sequence of SEQ ID NO: 193.

Embodiment 97. The bispecific antibody of Embodiment 52, wherein the heavy chain sequence comprises a sequence having at least 95% sequence identity to the amino acid sequence of SEQ ID NO: 47; and the light chain sequence comprises a sequence having at least 80% sequence identity to the amino acid sequence of SEQ ID NO: 194.

Embodiment 98. The bispecific antibody of Embodiment 52, wherein the heavy chain sequence comprises a sequence having at least 95% sequence identity to the amino acid sequence of SEQ ID NO: 48; and the light chain sequence comprises a sequence having at least 80% sequence identity to the amino acid sequence of SEQ ID NO: 195.

Embodiment 99. The bispecific antibody of Embodiment 52, wherein the heavy chain sequence comprises a sequence having at least 95% sequence identity to the amino acid sequence of SEQ ID NO: 49; and the light chain sequence comprises a sequence having at least 80% sequence identity to the amino acid sequence of SEQ ID NO: 196.

Embodiment 100. The bispecific antibody of Embodiment 52, wherein the heavy chain sequence comprises a sequence having at least 95% sequence identity to the amino acid sequence of SEQ ID NO: 50; and the light chain sequence comprises a sequence having at least 80% sequence identity to the amino acid sequence of SEQ ID NO: 197.

Embodiment 101. The bispecific antibody of Embodiment 52, wherein the heavy chain sequence comprises a sequence having at least 95% sequence identity to the amino acid sequence of SEQ ID NO: 51; and the light chain sequence comprises a sequence having at least 80% sequence identity to the amino acid sequence of SEQ ID NO: 198.

Embodiment 102. The bispecific antibody of Embodiment 52, wherein the heavy chain sequence comprises a sequence having at least 95% sequence identity to the amino acid sequence of SEQ ID NO: 52; and the light chain sequence comprises a sequence having at least 80% sequence identity to the amino acid sequence of SEQ ID NO: 199.

Embodiment 103. The bispecific antibody of Embodiment 52, wherein the heavy chain sequence comprises a sequence having at least 95% sequence identity to the amino acid sequence of SEQ ID NO: 53; and the light chain sequence comprises a sequence having at least 80% sequence identity to the amino acid sequence of SEQ ID NO: 200.

Embodiment 104. The bispecific antibody of Embodiment 52, wherein the heavy chain sequence comprises a sequence having at least 95% sequence identity to the amino acid sequence of SEQ ID NO: 54; and the light chain sequence comprises a sequence having at least 80% sequence identity to the amino acid sequence of SEQ ID NO: 201.

Embodiment 105. The bispecific antibody of Embodiment 52, wherein the heavy chain sequence comprises a sequence having at least 95% sequence identity to the amino acid sequence of SEQ ID NO: 55; and the light chain sequence comprises a sequence having at least 80% sequence identity to the amino acid sequence of SEQ ID NO: 202.

Embodiment 106. The bispecific antibody of Embodiment 52, wherein the heavy chain sequence comprises a sequence having at least 95% sequence identity to the amino acid sequence of SEQ ID NO: 56; and the light chain sequence comprises a sequence having at least 80% sequence identity to the amino acid sequence of SEQ ID NO: 203.

Embodiment 107. The bispecific antibody of Embodiment 52, wherein the heavy chain sequence comprises a sequence having at least 95% sequence identity to the amino acid sequence of SEQ ID NO: 57; and the light chain sequence comprises a sequence having at least 80% sequence identity to the amino acid sequence of SEQ ID NO: 204.

Embodiment 108. The bispecific antibody of Embodiment 52, wherein the heavy chain sequence comprises a sequence having at least 95% sequence identity to the amino acid sequence of SEQ ID NO: 58; and the light chain sequence comprises a sequence having at least 80% sequence identity to the amino acid sequence of SEQ ID NO: 205.

Embodiment 109. The bispecific antibody of Embodiment 52, wherein the heavy chain sequence comprises a sequence having at least 95% sequence identity to the amino acid sequence of SEQ ID NO: 59; and the light chain sequence comprises a sequence having at least 80% sequence identity to the amino acid sequence of SEQ ID NO: 206.

Embodiment 110. The bispecific antibody of Embodiment 52, wherein the heavy chain sequence comprises a sequence having at least 95% sequence identity to the amino acid sequence of SEQ ID NO: 61; and the light chain sequence comprises a sequence having at least 80% sequence identity to the amino acid sequence of SEQ ID NO: 208.

Embodiment 111. The bispecific antibody of Embodiment 52, wherein the heavy chain sequence comprises a sequence having at least 95% sequence identity to the amino acid sequence of SEQ ID NO: 63; and the light chain sequence comprises a sequence having at least 80% sequence identity to the amino acid sequence of SEQ ID NO: 210.

Embodiment 112. The bispecific antibody of Embodiment 52, wherein the heavy chain sequence comprises a sequence having at least 95% sequence identity to the amino acid sequence of SEQ ID NO: 64; and the light chain sequence comprises a sequence having at least 80% sequence identity to the amino acid sequence of SEQ ID NO: 211.

Embodiment 113. The bispecific antibody of Embodiment 52, wherein the heavy chain sequence comprises a sequence having at least 95% sequence identity to the amino acid sequence of SEQ ID NO: 68; and the light chain sequence comprises a sequence having at least 80% sequence identity to the amino acid sequence of SEQ ID NO: 215.

Embodiment 114. The bispecific antibody of Embodiment 52, wherein the heavy chain sequence comprises a sequence having at least 95% sequence identity to the amino acid sequence of SEQ ID NO: 69; and the light chain sequence comprises a sequence having at least 80% sequence identity to the amino acid sequence of SEQ ID NO: 216.

Embodiment 115. The bispecific antibody of Embodiment 52, wherein the heavy chain sequence comprises a sequence having at least 95% sequence identity to the amino acid sequence of SEQ ID NO: 73; and the light chain sequence comprises a sequence having at least 80% sequence identity to the amino acid sequence of SEQ ID NO: 220.

Embodiment 116. The bispecific antibody of Embodiment 52, wherein the heavy chain sequence comprises a sequence having at least 95% sequence identity to the amino acid sequence of SEQ ID NO: 74; and the light chain sequence comprises a sequence having at least 80% sequence identity to the amino acid sequence of SEQ ID NO: 221.

Embodiment 117. The bispecific antibody of Embodiment 52, wherein the heavy chain sequence comprises a sequence having at least 95% sequence identity to the amino acid sequence of SEQ ID NO: 78; and the light chain sequence comprises a sequence having at least 80% sequence identity to the amino acid sequence of SEQ ID NO: 225.

Embodiment 118. The bispecific antibody of Embodiment 52, wherein the heavy chain sequence comprises a sequence having at least 95% sequence identity to the amino acid sequence of SEQ ID NO: 79; and the light chain sequence comprises a sequence having at least 80% sequence identity to the amino acid sequence of SEQ ID NO: 226.

Embodiment 119. The bispecific antibody of Embodiment 52, wherein the heavy chain sequence comprises a sequence having at least 95% sequence identity to the amino acid sequence of SEQ ID NO: 80; and the light chain sequence comprises a sequence having at least 80% sequence identity to the amino acid sequence of SEQ ID NO: 227.

Embodiment 120. The bispecific antibody of Embodiment 52, wherein the heavy chain sequence comprises a sequence having at least 95% sequence identity to the amino acid sequence of SEQ ID NO: 81; and the light chain sequence comprises a sequence having at least 80% sequence identity to the amino acid sequence of SEQ ID NO: 228.

Embodiment 121. The bispecific antibody of Embodiment 52, wherein the heavy chain sequence comprises a sequence having at least 95% sequence identity to the amino acid sequence of SEQ ID NO: 82; and the light chain sequence comprises a sequence having at least 80% sequence identity to the amino acid sequence of SEQ ID NO: 229.

Embodiment 122. The bispecific antibody of Embodiment 52, wherein the heavy chain sequence comprises a sequence having at least 95% sequence identity to the amino acid sequence of SEQ ID NO: 83; and the light chain sequence comprises a sequence having at least 80% sequence identity to the amino acid sequence of SEQ ID NO: 230.

Embodiment 123. The bispecific antibody of Embodiment 52, wherein the heavy chain sequence comprises a sequence having at least 95% sequence identity to the amino acid sequence of SEQ ID NO: 84; and the light chain sequence comprises a sequence having at least 80% sequence identity to the amino acid sequence of SEQ ID NO: 231.

Embodiment 124. The bispecific antibody of Embodiment 52, wherein the heavy chain sequence comprises a sequence having at least 95% sequence identity to the amino acid sequence of SEQ ID NO: 85; and the light chain sequence comprises a sequence having at least 80% sequence identity to the amino acid sequence of SEQ ID NO: 232.

Embodiment 125. The bispecific antibody of Embodiment 52, wherein the heavy chain sequence comprises a sequence having at least 95% sequence identity to the amino acid sequence of SEQ ID NO: 86; and the light chain sequence comprises a sequence having at least 80% sequence identity to the amino acid sequence of SEQ ID NO: 233.

Embodiment 126. The bispecific antibody of Embodiment 52, wherein the heavy chain sequence comprises a sequence having at least 95% sequence identity to the amino acid sequence of SEQ ID NO: 87; and the light chain sequence comprises a sequence having at least 80% sequence identity to the amino acid sequence of SEQ ID NO: 234.

Embodiment 127. The bispecific antibody of Embodiment 52, wherein the heavy chain sequence comprises a sequence having at least 95% sequence identity to the amino acid sequence of SEQ ID NO: 88; and the light chain sequence comprises a sequence having at least 80% sequence identity to the amino acid sequence of SEQ ID NO: 235.

Embodiment 128. The bispecific antibody of Embodiment 52, wherein the heavy chain sequence comprises a sequence having at least 95% sequence identity to the amino acid sequence of SEQ ID NO: 89; and the light chain sequence comprises a sequence having at least 80% sequence identity to the amino acid sequence of SEQ ID NO: 236.

Embodiment 129. The bispecific antibody of Embodiment 52, wherein the heavy chain sequence comprises a sequence having at least 95% sequence identity to the amino acid sequence of SEQ ID NO: 90; and the light chain sequence comprises a sequence having at least 80% sequence identity to the amino acid sequence of SEQ ID NO: 237.

Embodiment 130. The bispecific antibody of Embodiment 52, wherein the heavy chain sequence comprises a sequence having at least 95% sequence identity to the amino acid sequence of SEQ ID NO: 91; and the light chain sequence comprises a sequence having at least 80% sequence identity to the amino acid sequence of SEQ ID NO: 238.

Embodiment 131. The bispecific antibody of Embodiment 52, wherein the heavy chain sequence comprises a sequence having at least 95% sequence identity to the amino acid sequence of SEQ ID NO: 92; and the light chain sequence comprises a sequence having at least 80% sequence identity to the amino acid sequence of SEQ ID NO: 239.

Embodiment 132. The bispecific antibody of Embodiment 52, wherein the heavy chain sequence comprises a sequence having at least 95% sequence identity to the amino acid sequence of SEQ ID NO: 93; and the light chain sequence comprises a sequence having at least 80% sequence identity to the amino acid sequence of SEQ ID NO: 240.

Embodiment 133. The bispecific antibody of Embodiment 52, wherein the heavy chain sequence comprises a sequence having at least 95% sequence identity to the amino acid sequence of SEQ ID NO: 94; and the light chain sequence comprises a sequence having at least 80% sequence identity to the amino acid sequence of SEQ ID NO: 241.

Embodiment 134. The bispecific antibody of Embodiment 52, wherein the heavy chain sequence comprises a sequence having at least 95% sequence identity to the amino acid sequence of SEQ ID NO: 95; and the light chain sequence comprises a sequence having at least 80% sequence identity to the amino acid sequence of SEQ ID NO: 242.

Embodiment 135. The bispecific antibody of Embodiment 52, wherein the heavy chain sequence comprises a sequence having at least 95% sequence identity to the amino acid sequence of SEQ ID NO: 96; and the light chain sequence comprises a sequence having at least 80% sequence identity to the amino acid sequence of SEQ ID NO: 243.

Embodiment 136. The bispecific antibody of Embodiment 52, wherein the heavy chain sequence comprises a sequence having at least 95% sequence identity to the amino acid sequence of SEQ ID NO: 97; and the light chain sequence comprises a sequence having at least 80% sequence identity to the amino acid sequence of SEQ ID NO: 244.

Embodiment 137. The bispecific antibody of Embodiment 52, wherein the heavy chain sequence comprises a sequence having at least 95% sequence identity to the amino acid sequence of SEQ ID NO: 130; and the light chain sequence comprises a sequence having at least 80% sequence identity to the amino acid sequence of SEQ ID NO: 277.

Embodiment 138. The bispecific antibody of Embodiment 52, wherein the heavy chain sequence comprises a sequence having at least 95% sequence identity to the amino acid sequence of SEQ ID NO: 131; and the light chain sequence comprises a sequence having at least 80% sequence identity to the amino acid sequence of SEQ ID NO: 278.

Embodiment 139. The bispecific antibody of Embodiment 52, wherein the heavy chain sequence comprises a sequence having at least 95% sequence identity to the amino acid sequence of SEQ ID NO: 132; and the light chain sequence comprises a sequence having at least 80% sequence identity to the amino acid sequence of SEQ ID NO: 279.

Embodiment 140. The bispecific antibody of Embodiment 52, wherein the heavy chain sequence comprises a sequence having at least 95% sequence identity to the amino acid sequence of SEQ ID NO: 133; and the light chain sequence comprises a sequence having at least 80% sequence identity to the amino acid sequence of SEQ ID NO: 280.

Embodiment 141. The bispecific antibody of Embodiment 52, wherein the heavy chain sequence comprises a sequence having at least 95% sequence identity to the amino acid sequence of SEQ ID NO: 134; and the light chain sequence comprises a sequence having at least 80% sequence identity to the amino acid sequence of SEQ ID NO: 281.

Embodiment 142. The bispecific antibody of Embodiment 52, wherein the heavy chain sequence comprises a sequence having at least 95% sequence identity to the amino acid sequence of SEQ ID NO: 135; and the light chain sequence comprises a sequence having at least 80% sequence identity to the amino acid sequence of SEQ ID NO: 282.

Embodiment 143. The bispecific antibody of Embodiment 52, wherein the heavy chain sequence comprises a sequence having at least 95% sequence identity to the amino acid sequence of SEQ ID NO: 136; and the light chain sequence comprises a sequence having at least 80% sequence identity to the amino acid sequence of SEQ ID NO: 283.

Embodiment 144. The bispecific antibody of Embodiment 52, wherein the heavy chain sequence comprises a sequence having at least 95% sequence identity to the amino acid sequence of SEQ ID NO: 137; and the light chain sequence comprises a sequence having at least 80% sequence identity to the amino acid sequence of SEQ ID NO: 284.

Embodiment 145. The bispecific antibody of Embodiment 52, wherein the heavy chain sequence comprises a sequence having at least 95% sequence identity to the amino acid sequence of SEQ ID NO: 138; and the light chain sequence comprises a sequence having at least 80% sequence identity to the amino acid sequence of SEQ ID NO: 285.

Embodiment 146. The bispecific antibody of Embodiment 52, wherein the heavy chain sequence comprises a sequence having at least 95% sequence identity to the amino acid sequence of SEQ ID NO: 139; and the light chain sequence comprises a sequence having at least 80% sequence identity to the amino acid sequence of SEQ ID NO: 286.

Embodiment 147. The bispecific antibody of Embodiment 52, wherein the heavy chain sequence comprises a sequence having at least 95% sequence identity to the amino acid sequence of SEQ ID NO: 140; and the light chain sequence comprises a sequence having at least 80% sequence identity to the amino acid sequence of SEQ ID NO: 287.

Embodiment 148. The bispecific antibody of Embodiment 52, wherein the heavy chain sequence comprises a sequence having at least 95% sequence identity to the amino acid sequence of SEQ ID NO: 141; and the light chain sequence comprises a sequence having at least 80% sequence identity to the amino acid sequence of SEQ ID NO: 288.

Embodiment 149. The bispecific antibody of Embodiment 52, wherein the heavy chain sequence comprises a sequence having at least 95% sequence identity to the amino acid sequence of SEQ ID NO: 144; and the light chain sequence comprises a sequence having at least 80% sequence identity to the amino acid sequence of SEQ ID NO: 291.

Embodiment 150. The bispecific antibody of Embodiment 52, wherein the heavy chain sequence comprises a sequence having at least 95% sequence identity to the amino acid sequence of SEQ ID NO: 145; and the light chain sequence comprises a sequence having at least 80% sequence identity to the amino acid sequence of SEQ ID NO: 292.

Embodiment 151. The bispecific antibody of Embodiment 52, wherein the heavy chain sequence comprises a sequence having at least 95% sequence identity to the amino acid sequence of SEQ ID NO: 146; and the light chain sequence comprises a sequence having at least 80% sequence identity to the amino acid sequence of SEQ ID NO: 293.

Embodiment 152. The bispecific antibody of Embodiment 52, wherein the heavy chain sequence comprises a sequence having at least 95% sequence identity to the amino acid sequence of SEQ ID NO: 147; and the light chain sequence comprises a sequence having at least 80% sequence identity to the amino acid sequence of SEQ ID NO: 294.

Embodiment 153. The bispecific antibody of Embodiment 52, wherein the heavy chain sequence comprises a sequence having at least 95% sequence identity to the amino acid sequence of SEQ ID NO: 656; and the light chain sequence comprises a sequence having at least 80% sequence identity to the amino acid sequence of SEQ ID NO: 679.

Embodiment 154. The bispecific antibody of Embodiment 52, wherein the heavy chain sequence comprises a sequence having at least 95% sequence identity to the amino acid sequence of SEQ ID NO: 657; and the light chain sequence comprises a sequence having at least 80% sequence identity to the amino acid sequence of SEQ ID NO: 680.

Embodiment 155. The bispecific antibody of Embodiment 52, wherein the heavy chain sequence comprises a sequence having at least 95% sequence identity to the amino acid sequence of SEQ ID NO: 658; and the light chain sequence comprises a sequence having at least 80% sequence identity to the amino acid sequence of SEQ ID NO: 681.

Embodiment 156. The bispecific antibody of Embodiment 52, wherein the heavy chain sequence comprises a sequence having at least 95% sequence identity to the amino acid sequence of SEQ ID NO: 659; and the light chain sequence comprises a sequence having at least 80% sequence identity to the amino acid sequence of SEQ ID NO: 682.

Embodiment 157. The bispecific antibody of Embodiment 52, wherein the heavy chain sequence comprises a sequence having at least 95% sequence identity to the amino acid sequence of SEQ ID NO: 660; and the light chain sequence comprises a sequence having at least 80% sequence identity to the amino acid sequence of SEQ ID NO: 683.

Embodiment 158. The bispecific antibody of Embodiment 52, wherein the heavy chain sequence comprises a sequence having at least 95% sequence identity to the amino acid sequence of SEQ ID NO: 661; and the light chain sequence comprises a sequence having at least 80% sequence identity to the amino acid sequence of SEQ ID NO: 684.

Embodiment 159. The bispecific antibody of Embodiment 52, wherein the heavy chain sequence comprises a sequence having at least 95% sequence identity to the amino acid sequence of SEQ ID NO: 662; and the light chain sequence comprises a sequence having at least 80% sequence identity to the amino acid sequence of SEQ ID NO: 685.

Embodiment 160. The bispecific antibody of Embodiment 52, wherein the heavy chain sequence comprises a sequence having at least 95% sequence identity to the amino acid sequence of SEQ ID NO: 663; and the light chain sequence comprises a sequence having at least 80% sequence identity to the amino acid sequence of SEQ ID NO: 686.

Embodiment 161. The bispecific antibody of Embodiment 52, wherein the heavy chain sequence comprises a sequence having at least 95% sequence identity to the amino acid sequence of SEQ ID NO: 664; and the light chain sequence comprises a sequence having at least 80% sequence identity to the amino acid sequence of SEQ ID NO: 687.

Embodiment 162. The bispecific antibody of Embodiment 52, wherein the heavy chain sequence comprises a sequence having at least 95% sequence identity to the amino acid sequence of SEQ ID NO: 665; and the light chain sequence comprises a sequence having at least 80% sequence identity to the amino acid sequence of SEQ ID NO: 688.

Embodiment 163. The bispecific antibody of Embodiment 52, wherein the heavy chain sequence comprises a sequence having at least 95% sequence identity to the amino acid sequence of SEQ ID NO: 666; and the light chain sequence comprises a sequence having at least 80% sequence identity to the amino acid sequence of SEQ ID NO: 689.

Embodiment 164. A bispecific antibody that binds vascular endothelial growth factor (VEGF) and programmed death receptor 1 (PD-1), comprising:
a) a heavy chain sequence comprising the amino acid sequence of any one of SEQ ID NOs: 98-127 and 129; and
b) a light chain sequence comprising a sequence having at least 80% sequence identity to the amino acid sequence of any one of SEQ ID NOs: 148-194, 679-701, and 714-737.

Embodiment 165. The bispecific antibody of Embodiment 164, wherein the heavy chain sequence comprises the amino acid sequence of SEQ ID NO: 98; and the light chain sequence comprises a sequence having at least 80% sequence identity to the amino acid sequence of SEQ ID NO: 245.

Embodiment 166. The bispecific antibody of Embodiment 164, wherein the heavy chain sequence comprises the amino acid sequence of SEQ ID NO: 99; and the light chain sequence comprises a sequence having at least 80% sequence identity to the amino acid sequence of SEQ ID NO: 246.

Embodiment 167. The bispecific antibody of Embodiment 164, wherein the heavy chain sequence comprises the amino acid sequence of SEQ ID NO: 100; and the light chain sequence comprises a sequence having at least 80% sequence identity to the amino acid sequence of SEQ ID NO: 247.

Embodiment 168. The bispecific antibody of Embodiment 164, wherein the heavy chain sequence comprises the amino acid sequence of SEQ ID NO: 101; and the light chain sequence comprises a sequence having at least 80% sequence identity to the amino acid sequence of SEQ ID NO: 248.

Embodiment 169. The bispecific antibody of Embodiment 164, wherein the heavy chain sequence comprises the amino acid sequence of SEQ ID NO: 102; and the light chain sequence comprises a sequence having at least 80% sequence identity to the amino acid sequence of SEQ ID NO: 249.

Embodiment 170. The bispecific antibody of Embodiment 164, wherein the heavy chain sequence comprises the amino acid sequence of SEQ ID NO: 103; and the light chain sequence comprises a sequence having at least 80% sequence identity to the amino acid sequence of SEQ ID NO: 250.

Embodiment 171. The bispecific antibody of Embodiment 164, wherein the heavy chain sequence comprises the amino acid sequence of SEQ ID NO: 104; and the light chain sequence comprises a sequence having at least 80% sequence identity to the amino acid sequence of SEQ ID NO: 251.

Embodiment 172. The bispecific antibody of Embodiment 164, wherein the heavy chain sequence comprises the amino acid sequence of SEQ ID NO: 105; and the light chain sequence comprises a sequence having at least 80% sequence identity to the amino acid sequence of SEQ ID NO: 252.

Embodiment 173. The bispecific antibody of Embodiment 164, wherein the heavy chain sequence comprises the amino acid sequence of SEQ ID NO: 106; and the light chain sequence comprises a sequence having at least 80% sequence identity to the amino acid sequence of SEQ ID NO: 253.

Embodiment 174. The bispecific antibody of Embodiment 164, wherein the heavy chain sequence comprises the amino acid sequence of SEQ ID NO: 107; and the light chain sequence comprises a sequence having at least 80% sequence identity to the amino acid sequence of SEQ ID NO: 254.

Embodiment 175. The bispecific antibody of Embodiment 164, wherein the heavy chain sequence comprises the amino acid sequence of SEQ ID NO: 108; and the light chain sequence comprises a sequence having at least 80% sequence identity to the amino acid sequence of SEQ ID NO: 255.

Embodiment 176. The bispecific antibody of Embodiment 164, wherein the heavy chain sequence comprises the amino acid sequence of SEQ ID NO: 109; and the light chain sequence comprises a sequence having at least 80% sequence identity to the amino acid sequence of SEQ ID NO: 256.

Embodiment 177. The bispecific antibody of Embodiment 164, wherein the heavy chain sequence comprises the amino acid sequence of SEQ ID NO: 110; and the light chain sequence comprises a sequence having at least 80% sequence identity to the amino acid sequence of SEQ ID NO: 257.

Embodiment 178. The bispecific antibody of Embodiment 164, wherein the heavy chain sequence comprises the amino acid sequence of SEQ ID NO: 111; and the light chain sequence comprises a sequence having at least 80% sequence identity to the amino acid sequence of SEQ ID NO: 258.

Embodiment 179. The bispecific antibody of Embodiment 164, wherein the heavy chain sequence comprises the amino acid sequence of SEQ ID NO: 112; and the light chain sequence comprises a sequence having at least 80% sequence identity to the amino acid sequence of SEQ ID NO: 259.

Embodiment 180. The bispecific antibody of Embodiment 164, wherein the heavy chain sequence comprises the amino acid sequence of SEQ ID NO: 113; and the light chain sequence comprises a sequence having at least 80% sequence identity to the amino acid sequence of SEQ ID NO: 260.

Embodiment 181. The bispecific antibody of Embodiment 164, wherein the heavy chain sequence comprises the amino acid sequence of SEQ ID NO: 114; and the light chain sequence comprises a sequence having at least 80% sequence identity to the amino acid sequence of SEQ ID NO: 261.

Embodiment 182. The bispecific antibody of Embodiment 164, wherein the heavy chain sequence comprises the amino acid sequence of SEQ ID NO: 115; and the light chain sequence comprises a sequence having at least 80% sequence identity to the amino acid sequence of SEQ ID NO: 262.

Embodiment 183. The bispecific antibody of Embodiment 164, wherein the heavy chain sequence comprises the amino acid sequence of SEQ ID NO: 116; and the light chain sequence comprises a sequence having at least 80% sequence identity to the amino acid sequence of SEQ ID NO: 263.

Embodiment 184. The bispecific antibody of Embodiment 164, wherein the heavy chain sequence comprises the amino acid sequence of SEQ ID NO: 117; and the light chain sequence comprises a sequence having at least 80% sequence identity to the amino acid sequence of SEQ ID NO: 264.

Embodiment 185. The bispecific antibody of Embodiment 164, wherein the heavy chain sequence comprises the amino acid sequence of SEQ ID NO: 118; and the light chain sequence comprises a sequence having at least 80% sequence identity to the amino acid sequence of SEQ ID NO: 265.

Embodiment 186. The bispecific antibody of Embodiment 164, wherein the heavy chain sequence comprises the amino acid sequence of SEQ ID NO: 119; and the light chain sequence comprises a sequence having at least 80% sequence identity to the amino acid sequence of SEQ ID NO: 266.

Embodiment 187. The bispecific antibody of Embodiment 164, wherein the heavy chain sequence comprises the amino acid sequence of SEQ ID NO: 120; and the light chain sequence comprises a sequence having at least 80% sequence identity to the amino acid sequence of SEQ ID NO: 267.

Embodiment 188. The bispecific antibody of Embodiment 164, wherein the heavy chain sequence comprises the amino acid sequence of SEQ ID NO: 121; and the light chain sequence comprises a sequence having at least 80% sequence identity to the amino acid sequence of SEQ ID NO: 268.

Embodiment 189. The bispecific antibody of Embodiment 164, wherein the heavy chain sequence comprises the amino acid sequence of SEQ ID NO: 122; and the light chain sequence comprises a sequence having at least 80% sequence identity to the amino acid sequence of SEQ ID NO: 269.

Embodiment 190. The bispecific antibody of Embodiment 164, wherein the heavy chain sequence comprises the amino acid sequence of SEQ ID NO: 123; and the light chain sequence comprises a sequence having at least 80% sequence identity to the amino acid sequence of SEQ ID NO: 270.

Embodiment 191. The bispecific antibody of Embodiment 164, wherein the heavy chain sequence comprises the amino acid sequence of SEQ ID NO: 124; and the light chain sequence comprises a sequence having at least 80% sequence identity to the amino acid sequence of SEQ ID NO: 271.

Embodiment 192. The bispecific antibody of Embodiment 164, wherein the heavy chain sequence comprises the amino acid sequence of SEQ ID NO: 125; and the light chain sequence comprises a sequence having at least 80% sequence identity to the amino acid sequence of SEQ ID NO: 272.

Embodiment 193. The bispecific antibody of Embodiment 164, wherein the heavy chain sequence comprises the amino acid sequence of SEQ ID NO: 126; and the light chain sequence comprises a sequence having at least 80% sequence identity to the amino acid sequence of SEQ ID NO: 273.

Embodiment 194. The bispecific antibody of Embodiment 164, wherein the heavy chain sequence comprises the amino acid sequence of SEQ ID NO: 127; and the light chain sequence comprises a sequence having at least 80% sequence identity to the amino acid sequence of SEQ ID NO: 274.

Embodiment 195. The bispecific antibody of Embodiment 164, wherein the heavy chain sequence comprises the amino acid sequence of SEQ ID NO: 129; and the light chain sequence comprises a sequence having at least 80% sequence identity to the amino acid sequence of SEQ ID NO: 276.

Embodiment 196. The bispecific antibody of any one of Embodiments 1-195, wherein the bispecific antibody is a humanized, human, or chimeric antibody.

Embodiment 197. The bispecific antibody of any one of Embodiments 1-196, wherein the bispecific antibody is a humanized antibody.

Embodiment 198. The bispecific antibody of any one of Embodiments 1-197, wherein the bispecific antibody comprises a heavy chain human constant region of a class selected from IgG, IgA, IgD, IgE, and IgM.

Embodiment 199. The bispecific antibody of Embodiment 198, wherein the human Fc region comprises a human heavy chain constant region of the class IgG and a subclass selected from IgG1, IgG2, IgG3, and IgG4.

Embodiment 200. The bispecific antibody of Embodiment 199, wherein the human Fc region comprises a human IgG1 Fc.

Embodiment 201. The bispecific antibody of Embodiment 199, wherein the human Fc region comprises a human IgG4 Fc.

Embodiment 202. The bispecific antibody of Embodiment 199, wherein the human Fc region comprises a human IgG2 Fc.

Embodiment 203. The bispecific antibody of any one of Embodiments 1-195 for use in the treatment of disease or condition.

Embodiment 204. The bispecific antibody of Embodiment 203, wherein the disease or disorder is cancer.

Embodiment 205. The bispecific antibody of Embodiment 204, wherein the cancer is a PD-1 expressing cancer.

Embodiment 206. The bispecific antibody of Embodiment 204, wherein the cancer is non-small cell lung cancer.

Embodiment 207. An isolated polynucleotide or set of polynucleotides encoding the bispecific antibody of any of Embodiments 1-195, a VH thereof, a VL thereof, a light chain thereof, a heavy chain thereof, or an antigen-binding portion thereof, and optionally, wherein the polynucleotide or set of polynucleotides comprises cDNA.

Embodiment 208. A vector or set of vectors comprising the polynucleotide or set of polynucleotides of Embodiment 207.

Embodiment 209. A host cell comprising the polynucleotide or set of polynucleotides of Embodiment 207 or the vector or set of vectors of Embodiment 208.

Embodiment 210. A method of producing an antibody, the method comprising expressing the bispecific antibody with the host cell of Embodiment 209 and isolating the expressed bispecific antibody.

Embodiment 211. A pharmaceutical composition comprising the bispecific antibody of any one of Embodiments 1-195 and a pharmaceutically acceptable excipient.

Embodiment 212. A kit comprising the bispecific antibody of any one of Embodiments 1-Embodiment 195 or a pharmaceutical composition of Embodiment 211 and instructions for use.

Embodiment 213. A method for treating a disease or condition in a mammalian subject in need thereof, the method comprising administering to the mammalian subject a therapeutically effective amount the bispecific antibody of any one of Embodiments 1-195 or a pharmaceutical composition of Embodiment 211.

Embodiment 214. The method of Embodiment 213, wherein the disease or disorder is cancer.

Embodiment 215. The method of Embodiment 214, wherein the cancer is a PD-1 expressing cancer.

Embodiment 216. The method of Embodiment 214, wherein the cancer is non-small cell lung cancer.

1.-135. (canceled)

136. A bispecific binding protein comprising:
(a) a PD-1 binding region comprising an anti-PD-1 immunoglobulin heavy chain variable domain (anti-PD-1 V_{H}) that comprises, according to Kabat numbering, a heavy chain complementarity determining region 1 (CDR-H1) comprising the amino acid sequence of SEQ ID NO: 445, a CDR-H2 comprising the amino acid sequence of SEQ ID NO: 446, and a CDR-H3 comprising the amino acid sequence of SEQ ID NO: 457; and an anti-PD-1 immunoglobulin light chain variable domain (anti-PD-1 V_{L}) that comprises, according to Kabat numbering, a light chain complementarity determining region 1 (CDR-L1) comprising the amino acid sequence of SEQ ID NO: 448, a CDR-L2 comprising the amino acid sequence of SEQ ID NO: 449, and a CDR-L3 comprising the amino acid sequence of SEQ ID NO: 450; and
(b) a VEGF binding region comprising an anti-VEGF immunoglobulin heavy chain variable domain (anti-VEGF V_{H}) that comprises, according to Kabat numbering, a heavy chain complementarity determining region 1 (CDR-H1) comprising the amino acid sequence of SEQ ID NO: 433, a CDR-H2 comprising the amino acid sequence of SEQ ID NO: 434, and a CDR-H3 comprising the amino acid sequence of SEQ ID NO: 435; and an anti-VEGF immunoglobulin light chain variable domain (anti-VEGF V_{L}) that comprises, according to Kabat numbering, a light chain complementarity determining region 1 (CDR-L1) comprising the amino acid sequence of SEQ ID NO: 436, a CDR-L2 comprising the amino acid sequence of SEQ ID NO: 437, and a CDR-L3 comprising the amino acid sequence of SEQ ID NO: 438.

137. The bispecific binding protein of claim 136, wherein the anti-PD-1 V_{H} comprises the amino acid sequence of SEQ ID NO: 327, and the anti-PD-1 V_{L} comprises the amino acid sequence of SEQ ID NO: 347.

138. The bispecific binding protein of claim 137, wherein the PD-1 binding region is an anti-PD-1 scFv that comprises the anti-PD-1 V_{H} and the anti-PD-1 V_{L}.

139. The bispecific binding protein of claim 138, wherein the anti-PD-1 scFv comprises the amino acid sequence of SEQ ID NO: 421.

140. The bispecific binding protein of claim 136, wherein the anti-VEGF V_{H} comprises the amino acid sequence of SEQ ID NO: 431, and the anti-VEGF V_{L} comprises the amino acid sequence of SEQ ID NO: 432.

141. The bispecific binding protein of claim 139, wherein the anti-VEGF V_{H} comprises the amino acid sequence of SEQ ID NO: 431, and the anti-VEGF V_{L} comprises the amino acid sequence of SEQ ID NO: 432.

142. The bispecific binding protein of claim 136, further comprising an IgG1 Fc region.

143. The bispecific binding protein of claim 141, wherein the VEGF binding region further comprises an IgG1 Fc region.

144. The bispecific binding protein of claim 143, wherein the IgG1 Fc region comprises L234A/L235A (LALA) substitutions.

145. The bispecific binding protein of claim 143, wherein the anti-PD-1 scFv is linked to the VEGF binding region.

146. The bispecific binding protein of claim 145, wherein the anti-PD-1 scFv is linked to the C-terminal of the VEGF binding region.

147. The bispecific binding protein of claim 146, wherein the anti-PD-1 scFv is linked to the VEGF binding region directly.

148. The bispecific binding protein of claim 146, wherein the anti-PD-1 scFv is linked to the VEGF binding region via a linker.

149. The bispecific binding protein of claim 148, wherein the linker comprises the amino acid sequence of SEQ ID NO: 1030.

150. The bispecific binding protein of claim 136, wherein the bispecific binding protein comprises a heavy chain that comprises the amino acid sequence of SEQ ID NO: 96, and a light chain that comprises the amino acid sequence of SEQ ID NO: 243.

151. The bispecific binding protein of claim 136, comprising two PD-1 binding regions and two VEGF binding regions.

152. A pharmaceutical composition comprising the bispecific binding protein of claim 136 and a pharmaceutically acceptable carrier.

153. A bispecific binding protein comprising a first polypeptide chain having the sequence of SEQ ID NO: 96 and a second polypeptide chain having the sequence of SEQ ID NO: 243, wherein the bispecific binding protein comprises two PD-1 binding regions and two VEGF-binding regions.

154. A pharmaceutical composition comprising the bispecific binding protein of claim 153 and a pharmaceutically acceptable carrier.
